SEQUENCE LISTING (149 sequences)

SEQ ID NO: 1 - protein, 15 aa, synthetic construct
  REGION 1..15: note = 6H9 LCDR1
RASQSVSTSSFSYMH

SEQ ID NO: 2 - protein, 10 aa, synthetic construct
  REGION 1..10: note = 7D4 LCDR1
SASSSVGYMH

SEQ ID NO: 3 - protein, 10 aa, synthetic construct
  REGION 1..10: note = 9D12 LCDR1, 4D10 LCDR1
SASSSVSYMH

SEQ ID NO: 4 - protein, 15 aa, synthetic construct
  REGION 1..15: note = P23B6 LCDR1
RASQSVSTSSYSYMH

SEQ ID NO: 5 - protein, 11 aa, synthetic construct
  REGION 1..11: note = 2C10 LCDR1
RASQDITTYLN

SEQ ID NO: 6 - protein, 17 aa, synthetic construct
  REGION 1..17: note = 4H11 LCDR1
KSSQSLLYSSNQKNYLA

SEQ ID NO: 7 - protein, 11 aa, synthetic construct
  REGION 1..11: note = 2G12 LCDR1, 5D5 LCDR1
RASQDIGSNLN

SEQ ID NO: 8 - protein, 7 aa, synthetic construct
  REGION 1..7: note = 6H9 LCDR2, P23B6 LCDR2
YASNLES

SEQ ID NO: 9 - protein, 7 aa, synthetic construct
  REGION 1..7: note = 7D4 LCDR2, 9D12 LCDR2, 4D10 LCDR2
EISKLAS

SEQ ID NO: 10 - protein, 7 aa, synthetic construct
  REGION 1..7: note = 2C10 LCDR2
YTSRLHS

SEQ ID NO: 11 - protein, 7 aa, synthetic construct
  REGION 1..7: note = 4H11 LCDR2
WASTRES

SEQ ID NO: 12 - protein, 7 aa, synthetic construct
  REGION 1..7: note = 2G12 LCDR2, 5D5 LCDR2
ATSSLDS

SEQ ID NO: 13 - protein, 9 aa, synthetic construct
  REGION 1..9: note = 6H9 LCDR3
QHNWEIPYT

SEQ ID NO: 14 - protein, 8 aa, synthetic construct
  REGION 1..8: note = 7D4 LCDR3, 9D12 LCDR3
QQWNYPLT

SEQ ID NO: 15 - protein, 9 aa, synthetic construct
  REGION 1..9: note = P23B6 LCDR3
QHSWEIPWT

SEQ ID NO: 16 - protein, 9 aa, synthetic construct
  REGION 1..9: note = 2C10 LCDR3
QQGNTLPRT

SEQ ID NO: 17 - protein, 9 aa, synthetic construct
  REGION 1..9: note = 4D10 LCDR3
QQWNYPLIT

SEQ ID NO: 18 - protein, 9 aa, synthetic construct
  REGION 1..9: note = 4H11 LCDR3
QQYYSYPST

SEQ ID NO: 19 - protein, 9 aa, synthetic construct
  REGION 1..9: note = 2G12 LCDR3, 5D5 LCDR3
LQYASSPYT

SEQ ID NO: 20 - protein, 6 aa, synthetic construct
  REGION 1..6: note = 6H9 HCDR1
SGYGWH

SEQ ID NO: 21 - protein, 5 aa, synthetic construct
  REGION 1..5: note = 7D4 HCDR1, 9D12 HCDR1
DTYMH

SEQ ID NO: 22 - protein, 5 aa, synthetic construct
  REGION 1..5: note = P23B6 HCDR1
SYWLN

SEQ ID NO: 23 - protein, 5 aa, synthetic construct
  REGION 1..5: note = 2C10 HCDR1
NYLIE

SEQ ID NO: 24 - protein, 5 aa, synthetic construct
  REGION 1..5: note = 4D10 HCDR1
SYVIH

SEQ ID NO: 25 - protein, 5 aa, synthetic construct
  REGION 1..5: note = 4H11 HCDR1
DYGMN

SEQ ID NO: 26 - protein, 5 aa, synthetic construct
  REGION 1..5: note = 2G12 HCDR1
HHGMN

SEQ ID NO: 27 - protein, 5 aa, synthetic construct
  REGION 1..5: note = 5D5 HCDR1
NYGMN

SEQ ID NO: 28 - protein, 16 aa, synthetic construct
  REGION 1..16: note = 6H9 HCDR2
YIHYSGSTDYSPSLKS

SEQ ID NO: 29 - protein, 17 aa, synthetic construct
  REGION 1..17: note = 7D4 HCDR2
RIDPSDGDTKYGPKFQD

SEQ ID NO: 30 - protein, 17 aa, synthetic construct
  REGION 1..17: note = 9D12 HCDR2
RIDLASGDSKYDPKFQG

SEQ ID NO: 31 - protein, 17 aa, synthetic construct
  REGION 1..17: note = P23B6 HCDR2
DIHPGRGSINYNEKFKS

SEQ ID NO: 32 - protein, 17 aa, synthetic construct
  REGION 1..17: note = 2C10 HCDR2
VLNPGSGATNYNEKFKG

SEQ ID NO: 33 - protein, 17 aa, synthetic construct
  REGION 1..17: note = 4D10 HCDR2
YFNPYNNGTNYNENFKG

SEQ ID NO: 34 - protein, 17 aa, synthetic construct
  REGION 1..17: note = 4D10 HCDR2-v1
YFNPYNIGTNYNENFKG

SEQ ID NO: 35 - protein, 17 aa, synthetic construct
  REGION 1..17: note = 4D10 HCDR2-v2
YFNPYNQGTNYNENFKG

SEQ ID NO: 36 - protein, 17 aa, synthetic construct
  REGION 1..17: note = 4D10 HCDR2-v3
YFNPYNVGTNYNENFKG

SEQ ID NO: 37 - protein, 17 aa, synthetic construct
  REGION 1..17: note = 4D10 HCDR2-v4
YFNPYNWGTNYNENFKG

SEQ ID NO: 38 - protein, 17 aa, synthetic construct
  REGION 1..17: note = 4H11 HCDR2, 5D5 HCDR2
WINTYTGEPTYADDFKG

SEQ ID NO: 39 - protein, 17 aa, synthetic construct
  REGION 1..17: note = 2G12 HCDR2
WINTYTGEPTYADDFRG

SEQ ID NO: 40 - protein, 8 aa, synthetic construct
  REGION 1..8: note = 6H9 HCDR3
DSSAWFAY

SEQ ID NO: 41 - protein, 12 aa, synthetic construct
  REGION 1..12: note = 7D4 HCDR3
AHFGGGGWYFDV

SEQ ID NO: 42 - protein, 12 aa, synthetic construct
  REGION 1..12: note = 9D12 HCDR3
AHFGGGNWYFDV

SEQ ID NO: 43 - protein, 8 aa, synthetic construct
  REGION 1..8: note = P23B6 HCDR3
ADGSWFVY

SEQ ID NO: 44 - protein, 11 aa, synthetic construct
  REGION 1..11: note = 2C10 HCDR3
GIYGGYFAMDY

SEQ ID NO: 45 - protein, 12 aa, synthetic construct
  REGION 1..12: note = 4D10 HCDR3
YYYASGSLYFDY

SEQ ID NO: 46 - protein, 12 aa, synthetic construct
  REGION 1..12: note = 4D10 HCDR3-v1
YFYASGSLYFDY

SEQ ID NO: 47 - protein, 12 aa, synthetic construct
  REGION 1..12: note = 4D10 HCDR3-v2
YVYASGSLYFDY

SEQ ID NO: 48 - protein, 12 aa, synthetic construct
  REGION 1..12: note = 4D10 HCDR3-v3
YWYASGSLYFDY

SEQ ID NO: 49 - protein, 9 aa, synthetic construct
  REGION 1..9: note = 4H11 HCDR3
KNIYGYFDV

SEQ ID NO: 50 - protein, 11 aa, synthetic construct
  REGION 1..11: note = 2G12 HCDR3
KDYYISGNFDY

SEQ ID NO: 51 - protein, 11 aa, synthetic construct
  REGION 1..11: note = 5D5 HCDR3
KDYYVSGNFDY

SEQ ID NO: 52 - protein, 10 aa, synthetic construct
  REGION 1..10: note = 4D10 LCDR1-v1
SASSFVSYMH

SEQ ID NO: 53 - protein, 10 aa, synthetic construct
  REGION 1..10: note = 4D10 LCDR1-v2
SASSHVSYMH

SEQ ID NO: 54 - protein, 7 aa, synthetic construct
  REGION 1..7: note = 4D10 LCDR2-v1
EIMKLAS

SEQ ID NO: 55 - protein, 7 aa, synthetic construct
  REGION 1..7: note = 4D10 LCDR2-v2
EISRLAS

SEQ ID NO: 56 - protein, 7 aa, synthetic construct
  REGION 1..7: note = 4D10 LCDR2-v3
EIMRLAS

SEQ ID NO: 57 - protein, 5 aa, synthetic construct
  REGION 1..5: note = 4D10 HCDR1-v1
SYVIM

SEQ ID NO: 58-60 - skipped (3 entries)

SEQ ID NO: 61 - protein, 111 aa, synthetic construct
  REGION 1..111: note = 6H9 VL
DIVLTQSPASLAVSLGQRATISCRASQSVSTSSFSYMHWYQQKPGQPPKLLIKYASNLESGVPARFSGSGSGTDFTLNIHPVEDEDPATYYCQHNWEIPYTFGGGTKLEIK

SEQ ID NO: 62 - protein, 105 aa, synthetic construct
  REGION 1..105: note = 7D4 VL
EIVLTQSPAITAASLGQKVTITCSASSSVGYMHWYQQKSGTSPKPWIYEISKLASGVPARFSGSGSGTSYSLTIRNMEAEDAAIYYCQQWNYPLTFGAGTKLELK

SEQ ID NO: 63 - protein, 105 aa, synthetic construct
  REGION 1..105: note = 9D12 VL
EIVLTQSPAITAASLGQKVTITCSASSSVSYMHWYQQKSGTSPKPWIYEISKLASGVPARFSGSGSGTSYSLTISSMEAEDAAIYYCQQWNYPLTFGAGTKLELK

SEQ ID NO: 64 - protein, 111 aa, synthetic construct
  REGION 1..111: note = P23B6 VL
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPWTFGGGTKLEIK

SEQ ID NO: 65 - protein, 107 aa, synthetic construct
  REGION 1..107: note = 2C10 VL
DIQMTQTTSSLSASLGDRVTISCRASQDITTYLNWYQQRPDGTVKLLIYYTSRLHSGVPSRFSGSGSGTEYSLTISNLEQEDIATYFCQQGNTLPRTFGGGTKLEIR

SEQ ID NO: 66 - protein, 106 aa, synthetic construct
  REGION 1..106: note = 4D10 VL
EIVLTQSPAIATASLGQKVTITCSASSSVSYMHWYQQKSGTSPKPWIYEISKLASGVPTRFSGSGSGTSYSLTISSMEAEDAAIYYCQQWNYPLITFGAGTKLELK

SEQ ID NO: 67 - protein, 113 aa, synthetic construct
  REGION 1..113: note = 4H11 VL
DIVMSQSPSSLAVSVGEKVTLSCKSSQSLLYSSNQKNYLAWYQQKPGQSPKLLIYWASTRESGVPDRFTGSGSGTDFTLTISSVKAEDLAVYYCQQYYSYPSTFGGGTKLEIK

SEQ ID NO: 68 - protein, 107 aa, synthetic construct
  REGION 1..107: note = 2G12 VL, 5D5 VL
DIQMTQSPSSLSASLGERVSLTCRASQDIGSNLNWLQQEPDGTIKRLIYATSSLDSGVPKRFSGSRSGSDYSLTISSLESEDFVDYYCLQYASSPYTFGGGTKLEIK

SEQ ID NO: 69 - skipped

SEQ ID NO: 70 - protein, 117 aa, synthetic construct
  REGION 1..117: note = 6H9 VH
DVQLQESGPDLVKPSQSLSLTCTVTGYSITSGYGWHWIRQFPGNKLEWMGYIHYSGSTDYSPSLKSRISITRDTSKNQFFLQLNSVTTEDTATYYCARDSSAWFAYWGQGTLVTVSA

SEQ ID NO: 71 - protein, 121 aa, synthetic construct
  REGION 1..121: note = 7D4 VH
EVQLQQSGAELVRPGASVKLSCTASGFNLKDTYMHWVKQRPEQGLEWIGRIDPSDGDTKYGPKFQDKTTITADTSSNTAYLQLSSLTSEDAAVYYCARAHFGGGGWYFDVWGAGTTVTVSS

SEQ ID NO: 72 - protein, 121 aa, synthetic construct
  REGION 1..121: note = 9D12 VH
EVQLQQSGAELVKPGASVKLSCTASGFNIKDTYMHWVKQRPEQGLEWIGRIDLASGDSKYDPKFQGKATITADTSSNTAYLQFSSLTSEDTAVYYCARAHFGGGNWYFDVWGAGTTVTVSS

SEQ ID NO: 73 - protein, 117 aa, synthetic construct
  REGION 1..117: note = P23B6 VH
QVQLLQPGAEPVKPGTSVKLSCKASGYNFTSYWLNWVKLRPGQGLEWIGDIHPGRGSINYNEKFKSKAILTVDTSSRTAYIQLSSLASEDSALYYCARADGSWFVYWGQGTLVTVSA

SEQ ID NO: 74 - protein, 120 aa, synthetic construct
  REGION 1..120: note = 2C10 VH
QVQLQQSGAELVRPGTSVKVSCKASGFAFTNYLIEWVKQRPGQGLEWIGVLNPGSGATNYNEKFKGKATLTADKSSSTAYMQLSSLTSDDSAVYFCARGIYGGYFAMDYWGQGTSVTVSS

SEQ ID NO: 75 - protein, 121 aa, synthetic construct
  REGION 1..121: note = 4D10 VH
EVQLQQSGPELVKPGASVKMSCKASGYTFSSYVIHWVRQKPGQGLEWIGYFNPYNNGTNYNENFKGKATLTSGKSSSTAYMELSSLTSEDSAVYYCARYYYASGSLYFDYWGQGTTLTVSS

SEQ ID NO: 76 - protein, 118 aa, synthetic construct
  REGION 1..118: note = 4H11 VH
QIQLVQSGPELKKPGETVKISCKASGYTLTDYGMNWVSQAPGKGLKWVGWINTYTGEPTYADDFKGRFAFSLDTSASTAYLQINNLKNEDTATYFCARKNIYGYFDVWGAGTTVTVSS

SEQ ID NO: 77 - protein, 120 aa, synthetic construct
  REGION 1..120: note = 2G12 VH
QIQLVQSGPELKKPGETVKISCKASGYSFTHHGMNWVKQAPGKDLKWMGWINTYTGEPTYADDFRGRFAFSLETSASTAYLQINNLKSEDTATYFCARKDYYISGNFDYWGQGTTLTVSS

SEQ ID NO: 78 - protein, 120 aa, synthetic construct
  REGION 1..120: note = 5D5 VH
QIQLVQSGPELKKPGETVKISCKASGYTLTNYGMNWVKQAPGTDLKWMGWINTYTGEPTYADDFKGRFAFSLETSASTAYLQINNLKNEDTATYFCARKDYYVSGNFDYWGQGTTLTVSS

SEQ ID NO: 79-80 - skipped (2 entries)

SEQ ID NO: 81 - protein, 105 aa, synthetic construct
  REGION 1..105: note = h9D12L0a
DIQLTQSPSFLSASVGDRVTITCSASSSVSYMHWYQQKPGKAPKLLIYEISKLASGVPSRFSGSGSGTEFTLTISSLQPEDFATYYCQQWNYPLTFGGGTKVEIK

SEQ ID NO: 82 - protein, 105 aa, synthetic construct
  REGION 1..105: note = h9D12L0b
DIQMTQSPSTLSASVGDRVTITCSASSSVSYMHWYQQKPGKAPKLLIYEISKLASGVPSRFSGSGSGTEFTLTISSLQPDDFATYYCQQWNYPLTFGGGTKVEIK

SEQ ID NO: 83 - protein, 105 aa, synthetic construct
  REGION 1..105: note = h9D12L1
DIQLTQSPSFLSASVGDRVTITCSASSSVSYMHWYQQKPGKAPKLLIYEISKLASGVPSRFSGSGSGTEFTLTISSMQPEDAATYYCQQWNYPLTFGGGTKVEIK

SEQ ID NO: 84 - protein, 105 aa, synthetic construct
  REGION 1..105: note = h9D12L2
DIQMTQSPSTLSASVGDRVTITCSASSSVSYMHWYQQKPGKAPKLLIYEISKLASGVPSRFSGSGSGTEFTLTISSMQPDDAATYYCQQWNYPLTFGGGTKVEIK

SEQ ID NO: 85 - protein, 105 aa, synthetic construct
  REGION 1..105: note = h9D12L3
DIQLTQSPSFLSASVGDRVTITCSASSSVSYMHWYQQKPGKAPKLLIYEISKLASGVPSRFSGSGSGTEFTLTISSLQPEDAATYYCQQWNYPLTFGGGTKVEIK

SEQ ID NO: 86 - protein, 105 aa, synthetic construct
  REGION 1..105: note = h9D12L4
DIQMTQSPSTLSASVGDRVTITCSASSSVSYMHWYQQKPGKAPKLLIYEISKLASGVPSRFSGSGSGTEFTLTISSLQPDDAATYYCQQWNYPLTFGGGTKVEIK

SEQ ID NO: 87 - protein, 105 aa, synthetic construct
  REGION 1..105: note = h9D12L5
DIQMTQSPSSLSASVGDRVTITCSASSSVSYMHWYQQKPGKAPKSLIYEISKLASGVPSRFSGSGSGTDFTLTISSMQPEDAATYYCQQWNYPLTFGQGTKLEIK

SEQ ID NO: 88 - protein, 105 aa, synthetic construct
  REGION 1..105: note = h9D12L6
EIVLTQSPATLSASPGERVTLSCSASSSVSYMHWYQQKPGQAPRLLIYEISKLASGIPARFSGSGSGTDFTLTISSMESEDAAVYYCQQWNYPLTFGQGTKLEIK

SEQ ID NO: 89 - protein, 105 aa, synthetic construct
  REGION 1..105: note = h9D12L7
DIQLTQSPSFLSASVGDRVTITCSASSSVSYMHWYQQKPGQAPKPWIYEISKLASGVPSRFSGSGSGTEYTLTISSMQSEDFAVYYCQQWNYPLTFGQGTKLEIK

SEQ ID NO: 90 - protein, 105 aa, synthetic construct
  REGION 1..105: note = h9D12L8
DIQLTQSPSFLSASVGDRVTITCSASSSVSYMHWYQQKPGQAPKLLIYEISKLASGVPSRFSGSGSGTEFTLTISSMQSEDFAVYYCQQWNYPLTFGQGTKLEIK

SEQ ID NO: 91 - protein, 107 aa, synthetic construct
  REGION 1..107: note = h2C10L0a
DIQMTQSPSSLSASVGDRVTITCRASQDITTYLNWYQQKPGKAPKLLIYYTSRLHSGVPSRFSGSGSGTDFTFTISSLQPEDIATYYCQQGNTLPRTFGQGTKVEIK

SEQ ID NO: 92 - protein, 107 aa, synthetic construct
  REGION 1..107: note = h2C10L0b
DIQMTQSPSSLSASVGDRVTITCRASQDITTYLNWYQQKPGKAPKLLIYYTSRLHSGVPSRFSGSGSGTDFTLTISSLQPEDFATYYCQQGNTLPRTFGQGTKVEIK

SEQ ID NO: 93 - protein, 107 aa, synthetic construct
  REGION 1..107: note = h2C10L1
DIQMTQSPSSLSASVGDRVTITCRASQDITTYLNWYQQKPGKAPKLLIYYTSRLHSGVPSRFSGSGSGTDFTLTISSLQPEDIATYYCQQGNTLPRTFGQGTKVEIK

SEQ ID NO: 94 - protein, 106 aa, synthetic construct
  REGION 1..106: note = h4D10L0a
DIQLTQSPSFLSASVGDRVTITCSASSSVSYMHWYQQKPGKAPKLLIYEISKLASGVPSRFSGSGSGTEFTLTISSLQPEDFATYYCQQWNYPLITFGGGTKVEIK

SEQ ID NO: 95 - protein, 106 aa, synthetic construct
  REGION 1..106: note = h4D10L0b
DIQMTQSPSTLSASVGDRVTITCSASSSVSYMHWYQQKPGKAPKLLIYEISKLASGVPSRFSGSGSGTEFTLTISSLQPDDFATYYCQQWNYPLITFGGGTKVEIK

SEQ ID NO: 96 - protein, 106 aa, synthetic construct
  REGION 1..106: note = h4D10L1
DIQLTQSPSFLSASVGDRVTITCSASSSVSYMHWYQQKPGKAPKPLIYEISKLASGVPSRFSGSGSGTEFTLTISSLQPEDAATYYCQQWNYPLITFGGGTKVEIK

SEQ ID NO: 97 - protein, 106 aa, synthetic construct
  REGION 1..106: note = h4D10L2
DIQMTQSPSTLSASVGDRVTITCSASSSVSYMHWYQQKPGKAPKPLIYEISKLASGVPSRFSGSGSGTEFTLTISSLQPDDAATYYCQQWNYPLITFGGGTKVEIK

SEQ ID NO: 98 - protein, 106 aa, synthetic construct
  REGION 1..106: note = h4D10L3
DIQLTQSPSFLSASVGDRVTITCSASSSVSYMHWYQQKPGKAPKLLIYEISKLASGVPSRFSGSGSGTEFTLTISSLQPEDAATYYCQQWNYPLITFGGGTKVEIK

SEQ ID NO: 99 - protein, 106 aa, synthetic construct
  REGION 1..106: note = h4D10L4
DIQMTQSPSTLSASVGDRVTITCSASSSVSYMHWYQQKPGKAPKLLIYEISKLASGVPSRFSGSGSGTEFTLTISSLQPDDAATYYCQQWNYPLITFGGGTKVEIK

SEQ ID NO: 100 - protein, 106 aa, synthetic construct
  REGION 1..106: note = h4D10L2-5D6-2
DIQMTQSPSTLSASVGDRVTITCSASSFVSYMHWYQQKPGKAPKPLIYEISKLASGVPSRFSGSGSGTEFTLTISSLQPDDAATYYCQQWNYPLITFGGGTKVEIK

SEQ ID NO: 101 - protein, 106 aa, synthetic construct
  REGION 1..106: note = h4D10L2-5G6
DIQMTQSPSTLSASVGDRVTITCSASSHVSYMHWYQQKPGKAPKPLIYEISKLASGVPSRFSGSGSGTEFTLTISSLQPDDAATYYCQQWNYPLITFGGGTKVEIK

SEQ ID NO: 102 - protein, 106 aa, synthetic construct
  REGION 1..106: note = h4D10L2-19H8
DIQMTQSPSTLSASVGDRVTITCSASSSVSYMHWYQQKPGKAPKPLIYEIMKLASGVPSRFSGSGSGTEFTLTISSLQPDDAATYYCQQWNYPLITFGGGTKVEIK

SEQ ID NO: 103 - protein, 106 aa, synthetic construct
  REGION 1..106: note = h4D10L2-20A3
DIQMTQSPSTLSASVGDRVTITCSASSSVSYMHWYQQKPGKAPKPLIYEISRLASGVPSRFSGSGSGTEFTLTISSLQPDDAATYYCQQWNYPLITFGGGTKVEIK

SEQ ID NO: 104 - protein, 106 aa, synthetic construct
  REGION 1..106: note = h4D10L2CM1
DIQMTQSPSTLSASVGDRVTITCSASSFVSYMHWYQQKPGKAPKPLIYEIMKLASGVPSRFSGSGSGTEFTLTISSLQPDDAATYYCQQWNYPLITFGGGTKVEIK

SEQ ID NO: 105 - protein, 106 aa, synthetic construct
  REGION 1..106: note = h4D10L2CM2
DIQMTQSPSTLSASVGDRVTITCSASSFVSYMHWYQQKPGKAPKPLIYEISRLASGVPSRFSGSGSGTEFTLTISSLQPDDAATYYCQQWNYPLITFGGGTKVEIK

SEQ ID NO: 106 - protein, 106 aa, synthetic construct
  REGION 1..106: note = h4D10L2CM3
DIQMTQSPSTLSASVGDRVTITCSASSFVSYMHWYQQKPGKAPKPLIYEIMRLASGVPSRFSGSGSGTEFTLTISSLQPDDAATYYCQQWNYPLITFGGGTKVEIK

SEQ ID NO: 107 - protein, 107 aa, synthetic construct
  REGION 1..107: note = h2C10L2
DIQMTQSTSSLSASVGDRVTITCRASQDITTYLNWYQQKPGGAPKLLIYYTSRLHSGVPSRFSGSGSGTDYTLTISSLQPEDIATYYCQQGNTLPRTFGGGTKLEIK

SEQ ID NO: 108 - protein, 107 aa, synthetic construct
  REGION 1..107: note = h2C10L3
DIQMTQSPSSLSASVGDRVTITCRASQDITTYLNWYQQKPGGAPKLLIYYTSRLHSGVPSRFSGSGSGTDYTLTISSLQPEDIATYYCQQGNTLPRTFGGGTKLEIK

SEQ ID NO: 109 - protein, 107 aa, synthetic construct
  REGION 1..107: note = h2C10L4
DIQMTQSPSSLSASVGDRVTITCRASQDITTYLNWYQQKPGGAPKLLIYYTSRLHSGVPSRFSGSGSGTDFTLTISSLQPEDIATYYCQQGNTLPRTFGGGTKLEIK

SEQ ID NO: 110 - skipped

SEQ ID NO: 111 - protein, 121 aa, synthetic construct
  REGION 1..121: note = 9D12H0a
EVQLVQSGAEVKKPGATVKISCKVSGFNIKDTYMHWVQQAPGKGLEWMGRIDLASGDSKYDPKFQGRVTITADTSTDTAYMELSSLRSEDTAVYYCARAHFGGGNWYFDVWGQGTTVTVSS

SEQ ID NO: 112 - protein, 121 aa, synthetic construct
  REGION 1..121: note = 9D12H0b
QVQLVQSGAEVKKPGASVKVSCKASGFNIKDTYMHWVRQAPGQGLEWMGRIDLASGDSKYDPKFQGRVTMTRDTSTSTVYMELSSLRSEDTAVYYCARAHFGGGNWYFDVWGQGTTVTVSS

SEQ ID NO: 113 - protein, 121 aa, synthetic construct
  REGION 1..121: note = h9D12H1
EVQLVQSGAEVKKPGATVKISCKASGFNIKDTYMHWVQQRPGKGLEWIGRIDLASGDSKYDPKFQGRVTITADTSTDTAYMELSSLRSEDTAVYYCARAHFGGGNWYFDVWGQGTTVTVSS

SEQ ID NO: 114 - protein, 121 aa, synthetic construct
  REGION 1..121: note = h9D12H2
QVQLVQSGAEVKKPGASVKVSCKASGFNIKDTYMHWVKQRPGQGLEWIGRIDLASGDSKYDPKFQGRVTMTADTSTSTVYMELSSLRSEDTAVYYCARAHFGGGNWYFDVWGQGTTVTVSS

SEQ ID NO: 115 - protein, 121 aa, synthetic construct
  REGION 1..121: note = h9D12H3
EVQLVQSGAEVKKPGATVKISCKVSGFNIKDTYMHWVQQAPGKGLEWIGRIDLASGDSKYDPKFQGRVTITADTSTDTAYMELSSLRSEDTAVYYCARAHFGGGNWYFDVWGQGTTVTVSS

SEQ ID NO: 116 - protein, 121 aa, synthetic construct
  REGION 1..121: note = h9D12H4
QVQLVQSGAEVKKPGASVKVSCKASGFNIKDTYMHWVRQAPGQGLEWIGRIDLASGDSKYDPKFQGRVTMTADTSTSTVYMELSSLRSEDTAVYYCARAHFGGGNWYFDVWGQGTTVTVSS

SEQ ID NO: 117 - protein, 120 aa, synthetic construct
  REGION 1..120: note = h2C10H0a
QVQLVQSGAEVKKPGASVKVSCKASGFAFTNYLIEWVRQAPGQGLEWMGVLNPGSGATNYNEKFKGRVTMTRDTSISTAYMELSRLRSDDTAVYYCARGIYGGYFAMDYWGQGTLVTVSS

SEQ ID NO: 118 - protein, 120 aa, synthetic construct
  REGION 1..120: note = h2C10H0b
QVQLVQSGAEVKKPGASVKVSCKASGFAFTNYLIEWVRQAPGQGLEWMGVLNPGSGATNYNEKFKGRVTMTRDTSTSTVYMELSSLRSEDTAVYYCARGIYGGYFAMDYWGQGTLVTVSS

SEQ ID NO: 119 - protein, 120 aa, synthetic construct
  REGION 1..120: note = h2C10H1
QVQLVQSGAEVVKPGASVKVSCKASGFAFTNYLIEWVRQAPGQGLEWIGVLNPGSGATNYNEKFKGRVTMTADTSISTAYMELSRLRSDDTAVYYCARGIYGGYFAMDYWGQGTLVTVSS

SEQ ID NO: 120 - protein, 120 aa, synthetic construct
  REGION 1..120: note = h2C10H2
QVQLVQSGAEVVKPGASVKVSCKASGFAFTNYLIEWVRQAPGQGLEWIGVLNPGSGATNYNEKFKGRVTITADTSTSTVYMELSSLRSEDTAVYYCARGIYGGYFAMDYWGQGTLVTVSS

SEQ ID NO: 121 - protein, 120 aa, synthetic construct
  REGION 1..120: note = h2C10H3
QVQLVQSGAEVKKPGASVKVSCKASGFAFTNYLIEWVRQAPGQGLEWMGVLNPGSGATNYNEKFKGRVTMTADTSISTAYMELSRLRSDDTAVYYCARGIYGGYFAMDYWGQGTLVTVSS

SEQ ID NO: 122 - protein, 120 aa, synthetic construct
  REGION 1..120: note = h2C10H4
QVQLVQSGAEVKKPGASVKVSCKASGFAFTNYLIEWVRQAPGQGLEWMGVLNPGSGATNYNEKFKGRVTMTADTSTSTVYMELSSLRSEDTAVYYCARGIYGGYFAMDYWGQGTLVTVSS

SEQ ID NO: 123 - protein, 121 aa, synthetic construct
  REGION 1..121: note = h4D10H0a
QVQLVQSGAEVKKPGASVKVSCKASGYTFSSYVIHWVRQAPGQGLEWMGYFNPYNNGTNYNENFKGRVTMTRDTSISTAYMELSRLRSDDTAVYYCARYYYASGSLYFDYWGQGTLVTVSS

SEQ ID NO: 124 - protein, 121 aa, synthetic construct
  REGION 1..121: note = h4D10H0b
QVQLVQSGAEVKKPGASVKVSCKASGYTFSSYVIHWVRQAPGQGLEWMGYFNPYNNGTNYNENFKGRVTMTRDTSTSTVYMELSSLRSEDTAVYYCARYYYASGSLYFDYWGQGTTVTVSS

SEQ ID NO: 125 - protein, 121 aa, synthetic construct
  REGION 1..121: note = h4D10H1
QVQLVQSGAEVVKPGASVKVSCKASGYTFSSYVIHWVRQAPGQGLEWIGYFNPYNNGTNYNENFKGRVTMTADTSISTAYMELSRLRSDDTAVYYCARYYYASGSLYFDYWGQGTLVTVSS

SEQ ID NO: 126 - protein, 121 aa, synthetic construct
  REGION 1..121: note = h4D10H2
QVQLVQSGAEVVKPGASVKVSCKASGYTFSSYVIHWVRQAPGQGLEWIGYFNPYNNGTNYNENFKGRVTMTADTSTSTVYMELSSLRSEDTAVYYCARYYYASGSLYFDYWGQGTTVTVSS

SEQ ID NO: 127 - protein, 121 aa, synthetic construct
  REGION 1..121: note = h4D10H3
QVQLVQSGAEVKKPGASVKVSCKASGYTFSSYVIHWVRQAPGQGLEWMGYFNPYNNGTNYNENFKGRVTMTADTSISTAYMELSRLRSDDTAVYYCARYYYASGSLYFDYWGQGTLVTVSS

SEQ ID NO: 128 - protein, 121 aa, Synthetic construct
  REGION 1..121: note = h4D10H4
QVQLVQSGAEVKKPGASVKVSCKASGYTFSSYVIHWVRQAPGQGLEWMGYFNPYNNGTNYNENFKGRVTMTADTSTSTVYMELSSLRSEDTAVYYCARYYYASGSLYFDYWGQGTTVTVSS

SEQ ID NO: 129 - protein, 526 aa, Homo sapiens
  REGION 1..526: note = human CEACAM1; isoform 1
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 130 - protein, 121 aa, synthetic construct
  REGION 1..121: note = h4D10H4-PTM-10E5
QVQLVQSGAEVKKPGASVKVSCKASGYTFSSYVIMWVRQAPGQGLEWMGYFNPYNQGTNYNENFKGRVTMTADTSTSTVYMELSSLRSEDTAVYYCARYFYASGSLYFDYWGQGTTVTVSS

SEQ ID NO: 131-133 - skipped (3 entries)

SEQ ID NO: 134 - protein, 107 aa, Homo sapiens [sequence omitted]

SEQ ID NO: 135 - protein, 106 aa, Homo sapiens
  REGION 1..106: note = IgGCLambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 136 - protein, 330 aa, Homo sapiens [sequence omitted]

SEQ ID NO: 137 - protein, 326 aa, Homo sapiens
  REGION 1..326: note = IgG2 CH
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 138 - protein, 377 aa, Homo sapiens
  REGION 1..377: note = IgG3 CH
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 139 - protein, 327 aa, Homo sapiens
  REGION 1..327: note = IgG4 CH
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 5 aa, synthetic construct
  REGION 1..5: note = linker
  REPEAT 1..5: note = (GGGGS)n, n=1, 2, 3,4, or 5
GGGGS

SEQ ID NO: 141 - protein, 5 aa, synthetic construct
  REGION 1..5: note = linker
  REPEAT 1..5: note = (EAAAK)n, n=1, 2, 3, 4, or 5
EAAAK

SEQ ID NO: 142 - protein, 5 aa, synthetic construct
  REGION 1..2: note = linker
  REPEAT 1..2: note = (PA)nPAP, n=0, 1, 2, 3, or 4
PAPAP

SEQ ID NO: 143 - protein, 121 aa, synthetic construct
  REGION 1..121: note = h4D10H4-v1
QVQLVQSGAEVKKPGASVKVSCKASGYTFSSYVIHWVRQAPGQGLEWMGYFNPYNQGTNYNENFKGRVTMTADTSTSTVYMELSSLRSEDTAVYYCARYYYASGSLYFDYWGQGTTVTVSS

SEQ ID NO: 144 - protein, 121 aa, synthetic construct
  REGION 1..121: note = h4D10H4-v2
QVQLVQSGAEVKKPGASVKVSCKASGYTFSSYVIHWVRQAPGQGLEWMGYFNPYNIGTNYNENFKGRVTMTADTSTSTVYMELSSLRSEDTAVYYCARYYYASGSLYFDYWGQGTTVTVSS

SEQ ID NO: 145 - protein, 121 aa, synthetic construct
  REGION 1..121: note = h4D10H4-v3
QVQLVQSGAEVKKPGASVKVSCKASGYTFSSYVIHWVRQAPGQGLEWMGYFNPYNVGTNYNENFKGRVTMTADTSTSTVYMELSSLRSEDTAVYYCARYYYASGSLYFDYWGQGTTVTVSS

SEQ ID NO: 146 - protein, 121 aa, synthetic construct
  REGION 1..121: note = h4D10H4-v4
QVQLVQSGAEVKKPGASVKVSCKASGYTFSSYVIHWVRQAPGQGLEWMGYFNPYNNGTNYNENFKGRVTMTADTSTSTVYMELSSLRSEDTAVYYCARYFYASGSLYFDYWGQGTTVTVSS

SEQ ID NO: 147 - protein, 121 aa, synthetic construct
  REGION 1..121: note = h4D10H4-CM1
QVQLVQSGAEVKKPGASVKVSCKASGYTFSSYVIHWVRQAPGQGLEWMGYFNPYNQGTNYNENFKGRVTMTADTSTSTVYMELSSLRSEDTAVYYCARYFYASGSLYFDYWGQGTTVTVSS

SEQ ID NO: 148 - protein, 121 aa, synthetic construct
  REGION 1..121: note = h4D10H4-CM2
QVQLVQSGAEVKKPGASVKVSCKASGYTFSSYVIHWVRQAPGQGLEWMGYFNPYNIGTNYNENFKGRVTMTADTSTSTVYMELSSLRSEDTAVYYCARYFYASGSLYFDYWGQGTTVTVSS

SEQ ID NO: 149 - protein, 121 aa, synthetic construct
  REGION 1..121: note = h4D10H4-CM3
QVQLVQSGAEVKKPGASVKVSCKASGYTFSSYVIHWVRQAPGQGLEWMGYFNPYNVGTNYNENFKGRVTMTADTSTSTVYMELSSLRSEDTAVYYCARYFYASGSLYFDYWGQGTTVTVSS

(19) United States
(12) Patent Application Publication
(10) Pub. No.: US 2026/0297187 A1
(43) Pub. Date: Oct. 1, 2026

(54) CEACAM1-TARGETING ANTIBODIES AND USES THEREOF

(71) Applicant: SUZHOU NEOLOGICS BIOSCIENCE CO., LTD., Suzhou (CN)

(72) Inventors: Xin Dong, Suzhou (CN); Yu Zhang, Suzhou (CN); Jinyu Dong, Suzhou (CN); Binbin Wang, Suzhou (CN); Dong Wang, Suzhou (CN); Tingting Bu, Suzhou (CN)

(21) Appl. No.: 19/474,696

(22) PCT Filed: Apr. 12, 2024
(86) PCT No.: PCT/CN2024/087425

§ 371 Date: Oct. 11, 2025

(30) Foreign Application Priority Data
Apr. 13, 2023 (WO) PCT/CN2023/088112

(51) Int. Cl. C07K 16/28; A61K 39/00; A61P 35/00; A61P 37/02

(52) U.S. Cl. CPC C07K 16/2803; A61P 35/00; A61P 37/02; A61K 2039/505; C07K 2317/24; C07K 2317/565; C07K 2317/77; C07K 2317/92

(57) ABSTRACT

Provided are anti-CEACAM1 antibodies and antigen-binding fragments, polynucleotides encoding the antibodies and antigen-binding fragments, and pharmaceutical compositions comprising the antibodies and antigen-binding fragments. Also provided are uses of the anti-CEACAM1 antibodies and antigen-binding fragments described herein in, for example, cancer treatment.

This application claims priority to PCT Patent Application No. PCT/CN2023/088112, filed Apr. 13, 2023, which is incorporated herein by reference in its entirety.

### 1. REFERENCE TO SEQUENCE LISTING SUBMITTED ELECTRONICALLY

This application incorporates by reference a Sequence Listing as an XML file entitled “135A006WO02_SL” created on Mar. 27, 2024 and having a size of 185,768 bytes.

### 2. FIELD

The present invention relates to molecular biology, cell biology, and immuno-oncology. Provided herein include anti-CEACAM1-antibodies and uses thereof in, for example, treating tumors or cancers.

### 3. BACKGROUND

Carcinoembryonic antigen-related cell adhesion molecule 1 (CEACAM1) is a member of the carcinoembryonic antigen (CEA) family of immunoglobulin (Ig) like transmembrane glycoproteins. CEACAM1 is expressed on tumor cells and immune cells such as T cells, NK cells, and certain macrophages, and is closely involved with immune regulation, tumorigenesis, and pathogen binding. Specifically, CEACAM1 appears to play a role in immune suppression and immune cell exhaustion, and therefore holds great promise as a therapeutic target for cancer. However, there has been limited success in the development of CEACAM1-targeting therapeutic options. Accordingly, there is an unmet need for additional therapeutic options for cancer patients, especially for CEACAM1-targeting agents. The compositions and methods provided herein meet these needs and provide other relative advantages.

### 4. SUMMARY

Provided herein are antibodies or antigen-binding fragments thereof that specifically bind to human CEACAM1, comprising: (a) a light chain variable region (VL) comprising (1) a light chain CDR1 (VL CDR1) having an amino acid sequence selected from the group consisting of SEQ ID NOs:1-7 and 52-53; (2) a light chain CDR2 (VL CDR2) having an amino acid sequence selected from the group consisting of SEQ ID NOs:8-12 and 54-56; and (3) a light chain CDR3 (VL CDR3) having an amino acid sequence selected from the group consisting of SEQ ID NOs:13-19; or a variant thereof having up to about 3 amino acid substitutions, additions, and/or deletions in the VL CDRs; and/or (b) a heavy chain variable region (VH) comprising (1) a heavy chain CDR1 (VH CDR1) having an amino acid sequence selected from the group consisting of SEQ ID NOs:20-27 and 57; (2) a heavy chain CDR2 (VH CDR2) having an amino acid sequence selected from the group consisting of SEQ ID NOs:28-39; and (3) a heavy chain CDR3 (VH CDR3) having an amino acid sequence selected from the group consisting of SEQ ID NOs:40-51; or a variant thereof having up to about 3 amino acid substitutions, additions, and/or deletions in the VH CDRs.

In some embodiments of the anti-CEACAM1 antibodies and antigen-binding fragments provided herein, (a) the VL CDR1, CDR2 and CDR3 have (1) the amino acid sequences of SEQ ID NOs:1, 8, and 13, respectively; (2) the amino acid sequences of SEQ ID NOs:2, 9, and 14, respectively; (3) the amino acid sequences of SEQ ID NOs:3, 9, and 14, respectively; (4) the amino acid sequences of SEQ ID NOs:4, 8, and 15, respectively; (5) the amino acid sequences of SEQ ID NOs:5, 10, and 16, respectively; (6) the amino acid sequences of SEQ ID NOs:3, 9, and 17, respectively; (7) the amino acid sequences of SEQ ID NOs:6, 11, and 18, respectively; or (8) the amino acid sequences of SEQ ID NOs:7, 12, and 19, respectively; or a variant thereof having up to about 3 amino acid substitutions, additions, and/or deletions in the VL CDRs; and/or (b) the VH CDR1, CDR2 and CDR3 have (1) the amino acid sequences of SEQ ID NOs:20, 28, and 40, respectively; (2) the amino acid sequences of SEQ ID NOs:21, 29, and 41, respectively; (3) the amino acid sequences of SEQ ID NOs:21, 30, and 42, respectively; (4) the amino acid sequences of SEQ ID NOs:22, 31, and 43, respectively; (5) the amino acid sequences of SEQ ID NOs:23, 32, and 44, respectively; (6) the amino acid sequences of SEQ ID NOs:24, 33, and 45, respectively; (7) the amino acid sequences of SEQ ID NOs:25, 38, and 49, respectively; (8) the amino acid sequences of SEQ ID NOs:26, 39, and 50, respectively; or (9) the amino acid sequences of SEQ ID NOs:27, 38, and 51, respectively; or a variant thereof having up to about 3 amino acid substitutions, additions, and/or deletions in the VH CDRs.

In some embodiments of the anti-CEACAM1 antibodies and antigen-binding fragments provided herein, (1) the VL CDR1, CDR2, and CDR3 have the amino acid sequences of SEQ ID NOs:1, 8, and 13, respectively; and/or the VH CDR1, CDR2, and CDR3 have the amino acid sequences of SEQ ID NOs:20, 28, and 40, respectively; (2) the VL CDR1, CDR2, and CDR3 have the amino acid sequences of SEQ ID NOs:2, 9, and 14, respectively; and/or the VH CDR1, CDR2, and CDR3 have the amino acid sequences of SEQ ID NOs:21, 29, and 41, respectively; (3) the VL CDR1, CDR2, and CDR3 have the amino acid sequences of SEQ ID NOs:3, 9, and 14, respectively; and/or the VH CDR1, CDR2, and CDR3 have the amino acid sequences of SEQ ID NOs:21, 30, and 42, respectively; (4) the VL CDR1, CDR2, and CDR3 have the amino acid sequences of SEQ ID NOs:4, 8, and 15, respectively; and/or the VH CDR1, CDR2, and CDR3 have the amino acid sequences of SEQ ID NOs:22, 31, and 43, respectively; (5) the VL CDR1, CDR2, and CDR3 have the amino acid sequences of SEQ ID NOs:5, 10, and 16, respectively; and/or the VH CDR1, CDR2, and CDR3 have the amino acid sequences of SEQ ID NOs:23, 32, and 44, respectively; (6) the VL CDR1, CDR2, and CDR3 have the amino acid sequences of SEQ ID NOs:3, 9, and 17, respectively; and/or the VH CDR1, CDR2, and CDR3 have the amino acid sequences of SEQ ID NOs:24, 33, and 45, respectively; (7) the VL CDR1, CDR2, and CDR3 have the amino acid sequences of SEQ ID NOs:6, 11, and 18, respectively; and/or the VH CDR1, CDR2, and CDR3 have the amino acid sequences of SEQ ID NOs:25, 38, and 49, respectively; (8) the VL CDR1, CDR2, and CDR3 have the amino acid sequences of SEQ ID NOs:7, 12, and 19, respectively; and/or the VH CDR1, CDR2, and CDR3 have the amino acid sequences of SEQ ID NOs:26, 39, and 50, respectively; or (9) the VL CDR1, CDR2, and CDR3 have the amino acid sequences of SEQ ID NOs:7, 12, and 19, respectively; and/or the VH CDR1, CDR2, and CDR3 have the amino acid sequences of SEQ ID NOs:27, 38, and 51, respectively.

In some embodiments, the anti-CEACAM1 antibodies and antigen-binding fragments provided herein comprise a VL CDR1, a VL CDR2, a VL CDR3, a VH CDR1, a VH CDR2 and a VH CDR3 having the amino acid sequences of SEQ ID NOs:3, 9, 14, 21, 30, and 42, respectively; or a variant thereof having up to about 3 amino acid substitutions, additions, and/or deletions in the CDRs. In some embodiments, the anti-CEACAM1 antibodies and antigen-binding fragments provided herein comprise a VL CDR1, a VL CDR2, a VL CDR3, a VH CDR1, a VH CDR2 and a VH CDR3 having the amino acid sequences of SEQ ID NOs:5, 10, 16, 23, 32, and 44, respectively; or a variant thereof having up to about 3 amino acid substitutions, additions, and/or deletions in the CDRs.

In some embodiments, the anti-CEACAM1 antibodies and antigen-binding fragments provided herein comprise a VL CDR1, a VL CDR2, a VL CDR3, a VH CDR1, a VH CDR2 and a VH CDR3 having the amino acid sequences of SEQ ID NOs:3, 9, 17, 24, 33, and 45, respectively; or a variant thereof having up to about 3 amino acid substitutions, additions, and/or deletions in the CDRs. In some embodiments, the VL CDR1 has an amino acid sequence selected from the group consisting of SEQ ID NOs:52-53, the VL CDR2 has an amino acid sequence selected from the group consisting of SEQ ID NOs:54-56, the VH CDR1 has the amino acid sequence of SEQ ID NO:57, the VH CDR2 has an amino acid sequence selected from the group consisting of SEQ ID NOs:34-37 and the VH CDR3 has an amino acid sequence selected from the group consisting of SEQ ID NOs:46-48.

Provided herein are also antibodies or antigen-binding fragments thereof that specifically bind to human CEACAM1, comprising: (a) a VL having at least 85%, at least 90%, at least 95%, at least 98%, or 100% sequence identity to an amino acid sequence selected from the group consisting of SEQ ID NOs:61-68; and/or (b) a VH having at least 85%, at least 90%, at least 95%, at least 98%, or 100% sequence identity to an amino acid sequence selected from the group consisting of SEQ ID NOs:70-78.

In some embodiments, the anti-CEACAM1 antibodies and antigen-binding fragments provided herein comprise a VL and a VH, wherein the VL and VH each have at least 85%, at least 90%, at least 95%, at least 98%, or 100% sequence identity to the amino acid sequences of (1) SEQ ID NOs:61 and 70, respectively; (2) SEQ ID NOs:62 and 71, respectively; (3) SEQ ID NOs:63 and 72, respectively; (4) SEQ ID NOs:64 and 73, respectively; (5) SEQ ID NOs:65 and 74, respectively; (6) SEQ ID NOs:66 and 75, respectively; (7) SEQ ID NOs:67 and 76, respectively; (8) SEQ ID NOs:68 and 77, respectively; or (9) SEQ ID NOs:68 and 78, respectively.

In some embodiments, the anti-CEACAM1 antibodies and antigen-binding fragments provided herein comprise a VL and a VH, wherein the VL and VH each have at least 85%, at least 90%, at least 95%, at least 98%, or 100% sequence identity to the amino acid sequences of SEQ ID NOs:63 and 72, respectively. In some embodiments, the anti-CEACAM1 antibodies and antigen-binding fragments provided herein comprise a VL and a VH, wherein the VL and VH each have at least 85%, at least 90%, at least 95%, at least 98%, or 100% sequence identity to the amino acid sequences of SEQ ID NOs:65 and 74, respectively.

In some embodiments, the anti-CEACAM1 antibodies and antigen-binding fragments provided herein comprise a VL and a VH, wherein the VL and VH each have at least 85%, at least 90%, at least 95%, at least 98%, or 100% sequence identity to the amino acid sequences of SEQ ID NOs:66 and 75, respectively. In some embodiments, the VL has the amino acid sequence of SEQ ID NO:66 with an amino acid substitution at S28, S51, K52, or any combination thereof. In some embodiments, the S28 substitution is S28F or S28H, the S51 substitution is S51M, and the K52 substitution is K52R. In some embodiments, the VH has the amino acid sequence of SEQ ID NO:75 with an amino acid substitution at H35, N56, Y100, or any combination thereof. In some embodiments, the H35 substitution is H35M, the N56 substitution is N56I, N56Q, N56V, or N56W, and the Y100 substitution is Y100F, Y100W, or Y100V.

Provided herein are also antibodies or antigen-binding fragments thereof that specifically bind to human CEACAM1, comprising: (a) a VL comprising VL CDR1, CDR2, and CDR3 from a VL having an amino acid sequence selected from the group consisting of SEQ ID NOs:61-68; and/or (b) a VH comprising VH CDR1, CDR2, and CDR3 from a VH having an amino acid sequence selected from the group consisting of SEQ ID NOs:70-78; or a variant thereof having up to about 3 amino acid substitutions, additions, and/or deletions in the CDRs.

In some embodiments, the anti-CEACAM1 antibodies and antigen-binding fragments provided herein comprise (1) a VL comprising VL CDR1, CDR2, and CDR3 from a VL having the amino acid sequence of SEQ ID NO:61, and/or a VH comprising VH CDR1, CDR2, and CDR3 from a VH having the amino acid sequence of SEQ ID NO:70; (2) a VL comprising VL CDR1, CDR2, and CDR3 from a VL having the amino acid sequence of SEQ ID NO:62, and/or a VH comprising VH CDR1, CDR2, and CDR3 from a VH having the amino acid sequence of SEQ ID NO:71; (3) a VL comprising VL CDR1, CDR2, and CDR3 from a VL having the amino acid sequence of SEQ ID NO:63, and/or a VH comprising VH CDR1, CDR2, and CDR3 from a VH having the amino acid sequence of SEQ ID NO:72; (4) a VL comprising VL CDR1, CDR2, and CDR3 from a VL having the amino acid sequence of SEQ ID NO:64, and/or a VH comprising VH CDR1, CDR2, and CDR3 from a VH having the amino acid sequence of SEQ ID NO:73; (5) a VL comprising VL CDR1, CDR2, and CDR3 from a VL having the amino acid sequence of SEQ ID NO:65, and/or a VH comprising VH CDR1, CDR2, and CDR3 from a VH having the amino acid sequence of SEQ ID NO:74; (6) a VL comprising VL CDR1, CDR2, and CDR3 from a VL having the amino acid sequence of SEQ ID NO:66, and/or a VH comprising VH CDR1, CDR2, and CDR3 from a VH having the amino acid sequence of SEQ ID NO:75; or (7) a VL comprising VL CDR1, CDR2, and CDR3 from a VL having the amino acid sequence of SEQ ID NO:67, and/or a VH comprising VH CDR1, CDR2, and CDR3 from a VH having the amino acid sequence of SEQ ID NO:76; or (8) a VL comprising VL CDR1, CDR2, and CDR3 from a VL having the amino acid sequence of SEQ ID NO:68, and/or a VH comprising VH CDR1, CDR2, and CDR3 from a VH having the amino acid sequence of SEQ ID NO:77; or (9) a VL comprising VL CDR1, CDR2, and CDR3 from a VL having the amino acid sequence of SEQ ID NO:68, and/or a VH comprising VH CDR1, CDR2, and CDR3 from a VH having the amino acid sequence of SEQ ID NO:78; or a variant thereof having up to about 3 amino acid substitutions, additions, and/or deletions in the CDRs.

In some embodiments, provided herein are antibodies and antigen-binding fragments that specifically bind to human CEACAM1 comprising a VL and a VH, wherein the VL comprises VL CDR1, CDR2, and CDR3 from a VL having the amino acid sequence of SEQ ID NO:63, and the VH comprises VH CDR1, CDR2, and CDR3 from a VH having the amino acid sequence of SEQ ID NO:72; or a variant thereof having up to about 3 amino acid substitutions, additions, and/or deletions in the CDRs. In some embodiments, provided herein are antibodies and antigen-binding fragments that specifically bind to human CEACAM1 comprising a VL and a VH, wherein the VL comprises VL CDR1, CDR2, and CDR3 from a VL having the amino acid sequence of SEQ ID NO:65, and the VH comprises VH CDR1, CDR2, and CDR3 from a VH having the amino acid sequence of SEQ ID NO:74; or a variant thereof having up to about 3 amino acid substitutions, additions, and/or deletions in the CDRs. In some embodiments, provided herein are antibodies and antigen-binding fragments that specifically bind to human CEACAM1 comprising a VL and a VH, wherein the VL comprises VL CDR1, CDR2, and CDR3 from a VL having the amino acid sequence of SEQ ID NO:66, and the VH comprises VH CDR1, CDR2, and CDR3 from a VH having the amino acid sequence of SEQ ID NO:75; or a variant thereof having up to about 3 amino acid substitutions, additions, and/or deletions in the CDRs.

In some embodiments, provided herein are antibodies and antigen-binding fragments that compete with an the anti-CEACAM1 antibodies and antigen-binding fragments provided herein for binding to human CEACAM1.

In some embodiments, the anti-CEACAM1 antibodies and antigen-binding fragments provided herein are chimeric antibodies or antigen-binding fragments, humanized antibodies or antigen-binding fragments, or human antibodies or antigen-binding fragments.

In some embodiments, the anti-CEACAM1 antibodies and antigen-binding fragments provided herein are humanized antibodies or antigen-binding fragments. In some embodiments, the humanized anti-CEACAM1 antibodies and antigen-binding fragments provided herein have (a) a VL having at least 85%, at least 90%, at least 95%, at least 98%, or 100% sequence identity to an amino acid sequence selected from the group consisting of SEQ ID NOs:81-90; and/or (b) a VH having at least 85%, at least 90%, at least 95%, at least 98%, or 100% sequence identity to an amino acid sequence selected from the group consisting of SEQ ID NOs:111-116.

In some embodiments, the humanized anti-CEACAM1 antibodies and antigen-binding fragments provided herein have (a) a VL having at least 85%, at least 90%, at least 95%, at least 98%, or 100% sequence identity to an amino acid sequence selected from the group consisting of SEQ ID NOs:91-93 and 107-109; and/or (b) a VH having at least 85%, at least 90%, at least 95%, at least 98%, or 100% sequence identity to an amino acid sequence selected from the group consisting of SEQ ID NOs:117-122.

In some embodiments, the humanized anti-CEACAM1 antibodies and antigen-binding fragments provided herein have (a) a VL having at least 85%, at least 90%, at least 95%, at least 98%, or 100% sequence identity to an amino acid sequence selected from the group consisting of SEQ ID NOs:94-99; and/or (b) a VH having at least 85%, at least 90%, at least 95%, at least 98%, or 100% sequence identity to an amino acid sequence selected from the group consisting of SEQ ID NOs:123-128. In some embodiments, the VL has an amino acid sequence selected from the group consisting of SEQ ID NOs:94-99, with an amino acid substitution at S28, S51, K52, or any combination thereof. In some embodiments, the S28 substitution is S28F or S28H, the S51 substitution is S51M, and the K52 substitution is K52R. In some embodiments, the VL has an amino acid sequence selected from the group consisting of SEQ ID NOs:100-106. In some embodiments, the VH has an amino acid sequence selected from the group consisting of SEQ ID NOs:123-128, with an amino acid substitution at H35, N56, Y100, or any combination thereof. In some embodiments, the H35 substitution is H35M, the N56 substitution is N56I, N56Q, N56V, or N56W, and the Y100 substitution is Y100F, Y100W, or Y100V. In some embodiments, the VH has an amino acid sequence selected from the group consisting of SEQ ID NOs:130 and 143-149.

In some embodiments, the anti-CEACAM1 antibodies and antigen-binding fragments provided herein bind to human CEACAM1 with a K_{D} that is 10⁻⁸ M or less. In some embodiments, the anti-CEACAM1 antibodies and antigen-binding fragments provided herein bind to human CEACAM1 with a K_{D} that ranges from 10⁻¹¹ M to 10⁻⁸ M.

In some embodiments, the anti-CEACAM1 antibodies and antigen-binding fragments provided herein promote internalization of CEACAM1 on cell surface.

In some embodiments, the anti-CEACAM1 antibodies and antigen-binding fragments provided herein activate T cells, NK cells, or both. In some embodiments, the anti-CEACAM1 antibodies and antigen-binding fragments provided herein enhance IFN-γ release by T cells. In some embodiments, the anti-CEACAM1 antibodies and antigen-binding fragments provided herein enhance the activity of T cells, NK cells, or both, against tumor cells.

In some embodiments, the anti-CEACAM1 antibodies provided herein are selected from the group consisting of IgG1 antibodies, IgG2 antibodies, IgG3 antibodies, and IgG4 antibodies. In some embodiments, the anti-CEACAM1 antibodies provided herein are IgG1 antibodies.

In some embodiments, the anti-CEACAM1 antigen-binding fragments are selected from the group consisting of Fab, Fab′, F(ab′)₂, Fv, scFv, (scFv)₂, single domain antibodies (sdAb), and heavy chain antibodies (HCAb).

In some embodiments, the anti-CEACAM1 antibodies and antigen-binding fragments are monoclonal antibodies and antigen-binding fragments.

In some embodiments, provided herein are polynucleotides encoding the antibodies or antigen-binding fragments disclosed herein.

In some embodiments, provided herein are vectors comprising the polynucleotides disclosed herein.

In some embodiments, provided herein cells comprising the polynucleotides disclosed herein, or the vectors disclosed herein.

In some embodiments, provided herein are methods of making antibodies or antigen-binding fragments thereof that specifically bind to human CEACAM1, comprising culturing the cells disclosed herein under conditions that allow expression of the antibodies or antigen-binding fragments. In some embodiments, the methods comprise isolating the antibodies or antigen-binding fragments from the culture.

In some embodiments, provided herein are pharmaceutical compositions comprising a therapeutically effective amount of the anti-CEACAM1 antibodies or antigen-binding fragments disclosed herein, and a pharmaceutically acceptable carrier.

In some embodiments, provided herein are methods of inducing or stimulating immune cell activation and/or proliferation, comprising contacting an immune cell with an effective amount of the anti-CEACAM1 antibodies or antigen-binding fragments disclosed herein. In some embodiments, provided herein are methods of reducing the suppression of an immune cell comprising contacting the immune cell with an effective amount of the anti-CEACAM1 antibodies or antigen-binding fragments disclosed herein. In some embodiments, the immune cell is a lymphocyte of T cell or NK cell.

In some embodiments, provided herein are methods of increasing IFN-γ production in a T cell comprising contacting the T cell with an effective amount of the anti-CEACAM1 antibodies or antigen-binding fragments disclosed herein.

In some embodiments, provided herein are methods of inhibiting cancer cell growth, angiogenesis and/or metastasis in a subject in need thereof, comprising administering to the subject a therapeutically effective amount of the anti-CEACAM1 antibodies or antigen-binding fragments disclosed herein.

In some embodiments, provided herein are methods of treating cancer in a subject in need thereof, comprising administering to the subject a therapeutically effective amount of the anti-CEACAM1 antibodies or antigen-binding fragments disclosed herein.

In some embodiments, the methods provided herein further comprise administering an additional therapy to the subject.

In some embodiments of the methods provided herein, the subject is a human.

In some embodiments, provided herein are uses of the anti-CEACAM1 antibodies or antigen-binding fragments disclosed herein to in cancer treatment. In some embodiments, provided herein are uses of the anti-CEACAM1 antibodies or antigen-binding fragments disclosed herein for the preparation of a medicament for the treatment of cancer.

In some embodiments, the cancer is a CEACAM1-expressing cancer, a CEACAM5-expressing cancer or a CEACAM6-expressing cancer. In some embodiments, the cancer is a hematological cancer. In some embodiments, the cancer is a solid tumor. In some embodiments, the solid tumor is pancreatic cancer. In some embodiments, the solid tumor is melanoma.

### 5. BRIEF DESCRIPTION OF DRAWINGS

FIG. 1 provides flow cytometry data showing specific binding of the candidate anti-CEACAM1 antibodies to CEACAM1-expressing cells.

FIGS. 2A-2B provide results of Gator analysis showing the binding of CEACAM1, CEACAM3, CEACAM5, CEACAM6 and CEACAM8 to the captured antibodies as indicated. FIG. 2A shows the binding of CEACAM1, CEACAM3, CEACAM5, and CEACAM6 to the captured antibodies. FIG. 2B shows the binding of CEACAM8 to the captured antibodies.

FIGS. 3A-3B provide flow cytometry results showing the internalization of candidate anti-CEACAM1 antibodies in HepG2 (FIG. 3A) or BXPC-3 cells (FIG. 3B).

FIGS. 4A-4B provide results of Jurkat-NFAT-CEACAM1 bioassay (FIG. 4A) and T cell stimulation assay (FIG. 4B) showing the effect of candidate anti-CEACAM1 antibodies on T cell activation. As shown in FIG. 4A, candidate anti-CEACAM1 antibodies augmented the NFAT activity in CEACAM1-overexpressing Jurkat stable cell line (activated by anti-CD3 and anti-CD28 antibodies). As shown in FIG. 4B, candidate anti-CEACAM1 antibodies enhanced IFN-γ secretion in activated T cells co-cultured with 293T stable cell line with overexpression of CEACAM1 and OKT3.

FIGS. 5A-5C provide results of NK killing assay showing the effect of candidate anti-CEACAM1 antibodies on the killing curves of NK-92 (FIG. 5A) and primary NK cells (FIG. 5B) against target cell HepG2, and T cells killing assay showing the effect of candidate anti-CEACAM1 antibodies on the specific cytotoxicity of activated T cells (FIG. 5C) against 293T-CEACAM1.

FIGS. 6A-6D provide results of Wound Healing/Cell-Migration Assay showing the effect of candidate anti-CEACAM1 antibodies on BXPC-3 migration in the presence of monocyte-derived macrophage (FIG. 6A) or in the absence thereof (FIG. 6B), and the effect of candidate anti-CEACAM1 antibodies on the occurrence of polyploidy tumor cells in the presence of macrophage (FIG. 6C) or in the absence thereof (FIG. 6D).

FIGS. 7A-7B provide in vivo results showing that the administration of candidate anti-CEACAM1 antibodies inhibited tumor growth. Provided are tumor growth curves in humanized mouse models for melanoma (FIG. 7A) and pancreatic cancer (FIG. 7B) treated with indicated anti-CEACAM1 antibodies.

FIGS. 8A-8B provide interaction data showing that candidate anti-CEACAM1 antibodies blocked the interaction between CEACAM1 and CEACAM6 (FIG. 8A) and between CEACAM1 and CEACAM5 (FIG. 8B).

FIGS. 9A-9B provide data of NK-mediated tumor killing assay. As shown, CEACAM1 expression was not detectable in CEACAM1-KO BXPC-3 cells, whereas CEACAM5 expression remained the same as that of the parent BXPC-3 cells (FIG. 9A). All three candidate antibodies (h2C10.G4.H0aL4, h4D10.G4.H4L2, and h2C10.G4.H0aL2) promoted the cytotoxicity of the NK cells against CEACAM1-KO BXPC-3 cells in dose-dependent manners (FIG. 9B).

FIGS. 10A-10B provide results of IHC staining assay showing the binding ability of candidate anti-CEACAM1 antibodies. FIG. 10A shows IHC staining of 2C10 and CM24 on sections of BxPC-3/PBMC tumor tissues. FIG. 10B shows quantified integrated option density.

### 6. DETAILED DESCRIPTION

The present disclosure provides novel antibodies, including antigen-binding fragments that specifically bind CEACAM1 (e.g., human CEACAM1). Pharmaceutical compositions comprising a therapeutically effective amount of such antibodies or antigen-binding fragments are also disclosed herein. Also disclosed herein are uses of such pharmaceutical compositions for regulating activities of immune cells and in, for example, cancer treatment.

CEACAM1, or CEA (carcinoembryonic antigen)-related cell adhesion molecule 1, is a highly glycosylated, cell surface anchored, intracellular, and intercellular signaling molecule with diverse functions, from cell differentiation and transformation to modulating immune responses associated with infection, inflammation, autoimmunity, transplantation, and cancer. CEACAM1 contain an N-terminal V set fold from the immunoglobulin (Ig) superfamily, up to three type 2 immunoglobulins, a transmembrane domain, and a cytoplasmic domain, which facilitate adhesion through homophilic and/or heterophilic (CEACAM1-CEACAM5) interactions.

CEACAM1 comprises several isoforms through alternative splicing, differing in the amount of extracellular immunoglobulin-like domains and the length of the cytoplasmic tail. The short tail isoform (CEACAM1-S) does not have any immunoreceptor tyrosine-based inhibitory motifs (ITIMs), but contains sequences that can bind to calmodulin, tropomyosin, and F-actin. The long tail variant (CEACAM1-L) has two ITIMs that negatively regulate signaling from various activating receptors, including the T cell antigen receptor (TCR).

CEACAM1 has several binding targets: intracellularly, it forms cis-dimers, which are essential in cytoplasmic signaling. CEACAM1 initially undergoes trans-homophilic (CEACAM1-CEACAM1), or trans-heterophilic (CEACAM-CEA Family Member) dimerization, and can bind to other proteins and microbes. ITIMs on CEACAM1-L bind to other extracellular ligands, including other CEACAM family members and CEACAM1, suppressing immune cells and allowing immune evasion by cancer cells. CEACAM1 also downregulates natural killer cells and is involved in developing and differentiating a variety of myeloid-derived immune cells. CEACAM1 is a regulator of TIM-3, binding to TIM-3 through its N-terminal domain, forming a heterodimer, and facilitating the maturation and cell surface expression of TIM-3, which inhibits T cell activation and prevents the development of exhaustion-resistant and hyperinflammatory T cells. CEACAM1 also plays a regulatory role in NK-cell-mediated cytolysis, evading NK cells by promoting intracellular retention of several NKG2D ligands.

High CEACAM1 expression occurs in a variety of cancers such as melanoma, colorectal, gastric, pancreatic, bladder, and thyroid cancer and is associated with worse tumor progression, metastasis and poor clinical prognosis. CEACAM1 expression has also been strongly correlated with distant metastasis of pancreatic adenocarcinoma. CEACAM1 expression on tumors promotes CEACAM1-mediated inhibition of T and NK cells. Consequently, inhibiting CEACAM1 activity can inhibit tumor cell metastasis and the formation of a cancer stem cell niche. Moreover, CEACAM1 exhibits angiogenic properties and stimulates proliferation, chemotaxis, and capillary-like tube formation of human microvascular endothelial cells. CEACAM1 could express in tumor microvessels. Therefore, CEACAM1 could be a target for the inhibition of tumor angiogenesis.

CEACAM1 is also expressed in certain immune system cells and plays a role in immune suppression and immune cell exhaustion. High CEACAM1 expression on tumor infiltrating lymphocytes (TILs) and other tumor infiltrating immune cells from gastric, lung, melanoma, colorectal cancer and glioma, for example, is associated with a poor prognosis. CEACAM1-L is the dominant isoform expressed in most T cells and acts as an inhibitory receptor downregulating T cell activation and suppressing T cell functions. As such, inhibition of CEACAM1 on T-cells can recover T cell activity and increase anti-tumor responses. CEACAM1 is further expressed on NK cells, which are lymphocytes involved in innate immunity, participating in early control of viral infection and immune-surveillance of tumors. When CEACAM1 is present on the surface of both NK and melanoma cells, the CEACAM1 interactions lead to an inhibition of NK-mediated killing, independent of MHC class I expression. As such, disruption of this homophilic CEACAM1 interaction can be beneficial for restoring the NK-mediated immune response.

CEACAM1 expression on subsets of macrophages is further associated with fibrosis in the tumor microenvironment. CEACAM1 also regulates other stromal cells in the tumor microenvironment such as the vascular endothelium. Therefore, inhibiting interactions of CEACAM1 with its binding partners can further inhibit fibrosis and angiogenesis.

Full length human CEACAM1 Isoform 1 is a 526 amino acid protein (Uniprot Accession No. P13688-1, SEQ ID NO:129; long), which contains an extracellular domain (amino acids 35-428), a transmembrane domain (amino acids 429-452), and a cytoplasmic domain (amino acids 453-526). The extracellular domain includes one Ig-like V-type domain (amino acids 35-142) and three Ig-like C2 domains (amino acids 145-232, 237-317, and 323-413).

(SEQ ID NO: 129)
MGHLSAPLHRVRVPWQGLLLTASLLTFWNPPTTAQLTTESMPFNVAEGKE
VLLLVHNLPQQLFGYSWYKGERVDGNRQIVGYAIGTQQATPGPANSGRE
TIYPNASLLIQNVTQNDTGFYTLQVIKSDLVNEEATGQFHVYPELPKPSI
SSNNSNPVEDKDAVAFTCEPETQDTTYLWWINNQSLPVSPRLQLSNGNRT
LTLLSVTRNDTGPYECEIQNPVSANRSDPVTLNVTYGPDTPTISPSDTYY
RPGANLSLSCYAASNPPAQYSWLINGTFQQSTQELFIPNITVNNSGSYTC
HANNSVTGCNRTTVKTIIVTELSPVVAKPQIKASKTTVTGDKDSVNLTCS
TNDTGISIRWFFKNQSLPSSERMKLSQGNTTLSINPVKREDAGTYWCEVE
NPISKNQSDPIMLNVNYNALPQENGLSPGAIAGIVIGVVALVALIAVALA
CFLHFGKTGRASDQRDLTEHKPSVSNHTQDHSNDPPNKMNEVTYSTLNFE
AQQPTQPTSASPSLTATEIIYSEVKKQ

More information about human CEACAM1 can be found on public databases with the following IDs: HGNC: 1814; NCBI Entrez Gene: 634; Ensembl: ENSG00000079385; OMIM®: 109770; UniProtKB/Swiss-Prot: P13688. Eleven (11) alternatively spliced transcript variants encoding different isoforms are described for the human CEACAM1 gene (Uniprot NOs: P13688-1 to P13688-11).

Provided herein are antibodies or antigen-binding fragments thereof that specifically bind to human CEACAM1. CEACAM1 is detected on leukocytes, epithelia, and endothelia and is involved in cell adhesion through homophilic or heterophilic binding. In various gastrointestinal cancers, NSCLC (non-small cell lung cancer), and melanoma, CEACAM1 is highly expressed on tumor cells and tumor stroma, with its levels increasing as the disease progresses. Notably, CEACAM1 has been found to dampen T and NK cell functions while promoting tumor angiogenesis and metastasis. The anti-CEACAM1 antibodies provided herein can specifically bind to both CEACAM1 and CEACAM5, which are highly expressed in multiple cancers. CEACAM1 positive cancer cells can induce the expression of CEACAM1 in immune cells such as T cells and NK cells, further suppressing their activities by such contact-mediated inhibition. It was discovered that by targeting CEACAM1, the anti-CEACAM1 antibodies provided herein could restore activities of T cells and NK cells that were suppressed by CEACAM1-mediated signaling, as well as inhibit tumor angiogenesis and metastasis.

Before the present disclosure is further described, it is to be understood that the disclosure is not limited to the particular embodiments set forth herein, and it is also to be understood that the terminology used herein is for the purpose of describing particular embodiments, and is not intended to be limiting.

### 6.1 Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “a” or “an” entity refers to one or more of that entity; for example, “an antibody,” is understood to represent one or more antibodies.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” is used to indicate that a value includes the inherent variation of error for the device, the method being employed to determine the value, or the variation that exists among the study subjects. The term “about” encompasses the exact number recited. In some embodiments, “about” means within plus or minus 10% of a given value or range. In certain embodiments, “about” means that the variation is ±5%, ±4%, ±3%, ±2%, l %, ±0.5%, ±0.2%, or ±0.1% of the value to which “about” refers. In some embodiments, “about” means that the variation is ±1%, ±0.5%, ±0.2%, or ±0.1% of the value to which “about” refers.

The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab, Fab′, F(ab′)2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments. A “bispecific” antibody is an artificial hybrid antibody having two different antigen binding sites, which recognize and specifically bind two different targets. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulin. In some instances, the Fv framework region residues of a human immunoglobulin are replaced with the corresponding residues in an antibody from a non-human species. In some instances, residues of the CDRs are replaced by residues from the CDRs of a non-human species (e.g., mouse, rat, hamster, camel) that have the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The term “human antibody” as used herein refers to an antibody produced by a human or an antibody having an amino acid sequence corresponding to an antibody produced by a human made using any of the techniques known in the art.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while p and s contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5^{th} ed. A variable region can be a human variable region.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al, J. Biol. Chem. 252:6609-6616 (1977); Kabat, Adv. Prot. Chem. 32: 1-75 (1978)). The Chothia definition is based on the location of the structural loop regions, which defines CDR region sequences as those residues that are not part of the conserved β-sheet framework, and thus are able to adapt different conformations (Chothia and Lesk, J. Mol. Biol. 196:901-917 (1987)). Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures (Al-Lazikani et al, J. Mol. Biol. 273:927-948 (1997); Morea et al, Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VHCDRI | 31-35 | 26-32 | linking B and C strands |
| VHCDR2 | 50-65 | 53-55 | linking C′ and C″ strands |
| VHCDR3 | 95-102 | 96-101 | linking F and G strands |
| VLCDRI | 24-34 | 26-32 | linking B and C strands |
| VLCDR2 | 50-56 | 50-52 | linking C′ and C″ strands |
| VLCDR3 | 89-97 | 91-96 | linking F and G strands |

¹Residue numbering follows the nomenclature
of Kabat et al., supra
²Residue numbering follows the nomenclature
of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest. The CDR regions can be analyzed by, for example, abysis website (http://abysis.org/).

The terms “epitope” and “antigenic determinant” are used interchangeably herein an refer to the site on the surface of a target molecule to which an antibody or antigen-binding fragment binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety and a target molecule (e.g., antigen). The binding of a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, et al., (1999) J. Mol Biol 293:865-881). The K_{D} or K_{D} value can also be measured by using biolayer interferometry (BLI) using, for example, the Gator system (Probe Life), or the Octet-96 system (Sartorius AG). The K_{D} or K_{D} value can also be measured by using surface plasmon resonance assays (SPR) by Biacore, using, for example, a BIAcore™-2000 or a BIAcore™-3000 BIAcore, Inc., Piscataway, NJ). The binding affinity can also be quantified with EC₅₀, which is the concentration of ligand at which half of the target is present in the bound state in a binding assay.

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The term “variant” as used herein in relation to a protein or a polypeptide with particular sequence features (the “reference protein” or “reference polypeptide”) refers to a different protein or polypeptide having one or more (such as, for example, about 1 to about 30, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. The changes to an amino acid sequence can be amino acid deletions. A variant can be a fragment of the reference protein or polypeptide. A functional variant of a protein or polypeptide maintains the basic structural and functional properties of the reference protein or polypeptide.

The terms “polynucleotide,” “nucleic acid,” and their grammatical equivalents as used interchangeably herein refers to a polymer or oligomer of nucleotides of any length. The nucleotides can be deoxyribonucleotides, ribonucleotides, modified nucleotides or bases (such as methylated, hydroxymethylated, or glycosylated), non-natural nucleotides, non-nucleotide building blocks that exhibit similar structure and/or function as natural nucleotides (i.e., “nucleotide analogs”), and/or any substrate that can be incorporated into a polymer by DNA or RNA polymerase. The nucleic acids or polynucleotides can be heterogenous or homogenous in composition, can be isolated from naturally occurring sources, or can be artificially or synthetically produced. In addition, the nucleic acids may be DNA or RNA, or a mixture thereof, and can exist permanently or transitionally in single-stranded or double-stranded form, including homoduplex, heteroduplex, and hybrid states. Nucleic acid structures also include, for instance, a DNA/RNA helix, peptide nucleic acid (PNA), morpholino nucleic acid (see, e.g., Braasch and Corey, Biochemistry, 4(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, Am. Chem. Soc., 122: 8595-8602 (2000)), and/or a ribozyme.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-100 bases or more, and in some embodiments the sequences are substantially identical over the full length of the sequences being compared, such as a nucleotide sequence encoding a protein of interest.

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an anti-CEACAM1 antibody or antigen-binding fragment as described herein), and it is further understood that expression levels can be optimized to obtain sufficient expression using methods well known in the art.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

The term “subject” as used herein refers to any animal (e.g., a mammal), including, but not limited to, humans, non-human primates, canines, felines, rodents, and the like, which is to be the recipient of a particular treatment. A subject can be a human. A subject can have a particular disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOS. It is understood that one skilled in the art can readily identify homologous sequences by reference to sequence sources, including but not limited to GenBank (ncbi.nlm.nih.gov/genbank/) and EMBL (embl.org/).

### 6.2 Anti-CEACAM1 Antibodies and Antigen-Binding Fragments

Provided herein are antibodies or antigen-binding fragments thereof that specifically bind CEACAM1 (e.g., human CEACAM1). In some embodiments, provided herein are anti-CEACAM1 antibodies. In some embodiments, the antibody is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, provided herein are antigen-binding fragments of an anti-CEACAM1 antibody. In some embodiments, antigen-binding fragments provided herein can be a single domain antibody (sdAb), a heavy chain antibody (HCAb), a Fab, a Fab′, a F(ab′)₂, a Fv, a single-chain variable fragment (scFv), a disulfide-linked scFv [(scFv)₂], or a diabody (dAb). In some embodiments, the antigen-binding fragment of an anti-CEACAM1 antibody is a single domain antibody (sdAb). In some embodiments, the antigen-binding fragment of an anti-CEACAM1 antibody is a heavy chain antibody (HCAb). In some embodiments, the antigen-binding fragment of an anti-CEACAM1 antibody is a Fab. In some embodiments, the antigen-binding fragment of an anti-CEACAM1 antibody is a Fab′. In some embodiments, the antigen-binding fragment of an anti-CEACAM1 antibody is a F(ab′)₂. In some embodiments, the antigen-binding fragment of an anti-CEACAM1 antibody is a Fv. In some embodiments, the antigen-binding fragment of an anti-CEACAM1 antibody is a scFv. In some embodiments, the antigen-binding fragment of an anti-CEACAM1 antibody is a disulfide-linked scFv [(scFv)₂]. In some embodiments, the antigen-binding fragment of an anti-CEACAM1 antibody is a diabody (dAb).

In some embodiments, the anti-CEACAM1 antibodies or antigen-binding fragments provided herein comprise recombinant antibodies or antigen-binding fragments. In some embodiments, the anti-CEACAM1 antibodies or antigen-binding fragments provided herein comprise monoclonal antibodies or antigen-binding fragments. In some embodiments, the anti-CEACAM1 antibodies or antigen-binding fragments provided herein comprise polyclonal antibodies or antigen-binding fragments. In some embodiments, the anti-CEACAM1 antibodies or antigen-binding fragments provided herein comprise camelid (e.g., camels, dromedary and llamas) antibodies or antigen-binding fragments. In some embodiments, the anti-CEACAM1 antibodies or antigen-binding fragments provided herein comprise chimeric antibodies or antigen-binding fragments. In some embodiments, the anti-CEACAM1 antibodies or antigen-binding fragments provided herein comprise humanized antibodies or antigen-binding fragments. In some embodiments, the anti-CEACAM1 antibodies or antigen-binding fragments provided herein comprise human antibodies or antigen-binding fragments. In some embodiments, the anti-CEACAM1 antibodies or antigen-binding fragments provided herein are anti-CEACAM1 human scFvs.

In some embodiments, the anti-CEACAM1 antibodies or antigen-binding fragments provided herein are isolated. In some embodiments, the anti-CEACAM1 antibodies or antigen-binding fragments provided herein are substantially pure.

In some embodiments, the anti-CEACAM1 antibody or antigen-binding fragment provided herein comprises a multispecific antibody or antigen-binding fragment. In some embodiments, the anti-CEACAM1 antibody or antigen-binding fragment provided herein comprises a bispecific antibody or antigen-binding fragment. In some embodiments, the bispecific antibody or antigen-binding fragment comprises an anti-CEACAM1 antibody or antigen-binding fragment provided herein. In some embodiments, the bispecific antibody or antigen-binding fragment comprises an anti-CEACAM1 scFv provided herein.

In some embodiments, the anti-CEACAM1 antibody or antigen-binding fragment provided herein comprises a monovalent antigen-binding site. In some embodiments, an anti-CEACAM1 antibody or antigen-binding fragment comprises a monospecific binding site. In some embodiments, an anti-CEACAM1 antibody or antigen-binding fragment comprises a bivalent binding site.

In some embodiments, an anti-CEACAM1 antibody or antigen-binding fragment is a monoclonal antibody or antigen-binding fragment. Monoclonal antibodies can be prepared by any method known to those of skill in the art. One exemplary approach is screening protein expression libraries, e.g., phage or ribosome display libraries. Phage display is described, for example, in Ladner et al., U.S. Pat. No. 5,223,409; Smith (1985) Science 228:1315-1317; and WO 92/18619. In some embodiments, recombinant monoclonal antibodies are isolated from phage display libraries expressing variable regions or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, provided herein is the anti-CEACAM1 antibody clone 6H9, 7D4, 9D12, P23B6, 2C10, 4D10, 4H11, 2G12, and 5D5. The sequence features are described below. The specific CDR sequences defined herein are generally based on Kabat definition. However, it is understood that a general reference to a heavy chain CDR or CDRs and/or a light chain CDR or CDRs of a specific antibody encompass all CDR definitions as known to those of skill in the art. In some embodiments, provided herein are anti-CEACAM1 antibodies having the VL CDRs and/or VH CDRs of antibody clone 6H9, 7D4, 9D12, P23B6, 2C10, 4D10, 4H11, 2G12, or 5D5 disclosed herein, wherein the CDRs are defined by Kabat, Chothia, IMGT, AbM, or Contact. In some embodiments, the CDRs are defined by Kabat (as exemplified in detail below). In some embodiments, the CDRs are defined by Chothia. In some embodiments, the CDRs are defined by IMGT. In some embodiments, the CDRs are defined by AbM. In some embodiments, the CDRs are defined by Contact.

TABLE 1
Amino acid sequences of light chain variable region CDRs (VL CDRs)

| Sequence | VL CDR1 | VL CDR2 | VL CDR3 |
|---|---|---|---|
| 6H9 | RASQSVSTSSFSYMH | YASNLES | QHNWEIPYT |
| | (SEQ ID NO: 1) | (SEQ ID NO: 8) | (SEQ ID NO: 13) |
| 7D4 | SASSSVGYMH | EISKLAS | QQWNYPLT |
| | (SEQ ID NO: 2) | (SEQ ID NO: 9) | (SEQ ID NO: 14) |
| 9D12 | SASSSVSYMH | EISKLAS | QQWNYPLT |
| | (SEQ ID NO: 3) | (SEQ ID NO: 9) | (SEQ ID NO: 14) |
| P23B6 | RASQSVSTSSYSYMH | YASNLES | QHSWEIPWT |
| | (SEQ ID NO: 4) | (SEQ ID NO: 8) | (SEQ ID NO: 15) |
| 2C10 | RASQDITTYLN | YTSRLHS | QQGNTLPRT |
| | (SEQ ID NO: 5) | (SEQ ID NO: 10) | (SEQ ID NO: 16) |
| 4D10 | SASSSVSYMH | EISKLAS | QQWNYPLIT |
| | (SEQ ID NO: 3) | (SEQ ID NO: 9) | |
| | SASSFVSYMH | EIMKLAS | |
| | (SEQ ID NO: 52) | (SEQ ID NO: 54) | (SEQ ID NO: 17) |
| | SASSHVSYMH | EISRLAS | |
| | (SEQ ID NO: 53) | (SEQ ID NO: 55) | |
| | | EIMRLAS | |
| | | (SEQ ID NO: 56) | |
| 4H11 | KSSQSLLYSSNQKNYL | WASTRES | QQYYSYPST |
| | A (SEQ ID NO: 6) | (SEQ ID NO: 11) | (SEQ ID NO: 18) |
| 2G12 | RASQDIGSNLN | ATSSLDS | LQYASSPYT |
| | (SEQ ID NO: 7) | (SEQ ID NO: 12) | (SEQ ID NO: 19) |
| 5D5 | RASQDIGSNLN | ATSSLDS | LQYASSPYT |
| | (SEQ ID NO: 7) | (SEQ ID NO: 12) | (SEQ ID NO: 19) |

TABLE 2
Amino acid sequences of heavy chain variable region CDRs (VH CDRs)

| Sequence | VH CDR1 | VH CDR2 | VH CDR3 |
|---|---|---|---|
| 6H9 | SGYGWH | YIHYSGSTDYSPSLKS | DSSAWFAY |
| | (SEQ ID NO: 20) | (SEQ ID NO: 28) | (SEQ ID NO: 40) |
| 7D4 | DTYMH | RIDPSDGDTKYGPKFQD | AHFGGGGWYFDV |
| | (SEQ ID NO: 21) | (SEQ ID NO: 29) | (SEQ ID NO: 41) |
| 9D12 | DTYMH | RIDLASGDSKYDPKFQG | AHFGGGNWYFDV |
| | (SEQ ID NO: 21) | (SEQ ID NO: 30) | (SEQ ID NO: 42) |
| P23B6 | SYWLN | DIHPGRGSINYNEKFKS | ADGSWFVY |
| | (SEQ ID NO: 22) | (SEQ ID NO: 31) | (SEQ ID NO: 43) |
| 2C10 | NYLIE | VLNPGSGATNYNEKFKG | GIYGGYFAMDY |
| | (SEQ ID NO: 23) | (SEQ ID NO: 32) | (SEQ ID NO: 44) |
| 4D10 | SYVIH | YFNPYNNGTNYNENFKG | YYYASGSLYFDY |
| | (SEQ ID NO: 24) | (SEQ ID NO: 33) | (SEQ ID NO: 45) |
| | SYVIM | YFNPYNIGTNYNENFKG | YFYASGSLYFDY |
| | (SEQ ID NO: 57) | (SEQ ID NO: 34) | (SEQ ID NO: 46) |
| | | YFNPYNQGTNYNENFKG | YVYASGSLYFDY |
| | | (SEQ ID NO: 35) | (SEQ ID NO: 47) |
| | | YFNPYNVGTNYNENFKG | YWYASGSLYFDY |
| | | (SEQ ID NO: 36) | (SEQ ID NO: 48) |
| | | YFNPYNWGTNYNENFKG | |
| | | (SEQ ID NO: 37) | |
| 4H11 | DYGMN | WINTYTGEPTYADDFKG | KNIYGYFDV |
| | (SEQ ID NO: 25) | (SEQ ID NO: 38) | (SEQ ID NO: 49) |
| 2G12 | HHGMN | WINTYTGEPTYADDFRG | KDYYISGNFDY |
| | (SEQ ID NO: 26) | (SEQ ID NO: 39) | (SEQ ID NO: 50) |
| 5D5 | NYGMN | WINTYTGEPTYADDFKG | KDYYVSGNFDY |
| | (SEQ ID NO: 27) | (SEQ ID NO: 38) | (SEQ ID NO: 51) |

In some embodiments, anti-CEACAM1 antibodies or antigen-binding fragments provided herein comprise one, two, three, four, five, and/or six CDRs of any one of the antibodies described herein. In some embodiments, anti-CEACAM1 antibodies or antigen-binding fragments provided herein comprise a light chain variable region (VL) comprising one, two, and/or three light chain CDRs (VL CDRs) from Table 1. In some embodiments, anti-CEACAM1 antibodies or antigen-binding fragments provided herein comprise a heavy chain variable region (VH) comprising one, two, and/or three heavy chain CDRs (VH CDRs) from Table 2. In some embodiments, anti-CEACAM1 antibodies or antigen-binding fragments provided herein comprise one, two, and/or three VL CDRs from Table 1 and one, two, and/or three VH CDRs from Table 2.

In some embodiments, provided herein are antibodies or antigen-binding fragments thereof that specifically bind CEACAM1, comprising a VL comprising (1) a VL CDR1 having an amino acid sequence selected from the group consisting of SEQ ID NOs:1-7 and 52-53; (2) a VL CDR2 having an amino acid sequence selected from the group consisting of SEQ ID NOs:8-12 and 54-56; and/or (3) a VL CDR3 having an amino acid sequence selected from the group consisting of SEQ ID NOs:13-19; or a variant thereof having up to about 3, about 5, about 8, about 10, about 12, or about 15 amino acid substitutions, additions, and/or deletions in the VL CDRs; and/or a VH comprising (1) a VH CDR1 having an amino acid sequence selected from the group consisting of SEQ ID NOs:20-27 and 57; (2) a VH CDR2 having an amino acid sequence selected from the group consisting of SEQ ID NOs:28-39; and/or (3) a VH CDR3 having an amino acid sequence selected from the group consisting of SEQ ID NOs:40-51; or a variant thereof having up to about 3, about 5, about 8, about 10, about 12, or about 15 amino acid substitutions, additions, and/or deletions in the VH CDRs. In some embodiments, the variant has up to about 5 amino acid substitutions, additions, and/or deletions in the VL CDRs. In some embodiments, the variant has up to about 3 amino acid substitutions, additions, and/or deletions in the VL CDRs. In some embodiments, the variant has up to about 5 amino acid substitutions, additions, and/or deletions in the VH CDRs. In some embodiments, the variant has up to about 3 amino acid substitutions, additions, and/or deletions in the VH CDRs.

In some embodiments, provided herein are antibodies or antigen-binding fragments thereof that specifically bind CEACAM1, comprising (a) a VL comprising (1) a VL CDR1 having an amino acid sequence selected from the group consisting of SEQ ID NOs:1-7 and 52-53; (2) a VL CDR2 having an amino acid sequence selected from the group consisting of SEQ ID NOs:8-12 and 54-56; and (3) a VL CDR3 having an amino acid sequence selected from the group consisting of SEQ ID NOs:13-19; or a variant thereof having up to about 3 amino acid substitutions, additions, and/or deletions in the VL CDRs; and (b) a VH comprising (1) a VH CDR1 having an amino acid sequence selected from the group consisting of SEQ ID NOs:20-27 and 57; (2) a VH CDR2 having an amino acid sequence selected from the group consisting of SEQ ID NOs:28-39; and (3) a VH CDR3 having an amino acid sequence selected from the group consisting of SEQ ID NOs:40-51; or a variant thereof having up to about 3 amino acid substitutions, additions, and/or deletions in the VH CDRs.

In some embodiments, provided herein are antibodies or antigen-binding fragments thereof that specifically bind CEACAM1 having a VL, wherein the VL comprises VL CDR1, CDR2 and CDR3 having the amino acid sequences of (1) SEQ ID NOs:1, 8, and 13, respectively; (2) SEQ ID NOs:2, 9, and 14, respectively; (3) SEQ ID NOs:3, 9, and 14, respectively; (4) SEQ ID NOs:4, 8, and 15, respectively; (5) SEQ ID NOs:5, 10, and 16, respectively; (6) SEQ ID NOs:3, 9, and 17, respectively; (7) SEQ ID NOs:6, 11, and 18, respectively; or (8) SEQ ID NOs:7, 12, and 19, respectively; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs. In some embodiments, the variant has up to about 3 amino acid substitutions, additions, and/or deletions in the VL CDRs.

In some embodiments, provided herein are antibodies or antigen-binding fragments thereof that specifically bind CEACAM1 having a VL, wherein the VL comprises VL CDR1, CDR2 and CDR3 having the amino acid sequences of SEQ ID NOs:3, 9, and 17 or a variant thereof having up to about 3, about 5, about 8, about 10, about 12, or about 15 amino acid substitutions, additions, and/or deletions in the VL CDRs. In some embodiments, the VL CDR1 can have the amino acid sequence of SEQ ID NO:3 with a substitution at S5 (which corresponds to S28 in the VL). In some embodiments, the VL CDR1 can have the amino acid sequence of SEQ ID NO:3 with a substitution selected from the group consisting of S5F and S5H. In some embodiments, the VL CDR2 can have the amino acid sequence of SEQ ID NO:9 with a substitution at S3 and/or K4 (which correspond to S51 and K52 respectively in the VL). In some embodiments, the VL CDR2 can have the amino acid sequence of SEQ ID NO:9 with a substitution of S3M, K4R, or both.

In some embodiments, provided herein are antibodies or antigen-binding fragments thereof that specifically bind CEACAM1 having a VL, wherein the VL comprises (1) a VL CDR1 having an amino acid sequence selected from the group consisting of SEQ ID NOs:3 and 52-53; (2) a VL CDR2 having an amino acid sequence selected from the group consisting of SEQ ID NOs:9 and 54-56; and (3) a VL CDR3 having the amino acid sequence of SEQ ID NO: 17.

In some embodiments, provided herein are antibodies or antigen-binding fragments thereof that specifically bind CEACAM1 having a VL, wherein the VL comprises VL CDR1, CDR2 and CDR3 having the amino acid sequences of (1) SEQ ID NOs:3, 9, and 17, respectively; (2) SEQ ID NOs:3, 54, and 17, respectively; (3) SEQ ID NOs:3, 55, and 17, respectively; (4) SEQ ID NOs:3, 56, and 17, respectively; (5) SEQ ID NOs:52, 9, and 17, respectively; (6) SEQ ID NOs:52, 54, and 17, respectively; (7) SEQ ID NOs:52, 55, and 17, respectively; (8) SEQ ID NOs:52, 56, and 17, respectively; (9) SEQ ID NOs:53, 9, and 17, respectively; (10) SEQ ID NOs:53, 54, and 17, respectively; (11) SEQ ID NOs:53, 55, and 17, respectively; or (12) SEQ ID NOs:53, 56, and 17, respectively.

In some embodiments, provided herein are antibodies or antigen-binding fragments thereof that specifically bind CEACAM1 having a VH, wherein the VH comprises VH CDR1, CDR2 and CDR3 having the amino acid sequences of (1) SEQ ID NOs:20, 28, and 40, respectively; (2) SEQ ID NOs:21, 29, and 41, respectively; (3) SEQ ID NOs:21, 30, and 42, respectively; (4) SEQ ID NOs:22, 31, and 43, respectively; (5) SEQ ID NOs:23, 32, and 44, respectively; (6) SEQ ID NOs:24, 33, and 45, respectively; (7) SEQ ID NOs:25, 38, and 49, respectively; (8) SEQ ID NOs:26, 39, and 50, respectively; or (9) SEQ ID NOs:27, 38, and 51, respectively; or a variant thereof having up to about 3, about 5, about 8, about 10, about 12, or about 15 amino acid substitutions, additions, and/or deletions in the VH CDRs. In some embodiments, the variant has up to about 5 amino acid substitutions, additions, and/or deletions in the VH CDRs. In some embodiments, the variant has up to about 3 amino acid substitutions, additions, and/or deletions in the VH CDRs.

In some embodiments, provided herein are antibodies or antigen-binding fragments thereof that specifically bind CEACAM1 having a VH, wherein the VH comprises VH CDR1, CDR2 and CDR3 having the amino acid sequences of SEQ ID NOs:24, 33, and 45 or a variant thereof having up to about 3, about 5, about 8, about 10, about 12, or about 15 amino acid substitutions, additions, and/or deletions in the VH CDRs. In some embodiments, the VH CDR1 can have the amino acid sequence of SEQ ID NO:24 with a substitution at H5 (which corresponds to H35 in the VH). In some embodiments, the VH CDR1 can have the amino acid sequence of SEQ ID NO:24 with a substitution of H5M. In some embodiments, the VH CDR2 can have the amino acid sequence of SEQ ID NO:33 with a substitution at N7 (which corresponds to N56 in the VH). In some embodiments, the VH CDR2 can have the amino acid sequence of SEQ ID NO:33 with a substitution selected from the group consisting of N7I, N7Q, N7V and N7W. In some embodiments, the VH CDR3 can have the amino acid sequence of SEQ ID NO:45 with a substitution at Y2 (which corresponds to Y100 in the VH). In some embodiments, the VH CDR3 can have the amino acid sequence of SEQ ID NO:45 with a substitution selected from the group consisting of Y2F, Y2V, and Y2W.

In some embodiments, provided herein are antibodies or antigen-binding fragments thereof that specifically bind CEACAM1 having a VH, wherein the VH comprises (1) a VH CDR1 having an amino acid sequence selected from the group consisting of SEQ ID NOs:24 and 57; (2) a VH CDR2 having an amino acid sequence selected from the group consisting of SEQ ID NOs:33-37; and (3) a VH CDR3 having an amino acid sequence selected from the group consisting of SEQ ID NOs:45-48.

In some embodiments, provided herein are antibodies or antigen-binding fragments thereof that specifically bind CEACAM1 having a VH, wherein the VH comprises VH CDR1, CDR2 and CDR3 having the amino acid sequences of (1) SEQ ID NOs:24, 33, and 45, respectively; (2) SEQ ID NOs:24, 33, and 46, respectively; (3) SEQ ID NOs:24, 33, and 47, respectively; (4) SEQ ID NOs:24, 33, and 48, respectively; (5) SEQ ID NOs:24, 34, and 45, respectively; (6) SEQ ID NOs:24, 34, and 46, respectively; (7) SEQ ID NOs:24, 34, and 47, respectively; (8) SEQ ID NOs:24, 34, and 48, respectively; (9) SEQ ID NOs:24, 35, and 45, respectively; (10) SEQ ID NOs:24, 35, and 46, respectively; (11) SEQ ID NOs:24, 35, and 47, respectively; (12) SEQ ID NOs:24, 35, and 48, respectively; (13) SEQ ID NOs:24, 36, and 45, respectively; (14) SEQ ID NOs:24, 36, and 46, respectively; (15) SEQ ID NOs:24, 36, and 47, respectively; (16) SEQ ID NOs:24, 36, and 48, respectively; (17) SEQ ID NOs:24, 37, and 45, respectively; (18) SEQ ID NOs:24, 37, and 46, respectively; (19) SEQ ID NOs:24, 37, and 47, respectively; (20) SEQ ID NOs:24, 37, and 48, respectively; (21) SEQ ID NOs:57, 33, and 45, respectively; (22) SEQ ID NOs:57, 33, and 46, respectively; (23) SEQ ID NOs:57, 33, and 47, respectively; (24) SEQ ID NOs:57, 33, and 48, respectively; (25) SEQ ID NOs:57, 34, and 45, respectively; (26) SEQ ID NOs:57, 34, and 46, respectively; (27) SEQ ID NOs:57, 34, and 47, respectively; (28) SEQ ID NOs:57, 34, and 48, respectively; (29) SEQ ID NOs:57, 35, and 45, respectively; (30) SEQ ID NOs:57, 35, and 46, respectively; (31) SEQ ID NOs:57, 35, and 47, respectively; (32) SEQ ID NOs:57, 35, and 48, respectively; (33) SEQ ID NOs:57, 36, and 45, respectively; (34) SEQ ID NOs:57, 36, and 46, respectively; (35) SEQ ID NOs:57, 36, and 47, respectively; (36) SEQ ID NOs:57, 36, and 48, respectively; (37) SEQ ID NOs:57, 37, and 45, respectively; (38) SEQ ID NOs:57, 37, and 46, respectively; (39) SEQ ID NOs:57, 37, and 47, respectively; or (40) SEQ ID NOs:57, 37, and 48, respectively.

In some embodiments, provided herein are antibodies or antigen-binding fragments thereof that specifically bind CEACAM1 having a VL and a VH, wherein (a) the VL comprises VL CDR1, CDR2 and CDR3 having the amino acid sequences of (1) SEQ ID NOs:1, 8, and 13, respectively; (2) SEQ ID NOs:2, 9, and 14, respectively; (3) SEQ ID NOs:3, 9, and 14, respectively; (4) SEQ ID NOs:4, 8, and 15, respectively; (5) SEQ ID NOs:5, 10, and 16, respectively; (6) SEQ ID NOs:3, 9, and 17, respectively; (7) SEQ ID NOs:3, 54, and 17, respectively; (8) SEQ ID NOs:3, 55, and 17, respectively; (9) SEQ ID NOs:3, 56, and 17, respectively; (10) SEQ ID NOs:52, 9, and 17, respectively; (11) SEQ ID NOs:52, 54, and 17, respectively; (12) SEQ ID NOs:52, 55, and 17, respectively; (13) SEQ ID NOs:52, 56, and 17, respectively; (14) SEQ ID NOs:53, 9, and 17, respectively; (15) SEQ ID NOs:53, 54, and 17, respectively; (16) SEQ ID NOs:53, 55, and 17, respectively; (17) SEQ ID NOs:53, 56, and 17, respectively; (18) SEQ ID NOs:6, 11, and 18, respectively; or (19) SEQ ID NOs:7, 12, and 19, respectively; or a variant thereof having up to about 3 amino acid substitutions, additions, and/or deletions in the VL CDRs; and (b) the VH comprises VH CDR1, CDR2 and CDR3 having the amino acid sequences of (1) SEQ ID NOs:20, 28, and 40, respectively; (2) SEQ ID NOs:21, 29, and 41, respectively; (3) SEQ ID NOs:21, 30, and 42, respectively; (4) SEQ ID NOs:22, 31, and 43, respectively; (5) SEQ ID NOs:23, 32, and 44, respectively; (6) SEQ ID NOs:24, 33, and 45, respectively; (7) SEQ ID NOs:24, 33, and 46, respectively; (8) SEQ ID NOs:24, 33, and 47, respectively; (9) SEQ ID NOs:24, 33, and 48, respectively; (10) SEQ ID NOs:24, 34, and 45, respectively; (11) SEQ ID NOs:24, 34, and 46, respectively; (12) SEQ ID NOs:24, 34, and 47, respectively; (13) SEQ ID NOs:24, 34, and 48, respectively; (14) SEQ ID NOs:24, 35, and 45, respectively; (15) SEQ ID NOs:24, 35, and 46, respectively; (16) SEQ ID NOs:24, 35, and 47, respectively; (17) SEQ ID NOs:24, 35, and 48, respectively; (18) SEQ ID NOs:24, 36, and 45, respectively; (19) SEQ ID NOs:24, 36, and 46, respectively; (20) SEQ ID NOs:24, 36, and 47, respectively; (21) SEQ ID NOs:24, 36, and 48, respectively; (22) SEQ ID NOs:24, 37, and 45, respectively; (23) SEQ ID NOs:24, 37, and 46, respectively; (24) SEQ ID NOs:24, 37, and 47, respectively; (25) SEQ ID NOs:24, 37, and 48, respectively; (26) SEQ ID NOs:57, 33, and 45, respectively; (27) SEQ ID NOs:57, 33, and 46, respectively; (28) SEQ ID NOs:57, 33, and 47, respectively; (29) SEQ ID NOs:57, 33, and 48, respectively; (30) SEQ ID NOs:57, 34, and 45, respectively; (31) SEQ ID NOs:57, 34, and 46, respectively; (32) SEQ ID NOs:57, 34, and 47, respectively; (33) SEQ ID NOs:57, 34, and 48, respectively; (34) SEQ ID NOs:57, 35, and 45, respectively; (35) SEQ ID NOs:57, 35, and 46, respectively; (36) SEQ ID NOs:57, 35, and 47, respectively; (37) SEQ ID NOs:57, 35, and 48, respectively; (38) SEQ ID NOs:57, 36, and 45, respectively; (39) SEQ ID NOs:57, 36, and 46, respectively; (40) SEQ ID NOs:57, 36, and 47, respectively; (41) SEQ ID NOs:57, 36, and 48, respectively; (42) SEQ ID NOs:57, 37, and 45, respectively; (43) SEQ ID NOs:57, 37, and 46, respectively; (44) SEQ ID NOs:57, 37, and 47, respectively; (45) SEQ ID NOs:57, 37, and 48, respectively; (46) SEQ ID NOs:25, 38, and 49, respectively; (47) SEQ ID NOs:26, 39, and 50, respectively; or (48) SEQ ID NOs:27, 38, and 51, respectively; or a variant thereof having up to about 3 amino acid substitutions, additions, and/or deletions in the VH CDRs.

In some embodiments, provided herein are antibodies or antigen-binding fragments thereof that specifically bind CEACAM1 having a VL and a VH, wherein the VL comprises VL CDR1, CDR2 and CDR3 and the VH comprises VH CDR1, CDR2 and CDR3, and wherein the VL CDR1, VL CDR2, VL CDR3, VH CDR1, VH CDR2 and VH CDR3 have the amino acid sequences of (1) SEQ ID NOs:1, 8, 13, 20, 28, and 40, respectively; (2) SEQ ID NOs:2, 9, 14, 21, 29, and 41, respectively; (3) SEQ ID NOs:3, 9, 14, 21, 30, and 42, respectively; (4) SEQ ID NOs:4, 8, 15, 22, 31, and 43, respectively; (5) SEQ ID NOs:5, 10, 16, 23, 32, and 44, respectively; (6) SEQ ID NOs:3, 9, 17, 24, 33, and 45, respectively; (7) SEQ ID NOs:3, 9, 17, 24, 33, and 46, respectively; (8) SEQ ID NOs:3, 9, 17, 24, 33, and 47, respectively; (9) SEQ ID NOs:3, 9, 17, 24, 33, and 48, respectively; (10) SEQ ID NOs:3, 9, 17, 24, 34, and 45, respectively; (11) SEQ ID NOs:3, 9, 17, 24, 34, and 46, respectively; (12) SEQ ID NOs:3, 9, 17, 24, 34, and 47, respectively; (13) SEQ ID NOs:3, 9, 17, 24, 34, and 48, respectively; (14) SEQ ID NOs:3, 9, 17, 24, 35, and 45, respectively; (15) SEQ ID NOs:3, 9, 17, 24, 35, and 46, respectively; (16) SEQ ID NOs:3, 9, 17, 24, 35, and 47, respectively; (17) SEQ ID NOs:3, 9, 17, 24, 35, and 48, respectively; (18) SEQ ID NOs:3, 9, 17, 24, 36, and 45, respectively; (19) SEQ ID NOs:3, 9, 17, 24, 36, and 46, respectively; (20) SEQ ID NOs:3, 9, 17, 24, 36, and 47, respectively; (21) SEQ ID NOs:3, 9, 17, 24, 36, and 48, respectively; (22) SEQ ID NOs:3, 9, 17, 24, 37, and 45, respectively; (23) SEQ ID NOs:3, 9, 17, 24, 37, and 46, respectively; (24) SEQ ID NOs:3, 9, 17, 24, 37, and 47, respectively; (25) SEQ ID NOs:3, 9, 17, 24, 37, and 48, respectively; (26) SEQ ID NOs:6, 11, 18, 25, 38, and 49, respectively; (27) SEQ ID NOs:7, 12, 19, 26, 39, and 50, respectively; or (28) SEQ ID NOs:7, 12, 19, 27, 38, and 51, respectively; or a variant thereof having up to about 5 amino acid substitutions, additions, and/or deletions in the CDRs. In some embodiments, the variant has up to about 3 amino acid substitutions, additions, and/or deletions in the CDRs.

In some embodiments, provided herein are antibodies or antigen-binding fragments thereof that specifically bind CEACAM1 having a VL and a VH, wherein (a) the VL comprises (1) a VL CDR1 having an amino acid sequence selected from the group consisting of SEQ ID NOs:3 and 52-53; (2) a VL CDR2 having an amino acid sequence selected from the group consisting of SEQ ID NOs:9 and 54-56; and (3) a VL CDR3 having the amino acid sequence of SEQ ID NO:17; and (b) the VH comprises (1) a VH CDR1 having an amino acid sequence selected from the group consisting of SEQ ID NOs:24 and 57; (2) a VH CDR2 having an amino acid sequence selected from the group consisting of SEQ ID NOs:33-37; and (3) a VH CDR3 having an amino acid sequence selected from the group consisting of SEQ ID NOs:45-48.

In some embodiments, provided herein are antibodies or antigen-binding fragments thereof that specifically bind CEACAM1 having a VL and a VH, wherein the VL comprises VL CDR1, CDR2 and CDR3 and the VH comprises VH CDR1, CDR2 and CDR3, and wherein the VL CDR1, VL CDR2, VL CDR3, VH CDR1, VH CDR2 and VH CDR3 have the amino acid sequences of (1) SEQ ID NOs:52, 9, 17, 24, 35, and 46, respectively; (2) SEQ ID NOs:53, 9, 17, 24, 35, and 46, respectively; (3) SEQ ID NOs:3, 54, 17, 24, 35, and 46, respectively; (4) SEQ ID NOs:3, 55, 17, 24, 35, and 46, respectively; (5) SEQ ID NOs:52, 54, 17, 24, 35, and 46, respectively; (6) SEQ ID NOs:52, 55, 17, 24, 35, and 46, respectively; (7) SEQ ID NOs:52, 56, 17, 24, 35, and 46, respectively; or (8) SEQ ID NOs:3, 9, 17, 57, 35, and 46, respectively; or a variant thereof having up to about 5 amino acid substitutions, additions, and/or deletions in the CDRs. In some embodiments, the variant has up to about 3 amino acid substitutions, additions, and/or deletions in the CDRs.

TABLE 3
Amino acid sequences of light chain variable regions
(VLs) and heavy chain variable region
(VHs) of anti-hCEACAMI antibodies

| Ab | VL | VH |
|---|---|---|
| 6H9 | DIVLTQSPASLAVSLGQRATISCRASQSV | DVQLQESGPDLVKPSQSLSLTCTVTGYSI |
| | STSSFSYMHWYQQKPGQPPKLLIKYASN | TSGYGWHWIRQFPGNKLEWMGYIHYSG |
| | LESGVPARFSGSGSGTDFTLNIHPVEDED | STDYSPSLKSRISITRDTSKNQFFLQLNSV |
| | PATYYCQHNWEIPYTFGGGTKLEIK | TTEDTATYYCARDSSAWFAYWGQGTL |
| | (SEQ ID NO: 61) | VTVSA |
| | | (SEQ ID NO: 70) |
| 7D4 | EIVLTQSPAITAASLGQKVTITCSASSSVG | EVQLQQSGAELVRPGASVKLSCTASGEN |
| | YMHWYQQKSGTSPKPWIYEISKLASGV | LKDTYMHWVKQRPEQGLEWIGRIDPSD |
| | PARFSGSGSGTSYSLTIRNMEAEDAAIYY | GDTKYGPKFQDKTTITADTSSNTAYLQL |
| | CQQWNYPLTFGAGTKLELK | SSLTSEDAAVYYCARAHFGGGGWYFDV |
| | (SEQ ID NO: 62) | WGAGTTVTVSS |
| | | (SEQ ID NO: 71) |
| 9D12 | EIVLTQSPAITAASLGQKVTITCSASSSVS | EVQLQQSGAELVKPGASVKLSCTASGFN |
| | YMHWYQQKSGTSPKPWIYEISKLASGV | IKDTYMHWVKQRPEQGLEWIGRIDLAS |
| | PARFSGSGSGTSYSLTISSMEAEDAAIYY | GDSKYDPKFQGKATITADTSSNTAYLQF |
| | CQQWNYPLTFGAGTKLELK | SSLTSEDTAVYYCARAHFGGGNWYFDV |
| | (SEQ ID NO: 63) | WGAGTTVTVSS |
| | | (SEQ ID NO: 72) |
| P23B6 | DIVLTQSPASLAVSLGQRATISCRASQSV | QVQLLQPGAEPVKPGTSVKLSCKASGY |
| | STSSYSYMHWYQQKPGQPPKLLIKYAS | NFTSYWLNWVKLRPGQGLEWIGDIHPG |
| | NLESGVPARFSGSGSGTDFTLNIHPVEEE | RGSINYNEKFKSKAILTVDTSSRTAYIQL |
| | DTATYYCQHSWEIPWTFGGGTKLEIK | SSLASEDSALYYCARADGSWFVYWGQG |
| | (SEQ ID NO: 64) | TLVTVSA |
| | | (SEQ ID NO: 73) |
| 2C10 | DIQMTQTTSSLSASLGDRVTISCRASQDI | QVQLQQSGAELVRPGTSVKVSCKASGF |
| | TTYLNWYQQRPDGTVKLLIYYTSRLHS | AFTNYLIEWVKQRPGQGLEWIGVLNPGS |
| | GVPSRFSGSGSGTEYSLTISNLEQEDIAT | GATNYNEKFKGKATLTADKSSSTAYMQ |
| | YFCQQGNTLPRTFGGGTKLEIR | LSSLTSDDSAVYFCARGIYGGYFAMDY |
| | (SEQ ID NO: 65) | WGQGTSVTVSS |
| | | (SEQ ID NO: 74) |
| 4D10 | EIVLTQSPAIATASLGQKVTITCSASSSVS | EVQLQQSGPELVKPGASVKMSCKASGY |
| | YMHWYQQKSGTSPKPWIYEISKLASGV | TFSSYVIHWVRQKPGQGLEWIGYFNPYN |
| | PTRFSGSGSGTSYSLTISSMEAEDAAIYY | NGTNYNENFKGKATLTSGKSSSTAYME |
| | CQQWNYPLITFGAGTKLELK | LSSLTSEDSAVYYCARYYYASGSLYFDY |
| | (SEQ ID NO: 66) | WGQGTTLTVSS |
| | | (SEQ ID NO: 75) |
| 4H11 | DIVMSQSPSSLAVSVGEKVTLSCKSSQSL | QIQLVQSGPELKKPGETVKISCKASGYTL |
| | LYSSNQKNYLAWYQQKPGQSPKLLIYW | TDYGMNWVSQAPGKGLKWVGWINTYT |
| | ASTRESGVPDRFTGSGSGTDFTLTISSVK | GEPTYADDFKGRFAFSLDTSASTAYLQI |
| | AEDLAVYYCQQYYSYPSTFGGGTKLEIK | NNLKNEDTATYFCARKNIYGYFDVWGA |
| | (SEQ ID NO: 67) | GTTVTVSS |
| | | (SEQ ID NO: 76) |
| 2G12 | DIQMTQSPSSLSASLGERVSLTCRASQDI | QIQLVQSGPELKKPGETVKISCKASGYSF |
| | GSNLNWLQQEPDGTIKRLIYATSSLDSG | THHGMNWVKQAPGKDLKWMGWINTY |
| | VPKRFSGSRSGSDYSLTISSLESEDFVDY | TGEPTYADDFRGRFAFSLETSASTAYLQI |
| | YCLQYASSPYTFGGGTKLEIK | NNLKSEDTATYFCARKDYYISGNFDYW |
| | (SEQ ID NO: 68) | GQGTTLTVSS |
| | | (SEQ ID NO: 77) |
| 5D5 | DIQMTQSPSSLSASLGERVSLTCRASQDI | QIQLVQSGPELKKPGETVKISCKASGYTL |
| | GSNLNWLQQEPDGTIKRLIYATSSLDSG | TNYGMNWVKQAPGTDLKWMGWINTY |
| | VPKRFSGSRSGSDYSLTISSLESEDFVDY | TGEPTYADDFKGRFAFSLETSASTAYLQI |
| | YCLQYASSPYTFGGGTKLEIK | NNLKNEDTATYFCARKDYYVSGNFDY |
| | (SEQ ID NO: 68) | WGQGTTLTVSS |
| | | (SEQ ID NO: 78) |

In some embodiments, provided herein are antibodies or antigen-binding fragments thereof that specifically bind CEACAM1 comprising a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:61-68. In some embodiments, provided herein are antibodies or antigen-binding fragments thereof that specifically bind CEACAM1 comprising a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:70-78.

In some embodiments, provided herein are antibodies or antigen-binding fragments thereof that specifically bind CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:61-68; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:70-78.

In some embodiments, provided herein are antibodies or antigen-binding fragments thereof that specifically bind CEACAM1 comprising a VL and a VH, wherein the VL and VH have the amino acid sequences of (1) SEQ ID NOs:61 and 70, respectively; (2) SEQ ID NOs:62 and 71, respectively; (3) SEQ ID NOs:63 and 72, respectively; (4) SEQ ID NOs:64 and 73, respectively; (5) SEQ ID NOs:65 and 74, respectively; (6) SEQ ID NOs:66 and 75, respectively; (7) SEQ ID NOs:67 and 76, respectively; (8) SEQ ID NOs:68 and 77, respectively; or (9) SEQ ID NOs:68 and 78, respectively; or a variant thereof having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the recited VL and/or VH sequences.

In some embodiments, provided herein are antibodies or antigen-binding fragments thereof that specifically bind CEACAM1 comprising a VL and a VH, wherein the VL has the amino acid sequence of SEQ ID NO:61, and the VH has an amino acid sequence selected from the group consisting of SEQ ID NOs:70-78. In some embodiments, the VL has the amino acid sequence of SEQ ID NO:62, and the VH has an amino acid sequence selected from the group consisting of SEQ ID NOs:70-78. In some embodiments, the VL has the amino acid sequence of SEQ ID NO:63, and the VH has an amino acid sequence selected from the group consisting of SEQ ID NOs:70-78. In some embodiments, the VL has the amino acid sequence of SEQ ID NO:64, and the VH has an amino acid sequence selected from the group consisting of SEQ ID NOs:70-78. In some embodiments, the VL has the amino acid sequence of SEQ ID NO:65, and the VH has an amino acid sequence selected from the group consisting of SEQ ID NOs:70-78. In some embodiments, the VL has the amino acid sequence of SEQ ID NO:66, and the VH has an amino acid sequence selected from the group consisting of SEQ ID NOs:70-78. In some embodiments, the VL has the amino acid sequence of SEQ ID NO:67, and the VH has an amino acid sequence selected from the group consisting of SEQ ID NOs:70-78. In some embodiments, the VL has the amino acid sequence of SEQ ID NO:68, and the VH has an amino acid sequence selected from the group consisting of SEQ ID NOs:70-78.

In some embodiments, provided herein are antibodies or antigen-binding fragments thereof that specifically bind CEACAM1 comprising a VL and a VH, wherein the VL has an amino acid sequence selected from the group consisting of SEQ ID NOs:61-68, and the VH has the amino acid sequence of SEQ ID NO:70. In some embodiments, the VL has an amino acid sequence selected from the group consisting of SEQ ID NOs:61-68, and the VH has the amino acid sequence of SEQ ID NO:71. In some embodiments, the VL has an amino acid sequence selected from the group consisting of SEQ ID NOs:61-68, and the VH has the amino acid sequence of SEQ ID NO:72. In some embodiments, the VL has an amino acid sequence selected from the group consisting of SEQ ID NOs:61-68, and the VH has the amino acid sequence of SEQ ID NO:73. In some embodiments, the VL has an amino acid sequence selected from the group consisting of SEQ ID NOs:61-68, and the VH has the amino acid sequence of SEQ ID NO:74. In some embodiments, the VL has an amino acid sequence selected from the group consisting of SEQ ID NOs:61-68, and the VH has the amino acid sequence of SEQ ID NO:75. In some embodiments, the VL has an amino acid sequence selected from the group consisting of SEQ ID NOs:61-68, and the VH has the amino acid sequence of SEQ ID NO:76. In some embodiments, the VL has an amino acid sequence selected from the group consisting of SEQ ID NOs:61-68, and the VH has the amino acid sequence of SEQ ID NO:77. In some embodiments, the VL has an amino acid sequence selected from the group consisting of SEQ ID NOs:61-68, and the VH has the amino acid sequence of SEQ ID NO:78.

In some embodiments, provided herein are antibodies or antigen-binding fragments thereof that specifically bind CEACAM1 comprising a VL and a VH, wherein the VL and VH have the amino acid sequences of SEQ ID NOs:61 and 70, respectively. In some embodiments, the VL and VH have the amino acid sequences of SEQ ID NOs:62 and 71, respectively. In some embodiments, the VL and VH have the amino acid sequences of SEQ ID NOs:63 and 72, respectively. In some embodiments, the VL and VH have the amino acid sequences of SEQ ID NOs:64 and 73, respectively. In some embodiments, the VL and VH have the amino acid sequences of SEQ ID NOs:65 and 74, respectively. In some embodiments, the VL and VH have the amino acid sequences of SEQ ID NOs:66 and 75, respectively. In some embodiments, the VL and VH have the amino acid sequences of SEQ ID NOs:67 and 76, respectively. In some embodiments, the VL and VH have the amino acid sequences of SEQ ID NOs:68 and 77, respectively. In some embodiments, the VL and VH have the amino acid sequences of SEQ ID NOs:68 and 78, respectively.

The anti-CEACAM1 antibodies or antigen-binding fragments thereof can comprise a combination of any VL disclosed herein and any VH disclosed herein. In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO:140). In some embodiments, the linker has the amino acid sequence of (EAAAK)n, n=1, 2, 3, 4, or 5 (SEQ ID NO:141). In some embodiments, the linker has the amino acid sequence of (PA)nP, n=1, 2, 3, 4, or 5 (SEQ ID NO:142).

In some embodiments, provided herein are antibodies or antigen-binding fragments thereof that specifically bind CEACAM1 comprising (a) a VL comprising VL CDRs 1, 2, and 3 from a VL having an amino acid sequence selected from the group consisting of SEQ ID NOs:61-68; and/or (b) a VH comprising VH CDRs 1, 2, and 3 from a VH having an amino acid sequence selected from the group consisting of SEQ ID NOs:70-78.

In some embodiments, the anti-CEACAM1 antibody or antigen-binding fragment thereof provided herein is derived from the antibody clone designated as 6H9. In some embodiments, the anti-CEACAM1 antibody or antigen-binding fragment thereof provided herein has a VL from 6H9 (SEQ ID NO:61). In some embodiments, the anti-CEACAM1 antibody or antigen-binding fragment thereof provided herein has a VH from 6H9 (SEQ ID NO:70). The anti-CEACAM1 antibody or antigen-binding fragment thereof provided herein can have both a VL and a VH from 6H9.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:61; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:70. In some embodiments, provided herein are antibodies or antigen-binding fragments thereof that specifically bind CEACAM1 comprising a VL and a VH, wherein the VL and VH have the amino acid sequences of SEQ ID NOs:61 and 70, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:61. The anti-CEACAM1 antibody or antigen-binding fragment thereof can have a VL having at least 85% sequence identity to SEQ ID NO:61. The anti-CEACAM1 antibody or antigen-binding fragment thereof can have a VL having at least 90% sequence identity to SEQ ID NO:61. The anti-CEACAM1 antibody or antigen-binding fragment thereof can have a VL having at least 95% sequence identity to SEQ ID NO:61. The anti-CEACAM1 antibody or antigen-binding fragment thereof can have a VL having at least 98% sequence identity to SEQ ID NO:61. The anti-CEACAM1 antibody or antigen-binding fragment thereof can have a VL having the amino acid sequence of SEQ ID NO:61.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:70. The anti-CEACAM1 antibody or antigen-binding fragment thereof can have a VH having at least 85% sequence identity to SEQ ID NO:70. The anti-CEACAM1 antibody or antigen-binding fragment thereof can have a VH having at least 90% sequence identity to SEQ ID NO:70. The anti-CEACAM1 antibody or antigen-binding fragment thereof can have a VH having at least 95% sequence identity to SEQ ID NO:70. The anti-CEACAM1 antibody or antigen-binding fragment thereof can have at least 98% sequence identity to SEQ ID NO:70. The anti-CEACAM1 antibody or antigen-binding fragment thereof can have a VH having the amino acid sequence of SEQ ID NO:70.

In some embodiments, the anti-CEACAM1 antibody or antigen-binding fragment thereof provided herein has a VL that comprises VL CDRs 1, 2, and 3 from the VL of 6H9 (SEQ ID NO:61). In some embodiments, the anti-CEACAM1 antibody or antigen-binding fragment thereof provided herein has a VH that comprises VH CDRs 1, 2, and 3 from the VH of 6H9 (SEQ ID NO:70). The anti-CEACAM1 antibody or antigen-binding fragment thereof provided herein can have a VL comprising VL CDRs 1, 2, and 3 from the VL of 6H9 (SEQ ID NO:61) and a VH comprising VH CDRs 1, 2, and 3 from the VH of 6H9 (SEQ ID NO:70), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:1, (2) a VL CDR2 having the amino acid sequence of SEQ ID NO:8, or (3) a VL CDR3 having the amino acid sequence of SEQ ID NO:13, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:1, 8, and 13, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:1, 8, and 13, respectively.

In some embodiments, provided herein are antibodies or antigen-binding fragments thereof that specifically bind CEACAM1 having a VH, comprising (1) a VH CDR1 having the amino acid sequence of SEQ ID NO:20, (2) a VH CDR2 having the amino acid sequence of SEQ ID NO:28, or (3) a VH CDR3 having the amino acid sequence of SEQ ID NO:40; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:20, 28, and 40, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:20, 28, and 40, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:1, 8, and 13, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:20, 28, and 40, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:1, 8, and 13, respectively; and (b) a VH that comprises VH CDR1, VH CDR2, and VH CDR3 having the amino acid sequences of SEQ ID NOs:20, 28, and 40, respectively.

In some embodiments, the anti-CEACAM1 antibody or antigen-binding fragment thereof provided herein is a variant of 6H9. In some embodiments, provided herein are humanized 6H9. In some embodiments, the 6H9 variant can have a VL that is a variant of the VL of 6H9 having up to about 3, about 5, about 8, about 10, about 12, or about 15 amino acid substitutions, additions, and/or deletions in SEQ ID NO:61. In some embodiments, the variant can have up to about 5 amino acid substitutions, additions, and/or deletions in SEQ ID NO:61. In some embodiments, the variant can have up to about 3 amino acid substitutions, additions, and/or deletions in SEQ ID NO:61. In some embodiments, the 6H9 variant can have a VH that is a variant of the VH of 6H9 having about 3, about 5, about 8, about 10, about 12, or about 15 amino acid substitutions, additions, and/or deletions in SEQ ID NO:70. In some embodiments, the variant can have up to about 5 amino acid substitutions, additions, and/or deletions in SEQ ID NO:70. In some embodiments, the variant can have up to about 3 amino acid substitutions, additions, and/or deletions in SEQ ID NO:70. The amino acid substitutions, additions, and/or deletions can be in the VH CDRs or VL CDRs. In some embodiments, the amino acid substitutions, additions, and/or deletions are not in the CDRs. In some embodiments, the variant of 6H9 has up to about 5 conservative amino acid substitutions. In some embodiments, the variant of 6H9 has up to 3 conservative amino acid substitutions.

In some embodiments, the anti-CEACAM1 antibody or antigen-binding fragment thereof provided herein is derived from the antibody clone designated as 7D4. In some embodiments, the anti-CEACAM1 antibody or antigen-binding fragment thereof provided herein has a VL from 7D4 (SEQ ID NO:62). In some embodiments, the anti-CEACAM1 antibody or antigen-binding fragment thereof provided herein has a VH from 7D4 (SEQ ID NO:71). The anti-CEACAM1 antibody or antigen-binding fragment thereof provided herein can have both a VL and a VH from 7D4.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:62; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:71. In some embodiments, provided herein are antibodies or antigen-binding fragments thereof that specifically bind CEACAM1 comprising a VL and a VH, wherein the VL and VH have the amino acid sequences of SEQ ID NOs:62 and 71, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:62. The anti-CEACAM1 antibody or antigen-binding fragment thereof can have a VL having at least 85% sequence identity to SEQ ID NO:62. The anti-CEACAM1 antibody or antigen-binding fragment thereof can have a VL having at least 90% sequence identity to SEQ ID NO:62. The anti-CEACAM1 antibody or antigen-binding fragment thereof can have a VL having at least 95% sequence identity to SEQ ID NO:62. The anti-CEACAM1 antibody or antigen-binding fragment thereof can have a VL having at least 98% sequence identity to SEQ ID NO:62. The anti-CEACAM1 antibody or antigen-binding fragment thereof can have a VL having the amino acid sequence of SEQ ID NO:62.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:71. The anti-CEACAM1 antibody or antigen-binding fragment thereof can have a VH having at least 85% sequence identity to SEQ ID NO:71. The anti-CEACAM1 antibody or antigen-binding fragment thereof can have a VH having at least 90% sequence identity to SEQ ID NO:71. The anti-CEACAM1 antibody or antigen-binding fragment thereof can have a VH having at least 95% sequence identity to SEQ ID NO:71. The anti-CEACAM1 antibody or antigen-binding fragment thereof can have at least 98% sequence identity to SEQ ID NO:71. The anti-CEACAM1 antibody or antigen-binding fragment thereof can have a VH having the amino acid sequence of SEQ ID NO:71.

In some embodiments, the anti-CEACAM1 antibody or antigen-binding fragment thereof provided herein has a VL that comprises VL CDRs 1, 2, and 3 from the VL of 7D4 (SEQ ID NO:62). In some embodiments, the anti-CEACAM1 antibody or antigen-binding fragment thereof provided herein has a VH that comprises VH CDRs 1, 2, and 3 from the VH of 7D4 (SEQ ID NO:71). The anti-CEACAM1 antibody or antigen-binding fragment thereof provided herein can have a VL comprising VL CDRs 1, 2, and 3 from the VL of 7D4 (SEQ ID NO:62) and a VH comprising VH CDRs 1, 2, and 3 from the VH of 7D4 (SEQ ID NO:71), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:2, (2) a VL CDR2 having the amino acid sequence of SEQ ID NO:9, or (3) a VL CDR3 having the amino acid sequence of SEQ ID NO:14, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:2, 9, and 14, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:2, 9, and 14, respectively.

In some embodiments, provided herein are antibodies or antigen-binding fragments thereof that specifically bind CEACAM1 having a VH, comprising (1) a VH CDR1 having the amino acid sequence of SEQ ID NO:21, (2) a VH CDR2 having the amino acid sequence of SEQ ID NO:29, or (3) a VH CDR3 having the amino acid sequence of SEQ ID NO:41; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:21, 29, and 41, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:21, 29, and 41, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:2, 9, and 14, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:21, 29, and 41, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:2, 9, and 14, respectively; and (b) a VH that comprises VH CDR1, VH CDR2, and VH CDR3 having the amino acid sequences of SEQ ID NOs:21, 29, and 41, respectively.

In some embodiments, the anti-CEACAM1 antibody or antigen-binding fragment thereof provided herein is a variant of 7D4. In some embodiments, provided herein are humanized 7D4. In some embodiments, the 7D4 variant can have a VL that is a variant of the VL of 7D4 having up to about 3, about 5, about 8, about 10, about 12, or about 15 amino acid substitutions, additions, and/or deletions in SEQ ID NO:62. In some embodiments, the variant can have up to about 5 amino acid substitutions, additions, and/or deletions in SEQ ID NO:62. In some embodiments, the variant can have up to about 3 amino acid substitutions, additions, and/or deletions in SEQ ID NO:62. In some embodiments, the 7D4 variant can have a VH that is a variant of the VH of 7D4 having about 3, about 5, about 8, about 10, about 12, or about 15 amino acid substitutions, additions, and/or deletions in SEQ ID NO:71. In some embodiments, the variant can have up to about 5 amino acid substitutions, additions, and/or deletions in SEQ ID NO:71. In some embodiments, the variant can have up to about 3 amino acid substitutions, additions, and/or deletions in SEQ ID NO:71. The amino acid substitutions, additions, and/or deletions can be in the VH CDRs or VL CDRs. In some embodiments, the amino acid substitutions, additions, and/or deletions are not in the CDRs. In some embodiments, the variant of 7D4 has up to about 5 conservative amino acid substitutions. In some embodiments, the variant of 7D4 has up to 3 conservative amino acid substitutions.

In some embodiments, the anti-CEACAM1 antibody or antigen-binding fragment thereof provided herein is derived from the antibody clone designated as 9D12. In some embodiments, the anti-CEACAM1 antibody or antigen-binding fragment thereof provided herein has a VL from 9D12 (SEQ ID NO:63). In some embodiments, the anti-CEACAM1 antibody or antigen-binding fragment thereof provided herein has a VH from 9D12 (SEQ ID NO:72). The anti-CEACAM1 antibody or antigen-binding fragment thereof provided herein can have both a VL and a VH from 9D12.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:63; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:72. In some embodiments, provided herein are antibodies or antigen-binding fragments thereof that specifically bind CEACAM1 comprising a VL and a VH, wherein the VL and VH have the amino acid sequences of SEQ ID NOs:63 and 72, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:63. The anti-CEACAM1 antibody or antigen-binding fragment thereof can have a VL having at least 85% sequence identity to SEQ ID NO:63. The anti-CEACAM1 antibody or antigen-binding fragment thereof can have a VL having at least 90% sequence identity to SEQ ID NO:63. The anti-CEACAM1 antibody or antigen-binding fragment thereof can have a VL having at least 95% sequence identity to SEQ ID NO:63. The anti-CEACAM1 antibody or antigen-binding fragment thereof can have a VL having at least 98% sequence identity to SEQ ID NO:63. The anti-CEACAM1 antibody or antigen-binding fragment thereof can have a VL having the amino acid sequence of SEQ ID NO:63.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:72. The anti-CEACAM1 antibody or antigen-binding fragment thereof can have a VH having at least 85% sequence identity to SEQ ID NO:72. The anti-CEACAM1 antibody or antigen-binding fragment thereof can have a VH having at least 90% sequence identity to SEQ ID NO:72. The anti-CEACAM1 antibody or antigen-binding fragment thereof can have a VH having at least 95% sequence identity to SEQ ID NO:72. The anti-CEACAM1 antibody or antigen-binding fragment thereof can have at least 98% sequence identity to SEQ ID NO:72. The anti-CEACAM1 antibody or antigen-binding fragment thereof can have a VH having the amino acid sequence of SEQ ID NO:72.

In some embodiments, the anti-CEACAM1 antibody or antigen-binding fragment thereof provided herein has a VL that comprises VL CDRs 1, 2, and 3 from the VL of 9D12 (SEQ ID NO:63). In some embodiments, the anti-CEACAM1 antibody or antigen-binding fragment thereof provided herein has a VH that comprises VH CDRs 1, 2, and 3 from the VH of 9D12 (SEQ ID NO:72). The anti-CEACAM1 antibody or antigen-binding fragment thereof provided herein can have a VL comprising VL CDRs 1, 2, and 3 from the VL of 9D12 (SEQ ID NO:63) and a VH comprising VH CDRs 1, 2, and 3 from the VH of 9D12 (SEQ ID NO:72), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:3, (2) a VL CDR2 having the amino acid sequence of SEQ ID NO:9, or (3) a VL CDR3 having the amino acid sequence of SEQ ID NO:14, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:3, 9, and 14, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:3, 9, and 14, respectively.

In some embodiments, provided herein are antibodies or antigen-binding fragments thereof that specifically bind CEACAM1 having a VH, comprising (1) a VH CDR1 having the amino acid sequence of SEQ ID NO:21, (2) a VH CDR2 having the amino acid sequence of SEQ ID NO:30, or (3) a VH CDR3 having the amino acid sequence of SEQ ID NO:42; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:21, 30, and 42, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:21, 30, and 42, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:3, 9, and 14, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:21, 30, and 42, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:3, 9, and 14, respectively; and (b) a VH that comprises VH CDR1, VH CDR2, and VH CDR3 having the amino acid sequences of SEQ ID NOs:21, 30, and 42, respectively.

In some embodiments, the anti-CEACAM1 antibody or antigen-binding fragment thereof provided herein is a variant of 9D12. In some embodiments, provided herein are humanized 9D12. In some embodiments, the 9D12 variant can have a VL that is a variant of the VL of 9D12 having up to about 3, about 5, about 8, about 10, about 12, or about 15 amino acid substitutions, additions, and/or deletions in SEQ ID NO:63. In some embodiments, the variant can have up to about 5 amino acid substitutions, additions, and/or deletions in SEQ ID NO:63. In some embodiments, the variant can have up to about 3 amino acid substitutions, additions, and/or deletions in SEQ ID NO:63. In some embodiments, the 9D12 variant can have a VH that is a variant of the VH of 9D12 having about 3, about 5, about 8, about 10, about 12, or about 15 amino acid substitutions, additions, and/or deletions in SEQ ID NO:72. In some embodiments, the variant can have up to about 5 amino acid substitutions, additions, and/or deletions in SEQ ID NO:72. In some embodiments, the variant can have up to about 3 amino acid substitutions, additions, and/or deletions in SEQ ID NO:72. The amino acid substitutions, additions, and/or deletions can be in the VH CDRs or VL CDRs. In some embodiments, the amino acid substitutions, additions, and/or deletions are not in the CDRs. In some embodiments, the variant of 9D12 has up to about 5 conservative amino acid substitutions. In some embodiments, the variant of 9D12 has up to 3 conservative amino acid substitutions.

TABLE 4
Amino acid sequences of humanized VLs and VHs of 9D12

| Domain | Sequences |
|---|---|
| h9D12L0a | DIQLTQSPSFLSASVGDRVTITCSASSSVSYMHWYQQKPGKAPKLLIYEISKLA |
| | SGVPSRFSGSGSGTEFTLTISSLQPEDFATYYCQQWNYPLTFGGGTKVEIK |
| | (SEQ ID NO: 81) |
| h9D12L0b | DIQMTQSPSTLSASVGDRVTITCSASSSVSYMHWYQQKPGKAPKLLIYEISKLA |
| | SGVPSRFSGSGSGTEFTLTISSLQPDDFATYYCQQWNYPLTFGGGTKVEIK |
| | (SEQ ID NO: 82) |
| h9D12L1 | DIQLTQSPSFLSASVGDRVTITCSASSSVSYMHWYQQKPGKAPKLLIYEISKLA |
| | SGVPSRFSGSGSGTEFTLTISSMQPEDAATYYCQQWNYPLTFGGGTKVEIK |
| | (SEQ ID NO: 83) |
| h9D12L2 | DIQMTQSPSTLSASVGDRVTITCSASSSVSYMHWYQQKPGKAPKLLIYEISKLA |
| | SGVPSRFSGSGSGTEFTLTISSMQPDDAATYYCQQWNYPLTFGGGTKVEIK |
| | (SEQ ID NO: 84) |
| h9D12L3 | DIQLTQSPSFLSASVGDRVTITCSASSSVSYMHWYQQKPGKAPKLLIYEISKLA |
| | SGVPSRFSGSGSGTEFTLTISSLQPEDAATYYCQQWNYPLTFGGGTKVEIK |
| | (SEQ ID NO: 85) |
| h9D12L4 | DIQMTQSPSTLSASVGDRVTITCSASSSVSYMHWYQQKPGKAPKLLIYEISKLA |
| | SGVPSRFSGSGSGTEFTLTISSLQPDDAATYYCQQWNYPLTFGGGTKVEIK |
| | (SEQ ID NO: 86) |
| h9D12L5 | DIQMTQSPSSLSASVGDRVTITCSASSSVSYMHWYQQKPGKAPKSLIYEISKLA |
| | SGVPSRFSGSGSGTDFTLTISSMQPEDAATYYCQQWNYPLTFGQGTKLEIK |
| | (SEQ ID NO: 87) |
| h9D12L6 | EIVLTQSPATLSASPGERVTLSCSASSSVSYMHWYQQKPGQAPRLLIYEISKLA |
| | SGIPARFSGSGSGTDFTLTISSMESEDAAVYYCQQWNYPLTFGQGTKLEIK |
| | (SEQ ID NO: 88) |
| h9D12L7 | DIQLTQSPSFLSASVGDRVTITCSASSSVSYMHWYQQKPGQAPKPWIYEISKLA |
| | SGVPSRFSGSGSGTEYTLTISSMQSEDFAVYYCQQWNYPLTFGQGTKLEIK |
| | (SEQ ID NO: 89) |
| h9D12L8 | DIQLTQSPSFLSASVGDRVTITCSASSSVSYMHWYQQKPGQAPKLLIYEISKLA |
| | SGVPSRFSGSGSGTEFTLTISSMQSEDFAVYYCQQWNYPLTFGQGTKLEIK |
| | (SEQ ID NO: 90) |
| 9D12H0a | EVQLVQSGAEVKKPGATVKISCKVSGFNIKDTYMHWVQQAPGKGLEWMGRI |
| | DLASGDSKYDPKFQGRVTITADTSTDTAYMELSSLRSEDTAVYYCARAHFGG |
| | GNWYFDVWGQGTTVTVSS (SEQ ID NO: 111) |
| 9D12H0b | QVQLVQSGAEVKKPGASVKVSCKASGFNIKDTYMHWVRQAPGQGLEWMGR |
| | IDLASGDSKYDPKFQGRVTMTRDTSTSTVYMELSSLRSEDTAVYYCARAHFG |
| | GGNWYFDVWGQGTTVTVSS (SEQ ID NO: 112) |
| h9D12H1 | EVQLVQSGAEVKKPGATVKISCKASGFNIKDTYMHWVQQRPGKGLEWIGRID |
| | LASGDSKYDPKFQGRVTITADTSTDTAYMELSSLRSEDTAVYYCARAHFGGG |
| | NWYFDVWGQGTTVTVSS (SEQ ID NO: 113) |
| h9D12H2 | QVQLVQSGAEVKKPGASVKVSCKASGFNIKDTYMHWVKQRPGQGLEWIGRI |
| | DLASGDSKYDPKFQGRVTMTADTSTSTVYMELSSLRSEDTAVYYCARAHFG |
| | GGNWYFDVWGQGTTVTVSS (SEQ ID NO: 114) |
| h9D12H3 | EVQLVQSGAEVKKPGATVKISCKVSGFNIKDTYMHWVQQAPGKGLEWIGRID |
| | LASGDSKYDPKFQGRVTITADTSTDTAYMELSSLRSEDTAVYYCARAHFGGG |
| | NWYFDVWGQGTTVTVSS (SEQ ID NO: 115) |
| h9D12H4 | QVQLVQSGAEVKKPGASVKVSCKASGFNIKDTYMHWVRQAPGQGLEWIGRI |
| | DLASGDSKYDPKFQGRVTMTADTSTSTVYMELSSLRSEDTAVYYCARAHFG |
| | GGNWYFDVWGQGTTVTVSS (SEQ ID NO: 116) |

In some embodiments, provided herein are humanized 9D12. In some embodiments, provided herein are humanized anti-CEACAM1 antibodies or antigen-binding fragments thereof comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:81; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:111.

In some embodiments, provided herein are humanized anti-CEACAM1 antibodies or antigen-binding fragments thereof comprising: (a) a VL having an amino acid sequence selected from the group consisting of SEQ ID NOs:81-90; and (b) a VH having an amino acid sequence selected from the group consisting of SEQ ID NOs:111-116.

In some embodiments, provided herein are humanized anti-CEACAM1 antibodies or antigen-binding fragments thereof comprising a VL and a VH, wherein the VL and VH have the amino acid sequences of SEQ ID NOs:81 and 111, respectively. In some embodiments, VL and VH have the amino acid sequences of SEQ ID NOs:81 and 112, respectively. In some embodiments, VL and VH have the amino acid sequences of SEQ ID NOs:81 and 113, respectively. In some embodiments, VL and VH have the amino acid sequences of SEQ ID NOs:81 and 114, respectively. In some embodiments, VL and VH have the amino acid sequences of SEQ ID NOs:81 and 115, respectively. In some embodiments, VL and VH have the amino acid sequences of SEQ ID NOs:81 and 116, respectively. In some embodiments, VL and VH have the amino acid sequences of SEQ ID NOs:82 and 111, respectively. In some embodiments, VL and VH have the amino acid sequences of SEQ ID NOs:82 and 112, respectively. In some embodiments, VL and VH have the amino acid sequences of SEQ ID NOs:82 and 113, respectively. In some embodiments, VL and VH have the amino acid sequences of SEQ ID NOs:82 and 114, respectively. In some embodiments, VL and VH have the amino acid sequences of SEQ ID NOs:82 and 115, respectively. In some embodiments, VL and VH have the amino acid sequences of SEQ ID NOs:82 and 116, respectively. In some embodiments, VL and VH have the amino acid sequences of SEQ ID NOs:83 and 111, respectively. In some embodiments, VL and VH have the amino acid sequences of SEQ ID NOs:83 and 112, respectively. In some embodiments, VL and VH have the amino acid sequences of SEQ ID NOs:83 and 113, respectively. In some embodiments, VL and VH have the amino acid sequences of SEQ ID NOs:83 and 114, respectively. In some embodiments, VL and VH have the amino acid sequences of SEQ ID NOs:83 and 115, respectively. In some embodiments, VL and VH have the amino acid sequences of SEQ ID NOs:83 and 116, respectively. In some embodiments, VL and VH have the amino acid sequences of SEQ ID NOs:84 and 111, respectively. In some embodiments, VL and VH have the amino acid sequences of SEQ ID NOs:84 and 112, respectively. In some embodiments, VL and VH have the amino acid sequences of SEQ ID NOs:84 and 113, respectively. In some embodiments, VL and VH have the amino acid sequences of SEQ ID NOs:84 and 114, respectively. In some embodiments, VL and VH have the amino acid sequences of SEQ ID NOs:84 and 115, respectively. In some embodiments, VL and VH have the amino acid sequences of SEQ ID NOs:84 and 116, respectively. In some embodiments, VL and VH have the amino acid sequences of SEQ ID NOs:85 and 111, respectively. In some embodiments, VL and VH have the amino acid sequences of SEQ ID NOs:85 and 112, respectively. In some embodiments, VL and VH have the amino acid sequences of SEQ ID NOs:85 and 113, respectively. In some embodiments, VL and VH have the amino acid sequences of SEQ ID NOs:85 and 114, respectively. In some embodiments, VL and VH have the amino acid sequences of SEQ ID NOs:85 and 115, respectively. In some embodiments, VL and VH have the amino acid sequences of SEQ ID NOs:85 and 116, respectively. In some embodiments, VL and VH have the amino acid sequences of SEQ ID NOs:86 and 111, respectively. In some embodiments, VL and VH have the amino acid sequences of SEQ ID NOs:86 and 112, respectively. In some embodiments, VL and VH have the amino acid sequences of SEQ ID NOs:86 and 113, respectively. In some embodiments, VL and VH have the amino acid sequences of SEQ ID NOs:86 and 114, respectively. In some embodiments, VL and VH have the amino acid sequences of SEQ ID NOs:86 and 115, respectively. In some embodiments, VL and VH have the amino acid sequences of SEQ ID NOs:86 and 116, respectively. In some embodiments, VL and VH have the amino acid sequences of SEQ ID NOs:87 and 111, respectively. In some embodiments, VL and VH have the amino acid sequences of SEQ ID NOs:87 and 112, respectively. In some embodiments, VL and VH have the amino acid sequences of SEQ ID NOs:87 and 113, respectively. In some embodiments, VL and VH have the amino acid sequences of SEQ ID NOs:87 and 114, respectively. In some embodiments, VL and VH have the amino acid sequences of SEQ ID NOs:87 and 115, respectively. In some embodiments, VL and VH have the amino acid sequences of SEQ ID NOs:87 and 116, respectively. In some embodiments, VL and VH have the amino acid sequences of SEQ ID NOs:88 and 111, respectively. In some embodiments, VL and VH have the amino acid sequences of SEQ ID NOs:88 and 112, respectively. In some embodiments, VL and VH have the amino acid sequences of SEQ ID NOs:88 and 113, respectively. In some embodiments, VL and VH have the amino acid sequences of SEQ ID NOs:88 and 114, respectively. In some embodiments, VL and VH have the amino acid sequences of SEQ ID NOs:88 and 115, respectively. In some embodiments, VL and VH have the amino acid sequences of SEQ ID NOs:88 and 116, respectively. In some embodiments, VL and VH have the amino acid sequences of SEQ ID NOs:89 and 111, respectively. In some embodiments, VL and VH have the amino acid sequences of SEQ ID NOs:89 and 112, respectively. In some embodiments, VL and VH have the amino acid sequences of SEQ ID NOs:89 and 113, respectively. In some embodiments, VL and VH have the amino acid sequences of SEQ ID NOs:89 and 114, respectively. In some embodiments, VL and VH have the amino acid sequences of SEQ ID NOs:89 and 115, respectively. In some embodiments, VL and VH have the amino acid sequences of SEQ ID NOs:89 and 116, respectively. In some embodiments, VL and VH have the amino acid sequences of SEQ ID NOs:90 and 111, respectively. In some embodiments, VL and VH have the amino acid sequences of SEQ ID NOs:90 and 112, respectively. In some embodiments, VL and VH have the amino acid sequences of SEQ ID NOs:90 and 113, respectively. In some embodiments, VL and VH have the amino acid sequences of SEQ ID NOs:90 and 114, respectively. In some embodiments, VL and VH have the amino acid sequences of SEQ ID NOs:90 and 115, respectively. In some embodiments, VL and VH have the amino acid sequences of SEQ ID NOs:90 and 116, respectively.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:81. The humanized anti-CEACAM1 antibody or antigen-binding fragment thereof can have a VL having at least 85% sequence identity to SEQ ID NO:81. The humanized anti-CEACAM1 antibody or antigen-binding fragment thereof can have a VL having at least 90% sequence identity to SEQ ID NO:81. The humanized anti-CEACAM1 antibody or antigen-binding fragment thereof can have a VL having at least 95% sequence identity to SEQ ID NO:81. The humanized anti-CEACAM1 antibody or antigen-binding fragment thereof can have a VL having at least 98% sequence identity to SEQ ID NO:81. In some embodiments, provided herein are humanized antibodies or antigen-binding fragments thereof that specifically bind CEACAM1 comprising a VL having the amino acid sequence of SEQ ID NO:81.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:82. The humanized anti-CEACAM1 antibody or antigen-binding fragment thereof can have a VL having at least 85% sequence identity to SEQ ID NO:82. The humanized anti-CEACAM1 antibody or antigen-binding fragment thereof can have a VL having at least 90% sequence identity to SEQ ID NO:82. The humanized anti-CEACAM1 antibody or antigen-binding fragment thereof can have a VL having at least 95% sequence identity to SEQ ID NO:82. The humanized anti-CEACAM1 antibody or antigen-binding fragment thereof can have a VL having at least 98% sequence identity to SEQ ID NO:82. In some embodiments, provided herein are humanized antibodies or antigen-binding fragments thereof that specifically bind CEACAM1 comprising a VL having the amino acid sequence of SEQ ID NO:82.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:83. The humanized anti-CEACAM1 antibody or antigen-binding fragment thereof can have a VL having at least 85% sequence identity to SEQ ID NO:83. The humanized anti-CEACAM1 antibody or antigen-binding fragment thereof can have a VL having at least 90% sequence identity to SEQ ID NO:83. The humanized anti-CEACAM1 antibody or antigen-binding fragment thereof can have a VL having at least 95% sequence identity to SEQ ID NO:83. The humanized anti-CEACAM1 antibody or antigen-binding fragment thereof can have a VL having at least 98% sequence identity to SEQ ID NO:83. In some embodiments, provided herein are humanized antibodies or antigen-binding fragments thereof that specifically bind CEACAM1 comprising a VL having the amino acid sequence of SEQ ID NO:83.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:84. The humanized anti-CEACAM1 antibody or antigen-binding fragment thereof can have a VL having at least 85% sequence identity to SEQ ID NO:84. The humanized anti-CEACAM1 antibody or antigen-binding fragment thereof can have a VL having at least 90% sequence identity to SEQ ID NO:84. The humanized anti-CEACAM1 antibody or antigen-binding fragment thereof can have a VL having at least 95% sequence identity to SEQ ID NO:84. The humanized anti-CEACAM1 antibody or antigen-binding fragment thereof can have a VL having at least 98% sequence identity to SEQ ID NO:84. In some embodiments, provided herein are humanized antibodies or antigen-binding fragments thereof that specifically bind CEACAM1 comprising a VL having the amino acid sequence of SEQ ID NO:84.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:85. The humanized anti-CEACAM1 antibody or antigen-binding fragment thereof can have a VL having at least 85% sequence identity to SEQ ID NO:85. The humanized anti-CEACAM1 antibody or antigen-binding fragment thereof can have a VL having at least 90% sequence identity to SEQ ID NO:85. The humanized anti-CEACAM1 antibody or antigen-binding fragment thereof can have a VL having at least 95% sequence identity to SEQ ID NO:85. The humanized anti-CEACAM1 antibody or antigen-binding fragment thereof can have a VL having at least 98% sequence identity to SEQ ID NO:85. In some embodiments, provided herein are humanized antibodies or antigen-binding fragments thereof that specifically bind CEACAM1 comprising a VL having the amino acid sequence of SEQ ID NO:85.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:86. The humanized anti-CEACAM1 antibody or antigen-binding fragment thereof can have a VL having at least 85% sequence identity to SEQ ID NO:86. The humanized anti-CEACAM1 antibody or antigen-binding fragment thereof can have a VL having at least 90% sequence identity to SEQ ID NO:86. The humanized anti-CEACAM1 antibody or antigen-binding fragment thereof can have a VL having at least 95% sequence identity to SEQ ID NO:86. The humanized anti-CEACAM1 antibody or antigen-binding fragment thereof can have a VL having at least 98% sequence identity to SEQ ID NO:86. In some embodiments, provided herein are humanized antibodies or antigen-binding fragments thereof that specifically bind CEACAM1 comprising a VL having the amino acid sequence of SEQ ID NO:86.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:87. The humanized anti-CEACAM1 antibody or antigen-binding fragment thereof can have a VL having at least 85% sequence identity to SEQ ID NO:87. The humanized anti-CEACAM1 antibody or antigen-binding fragment thereof can have a VL having at least 90% sequence identity to SEQ ID NO:87. The humanized anti-CEACAM1 antibody or antigen-binding fragment thereof can have a VL having at least 95% sequence identity to SEQ ID NO:87. The humanized anti-CEACAM1 antibody or antigen-binding fragment thereof can have a VL having at least 98% sequence identity to SEQ ID NO:87. In some embodiments, provided herein are humanized antibodies or antigen-binding fragments thereof that specifically bind CEACAM1 comprising a VL having the amino acid sequence of SEQ ID NO:87.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:88. The humanized anti-CEACAM1 antibody or antigen-binding fragment thereof can have a VL having at least 85% sequence identity to SEQ ID NO:88. The humanized anti-CEACAM1 antibody or antigen-binding fragment thereof can have a VL having at least 90% sequence identity to SEQ ID NO:88. The humanized anti-CEACAM1 antibody or antigen-binding fragment thereof can have a VL having at least 95% sequence identity to SEQ ID NO:88. The humanized anti-CEACAM1 antibody or antigen-binding fragment thereof can have a VL having at least 98% sequence identity to SEQ ID NO:88. In some embodiments, provided herein are humanized antibodies or antigen-binding fragments thereof that specifically bind CEACAM1 comprising a VL having the amino acid sequence of SEQ ID NO:88.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:89. The humanized anti-CEACAM1 antibody or antigen-binding fragment thereof can have a VL having at least 85% sequence identity to SEQ ID NO:89. The humanized anti-CEACAM1 antibody or antigen-binding fragment thereof can have a VL having at least 90% sequence identity to SEQ ID NO:89. The humanized anti-CEACAM1 antibody or antigen-binding fragment thereof can have a VL having at least 95% sequence identity to SEQ ID NO:89. The humanized anti-CEACAM1 antibody or antigen-binding fragment thereof can have a VL having at least 98% sequence identity to SEQ ID NO:89. In some embodiments, provided herein are humanized antibodies or antigen-binding fragments thereof that specifically bind CEACAM1 comprising a VL having the amino acid sequence of SEQ ID NO:89.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:90. The humanized anti-CEACAM1 antibody or antigen-binding fragment thereof can have a VL having at least 85% sequence identity to SEQ ID NO:90. The humanized anti-CEACAM1 antibody or antigen-binding fragment thereof can have a VL having at least 90% sequence identity to SEQ ID NO:90. The humanized anti-CEACAM1 antibody or antigen-binding fragment thereof can have a VL having at least 95% sequence identity to SEQ ID NO:90. The humanized anti-CEACAM1 antibody or antigen-binding fragment thereof can have a VL having at least 98% sequence identity to SEQ ID NO:90. In some embodiments, provided herein are humanized antibodies or antigen-binding fragments thereof that specifically bind CEACAM1 comprising a VL having the amino acid sequence of SEQ ID NO:90.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 11. The humanized anti-CEACAM1 antibody or antigen-binding fragment thereof can have a VH having at least 85% sequence identity to SEQ ID NO: 11. The humanized anti-CEACAM1 antibody or antigen-binding fragment thereof can have a VH having at least 90% sequence identity to SEQ ID NO:111. The humanized anti-CEACAM1 antibody or antigen-binding fragment thereof can have a VH having at least 95% sequence identity to SEQ ID NO:111. The humanized anti-CEACAM1 antibody or antigen-binding fragment thereof can have a VH having at least 98% sequence identity to SEQ ID NO:111. In some embodiments, provided herein are humanized antibodies or antigen-binding fragments thereof that specifically bind CEACAM1 comprising a VH having the amino acid sequence of SEQ ID NO:111.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 112. The humanized anti-CEACAM1 antibody or antigen-binding fragment thereof can have a VH having at least 85% sequence identity to SEQ ID NO:112. The humanized anti-CEACAM1 antibody or antigen-binding fragment thereof can have a VH having at least 90% sequence identity to SEQ ID NO: 112. The humanized anti-CEACAM1 antibody or antigen-binding fragment thereof can have a VH having at least 95% sequence identity to SEQ ID NO: 112. The humanized anti-CEACAM1 antibody or antigen-binding fragment thereof can have a VH having at least 98% sequence identity to SEQ ID NO:112. In some embodiments, provided herein are humanized antibodies or antigen-binding fragments thereof that specifically bind CEACAM1 comprising a VH having the amino acid sequence of SEQ ID NO:112.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 113. The humanized anti-CEACAM1 antibody or antigen-binding fragment thereof can have a VH having at least 85% sequence identity to SEQ ID NO: 113. The humanized anti-CEACAM1 antibody or antigen-binding fragment thereof can have a VH having at least 90% sequence identity to SEQ ID NO: 113. The humanized anti-CEACAM1 antibody or antigen-binding fragment thereof can have a VH having at least 95% sequence identity to SEQ ID NO: 113. The humanized anti-CEACAM1 antibody or antigen-binding fragment thereof can have a VH having at least 98% sequence identity to SEQ ID NO:113. In some embodiments, provided herein are humanized antibodies or antigen-binding fragments thereof that specifically bind CEACAM1 comprising a VH having the amino acid sequence of SEQ ID NO:113.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 114. The humanized anti-CEACAM1 antibody or antigen-binding fragment thereof can have a VH having at least 85% sequence identity to SEQ ID NO: 114. The humanized anti-CEACAM1 antibody or antigen-binding fragment thereof can have a VH having at least 90% sequence identity to SEQ ID NO:114. The humanized anti-CEACAM1 antibody or antigen-binding fragment thereof can have a VH having at least 95% sequence identity to SEQ ID NO:114. The humanized anti-CEACAM1 antibody or antigen-binding fragment thereof can have a VH having at least 98% sequence identity to SEQ ID NO:114. In some embodiments, provided herein are humanized antibodies or antigen-binding fragments thereof that specifically bind CEACAM1 comprising a VH having the amino acid sequence of SEQ ID NO:114.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 115. The humanized anti-CEACAM1 antibody or antigen-binding fragment thereof can have a VH having at least 85% sequence identity to SEQ ID NO:115. The humanized anti-CEACAM1 antibody or antigen-binding fragment thereof can have a VH having at least 90% sequence identity to SEQ ID NO:115. The humanized anti-CEACAM1 antibody or antigen-binding fragment thereof can have a VH having at least 95% sequence identity to SEQ ID NO:115. The humanized anti-CEACAM1 antibody or antigen-binding fragment thereof can have a VH having at least 98% sequence identity to SEQ ID NO:115. In some embodiments, provided herein are humanized antibodies or antigen-binding fragments thereof that specifically bind CEACAM1 comprising a VH having the amino acid sequence of SEQ ID NO:115.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 116. The humanized anti-CEACAM1 antibody or antigen-binding fragment thereof can have a VH having at least 85% sequence identity to SEQ ID NO:116. The humanized anti-CEACAM1 antibody or antigen-binding fragment thereof can have a VH having at least 90% sequence identity to SEQ ID NO: 116. The humanized anti-CEACAM1 antibody or antigen-binding fragment thereof can have a VH having at least 95% sequence identity to SEQ ID NO:116. The humanized anti-CEACAM1 antibody or antigen-binding fragment thereof can have a VH having at least 98% sequence identity to SEQ ID NO:116. In some embodiments, provided herein are humanized antibodies or antigen-binding fragments thereof that specifically bind CEACAM1 comprising a VH having the amino acid sequence of SEQ ID NO:116.

In some embodiments, the anti-CEACAM1 antibody or antigen-binding fragment thereof provided herein is derived from the antibody clone designated as P23B6. In some embodiments, the anti-CEACAM1 antibody or antigen-binding fragment thereof provided herein has a VL from P23B6 (SEQ ID NO:64). In some embodiments, the anti-CEACAM1 antibody or antigen-binding fragment thereof provided herein has a VH from P23B6 (SEQ ID NO:73). The anti-CEACAM1 antibody or antigen-binding fragment thereof provided herein can have both a VL and a VH from P23B6.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:64; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:73. In some embodiments, provided herein are antibodies or antigen-binding fragments thereof that specifically bind CEACAM1 comprising a VL and a VH, wherein the VL and VH have the amino acid sequences of SEQ ID NOs:64 and 73, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:64. The anti-CEACAM1 antibody or antigen-binding fragment thereof can have a VL having at least 85% sequence identity to SEQ ID NO:64. The anti-CEACAM1 antibody or antigen-binding fragment thereof can have a VL having at least 90% sequence identity to SEQ ID NO:64. The anti-CEACAM1 antibody or antigen-binding fragment thereof can have a VL having at least 95% sequence identity to SEQ ID NO:64. The anti-CEACAM1 antibody or antigen-binding fragment thereof can have a VL having at least 98% sequence identity to SEQ ID NO:64. The anti-CEACAM1 antibody or antigen-binding fragment thereof can have a VL having the amino acid sequence of SEQ ID NO:64.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:73. The anti-CEACAM1 antibody or antigen-binding fragment thereof can have a VH having at least 85% sequence identity to SEQ ID NO:73. The anti-CEACAM1 antibody or antigen-binding fragment thereof can have a VH having at least 90% sequence identity to SEQ ID NO:73. The anti-CEACAM1 antibody or antigen-binding fragment thereof can have a VH having at least 95% sequence identity to SEQ ID NO:73. The anti-CEACAM1 antibody or antigen-binding fragment thereof can have at least 98% sequence identity to SEQ ID NO:73. The anti-CEACAM1 antibody or antigen-binding fragment thereof can have a VH having the amino acid sequence of SEQ ID NO:73.

In some embodiments, the anti-CEACAM1 antibody or antigen-binding fragment thereof provided herein has a VL that comprises VL CDRs 1, 2, and 3 from the VL of P23B6 (SEQ ID NO:64). In some embodiments, the anti-CEACAM1 antibody or antigen-binding fragment thereof provided herein has a VH that comprises VH CDRs 1, 2, and 3 from the VH of P23B6 (SEQ ID NO:73). The anti-CEACAM1 antibody or antigen-binding fragment thereof provided herein can have a VL comprising VL CDRs 1, 2, and 3 from the VL of P23B6 (SEQ ID NO:64) and a VH comprising VH CDRs 1, 2, and 3 from the VH of P23B6 (SEQ ID NO:73), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:4, (2) a VL CDR2 having the amino acid sequence of SEQ ID NO:8, or (3) a VL CDR3 having the amino acid sequence of SEQ ID NO:15, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:4, 8, and 15, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:4, 8, and 15, respectively.

In some embodiments, provided herein are antibodies or antigen-binding fragments thereof that specifically bind CEACAM1 having a VH, comprising (1) a VH CDR1 having the amino acid sequence of SEQ ID NO:22, (2) a VH CDR2 having the amino acid sequence of SEQ ID NO:31, or (3) a VH CDR3 having the amino acid sequence of SEQ ID NO:43; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:22, 31, and 43, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:22, 31, and 43, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:4, 8, and 15, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:22, 31, and 43, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:4, 8, and 15, respectively; and (b) a VH that comprises VH CDR1, VH CDR2, and VH CDR3 having the amino acid sequences of SEQ ID NOs:22, 31, and 43, respectively.

In some embodiments, the anti-CEACAM1 antibody or antigen-binding fragment thereof provided herein is a variant of P23B6. In some embodiments, provided herein are humanized P23B6. In some embodiments, the P23B6 variant can have a VL that is a variant of the VL of P23B6 having up to about 3, about 5, about 8, about 10, about 12, or about 15 amino acid substitutions, additions, and/or deletions in SEQ ID NO:64. In some embodiments, the variant can have up to about 5 amino acid substitutions, additions, and/or deletions in SEQ ID NO:64. In some embodiments, the variant can have up to about 3 amino acid substitutions, additions, and/or deletions in SEQ ID NO:64. In some embodiments, the P23B6 variant can have a VH that is a variant of the VH of P23B6 having about 3, about 5, about 8, about 10, about 12, or about 15 amino acid substitutions, additions, and/or deletions in SEQ ID NO:73. In some embodiments, the variant can have up to about 5 amino acid substitutions, additions, and/or deletions in SEQ ID NO:73. In some embodiments, the variant can have up to about 3 amino acid substitutions, additions, and/or deletions in SEQ ID NO:73. The amino acid substitutions, additions, and/or deletions can be in the VH CDRs or VL CDRs. In some embodiments, the amino acid substitutions, additions, and/or deletions are not in the CDRs. In some embodiments, the variant of P23B6 has up to about 5 conservative amino acid substitutions. In some embodiments, the variant of P23B6 has up to 3 conservative amino acid substitutions.

In some embodiments, the anti-CEACAM1 antibody or antigen-binding fragment thereof provided herein is derived from the antibody clone designated as 2C10. In some embodiments, the anti-CEACAM1 antibody or antigen-binding fragment thereof provided herein has a VL from 2C10 (SEQ ID NO:65). In some embodiments, the anti-CEACAM1 antibody or antigen-binding fragment thereof provided herein has a VH from 2C10 (SEQ ID NO: 74). The anti-CEACAM1 antibody or antigen-binding fragment thereof provided herein can have both a VL and a VH from 2C10.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:65; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:74. In some embodiments, provided herein are antibodies or antigen-binding fragments thereof that specifically bind CEACAM1 comprising a VL and a VH, wherein the VL and VH have the amino acid sequences of SEQ ID NOs:65 and 74, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:65. The anti-CEACAM1 antibody or antigen-binding fragment thereof can have a VL having at least 85% sequence identity to SEQ ID NO:65. The anti-CEACAM1 antibody or antigen-binding fragment thereof can have a VL having at least 90% sequence identity to SEQ ID NO:65. The anti-CEACAM1 antibody or antigen-binding fragment thereof can have a VL having at least 95% sequence identity to SEQ ID NO:65. The anti-CEACAM1 antibody or antigen-binding fragment thereof can have a VL having at least 98% sequence identity to SEQ ID NO:65. The anti-CEACAM1 antibody or antigen-binding fragment thereof can have a VL having the amino acid sequence of SEQ ID NO:65.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:74. The anti-CEACAM1 antibody or antigen-binding fragment thereof can have a VH having at least 85% sequence identity to SEQ ID NO:74. The anti-CEACAM1 antibody or antigen-binding fragment thereof can have a VH having at least 90% sequence identity to SEQ ID NO:74. The anti-CEACAM1 antibody or antigen-binding fragment thereof can have a VH having at least 95% sequence identity to SEQ ID NO:74. The anti-CEACAM1 antibody or antigen-binding fragment thereof can have at least 98% sequence identity to SEQ ID NO:74. The anti-CEACAM1 antibody or antigen-binding fragment thereof can have a VH having the amino acid sequence of SEQ ID NO:74.

In some embodiments, the anti-CEACAM1 antibody or antigen-binding fragment thereof provided herein has a VL that comprises VL CDRs 1, 2, and 3 from the VL of 2C10 (SEQ ID NO:65). In some embodiments, the anti-CEACAM1 antibody or antigen-binding fragment thereof provided herein has a VH that comprises VH CDRs 1, 2, and 3 from the VH of 2C10 (SEQ ID NO:74). The anti-CEACAM1 antibody or antigen-binding fragment thereof provided herein can have a VL comprising VL CDRs 1, 2, and 3 from the VL of 2C10 (SEQ ID NO:65) and a VH comprising VH CDRs 1, 2, and 3 from the VH of 2C10 (SEQ ID NO:74), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:5, (2) a VL CDR2 having the amino acid sequence of SEQ ID NO:10, or (3) a VL CDR3 having the amino acid sequence of SEQ ID NO:16, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:5, 10, and 16, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:5, 10, and 16, respectively.

In some embodiments, provided herein are antibodies or antigen-binding fragments thereof that specifically bind CEACAM1 having a VH, comprising (1) a VH CDR1 having the amino acid sequence of SEQ ID NO:23, (2) a VH CDR2 having the amino acid sequence of SEQ ID NO:32, or (3) a VH CDR3 having the amino acid sequence of SEQ ID NO:44; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:23, 32, and 44, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:23, 32, and 44, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:5, 10, and 16, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:23, 32, and 44, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:5, 10, and 16, respectively; and (b) a VH that comprises VH CDR1, VH CDR2, and VH CDR3 having the amino acid sequences of SEQ ID NOs:23, 32, and 44, respectively.

In some embodiments, the anti-CEACAM1 antibody or antigen-binding fragment thereof provided herein is a variant of 2C10. In some embodiments, provided herein are humanized 2C10. In some embodiments, the 2C10 variant can have a VL that is a variant of the VL of 2C10 having up to about 3, about 5, about 8, about 10, about 12, or about 15 amino acid substitutions, additions, and/or deletions in SEQ ID NO:65. In some embodiments, the variant can have up to about 5 amino acid substitutions, additions, and/or deletions in SEQ ID NO:65. In some embodiments, the variant can have up to about 3 amino acid substitutions, additions, and/or deletions in SEQ ID NO:65. In some embodiments, the 2C10 variant can have a VH that is a variant of the VH of 2C10 having about 3, about 5, about 8, about 10, about 12, or about 15 amino acid substitutions, additions, and/or deletions in SEQ ID NO:74. In some embodiments, the variant can have up to about 5 amino acid substitutions, additions, and/or deletions in SEQ ID NO:74. In some embodiments, the variant can have up to about 3 amino acid substitutions, additions, and/or deletions in SEQ ID NO:74. The amino acid substitutions, additions, and/or deletions can be in the VH CDRs or VL CDRs. In some embodiments, the amino acid substitutions, additions, and/or deletions are not in the CDRs. In some embodiments, the variant of 2C10 has up to about 5 conservative amino acid substitutions. In some embodiments, the variant of 2C10 has up to 3 conservative amino acid substitutions.

TABLE 5
Amino acid sequences of humanized VLs and VHs

| Domain | Sequences |
|---|---|
| h2C10L0a | DIQMTQSPSSLSASVGDRVTITCRASQDITTYLNWYQQKPGKAPKLLIYYTSRL |
| | HSGVPSRFSGSGSGTDFTFTISSLQPEDIATYYCQQGNTLPRTFGQGTKVEIK |
| | (SEQ ID NO: 91) |
| h2C10L0b | DIQMTQSPSSLSASVGDRVTITCRASQDITTYLNWYQQKPGKAPKLLIYYTSRL |
| | HSGVPSRFSGSGSGTDFTLTISSLQPEDFATYYCQQGNTLPRTFGQGTKVEIK |
| | (SEQ ID NO: 92) |
| h2C10L1 | DIQMTQSPSSLSASVGDRVTITCRASQDITTYLNWYQQKPGKAPKLLIYYTSRL |
| | HSGVPSRFSGSGSGTDFTLTISSLQPEDIATYYCQQGNTLPRTFGQGTKVEIK |
| | (SEQ ID NO: 93) |
| h2C10L2 | DIQMTQSTSSLSASVGDRVTITCRASQDITTYLNWYQQKPGGAPKLLIYYTSR |
| | LHSGVPSRFSGSGSGTDYTLTISSLQPEDIATYYCQQGNTLPRTFGGGTKLEIK |
| | (SEQ ID NO: 107) |
| h2C10L3 | DIQMTQSPSSLSASVGDRVTITCRASQDITTYLNWYQQKPGGAPKLLIYYTSRL |
| | HSGVPSRFSGSGSGTDYTLTISSLQPEDIATYYCQQGNTLPRTFGGGTKLEIK |
| | (SEQ ID NO: 108) |
| h2C10L4 | DIQMTQSPSSLSASVGDRVTITCRASQDITTYLNWYQQKPGGAPKLLIYYTSRL |
| | HSGVPSRFSGSGSGTDFTLTISSLQPEDIATYYCQQGNTLPRTFGGGTKLEIK |
| | (SEQ ID NO: 109) |
| h2C10H0a | QVQLVQSGAEVKKPGASVKVSCKASGFAFTNYLIEWVRQAPGQGLEWMGVL |
| | NPGSGATNYNEKFKGRVTMTRDTSISTAYMELSRLRSDDTAVYYCARGIYGG |
| | YFAMDYWGQGTLVTVSS (SEQ ID NO: 117) |
| h2C10H0b | QVQLVQSGAEVKKPGASVKVSCKASGFAFTNYLIEWVRQAPGQGLEWMGVL |
| | NPGSGATNYNEKFKGRVTMTRDTSTSTVYMELSSLRSEDTAVYYCARGIYGG |
| | YFAMDYWGQGTLVTVSS (SEQ ID NO: 118) |
| h2C10H1 | QVQLVQSGAEVVKPGASVKVSCKASGFAFTNYLIEWVRQAPGQGLEWIGVL |
| | NPGSGATNYNEKFKGRVTMTADTSISTAYMELSRLRSDDTAVYYCARGIYGG |
| | YFAMDYWGQGTLVTVSS (SEQ ID NO: 119) |
| h2C10H2 | QVQLVQSGAEVVKPGASVKVSCKASGFAFTNYLIEWVRQAPGQGLEWIGVL |
| | NPGSGATNYNEKFKGRVTITADTSTSTVYMELSSLRSEDTAVYYCARGIYGG |
| | YFAMDYWGQGTLVTVSS (SEQ ID NO: 120) |
| h2C10H3 | QVQLVQSGAEVKKPGASVKVSCKASGFAFTNYLIEWVRQAPGQGLEWMGVL |
| | NPGSGATNYNEKFKGRVTMTADTSISTAYMELSRLRSDDTAVYYCARGIYGG |
| | YFAMDYWGQGTLVTVSS (SEQ ID NO: 121) |
| h2C10H4 | QVQLVQSGAEVKKPGASVKVSCKASGFAFTNYLIEWVRQAPGQGLEWMGVL |
| | NPGSGATNYNEKFKGRVTMTADTSTSTVYMELSSLRSEDTAVYYCARGIYGG |
| | YFAMDYWGQGTLVTVSS (SEQ ID NO: 122) |

In some embodiments, provided herein are humanized 2C10. In some embodiments, provided herein are humanized anti-CEACAM1 antibodies or antigen-binding fragments thereof comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:91; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 117.

In some embodiments, provided herein are humanized anti-CEACAM1 antibodies or antigen-binding fragments thereof comprising: (a) a VL having an amino acid sequence selected from the group consisting of SEQ ID NOs:91-93 and 107-109; and (b) a VH having an amino acid sequence selected from the group consisting of SEQ ID NOs:117-122.

In some embodiments, provided herein are humanized anti-CEACAM1 antibodies or antigen-binding fragments thereof comprising a VL and a VH, wherein the VL and VH have the amino acid sequences of SEQ ID NOs:91 and 117, respectively. In some embodiments, VL and VH have the amino acid sequences of SEQ ID NOs:91 and 118, respectively. In some embodiments, VL and VH have the amino acid sequences of SEQ ID NOs:91 and 119, respectively. In some embodiments, VL and VH have the amino acid sequences of SEQ ID NOs:91 and 120, respectively. In some embodiments, VL and VH have the amino acid sequences of SEQ ID NOs:91 and 121, respectively. In some embodiments, VL and VH have the amino acid sequences of SEQ ID NOs:91 and 122, respectively. In some embodiments, VL and VH have the amino acid sequences of SEQ ID NOs:92 and 117, respectively. In some embodiments, VL and VH have the amino acid sequences of SEQ ID NOs:92 and 118, respectively. In some embodiments, VL and VH have the amino acid sequences of SEQ ID NOs:92 and 119, respectively. In some embodiments, VL and VH have the amino acid sequences of SEQ ID NOs:92 and 120, respectively. In some embodiments, VL and VH have the amino acid sequences of SEQ ID NOs:92 and 121, respectively. In some embodiments, VL and VH have the amino acid sequences of SEQ ID NOs:92 and 122, respectively. In some embodiments, VL and VH have the amino acid sequences of SEQ ID NOs:93 and 117, respectively. In some embodiments, VL and VH have the amino acid sequences of SEQ ID NOs:93 and 118, respectively. In some embodiments, VL and VH have the amino acid sequences of SEQ ID NOs:93 and 119, respectively. In some embodiments, VL and VH have the amino acid sequences of SEQ ID NOs:93 and 120, respectively. In some embodiments, VL and VH have the amino acid sequences of SEQ ID NOs:93 and 121, respectively. In some embodiments, VL and VH have the amino acid sequences of SEQ ID NOs:93 and 122, respectively. In some embodiments, VL and VH have the amino acid sequences of SEQ ID NOs:107 and 117, respectively. In some embodiments, VL and VH have the amino acid sequences of SEQ ID NOs:107 and 118, respectively. In some embodiments, VL and VH have the amino acid sequences of SEQ ID NOs:107 and 119, respectively. In some embodiments, VL and VH have the amino acid sequences of SEQ ID NOs:107 and 120, respectively. In some embodiments, VL and VH have the amino acid sequences of SEQ ID NOs:107 and 121, respectively. In some embodiments, VL and VH have the amino acid sequences of SEQ ID NOs:107 and 122, respectively. In some embodiments, VL and VH have the amino acid sequences of SEQ ID NOs:108 and 117, respectively. In some embodiments, VL and VH have the amino acid sequences of SEQ ID NOs:108 and 118, respectively. In some embodiments, VL and VH have the amino acid sequences of SEQ ID NOs:108 and 119, respectively. In some embodiments, VL and VH have the amino acid sequences of SEQ ID NOs:108 and 120, respectively. In some embodiments, VL and VH have the amino acid sequences of SEQ ID NOs:108 and 121, respectively. In some embodiments, VL and VH have the amino acid sequences of SEQ ID NOs:108 and 122, respectively. In some embodiments, VL and VH have the amino acid sequences of SEQ ID NOs:109 and 117, respectively. In some embodiments, VL and VH have the amino acid sequences of SEQ ID NOs:109 and 118, respectively. In some embodiments, VL and VH have the amino acid sequences of SEQ ID NOs:109 and 119, respectively. In some embodiments, VL and VH have the amino acid sequences of SEQ ID NOs:109 and 120, respectively. In some embodiments, VL and VH have the amino acid sequences of SEQ ID NOs:109 and 121, respectively. In some embodiments, VL and VH have the amino acid sequences of SEQ ID NOs:109 and 122, respectively.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:91. The humanized anti-CEACAM1 antibody or antigen-binding fragment thereof can have a VL having at least 85% sequence identity to SEQ ID NO:91. The humanized anti-CEACAM1 antibody or antigen-binding fragment thereof can have a VL having at least 90% sequence identity to SEQ ID NO:91. The humanized anti-CEACAM1 antibody or antigen-binding fragment thereof can have a VL having at least 95% sequence identity to SEQ ID NO:91. The humanized anti-CEACAM1 antibody or antigen-binding fragment thereof can have a VL having at least 98% sequence identity to SEQ ID NO:91. In some embodiments, provided herein are humanized antibodies or antigen-binding fragments thereof that specifically bind CEACAM1 comprising a VL having the amino acid sequence of SEQ ID NO:91.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:92. The humanized anti-CEACAM1 antibody or antigen-binding fragment thereof can have a VL having at least 85% sequence identity to SEQ ID NO:92. The humanized anti-CEACAM1 antibody or antigen-binding fragment thereof can have a VL having at least 90% sequence identity to SEQ ID NO:92. The humanized anti-CEACAM1 antibody or antigen-binding fragment thereof can have a VL having at least 95% sequence identity to SEQ ID NO:92. The humanized anti-CEACAM1 antibody or antigen-binding fragment thereof can have a VL having at least 98% sequence identity to SEQ ID NO:92. In some embodiments, provided herein are humanized antibodies or antigen-binding fragments thereof that specifically bind CEACAM1 comprising a VL having the amino acid sequence of SEQ ID NO:92.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:93. The humanized anti-CEACAM1 antibody or antigen-binding fragment thereof can have a VL having at least 85% sequence identity to SEQ ID NO:93. The humanized anti-CEACAM1 antibody or antigen-binding fragment thereof can have a VL having at least 90% sequence identity to SEQ ID NO:93. The humanized anti-CEACAM1 antibody or antigen-binding fragment thereof can have a VL having at least 95% sequence identity to SEQ ID NO:93. The humanized anti-CEACAM1 antibody or antigen-binding fragment thereof can have a VL having at least 98% sequence identity to SEQ ID NO:93. In some embodiments, provided herein are humanized antibodies or antigen-binding fragments thereof that specifically bind CEACAM1 comprising a VL having the amino acid sequence of SEQ ID NO:93.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:107. The humanized anti-CEACAM1 antibody or antigen-binding fragment thereof can have a VL having at least 85% sequence identity to SEQ ID NO:107. The humanized anti-CEACAM1 antibody or antigen-binding fragment thereof can have a VL having at least 90% sequence identity to SEQ ID NO:107. The humanized anti-CEACAM1 antibody or antigen-binding fragment thereof can have a VL having at least 95% sequence identity to SEQ ID NO:107. The humanized anti-CEACAM1 antibody or antigen-binding fragment thereof can have a VL having at least 98% sequence identity to SEQ ID NO:107. In some embodiments, provided herein are humanized antibodies or antigen-binding fragments thereof that specifically bind CEACAM1 comprising a VL having the amino acid sequence of SEQ ID NO:107.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:108. The humanized anti-CEACAM1 antibody or antigen-binding fragment thereof can have a VL having at least 85% sequence identity to SEQ ID NO:108. The humanized anti-CEACAM1 antibody or antigen-binding fragment thereof can have a VL having at least 90% sequence identity to SEQ ID NO:108. The humanized anti-CEACAM1 antibody or antigen-binding fragment thereof can have a VL having at least 95% sequence identity to SEQ ID NO:108. The humanized anti-CEACAM1 antibody or antigen-binding fragment thereof can have a VL having at least 98% sequence identity to SEQ ID NO:108. In some embodiments, provided herein are humanized antibodies or antigen-binding fragments thereof that specifically bind CEACAM1 comprising a VL having the amino acid sequence of SEQ ID NO:108.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:109. The humanized anti-CEACAM1 antibody or antigen-binding fragment thereof can have a VL having at least 85% sequence identity to SEQ ID NO:109. The humanized anti-CEACAM1 antibody or antigen-binding fragment thereof can have a VL having at least 90% sequence identity to SEQ ID NO:109. The humanized anti-CEACAM1 antibody or antigen-binding fragment thereof can have a VL having at least 95% sequence identity to SEQ ID NO:109. The humanized anti-CEACAM1 antibody or antigen-binding fragment thereof can have a VL having at least 98% sequence identity to SEQ ID NO:109. In some embodiments, provided herein are humanized antibodies or antigen-binding fragments thereof that specifically bind CEACAM1 comprising a VL having the amino acid sequence of SEQ ID NO:109.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 117. The humanized anti-CEACAM1 antibody or antigen-binding fragment thereof can have a VH having at least 85% sequence identity to SEQ ID NO: 117. The humanized anti-CEACAM1 antibody or antigen-binding fragment thereof can have a VH having at least 90% sequence identity to SEQ ID NO:117. The humanized anti-CEACAM1 antibody or antigen-binding fragment thereof can have a VH having at least 95% sequence identity to SEQ ID NO:117. The humanized anti-CEACAM1 antibody or antigen-binding fragment thereof can have a VH having at least 98% sequence identity to SEQ ID NO:117. In some embodiments, provided herein are humanized antibodies or antigen-binding fragments thereof that specifically bind CEACAM1 comprising a VH having the amino acid sequence of SEQ ID NO:117.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:118. The humanized anti-CEACAM1 antibody or antigen-binding fragment thereof can have a VH having at least 85% sequence identity to SEQ ID NO:118. The humanized anti-CEACAM1 antibody or antigen-binding fragment thereof can have a VH having at least 90% sequence identity to SEQ ID NO:118. The humanized anti-CEACAM1 antibody or antigen-binding fragment thereof can have a VH having at least 95% sequence identity to SEQ ID NO:118. The humanized anti-CEACAM1 antibody or antigen-binding fragment thereof can have a VH having at least 98% sequence identity to SEQ ID NO:118. In some embodiments, provided herein are humanized antibodies or antigen-binding fragments thereof that specifically bind CEACAM1 comprising a VH having the amino acid sequence of SEQ ID NO:118.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 119. The humanized anti-CEACAM1 antibody or antigen-binding fragment thereof can have a VH having at least 85% sequence identity to SEQ ID NO:119. The humanized anti-CEACAM1 antibody or antigen-binding fragment thereof can have a VH having at least 90% sequence identity to SEQ ID NO:119. The humanized anti-CEACAM1 antibody or antigen-binding fragment thereof can have a VH having at least 95% sequence identity to SEQ ID NO:119. The humanized anti-CEACAM1 antibody or antigen-binding fragment thereof can have a VH having at least 98% sequence identity to SEQ ID NO:119. In some embodiments, provided herein are humanized antibodies or antigen-binding fragments thereof that specifically bind CEACAM1 comprising a VH having the amino acid sequence of SEQ ID NO:119.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:120. The humanized anti-CEACAM1 antibody or antigen-binding fragment thereof can have a VH having at least 85% sequence identity to SEQ ID NO:120. The humanized anti-CEACAM1 antibody or antigen-binding fragment thereof can have a VH having at least 90% sequence identity to SEQ ID NO:120. The humanized anti-CEACAM1 antibody or antigen-binding fragment thereof can have a VH having at least 95% sequence identity to SEQ ID NO:120. The humanized anti-CEACAM1 antibody or antigen-binding fragment thereof can have a VH having at least 98% sequence identity to SEQ ID NO:120. In some embodiments, provided herein are humanized antibodies or antigen-binding fragments thereof that specifically bind CEACAM1 comprising a VH having the amino acid sequence of SEQ ID NO:120.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:121. The humanized anti-CEACAM1 antibody or antigen-binding fragment thereof can have a VH having at least 85% sequence identity to SEQ ID NO:121. The humanized anti-CEACAM1 antibody or antigen-binding fragment thereof can have a VH having at least 90% sequence identity to SEQ ID NO:121. The humanized anti-CEACAM1 antibody or antigen-binding fragment thereof can have a VH having at least 95% sequence identity to SEQ ID NO:121. The humanized anti-CEACAM1 antibody or antigen-binding fragment thereof can have a VH having at least 98% sequence identity to SEQ ID NO:121. In some embodiments, provided herein are humanized antibodies or antigen-binding fragments thereof that specifically bind CEACAM1 comprising a VH having the amino acid sequence of SEQ ID NO:121.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:122. The humanized anti-CEACAM1 antibody or antigen-binding fragment thereof can have a VH having at least 85% sequence identity to SEQ ID NO:122. The humanized anti-CEACAM1 antibody or antigen-binding fragment thereof can have a VH having at least 90% sequence identity to SEQ ID NO:122. The humanized anti-CEACAM1 antibody or antigen-binding fragment thereof can have a VH having at least 95% sequence identity to SEQ ID NO:122. The humanized anti-CEACAM1 antibody or antigen-binding fragment thereof can have a VH having at least 98% sequence identity to SEQ ID NO:122. In some embodiments, provided herein are humanized antibodies or antigen-binding fragments thereof that specifically bind CEACAM1 comprising a VH having the amino acid sequence of SEQ ID NO:122.

In some embodiments, the anti-CEACAM1 antibody or antigen-binding fragment thereof provided herein is derived from the antibody clone designated as 4D10. In some embodiments, the anti-CEACAM1 antibody or antigen-binding fragment thereof provided herein has a VL from 4D10 (SEQ ID NO:66). In some embodiments, the anti-CEACAM1 antibody or antigen-binding fragment thereof provided herein has a VH from 4D10 (SEQ ID NO:75). The anti-CEACAM1 antibody or antigen-binding fragment thereof provided herein can have both a VL and a VH from 4D10.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:66; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:75. In some embodiments, provided herein are antibodies or antigen-binding fragments thereof that specifically bind CEACAM1 comprising a VL and a VH, wherein the VL and VH have the amino acid sequences of SEQ ID NOs:66 and 75, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:66. The anti-CEACAM1 antibody or antigen-binding fragment thereof can have a VL having at least 85% sequence identity to SEQ ID NO:66. The anti-CEACAM1 antibody or antigen-binding fragment thereof can have a VL having at least 90% sequence identity to SEQ ID NO:66. The anti-CEACAM1 antibody or antigen-binding fragment thereof can have a VL having at least 95% sequence identity to SEQ ID NO:66. The anti-CEACAM1 antibody or antigen-binding fragment thereof can have a VL having at least 98% sequence identity to SEQ ID NO:66. The anti-CEACAM1 antibody or antigen-binding fragment thereof can have a VL having the amino acid sequence of SEQ ID NO:66.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:75. The anti-CEACAM1 antibody or antigen-binding fragment thereof can have a VH having at least 85% sequence identity to SEQ ID NO:75. The anti-CEACAM1 antibody or antigen-binding fragment thereof can have a VH having at least 90% sequence identity to SEQ ID NO:75. The anti-CEACAM1 antibody or antigen-binding fragment thereof can have a VH having at least 95% sequence identity to SEQ ID NO:75. The anti-CEACAM1 antibody or antigen-binding fragment thereof can have at least 98% sequence identity to SEQ ID NO:75. The anti-CEACAM1 antibody or antigen-binding fragment thereof can have a VH having the amino acid sequence of SEQ ID NO:75.

In some embodiments, the anti-CEACAM1 antibody or antigen-binding fragment thereof provided herein has a VL that comprises VL CDRs 1, 2, and 3 from the VL of 4D10 (SEQ ID NO:66). In some embodiments, the anti-CEACAM1 antibody or antigen-binding fragment thereof provided herein has a VH that comprises VH CDRs 1, 2, and 3 from the VH of 4D10 (SEQ ID NO:75). The anti-CEACAM1 antibody or antigen-binding fragment thereof provided herein can have a VL comprising VL CDRs 1, 2, and 3 from the VL of 4D10 (SEQ ID NO:66) and a VH comprising VH CDRs 1, 2, and 3 from the VH of 4D10 (SEQ ID NO:75), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:3, (2) a VL CDR2 having the amino acid sequence of SEQ ID NO:9, or (3) a VL CDR3 having the amino acid sequence of SEQ ID NO:17, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:3, 9, and 17, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:3, 9, and 17, respectively.

In some embodiments, provided herein are antibodies or antigen-binding fragments thereof that specifically bind CEACAM1 having a VL, wherein the VL comprises VL CDR1, CDR2 and CDR3 having the amino acid sequences of SEQ ID NOs:3, 9, and 17 or a variant thereof having up to about 3, about 5, about 8, about 10, about 12, or about 15 amino acid substitutions, additions, and/or deletions in the VL CDRs. In some embodiments, the VL CDR1 can have the amino acid sequence of SEQ ID NO:3 with a substitution at S5 (which corresponds to S28 in the VL). In some embodiments, the VL CDR1 can have the amino acid sequence of SEQ ID NO:3 with a substitution selected from the group consisting of S5F and S5H. In some embodiments, the VL CDR2 can have the amino acid sequence of SEQ ID NO:9 with a substitution at S3 (which corresponds to S51 in the VL). In some embodiments, the VL CDR2 can have the amino acid sequence of SEQ ID NO:9 with the substitution of S3M. In some embodiments, the VL CDR2 can have the amino acid sequence of SEQ ID NO:9 with a substitution at K4 (which corresponds to K52 in the VL). In some embodiments, the VL CDR2 can have the amino acid sequence of SEQ ID NO:9 with the substitution of K4R.

In some embodiments, provided herein are antibodies or antigen-binding fragments thereof that specifically bind CEACAM1 having a VL, wherein the VL comprises (1) a VL CDR1 having an amino acid sequence selected from the group consisting of SEQ ID NOs:3 and 52-53; (2) a VL CDR2 having an amino acid sequence selected from the group consisting of SEQ ID NOs:9 and 54-56; and (3) a VL CDR3 having the amino acid sequence of SEQ ID NO:17.

In some embodiments, provided herein are antibodies or antigen-binding fragments thereof that specifically bind CEACAM1 having a VL, wherein the VL comprises VL CDR1, CDR2 and CDR3 having the amino acid sequences of (1) SEQ ID NOs:3, 9, and 17, respectively; (2) SEQ ID NOs:3, 54, and 17, respectively; (3) SEQ ID NOs:3, 55, and 17, respectively; (4) SEQ ID NOs:3, 56, and 17, respectively; (5) SEQ ID NOs:52, 9, and 17, respectively; (6) SEQ ID NOs:52, 54, and 17, respectively; (7) SEQ ID NOs:52, 55, and 17, respectively; (8) SEQ ID NOs:52, 56, and 17, respectively; (9) SEQ ID NOs:53, 9, and 17, respectively; (10) SEQ ID NOs:53, 54, and 17, respectively; (11) SEQ ID NOs:53, 55, and 17, respectively; or (12) SEQ ID NOs:53, 56, and 17, respectively, or a variant thereof having up to about 3 amino acid substitutions, additions, and/or deletions in the VL CDRs.

In some embodiments, provided herein are antibodies or antigen-binding fragments thereof that specifically bind CEACAM1 having a VH, comprising (1) a VH CDR1 having the amino acid sequence of SEQ ID NO:24, (2) a VH CDR2 having the amino acid sequence of SEQ ID NO:33, or (3) a VH CDR3 having the amino acid sequence of SEQ ID NO:45; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:24, 33, and 45, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:24, 33, and 45, respectively.

In some embodiments, provided herein are antibodies or antigen-binding fragments thereof that specifically bind CEACAM1 having a VH, wherein the VH comprises VH CDR1, CDR2 and CDR3 having the amino acid sequences of SEQ ID NOs:24, 33, and 45 or a variant thereof having up to about 3, about 5, about 8, about 10, about 12, or about 15 amino acid substitutions, additions, and/or deletions in the VH CDRs. In some embodiments, the VH CDR1 can have the amino acid sequence of SEQ ID NO:24 with a substitution at H5 (which corresponds to H35 in the VH). In some embodiments, the VH CDR1 can have the amino acid sequence of SEQ ID NO:24 with a substitution of H5M. In some embodiments, the VH CDR2 can have the amino acid sequence of SEQ ID NO:33 with a substitution at N7 (which corresponds to N56 in the VH). In some embodiments, the VH CDR2 can have the amino acid sequence of SEQ ID NO:33 with a substitution selected from the group consisting of N7I, N7Q, N7V, and N7W. In some embodiments, the VH CDR3 can have the amino acid sequence of SEQ ID NO:45 with a substitution at Y2 (which corresponds to Y100 in the VH). In some embodiments, the VH CDR3 can have the amino acid sequence of SEQ ID NO:45 with a substitution selected from the group consisting of Y2F, Y2V, and Y2W.

In some embodiments, provided herein are antibodies or antigen-binding fragments thereof that specifically bind CEACAM1 having a VH, wherein the VH comprises (1) a VH CDR1 having an amino acid sequence selected from the group consisting of SEQ ID NOs:24 and 57; (2) a VH CDR2 having an amino acid sequence selected from the group consisting of SEQ ID NOs:33-37; and (3) a VH CDR3 having an amino acid sequence selected from the group consisting of SEQ ID NOs:45-48.

In some embodiments, the anti-human CEACAM1 antibodies or antigen-binding fragments thereof have a VH comprising VH CDR1, VH CDR2, and VH CDR3 having the amino acid sequences of (1) SEQ ID NOs:24, 33, and 45, respectively; (2) SEQ ID NOs:24, 33, and 46, respectively; (3) SEQ ID NOs:24, 33, and 47, respectively; (4) SEQ ID NOs:24, 33, and 48, respectively; (5) SEQ ID NOs:24, 34, and 45, respectively; (6) SEQ ID NOs:24, 34, and 46, respectively; (7) SEQ ID NOs:24, 34, and 47, respectively; (8) SEQ ID NOs:24, 34, and 48, respectively; (9) SEQ ID NOs:24, 35, and 45, respectively; (10) SEQ ID NOs:24, 35, and 46, respectively; (11) SEQ ID NOs:24, 35, and 47, respectively; (12) SEQ ID NOs:24, 35, and 48, respectively; (13) SEQ ID NOs:24, 36, and 45, respectively; (14) SEQ ID NOs:24, 36, and 46, respectively; (15) SEQ ID NOs:24, 36, and 47, respectively; (16) SEQ ID NOs:24, 36, and 48, respectively; (17) SEQ ID NOs:24, 37, and 45, respectively; (18) SEQ ID NOs:24, 37, and 46, respectively; (19) SEQ ID NOs:24, 37, and 47, respectively; (20) SEQ ID NOs:24, 37, and 48, respectively; (21) SEQ ID NOs:57, 33, and 45, respectively; (22) SEQ ID NOs:57, 33, and 46, respectively; (23) SEQ ID NOs:57, 33, and 47, respectively; (24) SEQ ID NOs:57, 33, and 48, respectively; (25) SEQ ID NOs:57, 34, and 45, respectively; (26) SEQ ID NOs:57, 34, and 46, respectively; (27) SEQ ID NOs:57, 34, and 47, respectively; (28) SEQ ID NOs:57, 34, and 48, respectively; (29) SEQ ID NOs:57, 35, and 45, respectively; (30) SEQ ID NOs:57, 35, and 46, respectively; (31) SEQ ID NOs:57, 35, and 47, respectively; (32) SEQ ID NOs:57, 35, and 48, respectively; (33) SEQ ID NOs:57, 36, and 45, respectively; (34) SEQ ID NOs:57, 36, and 46, respectively; (35) SEQ ID NOs:57, 36, and 47, respectively; (36) SEQ ID NOs:57, 36, and 48, respectively; (37) SEQ ID NOs:57, 37, and 45, respectively; (38) SEQ ID NOs:57, 37, and 46, respectively; (39) SEQ ID NOs:57, 37, and 47, respectively; or (40) SEQ ID NOs:57, 37, and 48, respectively, or a variant thereof having up to about 3 amino acid substitutions, additions, and/or deletions in the VH CDRs.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:3, 9, and 17, respectively; (2) SEQ ID NOs:3, 54, and 17, respectively; (3) SEQ ID NOs:3, 55, and 17, respectively; (4) SEQ ID NOs:3, 56, and 17, respectively; (5) SEQ ID NOs:52, 9, and 17, respectively; (6) SEQ ID NOs:52, 54, and 17, respectively; (7) SEQ ID NOs:52, 55, and 17, respectively; (8) SEQ ID NOs:52, 56, and 17, respectively; (9) SEQ ID NOs:53, 9, and 17, respectively; (10) SEQ ID NOs:53, 54, and 17, respectively; (11) SEQ ID NOs:53, 55, and 17, respectively; (12) SEQ ID NOs:53, 56, and 17, respectively; or a variant thereof having up to about 3 amino acid substitutions, additions, and/or deletions in the VL CDRs and (b) a VH that comprises VH CDR1, VH CDR2, and VH CDR3 having the amino acid sequences of (1) SEQ ID NOs:24, 33, and 45, respectively; (2) SEQ ID NOs:24, 33, and 46, respectively; (3) SEQ ID NOs:24, 33, and 47, respectively; (4) SEQ ID NOs:24, 33, and 48, respectively; (5) SEQ ID NOs:24, 34, and 45, respectively; (6) SEQ ID NOs:24, 34, and 46, respectively; (7) SEQ ID NOs:24, 34, and 47, respectively; (8) SEQ ID NOs:24, 34, and 48, respectively; (9) SEQ ID NOs:24, 35, and 45, respectively; (10) SEQ ID NOs:24, 35, and 46, respectively; (11) SEQ ID NOs:24, 35, and 47, respectively; (12) SEQ ID NOs:24, 35, and 48, respectively; (13) SEQ ID NOs:24, 36, and 45, respectively; (14) SEQ ID NOs:24, 36, and 46, respectively; (15) SEQ ID NOs:24, 36, and 47, respectively; (16) SEQ ID NOs:24, 36, and 48, respectively; (17) SEQ ID NOs:24, 37, and 45, respectively; (18) SEQ ID NOs:24, 37, and 46, respectively; (19) SEQ ID NOs:24, 37, and 47, respectively; (20) SEQ ID NOs:24, 37, and 48, respectively; (21) SEQ ID NOs:57, 33, and 45, respectively; (22) SEQ ID NOs:57, 33, and 46, respectively; (23) SEQ ID NOs:57, 33, and 47, respectively; (24) SEQ ID NOs:57, 33, and 48, respectively; (25) SEQ ID NOs:57, 34, and 45, respectively; (26) SEQ ID NOs:57, 34, and 46, respectively; (27) SEQ ID NOs:57, 34, and 47, respectively; (28) SEQ ID NOs:57, 34, and 48, respectively; (29) SEQ ID NOs:57, 35, and 45, respectively; (30) SEQ ID NOs:57, 35, and 46, respectively; (31) SEQ ID NOs:57, 35, and 47, respectively; (32) SEQ ID NOs:57, 35, and 48, respectively; (33) SEQ ID NOs:57, 36, and 45, respectively; (34) SEQ ID NOs:57, 36, and 46, respectively; (35) SEQ ID NOs:57, 36, and 47, respectively; (36) SEQ ID NOs:57, 36, and 48, respectively; (37) SEQ ID NOs:57, 37, and 45, respectively; (38) SEQ ID NOs:57, 37, and 46, respectively; (39) SEQ ID NOs:57, 37, and 47, respectively; or (40) SEQ ID NOs:57, 37, and 48, respectively; or a variant thereof having up to about 3 amino acid substitutions, additions, and/or deletions in the VH CDRs.

In some embodiments, the anti-CEACAM1 antibody or antigen-binding fragment thereof provided herein is a variant of 4D10. In some embodiments, provided herein are humanized 4D10. In some embodiments, the 4D10 variant can have a VL that is a variant of the VL of 4D10 having up to about 3, about 5, about 8, about 10, about 12, or about 15 amino acid substitutions, additions, and/or deletions in SEQ ID NO:66. In some embodiments, the variant can have up to about 5 amino acid substitutions, additions, and/or deletions in SEQ ID NO:66. In some embodiments, the variant can have up to about 3 amino acid substitutions, additions, and/or deletions in SEQ ID NO:66. In some embodiments, the 4D10 variant can have a VH that is a variant of the VH of 4D10 having about 3, about 5, about 8, about 10, about 12, or about 15 amino acid substitutions, additions, and/or deletions in SEQ ID NO:75. In some embodiments, the variant can have up to about 5 amino acid substitutions, additions, and/or deletions in SEQ ID NO:75. In some embodiments, the variant can have up to about 3 amino acid substitutions, additions, and/or deletions in SEQ ID NO:75. The amino acid substitutions, additions, and/or deletions can be in the VH CDRs or VL CDRs. In some embodiments, the amino acid substitutions, additions, and/or deletions are not in the CDRs. In some embodiments, the variant of 4D10 has up to about 5 conservative amino acid substitutions. In some embodiments, the variant of 4D10 has up to 3 conservative amino acid substitutions.

In some embodiments, the VL can have the amino acid sequence of SEQ ID NO:66 with a substitution at S28. The substitution can be S28F, or S28H. In some embodiments, the VL can have the amino acid sequence of SEQ ID NO:66 with a substitution at S51. The substitution can be S51M. In some embodiments, the VL can have the amino acid sequence of SEQ ID NO:66 with a substitution at K52. The substitution can be K52R. In some embodiments, the VL can have the amino acid sequence of SEQ ID NO:66 with substitutions at S28, S51 and/or K52. The VL can have the amino acid sequence of SEQ ID NO:66 with (1) S28F and S51M substitutions, (2) S28F and K52R substitutions, (3) S28H and S51M substitutions, (4) S28H and K52R substitutions, (5) S51M and K52R substitutions, (6) S28F, S51M and K52R substitutions, or (7) S28H, S51M and K52R substitutions. The VL can have the amino acid sequence of SEQ ID NO:66 with S28F substitution. The VL can have the amino acid sequence of SEQ ID NO:66 with S28H substitution. The VL can have the amino acid sequence of SEQ ID NO:66 with S51M substitution. The VL can have the amino acid sequence of SEQ ID NO:66 with K52R substitution. The VL can have the amino acid sequence of SEQ ID NO:66 with S28F and S51M substitutions. The VL can have the amino acid sequence of SEQ ID NO:66 with S28F and K52R substitutions. The VL can have the amino acid sequence of SEQ ID NO:66 with S28H and S51M substitutions. The VL can have the amino acid sequence of SEQ ID NO:66 with S28H and K52R substitutions. The VL can have the amino acid sequence of SEQ ID NO:66 with S51M and K52R substitutions. The VL can have the amino acid sequence of SEQ ID NO:66 with S28F, S51M and K52R substitutions. The VL can have the amino acid sequence of SEQ ID NO:66 with S28H, S51M and K52R substitutions.

In some embodiments, the VH can have the amino acid sequence of SEQ ID NO:75 with a substitution at H35. The substitution can be H35M. In some embodiments, the VH can have the amino acid sequence of SEQ ID NO:75 with a substitution at N56. The substitution can be N56I, N56Q, N56V, or N56W. In some embodiments, the VH can have the amino acid sequence of SEQ ID NO:75 with a substitution at Y100. The substitution can be Y100F, Y100V, or Y100W. In some embodiments, the VH can have the amino acid sequence of SEQ ID NO:75 with substitutions at H35, N56 and/or Y100. The VH can have the amino acid sequence of SEQ ID NO:75 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions. The VH can have the amino acid sequence of SEQ ID NO:75 with H35M substitution. The VH can have the amino acid sequence of SEQ ID NO:75 with N56I substitution. The VH can have the amino acid sequence of SEQ ID NO:75 with N56Q substitution. The VH can have the amino acid sequence of SEQ ID NO:75 with N56V substitution. The VH can have the amino acid sequence of SEQ ID NO:75 with N56W substitution. The VH can have the amino acid sequence of SEQ ID NO:75 with Y100F substitution. The VH can have the amino acid sequence of SEQ ID NO:75 with Y100V substitution. The VH can have the amino acid sequence of SEQ ID NO:75 with Y100W substitution. The VH can have the amino acid sequence of SEQ ID NO:75 with H35M and N56I substitutions. The VH can have the amino acid sequence of SEQ ID NO:75 with H35M and N56Q substitutions. The VH can have the amino acid sequence of SEQ ID NO:75 with H35M and N56V substitutions. The VH can have the amino acid sequence of SEQ ID NO:75 with H35M and N56W substitutions. The VH can have the amino acid sequence of SEQ ID NO:75 with H35M and Y100F substitutions. The VH can have the amino acid sequence of SEQ ID NO:75 with H35M and Y100V substitutions. The VH can have the amino acid sequence of SEQ ID NO:75 with H35M and Y100W substitutions. The VH can have the amino acid sequence of SEQ ID NO:75 with N56I and Y100F substitutions. The VH can have the amino acid sequence of SEQ ID NO:75 with N56I and Y100V substitutions. The VH can have the amino acid sequence of SEQ ID NO:75 with N56I and Y100W substitutions. The VH can have the amino acid sequence of SEQ ID NO:75 with N56Q and Y100F substitutions. The VH can have the amino acid sequence of SEQ ID NO:75 with N56Q and Y100V substitutions. The VH can have the amino acid sequence of SEQ ID NO:75 with N56Q and Y100W substitutions. The VH can have the amino acid sequence of SEQ ID NO:75 with N56V and Y100F substitutions. The VH can have the amino acid sequence of SEQ ID NO:75 with N56V and Y100V substitutions. The VH can have the amino acid sequence of SEQ ID NO:75 with N56V and Y100W substitutions. The VH can have the amino acid sequence of SEQ ID NO:75 with N56W and Y100F substitutions. The VH can have the amino acid sequence of SEQ ID NO:75 with N56W and Y100V substitutions. The VH can have the amino acid sequence of SEQ ID NO:75 with N56W and Y100W substitutions. The VH can have the amino acid sequence of SEQ ID NO:75 with H35M, N56I and Y100F substitutions. The VH can have the amino acid sequence of SEQ ID NO:75 with H35M, N56I and Y100V substitutions. The VH can have the amino acid sequence of SEQ ID NO:75 with H35M, N56I and Y100W substitutions. The VH can have the amino acid sequence of SEQ ID NO:75 with H35M, N56Q and Y100F substitutions. The VH can have the amino acid sequence of SEQ ID NO:75 with H35M, N56Q and Y100V substitutions. The VH can have the amino acid sequence of SEQ ID NO:75 with H35M, N56Q and Y100W substitutions. The VH can have the amino acid sequence of SEQ ID NO:75 with H35M, N56V and Y100F substitutions. The VH can have the amino acid sequence of SEQ ID NO:75 with H35M, N56V and Y100V substitutions. The VH can have the amino acid sequence of SEQ ID NO:75 with H35M, N56V and Y100W substitutions. The VH can have the amino acid sequence of SEQ ID NO:75 with H35M, N56W and Y100F substitutions. The VH can have the amino acid sequence of SEQ ID NO:75 with H35M, N56W and Y100V substitutions. The VH can have the amino acid sequence of SEQ ID NO:75 with H35M, N56W and Y100W substitutions.

TABLE 6
Amino acid sequences of humanized VLs and VHs of 4D10

| Domain | Sequences |
|---|---|
| h4D10L0a | DIQLTQSPSFLSASVGDRVTITCSASSSVSYMHWYQQKPGKAPKLLIYEISKLASGV |
| | PSRFSGSGSGTEFTLTISSLQPEDFATYYCQQWNYPLITFGGGTKVEIK (SEQ ID |
| | NO: 94) |
| h4D10L0b | DIQMTQSPSTLSASVGDRVTITCSASSSVSYMHWYQQKPGKAPKLLIYEISKLASG |
| | VPSRFSGSGSGTEFTLTISSLQPDDFATYYCQQWNYPLITFGGGTKVEIK (SEQ ID |
| | NO: 95) |
| h4D10L1 | DIQLTQSPSFLSASVGDRVTITCSASSSVSYMHWYQQKPGKAPKPLIYEISKLASGV |
| | PSRFSGSGSGTEFTLTISSLQPEDAATYYCQQWNYPLITFGGGTKVEIK (SEQ ID |
| | NO: 96) |
| h4D10L2 | DIQMTQSPSTLSASVGDRVTITCSASSSVSYMHWYQQKPGKAPKPLIYEISKLASG |
| | VPSRFSGSGSGTEFTLTISSLQPDDAATYYCQQWNYPLITFGGGTKVEIK (SEQ ID |
| | NO: 97) |
| h4D10L3 | DIQLTQSPSFLSASVGDRVTITCSASSSVSYMHWYQQKPGKAPKLLIYEISKLASGV |
| | PSRFSGSGSGTEFTLTISSLQPEDAATYYCQQWNYPLITFGGGTKVEIK (SEQ ID |
| | NO: 98) |
| h4D10L4 | DIQMTQSPSTLSASVGDRVTITCSASSSVSYMHWYQQKPGKAPKLLIYEISKLASG |
| | VPSRFSGSGSGTEFTLTISSLQPDDAATYYCQQWNYPLITFGGGTKVEIK (SEQ ID |
| | NO: 99) |
| h4D10L2- | DIQMTQSPSTLSASVGDRVTITCSASSEVSYMHWYQQKPGKAPKPLIYEISKLASG |
| 5D6-2 | VPSRFSGSGSGTEFTLTISSLQPDDAATYYCQQWNYPLITFGGGTK VEIK (SEQ ID |
| | NO: 100) |
| h4D10L2- | DIQMTQSPSTLSASVGDRVTITCSASSHVSYMHWYQQKPGKAPKPLIYEISKLASG |
| 5G6 | VPSRFSGSGSGTEFTLTISSLQPDDAATYYCQQWNYPLITFGGGTKVEIK (SEQ ID |
| | NO: 101) |
| h4D10L2- | DIQMTQSPSTLSASVGDRVTITCSASSSVSYMHWYQQKPGKAPKPLIYEIMKLASG |
| 19H8 | VPSRFSGSGSGTEFTLTISSLQPDDAATYYCQQWNYPLITFGGGTKVEIK (SEQ ID |
| | NO: 102) |
| h4D10L2- | DIQMTQSPSTLSASVGDRVTITCSASSSVSYMHWYQQKPGKAPKPLIYEISRLASG |
| 20A3 | VPSRFSGSGSGTEFTLTISSLQPDDAATYYCQQWNYPLITFGGGTKVEIK (SEQ ID |
| | NO: 103) |
| h4D10L2 | DIQMTQSPSTLSASVGDRVTITCSASSEVSYMHWYQQKPGKAPKPLIYEIMKLASG |
| CM1 | VPSRFSGSGSGTEFTLTISSLQPDDAATYYCQQWNYPLITFGGGTKVEIK (SEQ ID |
| | NO: 104) |
| h4D10L2 | DIQMTQSPSTLSASVGDRVTITCSASSEVSYMHWYQQKPGKAPKPLIYEISRLASG |
| CM2 | VPSRFSGSGSGTEFTLTISSLQPDDAATYYCQQWNYPLITFGGGTKVEIK (SEQ ID |
| | NO: 105) |
| h4D10L2 | DIQMTQSPSTLSASVGDRVTITCSASSEVSYMHWYQQKPGKAPKPLIYEIMRLASG |
| CM3 | VPSRFSGSGSGTEFTLTISSLQPDDAATYYCQQWNYPLITFGGGTKVEIK (SEQ ID |
| | NO: 106) |
| h4D10H0a | QVQLVQSGAEVKKPGASVKVSCKASGYTFSSYVIHWVRQAPGQGLEWMGYFNP |
| | YNNGTNYNENFKGRVTMTRDTSISTAYMELSRLRSDDTAVYYCARYYYASGSLY |
| | FDYWGQGTLVTVSS (SEQ ID NO: 123) |
| h4D10H0b | QVQLVQSGAEVKKPGASVKVSCKASGYTFSSYVIHWVRQAPGQGLEWMGYFNP |
| | YNNGTNYNENFKGRVTMTRDTSTSTVYMELSSLRSEDTAVYYCARYYYASGSLY |
| | FDYWGQGTTVTVSS (SEQ ID NO: 124) |
| h4D10H1 | QVQLVQSGAEVVKPGASVKVSCKASGYTFSSYVIHWVRQAPGQGLEWIGYFNPY |
| | NNGTNYNENFKGRVTMTADTSISTAYMELSRLRSDDTAVYYCARYYYASGSLYF |
| | DYWGQGTLVTVSS (SEQ ID NO: 125) |
| h4D10H2 | QVQLVQSGAEVVKPGASVKVSCKASGYTFSSYVIHWVRQAPGQGLEWIGYFNPY |
| | NNGTNYNENFKGRVTMTADTSTSTVYMELSSLRSEDTAVYYCARYYYASGSLYF |
| | DYWGQGTTVTVSS (SEQ ID NO: 126) |
| h4D10H3 | QVQLVQSGAEVKKPGASVKVSCKASGYTFSSYVIHWVRQAPGQGLEWMGYFNP |
| | YNNGTNYNENFKGRVTMTADTSISTAYMELSRLRSDDTAVYYCARYYYASGSLY |
| | FDYWGQGTLVTVSS (SEQ ID NO: 127) |
| h4D10H4 | QVQLVQSGAEVKKPGASVKVSCKASGYTFSSYVIHWVRQAPGQGLEWMGYFNP |
| | YNNGTNYNENFKGRVTMTADTSTSTVYMELSSLRSEDTAVYYCARYYYASGSLY |
| | FDYWGQGTTVTVSS (SEQ ID NO: 128) |
| h4D10H4- | QVQLVQSGAEVKKPGASVKVSCKASGYTFSSYVIMWVRQAPGQGLEWMGYFNP |
| PTM- | YNQGTNYNENFKGRVTMTADTSTSTVYMELSSLRSEDTAVYYCARYFYASGSLY |
| 10E5 | FDYWGQGTTVTVSS (SEQ ID NO: 130) |
| h4D10H4- | QVQLVQSGAEVKKPGASVKVSCKASGYTFSSYVIHWVRQAPGQGLEWMGYF |
| v1 | NPYNQGTNYNENFKGRVTMTADTSTSTVYMELSSLRSEDTAVYYCARYYYAS |
| | GSLYFDYWGQGTTVTVSS (SEQ ID NO: 143) |
| h4D10H4- | QVQLVQSGAEVKKPGASVKVSCKASGYTFSSYVIHWVRQAPGQGLEWMGYF |
| v2 | NPYNIGTNYNENFKGRVTMTADTSTSTVYMELSSLRSEDTAVYYCARYYYAS |
| | GSLYFDYWGQGTTVTVSS (SEQ ID NO: 144) |
| h4D10H4- | QVQLVQSGAEVKKPGASVKVSCKASGYTFSSYVIHWVRQAPGQGLEWMGYF |
| v3 | NPYNVGTNYNENFKGRVTMTADTSTSTVYMELSSLRSEDTAVYYCARYYYAS |
| | GSLYFDYWGQGTTVTVSS (SEQ ID NO: 145) |
| h4D10H4- | QVQLVQSGAEVKKPGASVKVSCKASGYTFSSYVIHWVRQAPGQGLEWMGYF |
| v4 | NPYNNGTNYNENFKGRVTMTADTSTSTVYMELSSLRSEDTAVYYCARYEYAS |
| | GSLYFDYWGQGTTVTVSS (SEQ ID NO: 146) |
| h4D10H4- | QVQLVQSGAEVKKPGASVKVSCKASGYTFSSYVIHWVRQAPGQGLEWMGYF |
| CM1 | NPYNQGTNYNENFKGRVTMTADTSTSTVYMELSSLRSEDTAVYYCARYFYAS |
| | GSLYFDYWGQGTTVTVSS (SEQ ID NO: 147) |
| h4D10H4- | QVQLVQSGAEVKKPGASVKVSCKASGYTFSSYVIHWVRQAPGQGLEWMGYF |
| CM2 | NPYNIGTNYNENFKGRVTMTADTSTSTVYMELSSLRSEDTAVYYCARYFYASG |
| | SLYFDYWGQGTTVTVSS (SEQ ID NO: 148) |
| h4D10H4- | QVQLVQSGAEVKKPGASVKVSCKASGYTFSSYVIHWVRQAPGQGLEWMGYF |
| CM3 | NPYNVGTNYNENFKGRVTMTADTSTSTVYMELSSLRSEDTAVYYCARYFYAS |
| | GSLYFDYWGQGTTVTVSS (SEQ ID NO: 149) |

In some embodiments, provided herein are humanized 4D1. In some embodiments, provided herein are humanized anti-CEACAM11 antibodies or antigen-binding fragments thereof comprising: (a) a VL having at least 85%, at least 860%, at least 87%, at least 88%, at least 89%, at least 90%, at least 91%, at least 92%, at least 93%, at least 94%, at least 95%, at least 96%, at least 9700 at least 98%, at least 9900, or 10000 sequence identity to an amino acid sequence selected from the group consisting of SEQ ID NOs:94-99; and (b) a VH having at least 850%, at least 860%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs:123-128.

In some embodiments, provided herein are humanized anti-CEACAM1 antibodies or antigen-binding fragments thereof comprising: (a) a VL having an amino acid sequence selected from the group consisting of SEQ ID NOs:94-99; and (b) a VH having an amino acid sequence selected from the group consisting of SEQ ID NOs:123-128; or variants thereof having about 3, about 5, about 8, about 10, about 12, or about 15 amino acid substitutions, additions, and/or deletions in the VL or VH. In some embodiments, the VL variant can have up to about 5 amino acid substitutions, additions, and/or deletions in an amino acid sequence selected from the group consisting of SEQ ID NOs:94-99. In some embodiments, the VH variant can have up to about 5 amino acid substitutions, additions, and/or deletions in an amino acid sequence selected from the group consisting of SEQ ID NOs:123-128. In some embodiments, the variants can have up to 3 amino acid substitutions.

In some embodiments, the VL can have an amino acid sequence selected from the group consisting of SEQ ID NOs:94-99 with a substitution at S28. The substitution can be S28F, or S28H. In some embodiments, the VL can have an amino acid sequence selected from the group consisting of SEQ ID NOs:94-99 with a substitution at S51. The substitution can be S5IM. In some embodiments, the VL can have an amino acid sequence selected from the group consisting of SEQ ID NOs:94-99 with a substitution at K52. The substitution can be K52R. In some embodiments, the VL can have an amino acid sequence selected from the group consisting of SEQ ID NOs:94-99 with substitutions at S28, S51 and/or K52. The VL can have an amino acid sequence selected from the group consisting of SEQ ID NOs:94-99 with (1) S28F and S51M substitutions, (2) S28F and K52R substitutions, (3) S28H and S51M substitutions, (4) S28H and K52R substitutions, (5) S51M and K52R substitutions, (6) S28F, S51M and K52R substitutions, or (7) S28H, S51M and K52R substitutions.

In some embodiments, the VL can have the amino acid sequence of SEQ ID NO:97 with a substitution at S28. The substitution can be S28F, or S28H. In some embodiments, the VL can have the amino acid sequence of SEQ ID NO:97 with a substitution at S51. The substitution can be S5IM. In some embodiments, the VL can have the amino acid sequence of SEQ ID NO:97 with a substitution at K52. The substitution can be K52R. In some embodiments, the VL can have the amino acid sequence of SEQ ID NO:97 with substitutions at S28, S51 and/or K52. The VL can have the amino acid sequence of SEQ ID NO:97 with S28F substitution. In some embodiments, the VL can have the amino acid sequence of SEQ ID NO:100. The VL can have the amino acid sequence of SEQ ID NO:97 with S28H substitution. In some embodiments, the VL can have the amino acid sequence of SEQ ID NO:101. The VL can have the amino acid sequence of SEQ ID NO:97 with S51M substitution. In some embodiments, the VL can have the amino acid sequence of SEQ ID NO:102. The VL can have the amino acid sequence of SEQ ID NO:97 with K52R substitution. In some embodiments, the VL can have the amino acid sequence of SEQ ID NO:103. The VL can have the amino acid sequence of SEQ ID NO:97 with S28F and S51M substitutions. In some embodiments, the VL can have the amino acid sequence of SEQ ID NO:104. The VL can have the amino acid sequence of SEQ ID NO:97 with S28F and K52R substitutions. In some embodiments, the VL can have the amino acid sequence of SEQ ID NO:105. The VL can have the amino acid sequence of SEQ ID NO:97 with S28F, S51M and K52R substitutions. In some embodiments, the VL can have the amino acid sequence of SEQ ID NO:106.

In some embodiments, the VH can have an amino acid sequence selected from the group consisting of SEQ ID NOs:123-128 with a substitution at H35. The substitution can be H35M. In some embodiments, the VH can have an amino acid sequence selected from the group consisting of SEQ ID NOs:123-128 with a substitution at N56. The substitution can be N56I, N56Q, N56V, or N56W. In some embodiments, the VH can have an amino acid sequence selected from the group consisting of SEQ ID NOs:123-128 with a substitution at Y100. The substitution can be Y100F, Y100V, or Y100W. In some embodiments, the VH can have an amino acid sequence selected from the group consisting of SEQ ID NOs:123-128 with substitutions at H35, N56 and/or Y100. The VH can have an amino acid sequence selected from the group consisting of SEQ ID NOs:123-128 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions.

In some embodiments, the VH can have the amino acid sequence of SEQ ID NO:128 with a substitution at H35. The substitution can be H35M. In some embodiments, the VH can have the amino acid sequence of SEQ ID NO:128 with a substitution at N56. The substitution can be N56I, N56Q, N56V, or N56W. In some embodiments, the VH can have the amino acid sequence of SEQ ID NO: 128 with a substitution at Y100. The substitution can be Y100F, Y100V, or Y100W. In some embodiments, the VH can have the amino acid sequence of SEQ ID NO:128 with substitutions at H35, N56 and/or Y100. The VH can have the amino acid sequence of SEQ ID NO:128 with H35M, N56Q and Y100F substitutions. In some embodiments, the VH can have the amino acid sequence of SEQ ID NO:130. The VH can have the amino acid sequence of SEQ ID NO:128 with N56Q substitution. In some embodiments, the VH can have the amino acid sequence of SEQ ID NO:143. The VH can have the amino acid sequence of SEQ ID NO:128 with N56I substitution. In some embodiments, the VH can have the amino acid sequence of SEQ ID NO:144. The VH can have the amino acid sequence of SEQ ID NO:128 with N56V substitution. In some embodiments, the VH can have the amino acid sequence of SEQ ID NO:145. The VH can have the amino acid sequence of SEQ ID NO:128 with Y100F substitution. In some embodiments, the VH can have the amino acid sequence of SEQ ID NO:146. The VH can have the amino acid sequence of SEQ ID NO:128 with N56Q and Y100F substitutions. In some embodiments, the VH can have the amino acid sequence of SEQ ID NO:147. The VH can have the amino acid sequence of SEQ ID NO:128 with N56I and Y100F substitutions. In some embodiments, the VH can have the amino acid sequence of SEQ ID NO:148. The VH can have the amino acid sequence of SEQ ID NO: 128 with N56V and Y100F substitutions. In some embodiments, the VH can have the amino acid sequence of SEQ ID NO:149.

In some embodiments, provided herein are humanized anti-CEACAM1 antibodies or antigen-binding fragments thereof comprising a VL and a VH, wherein the VL and VH have the amino acid sequences of SEQ ID NOs:94 and 123, respectively. In some embodiments, VL and VH have the amino acid sequences of SEQ ID NOs:94 and 124, respectively. In some embodiments, VL and VH have the amino acid sequences of SEQ ID NOs:94 and 125, respectively. In some embodiments, VL and VH have the amino acid sequences of SEQ ID NOs:94 and 126, respectively. In some embodiments, VL and VH have the amino acid sequences of SEQ ID NOs:94 and 127, respectively. In some embodiments, VL and VH have the amino acid sequences of SEQ ID NOs:94 and 128, respectively. In some embodiments, VL and VH have the amino acid sequences of SEQ ID NOs:94 and 130, respectively. In some embodiments, VL and VH have the amino acid sequences of SEQ ID NOs:94 and 143, respectively. In some embodiments, VL and VH have the amino acid sequences of SEQ ID NOs:94 and 144, respectively. In some embodiments, VL and VH have the amino acid sequences of SEQ ID NOs:94 and 145, respectively. In some embodiments, VL and VH have the amino acid sequences of SEQ ID NOs:94 and 146, respectively. In some embodiments, VL and VH have the amino acid sequences of SEQ ID NOs:94 and 147, respectively. In some embodiments, VL and VH have the amino acid sequences of SEQ ID NOs:94 and 148, respectively. In some embodiments, VL and VH have the amino acid sequences of SEQ ID NOs:94 and 149, respectively. In some embodiments, VL and VH have the amino acid sequences of SEQ ID NOs:95 and 123, respectively. In some embodiments, VL and VH have the amino acid sequences of SEQ ID NOs:95 and 124, respectively. In some embodiments, VL and VH have the amino acid sequences of SEQ ID NOs:95 and 125, respectively. In some embodiments, VL and VH have the amino acid sequences of SEQ ID NOs:95 and 126, respectively. In some embodiments, VL and VH have the amino acid sequences of SEQ ID NOs:95 and 127, respectively. In some embodiments, VL and VH have the amino acid sequences of SEQ ID NOs:95 and 128, respectively. In some embodiments, VL and VH have the amino acid sequences of SEQ ID NOs:95 and 130, respectively. In some embodiments, VL and VH have the amino acid sequences of SEQ ID NOs:95 and 143, respectively. In some embodiments, VL and VH have the amino acid sequences of SEQ ID NOs:95 and 144, respectively. In some embodiments, VL and VH have the amino acid sequences of SEQ ID NOs:95 and 145, respectively. In some embodiments, VL and VH have the amino acid sequences of SEQ ID NOs:95 and 146, respectively. In some embodiments, VL and VH have the amino acid sequences of SEQ ID NOs:95 and 147, respectively. In some embodiments, VL and VH have the amino acid sequences of SEQ ID NOs:95 and 148, respectively. In some embodiments, VL and VH have the amino acid sequences of SEQ ID NOs:95 and 149, respectively. In some embodiments, VL and VH have the amino acid sequences of SEQ ID NOs:96 and 123, respectively. In some embodiments, VL and VH have the amino acid sequences of SEQ ID NOs:96 and 124, respectively. In some embodiments, VL and VH have the amino acid sequences of SEQ ID NOs:96 and 125, respectively. In some embodiments, VL and VH have the amino acid sequences of SEQ ID NOs:96 and 126, respectively. In some embodiments, VL and VH have the amino acid sequences of SEQ ID NOs:96 and 127, respectively. In some embodiments, VL and VH have the amino acid sequences of SEQ ID NOs:96 and 128, respectively. In some embodiments, VL and VH have the amino acid sequences of SEQ ID NOs:96 and 130, respectively. In some embodiments, VL and VH have the amino acid sequences of SEQ ID NOs:96 and 143, respectively. In some embodiments, VL and VH have the amino acid sequences of SEQ ID NOs:96 and 144, respectively. In some embodiments, VL and VH have the amino acid sequences of SEQ ID NOs:96 and 145, respectively. In some embodiments, VL and VH have the amino acid sequences of SEQ ID NOs:96 and 146, respectively. In some embodiments, VL and VH have the amino acid sequences of SEQ ID NOs:96 and 147, respectively. In some embodiments, VL and VH have the amino acid sequences of SEQ ID NOs:96 and 148, respectively. In some embodiments, VL and VH have the amino acid sequences of SEQ ID NOs:96 and 149, respectively. In some embodiments, VL and VH have the amino acid sequences of SEQ ID NOs:97 and 123, respectively. In some embodiments, VL and VH have the amino acid sequences of SEQ ID NOs:97 and 124, respectively. In some embodiments, VL and VH have the amino acid sequences of SEQ ID NOs:97 and 125, respectively. In some embodiments, VL and VH have the amino acid sequences of SEQ ID NOs:97 and 126, respectively. In some embodiments, VL and VH have the amino acid sequences of SEQ ID NOs:97 and 127, respectively. In some embodiments, VL and VH have the amino acid sequences of SEQ ID NOs:97 and 128, respectively. In some embodiments, VL and VH have the amino acid sequences of SEQ ID NOs:97 and 130, respectively. In some embodiments, VL and VH have the amino acid sequences of SEQ ID NOs:97 and 143, respectively. In some embodiments, VL and VH have the amino acid sequences of SEQ ID NOs:97 and 144, respectively. In some embodiments, VL and VH have the amino acid sequences of SEQ ID NOs:97 and 145, respectively. In some embodiments, VL and VH have the amino acid sequences of SEQ ID NOs:97 and 146, respectively. In some embodiments, VL and VH have the amino acid sequences of SEQ ID NOs:97 and 147, respectively. In some embodiments, VL and VH have the amino acid sequences of SEQ ID NOs:97 and 148, respectively. In some embodiments, VL and VH have the amino acid sequences of SEQ ID NOs:97 and 149, respectively. In some embodiments, VL and VH have the amino acid sequences of SEQ ID NOs:98 and 123, respectively. In some embodiments, VL and VH have the amino acid sequences of SEQ ID NOs:98 and 124, respectively. In some embodiments, VL and VH have the amino acid sequences of SEQ ID NOs:98 and 125, respectively. In some embodiments, VL and VH have the amino acid sequences of SEQ ID NOs:98 and 126, respectively. In some embodiments, VL and VH have the amino acid sequences of SEQ ID NOs:98 and 127, respectively. In some embodiments, VL and VH have the amino acid sequences of SEQ ID NOs:98 and 128, respectively. In some embodiments, VL and VH have the amino acid sequences of SEQ ID NOs:98 and 130, respectively. In some embodiments, VL and VH have the amino acid sequences of SEQ ID NOs:98 and 143, respectively. In some embodiments, VL and VH have the amino acid sequences of SEQ ID NOs:98 and 144, respectively. In some embodiments, VL and VH have the amino acid sequences of SEQ ID NOs:98 and 145, respectively. In some embodiments, VL and VH have the amino acid sequences of SEQ ID NOs:98 and 146, respectively. In some embodiments, VL and VH have the amino acid sequences of SEQ ID NOs:98 and 147, respectively. In some embodiments, VL and VH have the amino acid sequences of SEQ ID NOs:98 and 148, respectively. In some embodiments, VL and VH have the amino acid sequences of SEQ ID NOs:98 and 149, respectively. In some embodiments, VL and VH have the amino acid sequences of SEQ ID NOs:99 and 123, respectively. In some embodiments, VL and VH have the amino acid sequences of SEQ ID NOs:99 and 124, respectively. In some embodiments, VL and VH have the amino acid sequences of SEQ ID NOs:99 and 125, respectively. In some embodiments, VL and VH have the amino acid sequences of SEQ ID NOs:99 and 126, respectively. In some embodiments, VL and VH have the amino acid sequences of SEQ ID NOs:99 and 127, respectively. In some embodiments, VL and VH have the amino acid sequences of SEQ ID NOs:99 and 128, respectively. In some embodiments, VL and VH have the amino acid sequences of SEQ ID NOs:99 and 130, respectively. In some embodiments, VL and VH have the amino acid sequences of SEQ ID NOs:99 and 143, respectively. In some embodiments, VL and VH have the amino acid sequences of SEQ ID NOs:99 and 144, respectively. In some embodiments, VL and VH have the amino acid sequences of SEQ ID NOs:99 and 145, respectively. In some embodiments, VL and VH have the amino acid sequences of SEQ ID NOs:99 and 146, respectively. In some embodiments, VL and VH have the amino acid sequences of SEQ ID NOs:99 and 147, respectively. In some embodiments, VL and VH have the amino acid sequences of SEQ ID NOs:99 and 148, respectively. In some embodiments, VL and VH have the amino acid sequences of SEQ ID NOs:99 and 149, respectively. In some embodiments, VL and VH have the amino acid sequences of SEQ ID NOs:100 and 123, respectively. In some embodiments, VL and VH have the amino acid sequences of SEQ ID NOs:100 and 124, respectively. In some embodiments, VL and VH have the amino acid sequences of SEQ ID NOs:100 and 125, respectively. In some embodiments, VL and VH have the amino acid sequences of SEQ ID NOs:100 and 126, respectively. In some embodiments, VL and VH have the amino acid sequences of SEQ ID NOs:100 and 127, respectively. In some embodiments, VL and VH have the amino acid sequences of SEQ ID NOs:100 and 128, respectively. In some embodiments, VL and VH have the amino acid sequences of SEQ ID NOs:100 and 130, respectively. In some embodiments, VL and VH have the amino acid sequences of SEQ ID NOs:100 and 143, respectively. In some embodiments, VL and VH have the amino acid sequences of SEQ ID NOs:100 and 144, respectively. In some embodiments, VL and VH have the amino acid sequences of SEQ ID NOs:100 and 145, respectively. In some embodiments, VL and VH have the amino acid sequences of SEQ ID NOs:100 and 146, respectively. In some embodiments, VL and VH have the amino acid sequences of SEQ ID NOs:100 and 147, respectively. In some embodiments, VL and VH have the amino acid sequences of SEQ ID NOs:100 and 148, respectively. In some embodiments, VL and VH have the amino acid sequences of SEQ ID NOs:100 and 149, respectively. In some embodiments, VL and VH have the amino acid sequences of SEQ ID NOs:101 and 123, respectively. In some embodiments, VL and VH have the amino acid sequences of SEQ ID NOs:101 and 124, respectively. In some embodiments, VL and VH have the amino acid sequences of SEQ ID NOs:101 and 125, respectively. In some embodiments, VL and VH have the amino acid sequences of SEQ ID NOs:101 and 126, respectively. In some embodiments, VL and VH have the amino acid sequences of SEQ ID NOs:101 and 127, respectively. In some embodiments, VL and VH have the amino acid sequences of SEQ ID NOs:101 and 128, respectively. In some embodiments, VL and VH have the amino acid sequences of SEQ ID NOs:101 and 130, respectively. In some embodiments, VL and VH have the amino acid sequences of SEQ ID NOs:101 and 143, respectively. In some embodiments, VL and VH have the amino acid sequences of SEQ ID NOs:101 and 144, respectively. In some embodiments, VL and VH have the amino acid sequences of SEQ ID NOs:101 and 145, respectively. In some embodiments, VL and VH have the amino acid sequences of SEQ ID NOs:101 and 146, respectively. In some embodiments, VL and VH have the amino acid sequences of SEQ ID NOs:101 and 147, respectively. In some embodiments, VL and VH have the amino acid sequences of SEQ ID NOs:101 and 148, respectively. In some embodiments, VL and VH have the amino acid sequences of SEQ ID NOs:101 and 149, respectively. In some embodiments, VL and VH have the amino acid sequences of SEQ ID NOs:102 and 123, respectively. In some embodiments, VL and VH have the amino acid sequences of SEQ ID NOs:102 and 124, respectively. In some embodiments, VL and VH have the amino acid sequences of SEQ ID NOs:102 and 125, respectively. In some embodiments, VL and VH have the amino acid sequences of SEQ ID NOs:102 and 126, respectively. In some embodiments, VL and VH have the amino acid sequences of SEQ ID NOs:102 and 127, respectively. In some embodiments, VL and VH have the amino acid sequences of SEQ ID NOs:102 and 128, respectively. In some embodiments, VL and VH have the amino acid sequences of SEQ ID NOs:102 and 130, respectively. In some embodiments, VL and VH have the amino acid sequences of SEQ ID NOs:102 and 143, respectively. In some embodiments, VL and VH have the amino acid sequences of SEQ ID NOs:102 and 144, respectively. In some embodiments, VL and VH have the amino acid sequences of SEQ ID NOs:102 and 145, respectively. In some embodiments, VL and VH have the amino acid sequences of SEQ ID NOs:102 and 146, respectively. In some embodiments, VL and VH have the amino acid sequences of SEQ ID NOs:102 and 147, respectively. In some embodiments, VL and VH have the amino acid sequences of SEQ ID NOs:102 and 148, respectively. In some embodiments, VL and VH have the amino acid sequences of SEQ ID NOs:102 and 149, respectively. In some embodiments, VL and VH have the amino acid sequences of SEQ ID NOs:103 and 123, respectively. In some embodiments, VL and VH have the amino acid sequences of SEQ ID NOs:103 and 124, respectively. In some embodiments, VL and VH have the amino acid sequences of SEQ ID NOs:103 and 125, respectively. In some embodiments, VL and VH have the amino acid sequences of SEQ ID NOs:103 and 126, respectively. In some embodiments, VL and VH have the amino acid sequences of SEQ ID NOs:103 and 127, respectively. In some embodiments, VL and VH have the amino acid sequences of SEQ ID NOs:103 and 128, respectively. In some embodiments, VL and VH have the amino acid sequences of SEQ ID NOs:103 and 130, respectively. In some embodiments, VL and VH have the amino acid sequences of SEQ ID NOs:103 and 143, respectively. In some embodiments, VL and VH have the amino acid sequences of SEQ ID NOs:103 and 144, respectively. In some embodiments, VL and VH have the amino acid sequences of SEQ ID NOs:103 and 145, respectively. In some embodiments, VL and VH have the amino acid sequences of SEQ ID NOs:103 and 146, respectively. In some embodiments, VL and VH have the amino acid sequences of SEQ ID NOs:103 and 147, respectively. In some embodiments, VL and VH have the amino acid sequences of SEQ ID NOs:103 and 148, respectively. In some embodiments, VL and VH have the amino acid sequences of SEQ ID NOs:103 and 149, respectively. In some embodiments, VL and VH have the amino acid sequences of SEQ ID NOs:104 and 123, respectively. In some embodiments, VL and VH have the amino acid sequences of SEQ ID NOs:104 and 124, respectively. In some embodiments, VL and VH have the amino acid sequences of SEQ ID NOs:104 and 125, respectively. In some embodiments, VL and VH have the amino acid sequences of SEQ ID NOs:104 and 126, respectively. In some embodiments, VL and VH have the amino acid sequences of SEQ ID NOs:104 and 127, respectively. In some embodiments, VL and VH have the amino acid sequences of SEQ ID NOs:104 and 128, respectively. In some embodiments, VL and VH have the amino acid sequences of SEQ ID NOs:104 and 130, respectively. In some embodiments, VL and VH have the amino acid sequences of SEQ ID NOs:104 and 143, respectively. In some embodiments, VL and VH have the amino acid sequences of SEQ ID NOs:104 and 144, respectively. In some embodiments, VL and VH have the amino acid sequences of SEQ ID NOs:104 and 145, respectively. In some embodiments, VL and VH have the amino acid sequences of SEQ ID NOs:104 and 146, respectively. In some embodiments, VL and VH have the amino acid sequences of SEQ ID NOs:104 and 147, respectively. In some embodiments, VL and VH have the amino acid sequences of SEQ ID NOs:104 and 148, respectively. In some embodiments, VL and VH have the amino acid sequences of SEQ ID NOs:104 and 149, respectively. In some embodiments, VL and VH have the amino acid sequences of SEQ ID NOs:105 and 123, respectively. In some embodiments, VL and VH have the amino acid sequences of SEQ ID NOs:105 and 124, respectively. In some embodiments, VL and VH have the amino acid sequences of SEQ ID NOs:105 and 125, respectively. In some embodiments, VL and VH have the amino acid sequences of SEQ ID NOs:105 and 126, respectively. In some embodiments, VL and VH have the amino acid sequences of SEQ ID NOs:105 and 127, respectively. In some embodiments, VL and VH have the amino acid sequences of SEQ ID NOs:105 and 128, respectively. In some embodiments, VL and VH have the amino acid sequences of SEQ ID NOs:105 and 130, respectively. In some embodiments, VL and VH have the amino acid sequences of SEQ ID NOs:105 and 143, respectively. In some embodiments, VL and VH have the amino acid sequences of SEQ ID NOs:105 and 144, respectively. In some embodiments, VL and VH have the amino acid sequences of SEQ ID NOs:105 and 145, respectively. In some embodiments, VL and VH have the amino acid sequences of SEQ ID NOs:105 and 146, respectively. In some embodiments, VL and VH have the amino acid sequences of SEQ ID NOs:105 and 147, respectively. In some embodiments, VL and VH have the amino acid sequences of SEQ ID NOs:105 and 148, respectively. In some embodiments, VL and VH have the amino acid sequences of SEQ ID NOs:105 and 149, respectively. In some embodiments, VL and VH have the amino acid sequences of SEQ ID NOs:106 and 123, respectively. In some embodiments, VL and VH have the amino acid sequences of SEQ ID NOs:106 and 124, respectively. In some embodiments, VL and VH have the amino acid sequences of SEQ ID NOs:106 and 125, respectively. In some embodiments, VL and VH have the amino acid sequences of SEQ ID NOs:106 and 126, respectively. In some embodiments, VL and VH have the amino acid sequences of SEQ ID NOs:106 and 127, respectively. In some embodiments, VL and VH have the amino acid sequences of SEQ ID NOs:106 and 128, respectively. In some embodiments, VL and VH have the amino acid sequences of SEQ ID NOs:106 and 130, respectively. In some embodiments, VL and VH have the amino acid sequences of SEQ ID NOs:106 and 143, respectively. In some embodiments, VL and VH have the amino acid sequences of SEQ ID NOs:106 and 144, respectively. In some embodiments, VL and VH have the amino acid sequences of SEQ ID NOs:106 and 145, respectively. In some embodiments, VL and VH have the amino acid sequences of SEQ ID NOs:106 and 146, respectively. In some embodiments, VL and VH have the amino acid sequences of SEQ ID NOs:106 and 147, respectively. In some embodiments, VL and VH have the amino acid sequences of SEQ ID NOs:106 and 148, respectively. In some embodiments, VL and VH have the amino acid sequences of SEQ ID NOs:106 and 149, respectively.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:94. The humanized anti-CEACAM1 antibody or antigen-binding fragment thereof can have a VL having at least 85% sequence identity to SEQ ID NO:94. The humanized anti-CEACAM1 antibody or antigen-binding fragment thereof can have a VL having at least 90% sequence identity to SEQ ID NO:94. The humanized anti-CEACAM1 antibody or antigen-binding fragment thereof can have a VL having at least 95% sequence identity to SEQ ID NO:94. The humanized anti-CEACAM1 antibody or antigen-binding fragment thereof can have a VL having at least 98% sequence identity to SEQ ID NO:94. In some embodiments, provided herein are humanized antibodies or antigen-binding fragments thereof that specifically bind CEACAM1 comprising a VL having the amino acid sequence of SEQ ID NO:94.

In some embodiments, the VL can have the amino acid sequence of SEQ ID NO:94 with a substitution at S28. The substitution can be S28F, or S28H. In some embodiments, the VL can have the amino acid sequence of SEQ ID NO:94 with a substitution at 551. The substitution can be S5IM. In some embodiments, the VL can have the amino acid sequence of SEQ ID NO:94 with a substitution at K52. The substitution can be K52R. In some embodiments, the VL can have the amino acid sequence of SEQ ID NO:94 with substitutions at S28, S51 and/or K52. The VL can have the amino acid sequence of SEQ ID NO:94 with (1) S28F and S51M substitutions, (2) S28F and K52R substitutions, (3) S28H and S51M substitutions, (4) S28H and K52R substitutions, (5) S5IM and K52R substitutions, (6) S28F, S51M and K52R substitutions, or (7) S28H, S51M and K52R substitutions. The VL can have the amino acid sequence of SEQ ID NO:94 with S28F substitution. The VL can have the amino acid sequence of SEQ ID NO:94 with S28H substitution. The VL can have the amino acid sequence of SEQ ID NO:94 with S51M substitution. The VL can have the amino acid sequence of SEQ ID NO:94 with K52R substitution. The VL can have the amino acid sequence of SEQ ID NO:94 with S28F and S51M substitutions. The VL can have the amino acid sequence of SEQ ID NO:94 with S28F and K52R substitutions. The VL can have the amino acid sequence of SEQ ID NO:94 with S28H and S51M substitutions. The VL can have the amino acid sequence of SEQ ID NO:94 with S28H and K52R substitutions. The VL can have the amino acid sequence of SEQ ID NO:94 with S51M and K52R substitutions. The VL can have the amino acid sequence of SEQ ID NO:94 with S28F, S51M and K52R substitutions. The VL can have the amino acid sequence of SEQ ID NO:94 with S28H, S51M and K52R substitutions.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:95. The humanized anti-CEACAM1 antibody or antigen-binding fragment thereof can have a VL having at least 85% sequence identity to SEQ ID NO:95. The humanized anti-CEACAM1 antibody or antigen-binding fragment thereof can have a VL having at least 90% sequence identity to SEQ ID NO:95. The humanized anti-CEACAM1 antibody or antigen-binding fragment thereof can have a VL having at least 95% sequence identity to SEQ ID NO:95. The humanized anti-CEACAM1 antibody or antigen-binding fragment thereof can have a VL having at least 98% sequence identity to SEQ ID NO:95. In some embodiments, provided herein are humanized antibodies or antigen-binding fragments thereof that specifically bind CEACAM1 comprising a VL having the amino acid sequence of SEQ ID NO:95.

In some embodiments, the VL can have the amino acid sequence of SEQ ID NO:95 with a substitution at S28. The substitution can be S28F, or S28H. In some embodiments, the VL can have the amino acid sequence of SEQ ID NO:95 with a substitution at 551. The substitution can be S5IM. In some embodiments, the VL can have the amino acid sequence of SEQ ID NO:95 with a substitution at K52. The substitution can be K52R. In some embodiments, the VL can have the amino acid sequence of SEQ ID NO:95 with substitutions at S28, S51 and/or K52. The VL can have the amino acid sequence of SEQ ID NO:95 with (1) S28F and S51M substitutions, (2) S28F and K52R substitutions, (3) S28H and S51M substitutions, (4) S28H and K52R substitutions, (5) S5IM and K52R substitutions, (6) S28F, S51M and K52R substitutions, or (7) S28H, S51M and K52R substitutions. The VL can have the amino acid sequence of SEQ ID NO:95 with S28F substitution. The VL can have the amino acid sequence of SEQ ID NO:95 with S28H substitution. The VL can have the amino acid sequence of SEQ ID NO:95 with S51M substitution. The VL can have the amino acid sequence of SEQ ID NO:95 with K52R substitution. The VL can have the amino acid sequence of SEQ ID NO:95 with S28F and S51M substitutions. The VL can have the amino acid sequence of SEQ ID NO:95 with S28F and K52R substitutions. The VL can have the amino acid sequence of SEQ ID NO:95 with S28H and S51M substitutions. The VL can have the amino acid sequence of SEQ ID NO:95 with S28H and K52R substitutions. The VL can have the amino acid sequence of SEQ ID NO:95 with S51M and K52R substitutions. The VL can have the amino acid sequence of SEQ ID NO:95 with S28F, S51M and K52R substitutions. The VL can have the amino acid sequence of SEQ ID NO:95 with S28H, S51M and K52R substitutions.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:96. The humanized anti-CEACAM1 antibody or antigen-binding fragment thereof can have a VL having at least 85% sequence identity to SEQ ID NO:96. The humanized anti-CEACAM1 antibody or antigen-binding fragment thereof can have a VL having at least 90% sequence identity to SEQ ID NO:96. The humanized anti-CEACAM1 antibody or antigen-binding fragment thereof can have a VL having at least 95% sequence identity to SEQ ID NO:96. The humanized anti-CEACAM1 antibody or antigen-binding fragment thereof can have a VL having at least 98% sequence identity to SEQ ID NO:96. In some embodiments, provided herein are humanized antibodies or antigen-binding fragments thereof that specifically bind CEACAM1 comprising a VL having the amino acid sequence of SEQ ID NO:96.

In some embodiments, the VL can have the amino acid sequence of SEQ ID NO:96 with a substitution at S28. The substitution can be S28F, or S28H. In some embodiments, the VL can have the amino acid sequence of SEQ ID NO:96 with a substitution at 551. The substitution can be S5IM. In some embodiments, the VL can have the amino acid sequence of SEQ ID NO:96 with a substitution at K52. The substitution can be K52R. In some embodiments, the VL can have the amino acid sequence of SEQ ID NO:96 with substitutions at S28, S51 and/or K52. The VL can have the amino acid sequence of SEQ ID NO:96 with (1) S28F and S51M substitutions, (2) S28F and K52R substitutions, (3) S28H and S51M substitutions, (4) S28H and K52R substitutions, (5) S5IM and K52R substitutions, (6) S28F, S51M and K52R substitutions, or (7) S28H, S51M and K52R substitutions. The VL can have the amino acid sequence of SEQ ID NO:96 with S28F substitution. The VL can have the amino acid sequence of SEQ ID NO:96 with S28H substitution. The VL can have the amino acid sequence of SEQ ID NO:96 with S51M substitution. The VL can have the amino acid sequence of SEQ ID NO:96 with K52R substitution. The VL can have the amino acid sequence of SEQ ID NO:96 with S28F and S51M substitutions. The VL can have the amino acid sequence of SEQ ID NO:96 with S28F and K52R substitutions. The VL can have the amino acid sequence of SEQ ID NO:96 with S28H and S5M substitutions. The VL can have the amino acid sequence of SEQ ID NO:96 with S28H and K52R substitutions. The VL can have the amino acid sequence of SEQ ID NO:96 with S5M and K52R substitutions. The VL can have the amino acid sequence of SEQ ID NO:96 with S28F, S51M and K52R substitutions. The VL can have the amino acid sequence of SEQ ID NO:96 with S28H, S51M and K52R substitutions.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:97. The humanized anti-CEACAM1 antibody or antigen-binding fragment thereof can have a VL having at least 85% sequence identity to SEQ ID NO:97. The humanized anti-CEACAM1 antibody or antigen-binding fragment thereof can have a VL having at least 90% sequence identity to SEQ ID NO:97. The humanized anti-CEACAM1 antibody or antigen-binding fragment thereof can have a VL having at least 95% sequence identity to SEQ ID NO:97. The humanized anti-CEACAM1 antibody or antigen-binding fragment thereof can have a VL having at least 98% sequence identity to SEQ ID NO:97. In some embodiments, provided herein are humanized antibodies or antigen-binding fragments thereof that specifically bind CEACAM1 comprising a VL having the amino acid sequence of SEQ ID NO:97.

In some embodiments, the VL can have the amino acid sequence of SEQ ID NO:97 with a substitution at S28. The substitution can be S28F, or S28H. In some embodiments, the VL can have the amino acid sequence of SEQ ID NO:97 with a substitution at S51. The substitution can be S5IM. In some embodiments, the VL can have the amino acid sequence of SEQ ID NO:97 with a substitution at K52. The substitution can be K52R. In some embodiments, the VL can have the amino acid sequence of SEQ ID NO:97 with substitutions at S28, S51 and/or K52. The VL can have the amino acid sequence of SEQ ID NO:97 with (1) S28F and S51M substitutions, (2) S28F and K52R substitutions, (3) S28H and S51M substitutions, (4) S28H and K52R substitutions, (5) S5IM and K52R substitutions, (6) S28F, S51M and K52R substitutions, or (7) S28H, S51M and K52R substitutions. The VL can have the amino acid sequence of SEQ ID NO:97 with S28F substitution. The VL can have the amino acid sequence of SEQ ID NO:97 with S28H substitution. The VL can have the amino acid sequence of SEQ ID NO:97 with S51M substitution. The VL can have the amino acid sequence of SEQ ID NO:97 with K52R substitution. The VL can have the amino acid sequence of SEQ ID NO:97 with S28F and S51M substitutions. The VL can have the amino acid sequence of SEQ ID NO:97 with S28F and K52R substitutions. The VL can have the amino acid sequence of SEQ ID NO:97 with S28H and S51M substitutions. The VL can have the amino acid sequence of SEQ ID NO:97 with S28H and K52R substitutions. The VL can have the amino acid sequence of SEQ ID NO:97 with S51M and K52R substitutions. The VL can have the amino acid sequence of SEQ ID NO:97 with S28F, S51M and K52R substitutions. The VL can have the amino acid sequence of SEQ ID NO:97 with S28H, S51M and K52R substitutions.

In some embodiments, the VL can have the amino acid sequence of SEQ ID NO:97 with S28F substitution. In some embodiments, the VL can have the amino acid sequence of SEQ ID NO:100. In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:100. The humanized anti-CEACAM1 antibody or antigen-binding fragment thereof can have a VL having at least 85% sequence identity to SEQ ID NO:100. The humanized anti-CEACAM1 antibody or antigen-binding fragment thereof can have a VL having at least 90% sequence identity to SEQ ID NO:100. The humanized anti-CEACAM1 antibody or antigen-binding fragment thereof can have a VL having at least 95% sequence identity to SEQ ID NO:100. The humanized anti-CEACAM1 antibody or antigen-binding fragment thereof can have a VL having at least 98% sequence identity to SEQ ID NO:100. In some embodiments, provided herein are humanized antibodies or antigen-binding fragments thereof that specifically bind CEACAM1 comprising a VL having the amino acid sequence of SEQ ID NO:100.

In some embodiments, the VL can have the amino acid sequence of SEQ ID NO:97 with S28H substitution. In some embodiments, the VL can have the amino acid sequence of SEQ ID NO:101. In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:101. The humanized anti-CEACAM1 antibody or antigen-binding fragment thereof can have a VL having at least 85% sequence identity to SEQ ID NO:101. The humanized anti-CEACAM1 antibody or antigen-binding fragment thereof can have a VL having at least 90% sequence identity to SEQ ID NO:101. The humanized anti-CEACAM1 antibody or antigen-binding fragment thereof can have a VL having at least 95% sequence identity to SEQ ID NO:101. The humanized anti-CEACAM1 antibody or antigen-binding fragment thereof can have a VL having at least 98% sequence identity to SEQ ID NO:101. In some embodiments, provided herein are humanized antibodies or antigen-binding fragments thereof that specifically bind CEACAM1 comprising a VL having the amino acid sequence of SEQ ID NO:101.

In some embodiments, the VL can have the amino acid sequence of SEQ ID NO:97 with S51M substitution. In some embodiments, the VL can have the amino acid sequence of SEQ ID NO:102. In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:102. The humanized anti-CEACAM1 antibody or antigen-binding fragment thereof can have a VL having at least 85% sequence identity to SEQ ID NO:102. The humanized anti-CEACAM1 antibody or antigen-binding fragment thereof can have a VL having at least 90% sequence identity to SEQ ID NO:102. The humanized anti-CEACAM1 antibody or antigen-binding fragment thereof can have a VL having at least 95% sequence identity to SEQ ID NO:102. The humanized anti-CEACAM1 antibody or antigen-binding fragment thereof can have a VL having at least 98% sequence identity to SEQ ID NO:102. In some embodiments, provided herein are humanized antibodies or antigen-binding fragments thereof that specifically bind CEACAM1 comprising a VL having the amino acid sequence of SEQ ID NO:102.

In some embodiments, the VL can have the amino acid sequence of SEQ ID NO:97 with K52R substitution. In some embodiments, the VL can have the amino acid sequence of SEQ ID NO:103. In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:103. The humanized anti-CEACAM1 antibody or antigen-binding fragment thereof can have a VL having at least 85% sequence identity to SEQ ID NO:103. The humanized anti-CEACAM1 antibody or antigen-binding fragment thereof can have a VL having at least 90% sequence identity to SEQ ID NO:103. The humanized anti-CEACAM1 antibody or antigen-binding fragment thereof can have a VL having at least 95% sequence identity to SEQ ID NO:103. The humanized anti-CEACAM1 antibody or antigen-binding fragment thereof can have a VL having at least 98% sequence identity to SEQ ID NO:103. In some embodiments, provided herein are humanized antibodies or antigen-binding fragments thereof that specifically bind CEACAM1 comprising a VL having the amino acid sequence of SEQ ID NO:103.

In some embodiments, the VL can have the amino acid sequence of SEQ ID NO:97 with S28F and S51M substitutions. In some embodiments, the VL can have the amino acid sequence of SEQ ID NO:104. In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:104. The humanized anti-CEACAM1 antibody or antigen-binding fragment thereof can have a VL having at least 85% sequence identity to SEQ ID NO:104. The humanized anti-CEACAM1 antibody or antigen-binding fragment thereof can have a VL having at least 90% sequence identity to SEQ ID NO:104. The humanized anti-CEACAM1 antibody or antigen-binding fragment thereof can have a VL having at least 95% sequence identity to SEQ ID NO: 104. The humanized anti-CEACAM1 antibody or antigen-binding fragment thereof can have a VL having at least 98% sequence identity to SEQ ID NO:104. In some embodiments, provided herein are humanized antibodies or antigen-binding fragments thereof that specifically bind CEACAM1 comprising a VL having the amino acid sequence of SEQ ID NO:104.

In some embodiments, the VL can have the amino acid sequence of SEQ ID NO:97 with S28F and K52R substitutions. In some embodiments, the VL can have the amino acid sequence of SEQ ID NO:105. In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:105. The humanized anti-CEACAM1 antibody or antigen-binding fragment thereof can have a VL having at least 85% sequence identity to SEQ ID NO:105. The humanized anti-CEACAM1 antibody or antigen-binding fragment thereof can have a VL having at least 90% sequence identity to SEQ ID NO:105. The humanized anti-CEACAM1 antibody or antigen-binding fragment thereof can have a VL having at least 95% sequence identity to SEQ ID NO:105. The humanized anti-CEACAM1 antibody or antigen-binding fragment thereof can have a VL having at least 98% sequence identity to SEQ ID NO:105. In some embodiments, provided herein are humanized antibodies or antigen-binding fragments thereof that specifically bind CEACAM1 comprising a VL having the amino acid sequence of SEQ ID NO:105.

In some embodiments, the VL can have the amino acid sequence of SEQ ID NO:97 with S28F, S51M and K52R substitutions. In some embodiments, the VL can have the amino acid sequence of SEQ ID NO:106. In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:106. The humanized anti-CEACAM1 antibody or antigen-binding fragment thereof can have a VL having at least 85% sequence identity to SEQ ID NO:106. The humanized anti-CEACAM1 antibody or antigen-binding fragment thereof can have a VL having at least 90% sequence identity to SEQ ID NO:106. The humanized anti-CEACAM1 antibody or antigen-binding fragment thereof can have a VL having at least 95% sequence identity to SEQ ID NO:106. The humanized anti-CEACAM1 antibody or antigen-binding fragment thereof can have a VL having at least 98% sequence identity to SEQ ID NO:106. In some embodiments, provided herein are humanized antibodies or antigen-binding fragments thereof that specifically bind CEACAM1 comprising a VL having the amino acid sequence of SEQ ID NO:106.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:98. The humanized anti-CEACAM1 antibody or antigen-binding fragment thereof can have a VL having at least 85% sequence identity to SEQ ID NO:98. The humanized anti-CEACAM1 antibody or antigen-binding fragment thereof can have a VL having at least 90% sequence identity to SEQ ID NO:98. The humanized anti-CEACAM1 antibody or antigen-binding fragment thereof can have a VL having at least 95% sequence identity to SEQ ID NO:98. The humanized anti-CEACAM1 antibody or antigen-binding fragment thereof can have a VL having at least 98% sequence identity to SEQ ID NO:98. In some embodiments, provided herein are humanized antibodies or antigen-binding fragments thereof that specifically bind CEACAM1 comprising a VL having the amino acid sequence of SEQ ID NO:98.

In some embodiments, the VL can have the amino acid sequence of SEQ ID NO:98 with a substitution at S28. The substitution can be S28F, or S28H. In some embodiments, the VL can have the amino acid sequence of SEQ ID NO:98 with a substitution at S51. The substitution can be S51M. In some embodiments, the VL can have the amino acid sequence of SEQ ID NO:98 with a substitution at K52. The substitution can be K52R. In some embodiments, the VL can have the amino acid sequence of SEQ ID NO:98 with substitutions at S28, S51 and/or K52. The VL can have the amino acid sequence of SEQ ID NO:98 with (1) S28F and S51M substitutions, (2) S28F and K52R substitutions, (3) S28H and S51M substitutions, (4) S28H and K52R substitutions, (5) S5IM and K52R substitutions, (6) S28F, S51M and K52R substitutions, or (7) S28H, S51M and K52R substitutions. The VL can have the amino acid sequence of SEQ ID NO:98 with S28F substitution. The VL can have the amino acid sequence of SEQ ID NO:98 with S28H substitution. The VL can have the amino acid sequence of SEQ ID NO:98 with S51M substitution. The VL can have the amino acid sequence of SEQ ID NO:98 with K52R substitution. The VL can have the amino acid sequence of SEQ ID NO:98 with S28F and S51M substitutions. The VL can have the amino acid sequence of SEQ ID NO:98 with S28F and K52R substitutions. The VL can have the amino acid sequence of SEQ ID NO:98 with S28H and S51M substitutions. The VL can have the amino acid sequence of SEQ ID NO:98 with S28H and K52R substitutions. The VL can have the amino acid sequence of SEQ ID NO:98 with S51M and K52R substitutions. The VL can have the amino acid sequence of SEQ ID NO:98 with S28F, S51M and K52R substitutions. The VL can have the amino acid sequence of SEQ ID NO:98 with S28H, S51M and K52R substitutions.

In some embodiments, provided herein are humanized antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:99. The humanized anti-CEACAM1 antibody or antigen-binding fragment thereof can have a VL having at least 85% sequence identity to SEQ ID NO:99. The humanized anti-CEACAM1 antibody or antigen-binding fragment thereof can have a VL having at least 90% sequence identity to SEQ ID NO:99. The humanized anti-CEACAM1 antibody or antigen-binding fragment thereof can have a VL having at least 95% sequence identity to SEQ ID NO:99. The humanized anti-CEACAM1 antibody or antigen-binding fragment thereof can have a VL having at least 98% sequence identity to SEQ ID NO:99. In some embodiments, provided herein are humanized antibodies or antigen-binding fragments thereof that specifically bind CEACAM1 comprising a VL having the amino acid sequence of SEQ ID NO:99.

In some embodiments, the VL can have the amino acid sequence of SEQ ID NO:99 with a substitution at S28. The substitution can be S28F, or S28H. In some embodiments, the VL can have the amino acid sequence of SEQ ID NO:99 with a substitution at S51. The substitution can be S5IM. In some embodiments, the VL can have the amino acid sequence of SEQ ID NO:99 with a substitution at K52. The substitution can be K52R. In some embodiments, the VL can have the amino acid sequence of SEQ ID NO:99 with substitutions at S28, S51 and/or K52. The VL can have the amino acid sequence of SEQ ID NO:99 with (1) S28F and S51M substitutions, (2) S28F and K52R substitutions, (3) S28H and S51M substitutions, (4) S28H and K52R substitutions, (5) S5IM and K52R substitutions, (6) S28F, S51M and K52R substitutions, or (7) S28H, S51M and K52R substitutions. The VL can have the amino acid sequence of SEQ ID NO:99 with S28F substitution. The VL can have the amino acid sequence of SEQ ID NO:99 with S28H substitution. The VL can have the amino acid sequence of SEQ ID NO:99 with S51M substitution. The VL can have the amino acid sequence of SEQ ID NO:99 with K52R substitution. The VL can have the amino acid sequence of SEQ ID NO:99 with S28F and S51M substitutions. The VL can have the amino acid sequence of SEQ ID NO:99 with S28F and K52R substitutions. The VL can have the amino acid sequence of SEQ ID NO:99 with S28H and S51M substitutions. The VL can have the amino acid sequence of SEQ ID NO:99 with S28H and K52R substitutions. The VL can have the amino acid sequence of SEQ ID NO:99 with S51M and K52R substitutions. The VL can have the amino acid sequence of SEQ ID NO:99 with S28F, S51M and K52R substitutions. The VL can have the amino acid sequence of SEQ ID NO:99 with S28H, S51M and K52R substitutions.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:123. The humanized anti-CEACAM1 antibody or antigen-binding fragment thereof can have a VH having at least 85% sequence identity to SEQ ID NO:123. The humanized anti-CEACAM1 antibody or antigen-binding fragment thereof can have a VH having at least 90% sequence identity to SEQ ID NO:123. The humanized anti-CEACAM1 antibody or antigen-binding fragment thereof can have a VH having at least 95% sequence identity to SEQ ID NO:123. The humanized anti-CEACAM1 antibody or antigen-binding fragment thereof can have a VH having at least 98% sequence identity to SEQ ID NO:123. In some embodiments, provided herein are humanized antibodies or antigen-binding fragments thereof that specifically bind CEACAM1 comprising a VH having the amino acid sequence of SEQ ID NO:123.

In some embodiments, the VH can have the amino acid sequence of SEQ ID NO:123 with a substitution at H35. The substitution can be H35M. In some embodiments, the VH can have the amino acid sequence of SEQ ID NO: 123 with a substitution at N56. The substitution can be N56I, N56Q, N56V, or N56W. In some embodiments, the VH can have the amino acid sequence of SEQ ID NO: 123 with a substitution at Y100. The substitution can be Y100F, Y100V, or Y100W. In some embodiments, the VH can have the amino acid sequence of SEQ ID NO:123 with substitutions at H35, N56 and/or Y100. The VH can have the amino acid sequence of SEQ ID NO:123 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions. The VH can have the amino acid sequence of SEQ ID NO:123 with H35M substitution. The VH can have the amino acid sequence of SEQ ID NO:123 with N56I substitution. The VH can have the amino acid sequence of SEQ ID NO:123 with N56Q substitution. The VH can have the amino acid sequence of SEQ ID NO:123 with N56V substitution. The VH can have the amino acid sequence of SEQ ID NO:123 with N56W substitution. The VH can have the amino acid sequence of SEQ ID NO:123 with Y100F substitution. The VH can have the amino acid sequence of SEQ ID NO:123 with Y100V substitution. The VH can have the amino acid sequence of SEQ ID NO:123 with Y100W substitution. The VH can have the amino acid sequence of SEQ ID NO: 123 with H35M and N56I substitutions. The VH can have the amino acid sequence of SEQ ID NO:123 with H35M and N56Q substitutions. The VH can have the amino acid sequence of SEQ ID NO: 123 with H35M and N56V substitutions. The VH can have the amino acid sequence of SEQ ID NO:123 with H35M and N56W substitutions. The VH can have the amino acid sequence of SEQ ID NO:123 with H35M and Y100F substitutions. The VH can have the amino acid sequence of SEQ ID NO: 123 with H35M and Y100V substitutions. The VH can have the amino acid sequence of SEQ ID NO:123 with H35M and Y100W substitutions. The VH can have the amino acid sequence of SEQ ID NO: 123 with N56I and Y100F substitutions. The VH can have the amino acid sequence of SEQ ID NO: 123 with N56I and Y100V substitutions. The VH can have the amino acid sequence of SEQ ID NO:123 with N56I and Y100W substitutions. The VH can have the amino acid sequence of SEQ ID NO: 123 with N56Q and Y100F substitutions. The VH can have the amino acid sequence of SEQ ID NO:123 with N56Q and Y100V substitutions. The VH can have the amino acid sequence of SEQ ID NO:123 with N56Q and Y100W substitutions. The VH can have the amino acid sequence of SEQ ID NO: 123 with N56V and Y100F substitutions. The VH can have the amino acid sequence of SEQ ID NO:123 with N56V and Y100V substitutions. The VH can have the amino acid sequence of SEQ ID NO: 123 with N56V and Y100W substitutions. The VH can have the amino acid sequence of SEQ ID NO:123 with N56W and Y100F substitutions. The VH can have the amino acid sequence of SEQ ID NO:123 with N56W and Y100V substitutions. The VH can have the amino acid sequence of SEQ ID NO: 123 with N56W and Y100W substitutions. The VH can have the amino acid sequence of SEQ ID NO:123 with H35M, N56I and Y100F substitutions. The VH can have the amino acid sequence of SEQ ID NO: 123 with H35M, N56I and Y100V substitutions. The VH can have the amino acid sequence of SEQ ID NO:123 with H35M, N56I and Y100W substitutions. The VH can have the amino acid sequence of SEQ ID NO:123 with H35M, N56Q and Y100F substitutions. The VH can have the amino acid sequence of SEQ ID NO:123 with H35M, N56Q and Y100V substitutions. The VH can have the amino acid sequence of SEQ ID NO:123 with H35M, N56Q and Y100W substitutions. The VH can have the amino acid sequence of SEQ ID NO:123 with H35M, N56V and Y100F substitutions. The VH can have the amino acid sequence of SEQ ID NO:123 with H35M, N56V and Y100V substitutions. The VH can have the amino acid sequence of SEQ ID NO:123 with H35M, N56V and Y100W substitutions. The VH can have the amino acid sequence of SEQ ID NO:123 with H35M, N56W and Y100F substitutions. The VH can have the amino acid sequence of SEQ ID NO: 123 with H35M, N56W and Y100V substitutions. The VH can have the amino acid sequence of SEQ ID NO:123 with H35M, N56W and Y100W substitutions.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:124. The humanized anti-CEACAM1 antibody or antigen-binding fragment thereof can have a VH having at least 85% sequence identity to SEQ ID NO:124. The humanized anti-CEACAM1 antibody or antigen-binding fragment thereof can have a VH having at least 90% sequence identity to SEQ ID NO:124. The humanized anti-CEACAM1 antibody or antigen-binding fragment thereof can have a VH having at least 95% sequence identity to SEQ ID NO:124. The humanized anti-CEACAM1 antibody or antigen-binding fragment thereof can have a VH having at least 98% sequence identity to SEQ ID NO:124. In some embodiments, provided herein are humanized antibodies or antigen-binding fragments thereof that specifically bind CEACAM1 comprising a VH having the amino acid sequence of SEQ ID NO:124.

In some embodiments, the VH can have the amino acid sequence of SEQ ID NO: 124 with a substitution at H35. The substitution can be H35M. In some embodiments, the VH can have the amino acid sequence of SEQ ID NO: 124 with a substitution at N56. The substitution can be N56I, N56Q, N56V, or N56W. In some embodiments, the VH can have the amino acid sequence of SEQ ID NO:124 with a substitution at Y100. The substitution can be Y100F, Y100V, or Y100W. In some embodiments, the VH can have the amino acid sequence of SEQ ID NO:124 with substitutions at H35, N56 and/or Y100. The VH can have the amino acid sequence of SEQ ID NO:124 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions. The VH can have the amino acid sequence of SEQ ID NO:124 with H35M substitution. The VH can have the amino acid sequence of SEQ ID NO:124 with N56I substitution. The VH can have the amino acid sequence of SEQ ID NO:124 with N56Q substitution. The VH can have the amino acid sequence of SEQ ID NO:124 with N56V substitution. The VH can have the amino acid sequence of SEQ ID NO:124 with N56W substitution. The VH can have the amino acid sequence of SEQ ID NO:124 with Y100F substitution. The VH can have the amino acid sequence of SEQ ID NO:124 with Y100V substitution. The VH can have the amino acid sequence of SEQ ID NO:124 with Y100W substitution. The VH can have the amino acid sequence of SEQ ID NO: 124 with H35M and N56I substitutions. The VH can have the amino acid sequence of SEQ ID NO: 124 with H35M and N56Q substitutions. The VH can have the amino acid sequence of SEQ ID NO:124 with H35M and N56V substitutions. The VH can have the amino acid sequence of SEQ ID NO: 124 with H35M and N56W substitutions. The VH can have the amino acid sequence of SEQ ID NO:124 with H35M and Y100F substitutions. The VH can have the amino acid sequence of SEQ ID NO:124 with H35M and Y100V substitutions. The VH can have the amino acid sequence of SEQ ID NO: 124 with H35M and Y100W substitutions. The VH can have the amino acid sequence of SEQ ID NO:124 with N56I and Y100F substitutions. The VH can have the amino acid sequence of SEQ ID NO: 124 with N56I and Y100V substitutions. The VH can have the amino acid sequence of SEQ ID NO:124 with N56I and Y100W substitutions. The VH can have the amino acid sequence of SEQ ID NO:124 with N56Q and Y100F substitutions. The VH can have the amino acid sequence of SEQ ID NO: 124 with N56Q and Y100V substitutions. The VH can have the amino acid sequence of SEQ ID NO:124 with N56Q and Y100W substitutions. The VH can have the amino acid sequence of SEQ ID NO: 124 with N56V and Y100F substitutions. The VH can have the amino acid sequence of SEQ ID NO:124 with N56V and Y100V substitutions. The VH can have the amino acid sequence of SEQ ID NO:124 with N56V and Y100W substitutions. The VH can have the amino acid sequence of SEQ ID NO: 124 with N56W and Y100F substitutions. The VH can have the amino acid sequence of SEQ ID NO:124 with N56W and Y100V substitutions. The VH can have the amino acid sequence of SEQ ID NO:124 with N56W and Y100W substitutions. The VH can have the amino acid sequence of SEQ ID NO:124 with H35M, N56I and Y100F substitutions. The VH can have the amino acid sequence of SEQ ID NO:124 with H35M, N56I and Y100V substitutions. The VH can have the amino acid sequence of SEQ ID NO:124 with H35M, N56I and Y100W substitutions. The VH can have the amino acid sequence of SEQ ID NO:124 with H35M, N56Q and Y100F substitutions. The VH can have the amino acid sequence of SEQ ID NO:124 with H35M, N56Q and Y100V substitutions. The VH can have the amino acid sequence of SEQ ID NO:124 with H35M, N56Q and Y100W substitutions. The VH can have the amino acid sequence of SEQ ID NO:124 with H35M, N56V and Y100F substitutions. The VH can have the amino acid sequence of SEQ ID NO:124 with H35M, N56V and Y100V substitutions. The VH can have the amino acid sequence of SEQ ID NO:124 with H35M, N56V and Y100W substitutions. The VH can have the amino acid sequence of SEQ ID NO:124 with H35M, N56W and Y100F substitutions. The VH can have the amino acid sequence of SEQ ID NO:124 with H35M, N56W and Y100V substitutions. The VH can have the amino acid sequence of SEQ ID NO:124 with H35M, N56W and Y100W substitutions.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:125. The humanized anti-CEACAM1 antibody or antigen-binding fragment thereof can have a VH having at least 85% sequence identity to SEQ ID NO:125. The humanized anti-CEACAM1 antibody or antigen-binding fragment thereof can have a VH having at least 90% sequence identity to SEQ ID NO:125. The humanized anti-CEACAM1 antibody or antigen-binding fragment thereof can have a VH having at least 95% sequence identity to SEQ ID NO:125. The humanized anti-CEACAM1 antibody or antigen-binding fragment thereof can have a VH having at least 98% sequence identity to SEQ ID NO:125. In some embodiments, provided herein are humanized antibodies or antigen-binding fragments thereof that specifically bind CEACAM1 comprising a VH having the amino acid sequence of SEQ ID NO:125.

In some embodiments, the VH can have the amino acid sequence of SEQ ID NO: 125 with a substitution at H35. The substitution can be H35M. In some embodiments, the VH can have the amino acid sequence of SEQ ID NO:125 with a substitution at N56. The substitution can be N56I, N56Q, N56V, or N56W. In some embodiments, the VH can have the amino acid sequence of SEQ ID NO:125 with a substitution at Y100. The substitution can be Y100F, Y100V, or Y100W. In some embodiments, the VH can have the amino acid sequence of SEQ ID NO:125 with substitutions at H35, N56 and/or Y100. The VH can have the amino acid sequence of SEQ ID NO:125 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions. The VH can have the amino acid sequence of SEQ ID NO:125 with H35M substitution. The VH can have the amino acid sequence of SEQ ID NO:125 with N56I substitution. The VH can have the amino acid sequence of SEQ ID NO:125 with N56Q substitution. The VH can have the amino acid sequence of SEQ ID NO:125 with N56V substitution. The VH can have the amino acid sequence of SEQ ID NO: 125 with N56W substitution. The VH can have the amino acid sequence of SEQ ID NO:125 with Y100F substitution. The VH can have the amino acid sequence of SEQ ID NO:125 with Y100V substitution. The VH can have the amino acid sequence of SEQ ID NO: 125 with Y100W substitution. The VH can have the amino acid sequence of SEQ ID NO:125 with H35M and N56I substitutions. The VH can have the amino acid sequence of SEQ ID NO: 125 with H35M and N56Q substitutions. The VH can have the amino acid sequence of SEQ ID NO:125 with H35M and N56V substitutions. The VH can have the amino acid sequence of SEQ ID NO:125 with H35M and N56W substitutions. The VH can have the amino acid sequence of SEQ ID NO: 125 with H35M and Y100F substitutions. The VH can have the amino acid sequence of SEQ ID NO:125 with H35M and Y100V substitutions. The VH can have the amino acid sequence of SEQ ID NO: 125 with H35M and Y100W substitutions. The VH can have the amino acid sequence of SEQ ID NO:125 with N56I and Y100F substitutions. The VH can have the amino acid sequence of SEQ ID NO:125 with N56I and Y100V substitutions. The VH can have the amino acid sequence of SEQ ID NO: 125 with N56I and Y100W substitutions. The VH can have the amino acid sequence of SEQ ID NO:125 with N56Q and Y100F substitutions. The VH can have the amino acid sequence of SEQ ID NO:125 with N56Q and Y100V substitutions. The VH can have the amino acid sequence of SEQ ID NO:125 with N56Q and Y100W substitutions. The VH can have the amino acid sequence of SEQ ID NO:125 with N56V and Y100F substitutions. The VH can have the amino acid sequence of SEQ ID NO: 125 with N56V and Y100V substitutions. The VH can have the amino acid sequence of SEQ ID NO:125 with N56V and Y100W substitutions. The VH can have the amino acid sequence of SEQ ID NO: 125 with N56W and Y100F substitutions. The VH can have the amino acid sequence of SEQ ID NO:125 with N56W and Y100V substitutions. The VH can have the amino acid sequence of SEQ ID NO:125 with N56W and Y100W substitutions. The VH can have the amino acid sequence of SEQ ID NO: 125 with H35M, N56I and Y100F substitutions. The VH can have the amino acid sequence of SEQ ID NO:125 with H35M, N56I and Y100V substitutions. The VH can have the amino acid sequence of SEQ ID NO:125 with H35M, N56I and Y100W substitutions. The VH can have the amino acid sequence of SEQ ID NO:125 with H35M, N56Q and Y100F substitutions. The VH can have the amino acid sequence of SEQ ID NO:125 with H35M, N56Q and Y100V substitutions. The VH can have the amino acid sequence of SEQ ID NO: 125 with H35M, N56Q and Y100W substitutions. The VH can have the amino acid sequence of SEQ ID NO:125 with H35M, N56V and Y100F substitutions. The VH can have the amino acid sequence of SEQ ID NO:125 with H35M, N56V and Y100V substitutions. The VH can have the amino acid sequence of SEQ ID NO:125 with H35M, N56V and Y100W substitutions. The VH can have the amino acid sequence of SEQ ID NO: 125 with H35M, N56W and Y100F substitutions. The VH can have the amino acid sequence of SEQ ID NO:125 with H35M, N56W and Y100V substitutions. The VH can have the amino acid sequence of SEQ ID NO:125 with H35M, N56W and Y100W substitutions.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:126. The humanized anti-CEACAM1 antibody or antigen-binding fragment thereof can have a VH having at least 85% sequence identity to SEQ ID NO:126. The humanized anti-CEACAM1 antibody or antigen-binding fragment thereof can have a VH having at least 90% sequence identity to SEQ ID NO:126. The humanized anti-CEACAM1 antibody or antigen-binding fragment thereof can have a VH having at least 95% sequence identity to SEQ ID NO: 126. The humanized anti-CEACAM1 antibody or antigen-binding fragment thereof can have a VH having at least 98% sequence identity to SEQ ID NO:126. In some embodiments, provided herein are humanized antibodies or antigen-binding fragments thereof that specifically bind CEACAM1 comprising a VH having the amino acid sequence of SEQ ID NO:126.

In some embodiments, the VH can have the amino acid sequence of SEQ ID NO: 126 with a substitution at H35. The substitution can be H35M. In some embodiments, the VH can have the amino acid sequence of SEQ ID NO: 126 with a substitution at N56. The substitution can be N56I, N56Q, N56V, or N56W. In some embodiments, the VH can have the amino acid sequence of SEQ ID NO:126 with a substitution at Y100. The substitution can be Y100F, Y100V, or Y100W. In some embodiments, the VH can have the amino acid sequence of SEQ ID NO:126 with substitutions at H35, N56 and/or Y100. The VH can have the amino acid sequence of SEQ ID NO:126 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions. The VH can have the amino acid sequence of SEQ ID NO:126 with H35M substitution. The VH can have the amino acid sequence of SEQ ID NO:126 with N56I substitution. The VH can have the amino acid sequence of SEQ ID NO:126 with N56Q substitution. The VH can have the amino acid sequence of SEQ ID NO:126 with N56V substitution. The VH can have the amino acid sequence of SEQ ID NO: 126 with N56W substitution. The VH can have the amino acid sequence of SEQ ID NO:126 with Y100F substitution. The VH can have the amino acid sequence of SEQ ID NO:126 with Y100V substitution. The VH can have the amino acid sequence of SEQ ID NO:126 with Y100W substitution. The VH can have the amino acid sequence of SEQ ID NO:126 with H35M and N56I substitutions. The VH can have the amino acid sequence of SEQ ID NO: 126 with H35M and N56Q substitutions. The VH can have the amino acid sequence of SEQ ID NO:126 with H35M and N56V substitutions. The VH can have the amino acid sequence of SEQ ID NO:126 with H35M and N56W substitutions. The VH can have the amino acid sequence of SEQ ID NO:126 with H35M and Y100F substitutions. The VH can have the amino acid sequence of SEQ ID NO:126 with H35M and Y100V substitutions. The VH can have the amino acid sequence of SEQ ID NO: 126 with H35M and Y100W substitutions. The VH can have the amino acid sequence of SEQ ID NO:126 with N56I and Y100F substitutions. The VH can have the amino acid sequence of SEQ ID NO: 126 with N56I and Y100V substitutions. The VH can have the amino acid sequence of SEQ ID NO:126 with N56I and Y100W substitutions. The VH can have the amino acid sequence of SEQ ID NO:126 with N56Q and Y100F substitutions. The VH can have the amino acid sequence of SEQ ID NO: 126 with N56Q and Y100V substitutions. The VH can have the amino acid sequence of SEQ ID NO:126 with N56Q and Y100W substitutions. The VH can have the amino acid sequence of SEQ ID NO: 126 with N56V and Y100F substitutions. The VH can have the amino acid sequence of SEQ ID NO:126 with N56V and Y100V substitutions. The VH can have the amino acid sequence of SEQ ID NO:126 with N56V and Y100W substitutions. The VH can have the amino acid sequence of SEQ ID NO: 126 with N56W and Y100F substitutions. The VH can have the amino acid sequence of SEQ ID NO:126 with N56W and Y100V substitutions. The VH can have the amino acid sequence of SEQ ID NO: 126 with N56W and Y100W substitutions. The VH can have the amino acid sequence of SEQ ID NO: 126 with H35M, N56I and Y100F substitutions. The VH can have the amino acid sequence of SEQ ID NO:126 with H35M, N56I and Y100V substitutions. The VH can have the amino acid sequence of SEQ ID NO:126 with H35M, N56I and Y100W substitutions. The VH can have the amino acid sequence of SEQ ID NO:126 with H35M, N56Q and Y100F substitutions. The VH can have the amino acid sequence of SEQ ID NO:126 with H35M, N56Q and Y100V substitutions. The VH can have the amino acid sequence of SEQ ID NO: 126 with H35M, N56Q and Y100W substitutions. The VH can have the amino acid sequence of SEQ ID NO:126 with H35M, N56V and Y100F substitutions. The VH can have the amino acid sequence of SEQ ID NO:126 with H35M, N56V and Y100V substitutions. The VH can have the amino acid sequence of SEQ ID NO:126 with H35M, N56V and Y100W substitutions. The VH can have the amino acid sequence of SEQ ID NO: 126 with H35M, N56W and Y100F substitutions. The VH can have the amino acid sequence of SEQ ID NO:126 with H35M, N56W and Y100V substitutions. The VH can have the amino acid sequence of SEQ ID NO:126 with H35M, N56W and Y100W substitutions.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:127. The humanized anti-CEACAM1 antibody or antigen-binding fragment thereof can have a VH having at least 85% sequence identity to SEQ ID NO:127. The humanized anti-CEACAM1 antibody or antigen-binding fragment thereof can have a VH having at least 90% sequence identity to SEQ ID NO:127. The humanized anti-CEACAM1 antibody or antigen-binding fragment thereof can have a VH having at least 95% sequence identity to SEQ ID NO:127. The humanized anti-CEACAM1 antibody or antigen-binding fragment thereof can have a VH having at least 98% sequence identity to SEQ ID NO:127. In some embodiments, provided herein are humanized antibodies or antigen-binding fragments thereof that specifically bind CEACAM1 comprising a VH having the amino acid sequence of SEQ ID NO:127.

In some embodiments, the VH can have the amino acid sequence of SEQ ID NO: 127 with a substitution at H35. The substitution can be H35M. In some embodiments, the VH can have the amino acid sequence of SEQ ID NO:127 with a substitution at N56. The substitution can be N56I, N56Q, N56V, or N56W. In some embodiments, the VH can have the amino acid sequence of SEQ ID NO:127 with a substitution at Y100. The substitution can be Y100F, Y100V, or Y100W. In some embodiments, the VH can have the amino acid sequence of SEQ ID NO:127 with substitutions at H35, N56 and/or Y100. The VH can have the amino acid sequence of SEQ ID NO:127 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions. The VH can have the amino acid sequence of SEQ ID NO:127 with H35M substitution. The VH can have the amino acid sequence of SEQ ID NO:127 with N56I substitution. The VH can have the amino acid sequence of SEQ ID NO:127 with N56Q substitution. The VH can have the amino acid sequence of SEQ ID NO:127 with N56V substitution. The VH can have the amino acid sequence of SEQ ID NO:127 with N56W substitution. The VH can have the amino acid sequence of SEQ ID NO:127 with Y100F substitution. The VH can have the amino acid sequence of SEQ ID NO:127 with Y100V substitution. The VH can have the amino acid sequence of SEQ ID NO: 127 with Y100W substitution. The VH can have the amino acid sequence of SEQ ID NO:127 with H35M and N56I substitutions. The VH can have the amino acid sequence of SEQ ID NO: 127 with H35M and N56Q substitutions. The VH can have the amino acid sequence of SEQ ID NO:127 with H35M and N56V substitutions. The VH can have the amino acid sequence of SEQ ID NO:127 with H35M and N56W substitutions. The VH can have the amino acid sequence of SEQ ID NO: 127 with H35M and Y100F substitutions. The VH can have the amino acid sequence of SEQ ID NO:127 with H35M and Y100V substitutions. The VH can have the amino acid sequence of SEQ ID NO: 127 with H35M and Y100W substitutions. The VH can have the amino acid sequence of SEQ ID NO:127 with N56I and Y100F substitutions. The VH can have the amino acid sequence of SEQ ID NO:127 with N56I and Y100V substitutions. The VH can have the amino acid sequence of SEQ ID NO: 127 with N56I and Y100W substitutions. The VH can have the amino acid sequence of SEQ ID NO:127 with N56Q and Y100F substitutions. The VH can have the amino acid sequence of SEQ ID NO: 127 with N56Q and Y100V substitutions. The VH can have the amino acid sequence of SEQ ID NO: 127 with N56Q and Y100W substitutions. The VH can have the amino acid sequence of SEQ ID NO:127 with N56V and Y100F substitutions. The VH can have the amino acid sequence of SEQ ID NO: 127 with N56V and Y100V substitutions. The VH can have the amino acid sequence of SEQ ID NO:127 with N56V and Y100W substitutions. The VH can have the amino acid sequence of SEQ ID NO:127 with N56W and Y100F substitutions. The VH can have the amino acid sequence of SEQ ID NO: 127 with N56W and Y100V substitutions. The VH can have the amino acid sequence of SEQ ID NO:127 with N56W and Y100W substitutions. The VH can have the amino acid sequence of SEQ ID NO: 127 with H35M, N56I and Y100F substitutions. The VH can have the amino acid sequence of SEQ ID NO:127 with H35M, N56I and Y100V substitutions. The VH can have the amino acid sequence of SEQ ID NO:127 with H35M, N56I and Y100W substitutions. The VH can have the amino acid sequence of SEQ ID NO:127 with H35M, N56Q and Y100F substitutions. The VH can have the amino acid sequence of SEQ ID NO:127 with H35M, N56Q and Y100V substitutions. The VH can have the amino acid sequence of SEQ ID NO: 127 with H35M, N56Q and Y100W substitutions. The VH can have the amino acid sequence of SEQ ID NO:127 with H35M, N56V and Y100F substitutions. The VH can have the amino acid sequence of SEQ ID NO:127 with H35M, N56V and Y100V substitutions. The VH can have the amino acid sequence of SEQ ID NO:127 with H35M, N56V and Y100W substitutions. The VH can have the amino acid sequence of SEQ ID NO:127 with H35M, N56W and Y100F substitutions. The VH can have the amino acid sequence of SEQ ID NO:127 with H35M, N56W and Y100V substitutions. The VH can have the amino acid sequence of SEQ ID NO:127 with H35M, N56W and Y100W substitutions.

In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:128. The humanized anti-CEACAM1 antibody or antigen-binding fragment thereof can have a VH having at least 85% sequence identity to SEQ ID NO:128. The humanized anti-CEACAM1 antibody or antigen-binding fragment thereof can have a VH having at least 90% sequence identity to SEQ ID NO:128. The humanized anti-CEACAM1 antibody or antigen-binding fragment thereof can have a VH having at least 95% sequence identity to SEQ ID NO:128. The humanized anti-CEACAM1 antibody or antigen-binding fragment thereof can have a VH having at least 98% sequence identity to SEQ ID NO:128. In some embodiments, provided herein are humanized antibodies or antigen-binding fragments thereof that specifically bind CEACAM1 comprising a VH having the amino acid sequence of SEQ ID NO:128.

In some embodiments, the VH can have the amino acid sequence of SEQ ID NO:128 with a substitution at H35. The substitution can be H35M. In some embodiments, the VH can have the amino acid sequence of SEQ ID NO:128 with a substitution at N56. The substitution can be N56I, N56Q, N56V, or N56W. In some embodiments, the VH can have the amino acid sequence of SEQ ID NO: 128 with a substitution at Y100. The substitution can be Y100F, Y100V, or Y100W. In some embodiments, the VH can have the amino acid sequence of SEQ ID NO:128 with substitutions at H35, N56 and/or Y100. The VH can have the amino acid sequence of SEQ ID NO:128 with (1) H35M and N56I substitutions, (2) H35M and N56Q substitutions, (3) H35M and N56V substitutions, (4) H35M and N56W substitutions, (5) H35M and Y100F substitutions, (6) H35M and Y100V substitutions, (7) H35M and Y100W substitutions, (8) N56I and Y100F substitutions, (9) N56I and Y100V substitutions, (10) N56I and Y100W substitutions, (11) N56Q and Y100F substitutions, (12) N56Q and Y100V substitutions, (13) N56Q and Y100W substitutions, (14) N56V and Y100F substitutions, (15) N56V and Y100V substitutions, (16) N56V and Y100W substitutions, (17) N56W and Y100F substitutions, (18) N56W and Y100V substitutions, (19) N56W and Y100W substitutions, (20) H35M, N56I and Y100F substitutions, (21) H35M, N56I and Y100V substitutions, (22) H35M, N56I and Y100W substitutions, (23) H35M, N56Q and Y100F substitutions, (24) H35M, N56Q and Y100V substitutions, (25) H35M, N56Q and Y100W substitutions, (26) H35M, N56V and Y100F substitutions, (27) H35M, N56V and Y100V substitutions, (28) H35M, N56V and Y100W substitutions, (29) H35M, N56W and Y100F substitutions, (30) H35M, N56W and Y100V substitutions, or (31) H35M, N56W and Y100W substitutions. The VH can have the amino acid sequence of SEQ ID NO:128 with H35M substitution. The VH can have the amino acid sequence of SEQ ID NO:128 with N56I substitution. The VH can have the amino acid sequence of SEQ ID NO:128 with N56Q substitution. The VH can have the amino acid sequence of SEQ ID NO:128 with N56V substitution. The VH can have the amino acid sequence of SEQ ID NO:128 with N56W substitution. The VH can have the amino acid sequence of SEQ ID NO:128 with Y100F substitution. The VH can have the amino acid sequence of SEQ ID NO:128 with Y100V substitution. The VH can have the amino acid sequence of SEQ ID NO: 128 with Y100W substitution. The VH can have the amino acid sequence of SEQ ID NO:128 with H35M and N56I substitutions. The VH can have the amino acid sequence of SEQ ID NO:128 with H35M and N56Q substitutions. The VH can have the amino acid sequence of SEQ ID NO: 128 with H35M and N56V substitutions. The VH can have the amino acid sequence of SEQ ID NO:128 with H35M and N56W substitutions. The VH can have the amino acid sequence of SEQ ID NO: 128 with H35M and Y100F substitutions. The VH can have the amino acid sequence of SEQ ID NO: 128 with H35M and Y100V substitutions. The VH can have the amino acid sequence of SEQ ID NO:128 with H35M and Y100W substitutions. The VH can have the amino acid sequence of SEQ ID NO: 128 with N56I and Y100F substitutions. The VH can have the amino acid sequence of SEQ ID NO:128 with N56I and Y100V substitutions. The VH can have the amino acid sequence of SEQ ID NO:128 with N56I and Y100W substitutions. The VH can have the amino acid sequence of SEQ ID NO: 128 with N56Q and Y100F substitutions. The VH can have the amino acid sequence of SEQ ID NO:128 with N56Q and Y100V substitutions. The VH can have the amino acid sequence of SEQ ID NO: 128 with N56Q and Y100W substitutions. The VH can have the amino acid sequence of SEQ ID NO:128 with N56V and Y100F substitutions. The VH can have the amino acid sequence of SEQ ID NO:128 with N56V and Y100V substitutions. The VH can have the amino acid sequence of SEQ ID NO: 128 with N56V and Y100W substitutions. The VH can have the amino acid sequence of SEQ ID NO:128 with N56W and Y100F substitutions. The VH can have the amino acid sequence of SEQ ID NO: 128 with N56W and Y100V substitutions. The VH can have the amino acid sequence of SEQ ID NO:128 with N56W and Y100W substitutions. The VH can have the amino acid sequence of SEQ ID NO:128 with H35M, N56I and Y100F substitutions. The VH can have the amino acid sequence of SEQ ID NO:128 with H35M, N56I and Y100V substitutions. The VH can have the amino acid sequence of SEQ ID NO:128 with H35M, N56I and Y100W substitutions. The VH can have the amino acid sequence of SEQ ID NO:128 with H35M, N56Q and Y100F substitutions. The VH can have the amino acid sequence of SEQ ID NO:128 with H35M, N56Q and Y100V substitutions. The VH can have the amino acid sequence of SEQ ID NO:128 with H35M, N56Q and Y100W substitutions. The VH can have the amino acid sequence of SEQ ID NO:128 with H35M, N56V and Y100F substitutions. The VH can have the amino acid sequence of SEQ ID NO:128 with H35M, N56V and Y100V substitutions. The VH can have the amino acid sequence of SEQ ID NO:128 with H35M, N56V and Y100W substitutions. The VH can have the amino acid sequence of SEQ ID NO:128 with H35M, N56W and Y100F substitutions. The VH can have the amino acid sequence of SEQ ID NO: 128 with H35M, N56W and Y100V substitutions. The VH can have the amino acid sequence of SEQ ID NO:128 with H35M, N56W and Y100W substitutions.

In some embodiments, the VH can have the amino acid sequence of SEQ ID NO:128 with H35M, N56Q and Y100F substitutions. In some embodiments, the VH can have the amino acid sequence of SEQ ID NO:130. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:130. The humanized anti-CEACAM1 antibody or antigen-binding fragment thereof can have a VH having at least 85% sequence identity to SEQ ID NO:130. The humanized anti-CEACAM1 antibody or antigen-binding fragment thereof can have a VH having at least 90% sequence identity to SEQ ID NO:130. The humanized anti-CEACAM1 antibody or antigen-binding fragment thereof can have a VH having at least 95% sequence identity to SEQ ID NO:130. The humanized anti-CEACAM1 antibody or antigen-binding fragment thereof can have a VH having at least 98% sequence identity to SEQ ID NO:130. In some embodiments, provided herein are humanized antibodies or antigen-binding fragments thereof that specifically bind CEACAM1 comprising a VH having the amino acid sequence of SEQ ID NO:130.

In some embodiments, the VH can have the amino acid sequence of SEQ ID NO: 128 with N56Q substitution. In some embodiments, the VH can have the amino acid sequence of SEQ ID NO:143. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:143. The humanized anti-CEACAM1 antibody or antigen-binding fragment thereof can have a VH having at least 85% sequence identity to SEQ ID NO:143. The humanized anti-CEACAM1 antibody or antigen-binding fragment thereof can have a VH having at least 90% sequence identity to SEQ ID NO:143. The humanized anti-CEACAM1 antibody or antigen-binding fragment thereof can have a VH having at least 95% sequence identity to SEQ ID NO:143. The humanized anti-CEACAM1 antibody or antigen-binding fragment thereof can have a VH having at least 98% sequence identity to SEQ ID NO:143. In some embodiments, provided herein are humanized antibodies or antigen-binding fragments thereof that specifically bind CEACAM1 comprising a VH having the amino acid sequence of SEQ ID NO:143.

In some embodiments, the VH can have the amino acid sequence of SEQ ID NO: 128 with N56I substitution. In some embodiments, the VH can have the amino acid sequence of SEQ ID NO:144. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:144. The humanized anti-CEACAM1 antibody or antigen-binding fragment thereof can have a VH having at least 85% sequence identity to SEQ ID NO:144. The humanized anti-CEACAM1 antibody or antigen-binding fragment thereof can have a VH having at least 90% sequence identity to SEQ ID NO:144. The humanized anti-CEACAM1 antibody or antigen-binding fragment thereof can have a VH having at least 95% sequence identity to SEQ ID NO:144. The humanized anti-CEACAM1 antibody or antigen-binding fragment thereof can have a VH having at least 98% sequence identity to SEQ ID NO:144. In some embodiments, provided herein are humanized antibodies or antigen-binding fragments thereof that specifically bind CEACAM1 comprising a VH having the amino acid sequence of SEQ ID NO:144.

In some embodiments, the VH can have the amino acid sequence of SEQ ID NO: 128 with N56V substitution. In some embodiments, the VH can have the amino acid sequence of SEQ ID NO:145. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:145. The humanized anti-CEACAM1 antibody or antigen-binding fragment thereof can have a VH having at least 85% sequence identity to SEQ ID NO:145. The humanized anti-CEACAM1 antibody or antigen-binding fragment thereof can have a VH having at least 90% sequence identity to SEQ ID NO:145. The humanized anti-CEACAM1 antibody or antigen-binding fragment thereof can have a VH having at least 95% sequence identity to SEQ ID NO:145. The humanized anti-CEACAM1 antibody or antigen-binding fragment thereof can have a VH having at least 98% sequence identity to SEQ ID NO:145. In some embodiments, provided herein are humanized antibodies or antigen-binding fragments thereof that specifically bind CEACAM1 comprising a VH having the amino acid sequence of SEQ ID NO:145.

In some embodiments, the VH can have the amino acid sequence of SEQ ID NO: 128 with Y100F substitution. In some embodiments, the VH can have the amino acid sequence of SEQ ID NO:146. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:146. The humanized anti-CEACAM1 antibody or antigen-binding fragment thereof can have a VH having at least 85% sequence identity to SEQ ID NO:146. The humanized anti-CEACAM1 antibody or antigen-binding fragment thereof can have a VH having at least 90% sequence identity to SEQ ID NO:146. The humanized anti-CEACAM1 antibody or antigen-binding fragment thereof can have a VH having at least 95% sequence identity to SEQ ID NO:146. The humanized anti-CEACAM1 antibody or antigen-binding fragment thereof can have a VH having at least 98% sequence identity to SEQ ID NO:146. In some embodiments, provided herein are humanized antibodies or antigen-binding fragments thereof that specifically bind CEACAM1 comprising a VH having the amino acid sequence of SEQ ID NO:146.

In some embodiments, the VH can have the amino acid sequence of SEQ ID NO:128 with N56Q and Y100F substitutions. In some embodiments, the VH can have the amino acid sequence of SEQ ID NO:147. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:147. The humanized anti-CEACAM1 antibody or antigen-binding fragment thereof can have a VH having at least 85% sequence identity to SEQ ID NO:147. The humanized anti-CEACAM1 antibody or antigen-binding fragment thereof can have a VH having at least 90% sequence identity to SEQ ID NO:147. The humanized anti-CEACAM1 antibody or antigen-binding fragment thereof can have a VH having at least 95% sequence identity to SEQ ID NO: 147. The humanized anti-CEACAM1 antibody or antigen-binding fragment thereof can have a VH having at least 98% sequence identity to SEQ ID NO:147. In some embodiments, provided herein are humanized antibodies or antigen-binding fragments thereof that specifically bind CEACAM1 comprising a VH having the amino acid sequence of SEQ ID NO:147.

In some embodiments, the VH can have the amino acid sequence of SEQ ID NO: 128 with N56I and Y100F substitutions. In some embodiments, the VH can have the amino acid sequence of SEQ ID NO:148. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:148. The humanized anti-CEACAM1 antibody or antigen-binding fragment thereof can have a VH having at least 85% sequence identity to SEQ ID NO:148. The humanized anti-CEACAM1 antibody or antigen-binding fragment thereof can have a VH having at least 90% sequence identity to SEQ ID NO:148. The humanized anti-CEACAM1 antibody or antigen-binding fragment thereof can have a VH having at least 95% sequence identity to SEQ ID NO:148. The humanized anti-CEACAM1 antibody or antigen-binding fragment thereof can have a VH having at least 98% sequence identity to SEQ ID NO:148. In some embodiments, provided herein are humanized antibodies or antigen-binding fragments thereof that specifically bind CEACAM1 comprising a VH having the amino acid sequence of SEQ ID NO:148.

In some embodiments, the VH can have the amino acid sequence of SEQ ID NO: 128 with N56V and Y100F substitutions. In some embodiments, the VH can have the amino acid sequence of SEQ ID NO:149. In some embodiments, provided herein are humanized antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:149. The humanized anti-CEACAM1 antibody or antigen-binding fragment thereof can have a VH having at least 85% sequence identity to SEQ ID NO:149. The humanized anti-CEACAM1 antibody or antigen-binding fragment thereof can have a VH having at least 90% sequence identity to SEQ ID NO:149. The humanized anti-CEACAM1 antibody or antigen-binding fragment thereof can have a VH having at least 95% sequence identity to SEQ ID NO:149. The humanized anti-CEACAM1 antibody or antigen-binding fragment thereof can have a VH having at least 98% sequence identity to SEQ ID NO:149. In some embodiments, provided herein are humanized antibodies or antigen-binding fragments thereof that specifically bind CEACAM1 comprising a VH having the amino acid sequence of SEQ ID NO:149.

In some embodiments, the anti-CEACAM1 antibody or antigen-binding fragment thereof provided herein is derived from the antibody clone designated as 4H11. In some embodiments, the anti-CEACAM1 antibody or antigen-binding fragment thereof provided herein has a VL from 4H11 (SEQ ID NO:67). In some embodiments, the anti-CEACAM1 antibody or antigen-binding fragment thereof provided herein has a VH from 4H11 (SEQ ID NO:76). The anti-CEACAM1 antibody or antigen-binding fragment thereof provided herein can have both a VL and a VH from 4H11.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:67; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:76. In some embodiments, provided herein are antibodies or antigen-binding fragments thereof that specifically bind CEACAM1 comprising a VL and a VH, wherein the VL and VH have the amino acid sequences of SEQ ID NOs:67 and 76, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:67. The anti-CEACAM1 antibody or antigen-binding fragment thereof can have a VL having at least 85% sequence identity to SEQ ID NO:67. The anti-CEACAM1 antibody or antigen-binding fragment thereof can have a VL having at least 90% sequence identity to SEQ ID NO:67. The anti-CEACAM1 antibody or antigen-binding fragment thereof can have a VL having at least 95% sequence identity to SEQ ID NO:67. The anti-CEACAM1 antibody or antigen-binding fragment thereof can have a VL having at least 98% sequence identity to SEQ ID NO:67. The anti-CEACAM1 antibody or antigen-binding fragment thereof can have a VL having the amino acid sequence of SEQ ID NO:67.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:76. The anti-CEACAM1 antibody or antigen-binding fragment thereof can have a VH having at least 85% sequence identity to SEQ ID NO:76. The anti-CEACAM1 antibody or antigen-binding fragment thereof can have a VH having at least 90% sequence identity to SEQ ID NO:76. The anti-CEACAM1 antibody or antigen-binding fragment thereof can have a VH having at least 95% sequence identity to SEQ ID NO:76. The anti-CEACAM1 antibody or antigen-binding fragment thereof can have at least 98% sequence identity to SEQ ID NO:76. The anti-CEACAM1 antibody or antigen-binding fragment thereof can have a VH having the amino acid sequence of SEQ ID NO:76.

In some embodiments, the anti-CEACAM1 antibody or antigen-binding fragment thereof provided herein has a VL that comprises VL CDRs 1, 2, and 3 from the VL of 4H11 (SEQ ID NO:67). In some embodiments, the anti-CEACAM1 antibody or antigen-binding fragment thereof provided herein has a VH that comprises VH CDRs 1, 2, and 3 from the VH of 4H11 (SEQ ID NO:76). The anti-CEACAM1 antibody or antigen-binding fragment thereof provided herein can have a VL comprising VL CDRs 1, 2, and 3 from the VL of 4H11 (SEQ ID NO:67) and a VH comprising VH CDRs 1, 2, and 3 from the VH of 4H11 (SEQ ID NO:76), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:6, (2) a VL CDR2 having the amino acid sequence of SEQ ID NO: 11, or (3) a VL CDR3 having the amino acid sequence of SEQ ID NO:18, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:6, 11, and 18, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:6, 11, and 18, respectively.

In some embodiments, provided herein are antibodies or antigen-binding fragments thereof that specifically bind CEACAM1 having a VH, comprising (1) a VH CDR1 having the amino acid sequence of SEQ ID NO:25, (2) a VH CDR2 having the amino acid sequence of SEQ ID NO:38, or (3) a VH CDR3 having the amino acid sequence of SEQ ID NO:49; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:25, 38, and 49, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:25, 38, and 49, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:6, 11, and 18, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:25, 38, and 49, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:6, 11, and 18, respectively; and (b) a VH that comprises VH CDR1, VH CDR2, and VH CDR3 having the amino acid sequences of SEQ ID NOs:25, 38, and 49, respectively.

In some embodiments, the anti-CEACAM1 antibody or antigen-binding fragment thereof provided herein is a variant of 4H11. In some embodiments, provided herein are humanized 4H11. In some embodiments, the 4H11 variant can have a VL that is a variant of the VL of 4H11 having up to about 3, about 5, about 8, about 10, about 12, or about 15 amino acid substitutions, additions, and/or deletions in SEQ ID NO:67. In some embodiments, the variant can have up to about 5 amino acid substitutions, additions, and/or deletions in SEQ ID NO:67. In some embodiments, the variant can have up to about 3 amino acid substitutions, additions, and/or deletions in SEQ ID NO:67. In some embodiments, the 4H11 variant can have a VH that is a variant of the VH of 4H11 having about 3, about 5, about 8, about 10, about 12, or about 15 amino acid substitutions, additions, and/or deletions in SEQ ID NO:76. In some embodiments, the variant can have up to about 5 amino acid substitutions, additions, and/or deletions in SEQ ID NO:76. In some embodiments, the variant can have up to about 3 amino acid substitutions, additions, and/or deletions in SEQ ID NO:76. The amino acid substitutions, additions, and/or deletions can be in the VH CDRs or VL CDRs. In some embodiments, the amino acid substitutions, additions, and/or deletions are not in the CDRs. In some embodiments, the variant of 4H11 has up to about 5 conservative amino acid substitutions. In some embodiments, the variant of 4H11 has up to 3 conservative amino acid substitutions.

In some embodiments, the anti-CEACAM1 antibody or antigen-binding fragment thereof provided herein is derived from the antibody clone designated as 2G12. In some embodiments, the anti-CEACAM1 antibody or antigen-binding fragment thereof provided herein has a VL from 2G12 (SEQ ID NO:68). In some embodiments, the anti-CEACAM1 antibody or antigen-binding fragment thereof provided herein has a VH from 2G12 (SEQ ID NO:77). The anti-CEACAM1 antibody or antigen-binding fragment thereof provided herein can have both a VL and a VH from 2G12.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:68; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:77. In some embodiments, provided herein are antibodies or antigen-binding fragments thereof that specifically bind CEACAM1 comprising a VL and a VH, wherein the VL and VH have the amino acid sequences of SEQ ID NOs:68 and 77, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:68. The anti-CEACAM1 antibody or antigen-binding fragment thereof can have a VL having at least 85% sequence identity to SEQ ID NO:68. The anti-CEACAM1 antibody or antigen-binding fragment thereof can have a VL having at least 90% sequence identity to SEQ ID NO:68. The anti-CEACAM1 antibody or antigen-binding fragment thereof can have a VL having at least 95% sequence identity to SEQ ID NO:68. The anti-CEACAM1 antibody or antigen-binding fragment thereof can have a VL having at least 98% sequence identity to SEQ ID NO:68. The anti-CEACAM1 antibody or antigen-binding fragment thereof can have a VL having the amino acid sequence of SEQ ID NO:68.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:77. The anti-CEACAM1 antibody or antigen-binding fragment thereof can have a VH having at least 85% sequence identity to SEQ ID NO:77. The anti-CEACAM1 antibody or antigen-binding fragment thereof can have a VH having at least 90% sequence identity to SEQ ID NO:77. The anti-CEACAM1 antibody or antigen-binding fragment thereof can have a VH having at least 95% sequence identity to SEQ ID NO:77. The anti-CEACAM1 antibody or antigen-binding fragment thereof can have at least 98% sequence identity to SEQ ID NO:77. The anti-CEACAM1 antibody or antigen-binding fragment thereof can have a VH having the amino acid sequence of SEQ ID NO:77.

In some embodiments, the anti-CEACAM1 antibody or antigen-binding fragment thereof provided herein has a VL that comprises VL CDRs 1, 2, and 3 from the VL of 2G12 (SEQ ID NO:68). In some embodiments, the anti-CEACAM1 antibody or antigen-binding fragment thereof provided herein has a VH that comprises VH CDRs 1, 2, and 3 from the VH of 2G12 (SEQ ID NO:77). The anti-CEACAM1 antibody or antigen-binding fragment thereof provided herein can have a VL comprising VL CDRs 1, 2, and 3 from the VL of 2G12 (SEQ ID NO:68) and a VH comprising VH CDRs 1, 2, and 3 from the VH of 2G12 (SEQ ID NO:77), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:7, (2) a VL CDR2 having the amino acid sequence of SEQ ID NO:12, or (3) a VL CDR3 having the amino acid sequence of SEQ ID NO:19, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:7, 12, and 19, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:7, 12, and 19, respectively.

In some embodiments, provided herein are antibodies or antigen-binding fragments thereof that specifically bind CEACAM1 having a VH, comprising (1) a VH CDR1 having the amino acid sequence of SEQ ID NO:26, (2) a VH CDR2 having the amino acid sequence of SEQ ID NO:39, or (3) a VH CDR3 having the amino acid sequence of SEQ ID NO:50; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:26, 39, and 50, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:26, 39, and 50, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:7, 12, and 19, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:26, 39, and 50, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:7, 12, and 19, respectively; and (b) a VH that comprises VH CDR1, VH CDR2, and VH CDR3 having the amino acid sequences of SEQ ID NOs:26, 39, and 50, respectively.

In some embodiments, the anti-CEACAM1 antibody or antigen-binding fragment thereof provided herein is a variant of 2G12. In some embodiments, provided herein are humanized 2G12. In some embodiments, the 2G12 variant can have a VL that is a variant of the VL of 2G12 having up to about 3, about 5, about 8, about 10, about 12, or about 15 amino acid substitutions, additions, and/or deletions in SEQ ID NO:68. In some embodiments, the variant can have up to about 5 amino acid substitutions, additions, and/or deletions in SEQ ID NO:68. In some embodiments, the variant can have up to about 3 amino acid substitutions, additions, and/or deletions in SEQ ID NO:68. In some embodiments, the 2G12 variant can have a VH that is a variant of the VH of 2G12 having about 3, about 5, about 8, about 10, about 12, or about 15 amino acid substitutions, additions, and/or deletions in SEQ ID NO:77. In some embodiments, the variant can have up to about 5 amino acid substitutions, additions, and/or deletions in SEQ ID NO:77. In some embodiments, the variant can have up to about 3 amino acid substitutions, additions, and/or deletions in SEQ ID NO:77. The amino acid substitutions, additions, and/or deletions can be in the VH CDRs or VL CDRs. In some embodiments, the amino acid substitutions, additions, and/or deletions are not in the CDRs. In some embodiments, the variant of 2G12 has up to about 5 conservative amino acid substitutions. In some embodiments, the variant of 2G12 has up to 3 conservative amino acid substitutions.

In some embodiments, the anti-CEACAM1 antibody or antigen-binding fragment thereof provided herein is derived from the antibody clone designated as 5D5. In some embodiments, the anti-CEACAM1 antibody or antigen-binding fragment thereof provided herein has a VL from 5D5 (SEQ ID NO:68). In some embodiments, the anti-CEACAM1 antibody or antigen-binding fragment thereof provided herein has a VH from 5D5 (SEQ ID NO:78). The anti-CEACAM1 antibody or antigen-binding fragment thereof provided herein can have both a VL and a VH from 5D5.

In some embodiments, provided herein are antibodies or antigen-binding fragments thereof that specifically bind to human CEACAM1 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:68; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO:78. In some embodiments, provided herein are antibodies or antigen-binding fragments thereof that specifically bind CEACAM1 comprising a VL and a VH, wherein the VL and VH have the amino acid sequences of SEQ ID NOs:68 and 78, respectively.

In some embodiments, provided herein are antibodies or antigen-binding fragments thereof that specifically bind CEACAM1 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:68. The anti-CEACAM1 antibody or antigen-binding fragment thereof can have a VL having at least 85% sequence identity to SEQ ID NO:68. The anti-CEACAM1 antibody or antigen-binding fragment thereof can have a VL having at least 90% sequence identity to SEQ ID NO:68. The anti-CEACAM1 antibody or antigen-binding fragment thereof can have a VL having at least 95% sequence identity to SEQ ID NO:68. The anti-CEACAM1 antibody or antigen-binding fragment thereof can have a VL having at least 98% sequence identity to SEQ ID NO:68. The anti-CEACAM1 antibody or antigen-binding fragment thereof can have a VL having the amino acid sequence of SEQ ID NO:68.

In some embodiments, provided herein are antibodies or antigen-binding fragments thereof that specifically bind CEACAM1 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:78. The anti-CEACAM1 antibody or antigen-binding fragment thereof can have a VH having at least 85% sequence identity to SEQ ID NO:78. The anti-CEACAM1 antibody or antigen-binding fragment thereof can have a VH having at least 90% sequence identity to SEQ ID NO:78. The anti-CEACAM1 antibody or antigen-binding fragment thereof can have a VH having at least 95% sequence identity to SEQ ID NO:78. The anti-CEACAM1 antibody or antigen-binding fragment thereof can have at least 98% sequence identity to SEQ ID NO:78. The anti-CEACAM1 antibody or antigen-binding fragment thereof can have a VH having the amino acid sequence of SEQ ID NO:78.

In some embodiments, the anti-CEACAM1 antibody or antigen-binding fragment thereof provided herein has a VL that comprises VL CDRs 1, 2, and 3 from the VL of 5D5 (SEQ ID NO:68). In some embodiments, the anti-CEACAM1 antibody or antigen-binding fragment thereof provided herein has a VH that comprises VH CDRs 1, 2, and 3 from the VH of 5D5 (SEQ ID NO:78). The anti-CEACAM1 antibody or antigen-binding fragment thereof provided herein can have a VL comprising VL CDRs 1, 2, and 3 from the VL of 5D5 (SEQ ID NO:68) and a VH comprising VH CDRs 1, 2, and 3 from the VH of 5D5 (SEQ ID NO:78), respectively. The CDRs can be defined by any system known in the art. In some embodiments, the CDRs are defined by Kabat, Chothia, IMGT, AbM, or Contact.

In some embodiments, provided herein are antibodies or antigen-binding fragments thereof that specifically bind CEACAM1 having a VL, comprising (1) a VL CDR1 having the amino acid sequence of SEQ ID NO:7, (2) a VL CDR2 having the amino acid sequence of SEQ ID NO:12, or (3) a VL CDR3 having the amino acid sequence of SEQ ID NO:19, or a variant thereof having up to about 3 amino acid substitutions, additions, and/or deletions in the VL CDRs. In some embodiments, the anti-human CEACAM1 antibodies or antigen-binding fragments thereof have a VL comprising VL CDR1, VL CDR2, and VL CDR3 having the amino acid sequences of SEQ ID NOs:7, 12, and 19, respectively, or a variant thereof having up to about 3 amino acid substitutions, additions, and/or deletions in the VL CDRs. The VL can have VL CDR1, VL CDR2, and VL CDR3 having the amino acid sequences of SEQ ID NOs:7, 12, and 19, respectively.

In some embodiments, provided herein are antibodies or antigen-binding fragments thereof that specifically bind CEACAM1 having a VH, comprising (1) a VH CDR1 having the amino acid sequence of SEQ ID NO:27, (2) a VH CDR2 having the amino acid sequence of SEQ ID NO:38, or (3) a VH CDR3 having the amino acid sequence of SEQ ID NO:51; or a variant thereof having up to about 3 amino acid substitutions, additions, and/or deletions in the VH CDRs. In some embodiments, the anti-human CEACAM1 antibodies or antigen-binding fragments thereof have a VH comprising VH CDR1, VH CDR2, and VH CDR3 having the amino acid sequences of SEQ ID NOs:27, 38, and 51, respectively, or a variant thereof having up to about 3 amino acid substitutions, additions, and/or deletions in the VH CDRs. The VH can have VH CDR1, VH CDR2, and VH CDR3 having the amino acid sequences of SEQ ID NOs:27, 38, and 51, respectively.

In some embodiments, provided herein are antibodies or antigen-binding fragments thereof that specifically bind CEACAM1 comprising (a) a VL that comprises VL CDR1, VL CDR2, and VL CDR3 having the amino acid sequences of SEQ ID NOs:7, 12, and 19, respectively; or a variant thereof having up to about 3 amino acid substitutions, additions, and/or deletions in the VL CDRs and (b) a VH that comprises VH CDR1, VH CDR2, and VH CDR3 having the amino acid sequences of SEQ ID NOs:27, 38, and 51, respectively; or a variant thereof having up to about 3 amino acid substitutions, additions, and/or deletions in the VH CDRs. In some embodiments, the anti-human CEACAM1 antibodies or antigen-binding fragments thereof comprise (a) a VL that comprises VL CDR1, VL CDR2, and VL CDR3 having the amino acid sequences of SEQ ID NOs:7, 12, and 19, respectively; and (b) a VH that comprises VH CDR1, VH CDR2, and VH CDR3 having the amino acid sequences of SEQ ID NOs:27, 38, and 51, respectively.

In some embodiments, the anti-CEACAM1 antibody or antigen-binding fragment thereof provided herein is a variant of 5D5. In some embodiments, provided herein are humanized 5D5. In some embodiments, the 5D5 variant can have a VL that is a variant of the VL of 5D5 having up to about 3, about 5, about 8, about 10, about 12, or about 15 amino acid substitutions, additions, and/or deletions in SEQ ID NO:68. In some embodiments, the variant can have up to about 5 amino acid substitutions, additions, and/or deletions in SEQ ID NO:68. In some embodiments, the variant can have up to about 3 amino acid substitutions, additions, and/or deletions in SEQ ID NO:68. In some embodiments, the 5D5 variant can have a VH that is a variant of the VH of 5D5 having about 3, about 5, about 8, about 10, about 12, or about 15 amino acid substitutions, additions, and/or deletions in SEQ ID NO:78. In some embodiments, the variant can have up to about 5 amino acid substitutions, additions, and/or deletions in SEQ ID NO:78. In some embodiments, the variant can have up to about 3 amino acid substitutions, additions, and/or deletions in SEQ ID NO:78. The amino acid substitutions, additions, and/or deletions can be in the VH CDRs or VL CDRs. In some embodiments, the amino acid substitutions, additions, and/or deletions are not in the CDRs. In some embodiments, the variant of 5D5 has up to about 5 conservative amino acid substitutions. In some embodiments, the variant of 5D5 has up to 3 conservative amino acid substitutions.

In some embodiments, anti-CEACAM1 antibodies provided herein are IgA, IgD, IgE, IgG, or IgM antibodies. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, anti-CEACAM1 antibodies provided herein comprise a light chain and a heavy chain. The light chain can comprise a light chain constant domain (CL) and a light chain variable domain (VL). The heavy chain can comprise a heavy chain variable domain (VH) and a heavy chain constant domain (CH). The VL/VH can be any VL/VH disclosed herein. In some embodiments, the light chain constant region (CL) is kappa CL (CK; SEQ ID NO:134). In some embodiments, the light chain constant region (CL) is lambda CL (Ck; SEQ ID NO:135). In some embodiments, the heavy chain can comprise a heavy chain constant domain (CH) from human IgA. In some embodiments, the heavy chain can comprise a heavy chain constant domain (CH) from human IgD. In some embodiments, the heavy chain can comprise a heavy chain constant domain (CH) from human IgE. In some embodiments, the heavy chain can comprise a heavy chain constant domain (CH) from human IgG. In some embodiments, the heavy chain can comprise a heavy chain constant domain (CH) from human IgM. In some embodiments, the heavy chain can comprise a heavy chain constant domain (CH) from human IgG1 (e.g., SEQ ID NO:136). In some embodiments, the heavy chain can comprise a heavy chain constant domain (CH) from human IgG2 (e.g., SEQ ID NO:137). In some embodiments, the heavy chain can comprise a heavy chain constant domain (CH) from human IgG3 (e.g., SEQ ID NO:138). In some embodiments, the heavy chain can comprise a heavy chain constant domain (CH) from human IgG4 (e.g., SEQ ID NO:139). Expressly contemplated here are any and all combinations of the VL/VH pairs disclosed herein that specifically bind CEACAM1 (e.g., human CEACAM1) and the CL/CH disclosed herein or otherwise known in the art.

| Light Chain Constant | |
|---|---|
| Region | Sequences |
| kappa CL (Cκ) | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNR |
| | GEC (SEQ ID NO: 134) |
| lambda CL (Cλ) | GQPKAAPSVTLFPPSSEELQANKATLVCLISDFYPGAVTVAWKADSSPVKAG |
| | VETTTPSKQSNNKYAASSYLSLTPEQWKSHRSYSCQVTHEGSTVEKTVAPTE |
| | CS (SEQ ID NO: 135) |

| Heavy Chain Constant | |
|---|---|
| Region | Sequences |
| IgG1 CH | ASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMH |
| | EALHNHYTQKSLSLSPGK (SEQ ID NO: 136) |
| IgG2 CH | ASTKGPSVFPLAPCSRSTSESTAALGCLVKDYFPEPVTVSWNSGALTSGVHTF |
| | PAVLQSSGLYSLSSVVTVPSSNFGTQTYTCNVDHKPSNTKVDKTVERKCCVE |
| | CPPCPAPPVAGPSVFLFPPKPKDTLMISRTPEVTCVVVDVSHEDPEVQFNWYV |
| | DGVEVHNAKTKPREEQFNSTFRVVSVLTVVHQDWLNGKEYKCKVSNKGLP |
| | APIEKTISKTKGQPREPQVYTLPPSREEMTKNQVSLTCLVKGFYPSDISVEWES |
| | NGQPENNYKTTPPMLDSDGSFFLYSKLTVDKSRWQQGNVFSCSVMHEALHN |
| | HYTQKSLSLSPGK (SEQ ID NO: 137) |
| IgG3 CH | ASTKGPSVFPLAPCSRSTSGGTAALGCLVKDYFPEPVTVSWNSGALTSGVHT |
| | FPAVLQSSGLYSLSSVVTVPSSSLGTQTYTCNVNHKPSNTKVDKRVELKTPL |
| | GDTTHTCPRCPEPKSCDTPPPCPRCPEPKSCDTPPPCPRCPEPKSCDTPPPCPRC |
| | PAPELLGGPSVFLFPPKPKDTLMISRTPEVTCVVVDVSHEDPEVQFKWYVDG |
| | VEVHNAKTKPREEQYNSTFRVVSVLTVLHQDWLNGKEYKCKVSNKALPAPI |
| | EKTISKTKGQPREPQVYTLPPSREEMTKNQVSLTCLVKGFYPSDIAVEWESSG |
| | QPENNYNTTPPMLDSDGSFFLYSKLTVDKSRWQQGNIFSCSVMHEALHNRFT |
| | QKSLSLSPGK (SEQ ID NO: 138) |
| IgG4 CH | ASTKGPSVFPLAPCSRSTSESTAALGCLVKDYFPEPVTVSWNSGALTSGVHTF |
| | PAVLQSSGLYSLSSVVTVPSSSLGTKTYTCNVDHKPSNTKVDKRVESKYGPP |
| | CPSCPAPEFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVEW |
| | ESNGQPENNYKTTPPVLDSDGSFFLYSRLTVDKSRWQEGNVFSCSVMHEAL |
| | HNHYTQKSLSLSLGK (SEQ ID NO: 139) |

In some embodiments, the antibodies provided herein have a light chain constant region (CL) having at least 85%, at least 90%, at least 95%, at least 98%, or at least 99% sequence identity to SEQ ID NO:134. In some embodiments, the antibodies provided herein have a CL having the amino acid sequence of SEQ ID NO:134. In some embodiments, the antibodies provided herein have a light chain constant region (CL) having at least 85%, at least 90%, at least 95%, at least 98%, or at least 99% sequence identity to SEQ ID NO:135. In some embodiments, the antibodies provided herein have a CL having the amino acid sequence of SEQ ID NO:135. In some embodiments, the antibodies provided herein have a heavy chain constant region (CH) having at least 85%, at least 90%, at least 95%, at least 98%, or at least 99% sequence identity to SEQ ID NO:136. In some embodiments, the antibodies provided herein have a CH having the amino acid sequence of SEQ ID NO:136. In some embodiments, the antibodies provided herein have a CH having at least 85%, at least 90%, at least 95%, at least 98%, or at least 99% sequence identity to SEQ ID NO:137. In some embodiments, the antibodies provided herein have a CH having the amino acid sequence of SEQ ID NO:137. In some embodiments, the antibodies provided herein have a CH having at least 85%, at least 90%, at least 95%, at least 98%, or at least 99% sequence identity to SEQ ID NO:138. In some embodiments, the antibodies provided herein have a CH having the amino acid sequence of SEQ ID NO:138. In some embodiments, the antibodies provided herein have a CH having at least 85%, at least 90%, at least 95%, at least 98%, or at least 99% sequence identity to SEQ ID NO:139. In some embodiments, the antibodies provided herein have a CH having the amino acid sequence of SEQ ID NO:139.

In some embodiments, provided herein are also antibodies or antigen-binding fragments that compete with the antibody or antigen-binding fragment provided above for binding to CEACAM1 (e.g., human CEACAM1). Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some embodiments, an anti-CEACAM1 antibody or antigen-binding fragment competes with, and inhibits binding of another antibody or antigen-binding fragment to CEACAM1 by at least 50%, 60%, 70%, 80%, 90% or 100%. Competition assays can be conducted as described, for example, in Ed Harlow and David Lane, Cold Spring Harb Protoc; 2006; doi: 10.H01/pdb.prot4277 or in Chapter 11 of “Using Antibodies” by Ed Harlow and David Lane, Cold Spring Harbor Laboratory Press, Cold Spring Harbor, NY, USA 1999.

In some embodiments, provided herein are antibodies or antigen-binding fragments that compete with an anti-CEACAM1 antibody or antigen-binding fragment disclosed herein for binding to CEACAM1 (e.g., human CEACAM1). In some embodiments, provided herein are antibodies or antigen-binding fragments that compete with chimeric 6H9 for binding to CEACAM1 (e.g., human CEACAM1). In some embodiments, provided herein are antibodies or antigen-binding fragments that compete with a humanized 6H9 disclosed herein for binding to CEACAM1 (e.g., human CEACAM1). In some embodiments, provided herein are antibodies or antigen-binding fragments that compete with chimeric 7D4 for binding to CEACAM1 (e.g., human CEACAM1). In some embodiments, provided herein are antibodies or antigen-binding fragments that compete with a humanized 7D4 disclosed herein for binding to CEACAM1 (e.g., human CEACAM1). In some embodiments, provided herein are antibodies or antigen-binding fragments that compete with chimeric 9D12 for binding to CEACAM1 (e.g., human CEACAM1). In some embodiments, provided herein are antibodies or antigen-binding fragments that compete with a humanized 9D12 disclosed herein for binding to CEACAM1 (e.g., human CEACAM1). In some embodiments, provided herein are antibodies or antigen-binding fragments that compete with chimeric P23B6 for binding to CEACAM1 (e.g., human CEACAM1). In some embodiments, provided herein are antibodies or antigen-binding fragments that compete with a humanized P23B6 disclosed herein for binding to CEACAM1 (e.g., human CEACAM1). In some embodiments, provided herein are antibodies or antigen-binding fragments that compete with chimeric 2C10 for binding to CEACAM1 (e.g., human CEACAM1). In some embodiments, provided herein are antibodies or antigen-binding fragments that compete with a humanized 2C10 disclosed herein for binding to CEACAM1 (e.g., human CEACAM1). In some embodiments, provided herein are antibodies or antigen-binding fragments that compete with chimeric 4D10 for binding to CEACAM1 (e.g., human CEACAM1). In some embodiments, provided herein are antibodies or antigen-binding fragments that compete with a humanized 4D10 disclosed herein for binding to CEACAM1 (e.g., human CEACAM1). In some embodiments, provided herein are antibodies or antigen-binding fragments that compete with chimeric 4H11 for binding to CEACAM1 (e.g., human CEACAM1). In some embodiments, provided herein are antibodies or antigen-binding fragments that compete with a humanized 4H11 disclosed herein for binding to CEACAM1 (e.g., human CEACAM1). In some embodiments, provided herein are antibodies or antigen-binding fragments that compete with chimeric 2G12 for binding to CEACAM1 (e.g., human CEACAM1). In some embodiments, provided herein are antibodies or antigen-binding fragments that compete with a humanized 2G12 disclosed herein for binding to CEACAM1 (e.g., human CEACAM1). In some embodiments, provided herein are antibodies or antigen-binding fragments that compete with chimeric 5D5 for binding to CEACAM1 (e.g., human CEACAM1). In some embodiments, provided herein are antibodies or antigen-binding fragments that compete with a humanized 5D5 disclosed herein for binding to CEACAM1 (e.g., human CEACAM1).

Epitope mapping is a method of identifying the binding site, region, or epitope on a target protein where an antibody binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, anti-CEACAM1 antibodies or antigen-binding fragments described herein are characterized by assays including, but not limited to, N-terminal sequencing, amino acid analysis, HPLC, mass spectrometry, ion exchange chromatography, and papain digestion.

The anti-CEACAM1 antibodies or antigen-binding fragments of the present disclosure can be analyzed for their physical, chemical and/or biological properties by various methods known in the art. In some embodiments, an anti-CEACAM1 antibody is tested for its ability to bind CEACAM1 (e.g., human CEACAM1). Binding assays include, but are not limited to, BLI, SPR (e.g., Biacore), ELISA, and FACS. In addition, antibodies can be evaluated for solubility, stability, thermostability, viscosity, expression levels, expression quality, and/or purification efficiency.

In some embodiments, anti-CEACAM1 antibodies or antigen-binding fragments described herein bind to human CEACAM1 with high affinity, for example, with a K_{D} of 10⁻⁷ M or less, 5×10⁻⁸ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, or 10⁻¹¹ M or less. In some embodiments, anti-CEACAM1 antibodies or antigen-binding fragments described herein bind to human CEACAM1 with a K_{D} of 5×10⁻⁹ M or less. In some embodiments, anti-CEACAM1 antibodies or antigen-binding fragments described herein bind to human CEACAM1 with high affinity, for example, with a K_{D} of about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, or about 10⁻¹¹ M. In some embodiments, anti-CEACAM1 antibodies or antigen-binding fragments described herein bind to human CEACAM1 with a K_{D} of about 10⁻⁷ M. In some embodiments, anti-CEACAM1 antibodies or antigen-binding fragments described herein bind to human CEACAM1 with a K_{D} ranging from 10⁻¹¹ M to 10⁻⁷ M, from 5×10⁻¹¹ M to 10⁻⁷ M, from 10⁻¹⁰ M to 10⁻⁷ M, from 5×10⁻¹⁰ M to 10⁻⁷ M, from 10⁻⁹ M to 10⁻⁷ M, from 5×10⁻⁹ M to 10⁻⁷ M, from 10⁻¹¹ to 10⁻¹¹ M, from 5×10⁻¹¹ M to 10⁻⁸ M, from 10⁻¹⁰ M to 10⁻⁸ M, from 5×10⁻¹⁰ M to 10⁻⁸ M, from 10⁻⁹ M to 10⁻⁸ M, from 5×10⁻⁹ M to 10⁻⁸ M, from 10⁻¹¹ M to 5×10⁻⁹ M, from 5×10⁻¹¹ M to 5×10⁻⁹ M, from 10⁻¹⁰ M to 5×10⁻⁹ M, from 5×10⁻¹⁰ M to 5×10⁻⁹ M, from 10⁻⁹ M to 5×10⁻⁹ M, from 10⁻¹¹ M to 10⁻⁹ M, from 5×10⁻¹¹ M to 10⁻⁹ M, from 10⁻¹⁰ M to 10⁻⁹ M, or from 5×10⁻¹⁰ M to 10⁻⁹ M. In some embodiments, anti-CEACAM1 antibodies or antigen-binding fragments described herein bind to human CEACAM1 with high affinity, for example, with a K_{D} from 10⁻¹⁰ M to 5×10⁻⁹ M. In some embodiments, the K_{D} is determined by BLI. In some embodiments, the K_{D} is determined by SPR. In some embodiments, anti-CEACAM1 antibodies or antigen-binding fragments described herein bind to human CEACAM1 with high affinity, for example, with a K_{D} of 10⁻⁷ M or less, 5×10⁻⁸ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, or 10⁻¹¹ M or less; or ranging from 10⁻¹¹ M to 10⁻⁷ M, from 5×10⁻¹¹ M to 10⁻⁷ M, from 10⁻¹⁰ M to 10⁻⁷ M, from 5×10⁻¹⁰ M to 10⁻⁷ M, from 10⁻⁹ M to 10⁻⁷ M, from 5×10⁻⁹ M to 10⁻⁷ M, from 10⁻¹¹ M to 10⁻⁸ M, from 5×10⁻¹¹ M to 10⁻⁸ M, from 10⁻¹⁰ M to 10⁻⁸ M, from 5×10⁻¹⁰ M to 10⁻⁸ M, from 10⁻⁹ M to 10⁻⁸ M, from 5×10⁻⁹ M to 10⁻⁸ M, from 10⁻¹¹ M to 5×10⁻⁹ M, from 5×10⁻¹¹ M to 5×10⁻⁹ M, from 10⁻¹⁰ M to 5×10⁻⁹ M, from 5×10⁻¹⁰ M to 5×10⁻⁹ M, from 10⁻⁹ M to 5×10⁻⁹ M, from 10⁻¹¹ M to 10⁻⁹ M, from 5×10⁻¹¹ M to 10⁻⁹ M, from 10⁻¹⁰ M to 10⁻⁹ M, or from 5×10⁻¹⁰ M to 10⁻⁹ M, as measured by BLI.

In some embodiments, anti-CEACAM1 antibodies or antigen-binding fragments described herein do not bind to other CEACAM family members. In some embodiments, the affinities of the anti-CEACAM1 antibodies or antigen-binding fragments described herein to other CEACAM family member proteins are comparable to those of an isotype antibody (e.g., hIgG1). Other CEACAM family member proteins include, but are not limited to: CEACAM3, CEACAM5, CEACAM6 and CEACAM8. In some embodiments, anti-CEACAM1 antibodies or antigen-binding fragments described herein do not bind to one or more of CEACAM family member proteins selected from the group consisting of CEACAM3, CEACAM5, CEACAM6 and CEACAM8. In some embodiments, the anti-CEACAM1 antibody or antigen-binding fragment described herein does not bind to any of CEACAM family member proteins: CEACAM3, CEACAM5, CEACAM6 and CEACAM8.

In some embodiments, anti-CEACAM1 antibodies or antigen-binding fragments described herein can internalize or downregulate cell surface CEACAM1 in CEACAM1-expressing cells.

In some embodiments, anti-CEACAM1 antibodies or antigen-binding fragments described herein can block the interaction between CEACAM1 and CEACAM5. In some embodiments, anti-CEACAM1 antibodies or antigen-binding fragments described herein can block the interaction between CEACAM1 and CEACAM6. In some embodiments, the anti-CEACAM1 antibodies or antigen-binding fragments described herein can promote the killing efficiency of immune cells against CEACAM1-expressing cancer cells. In some embodiments, the anti-CEACAM1 antibodies or antigen-binding fragments described herein can promote the killing efficiency of immune cells against CEACAM5-expressing cancer cells. In some embodiments, the anti-CEACAM1 antibodies or antigen-binding fragments described herein can promote the killing efficiency of immune cells against CEACAM6-expressing cancer cells.

In some embodiments, anti-CEACAM1 antibodies or antigen-binding fragments described herein stimulate or enhance an immune response. In some embodiments, anti-CEACAM1 antibodies or antigen-binding fragments described herein can activate T cells. In some embodiments, anti-CEACAM1 antibodies or antigen-binding fragments described herein can activate CEACAM1-expressing T cells. In some embodiments, anti-CEACAM1 antibodies or antigen-binding fragments described herein can activate PBMC-derived T cells.

For example, the anti-CEACAM1 antibodies or antigen-binding fragments can activate or costimulate cells, as evidenced, e.g., by enhanced cytokine (e.g., IFN-γ) secretion and/or enhanced proliferation, which can result from the inhibition of CEACAM1-mediated T cell inhibitory activity. In some embodiments, T cell activation or co-stimulation by a CEACAM1 antibody or antigen-binding fragment occurs in the presence of CD3 stimulation. In some embodiments, an anti-CEACAM1 antibody increases IFN-γ secretion by a factor of 50%, 100% (i.e., 2 fold), 3 fold, 4 fold, 5 fold or more, optionally with a maximum of up to 10 fold, 30 fold, 100 fold, as measured, e.g., on primary human T cells. In some embodiments, anti-CEACAM1 antibodies or antigen-binding fragments described herein block or reduce the inhibitory effects of CEACAM1.

In some embodiments, anti-CEACAM1 antibodies or antigen-binding fragments described herein can activate immune cells. In some embodiments, anti-CEACAM1 antibodies or antigen-binding fragments described herein can stimulate or enhance the tumor killing activity of immune cells. Immune cells that can be activated by anti-CEACAM1 antibodies or antigen-binding fragments described herein include lymphocytes (e.g., T cells, NK cells, and NKT cells) and myeloid cells (e.g., monocytes, dendritic cells and neutrophils). In some embodiments, anti-CEACAM1 antibodies or antigen-binding fragments described herein activate T cells by, for example, stimulating or enhancing the tumor killing activity of the T cells. In some embodiments, anti-CEACAM1 antibodies or antigen-binding fragments described herein activate NK cells by, for example, stimulating or enhancing the tumor killing activity of the NK cells.

In some embodiments, anti-CEACAM1 antibodies or antigen-binding fragments described herein inhibit macrophage-mediated tumor aggression. Macrophages recruited to tumors and influenced by the presence of cancer are often referred to as tumor-associated macrophages (TAMs). TAMs participate in the formation of the tumor microenvironment. TAMs are widely present in various tumors, such as solid tumor and hematological tumor. TAMs can promote tumor growth, invasion, and metastasis. It has been found that macrophage-cancer cell interaction via CEACAM1 promotes cancer metastasis. It has also been found that macrophages could induce polyploidy in metastatic cells to promote tumor aggressiveness. Besides, CEACAM1 plays a key role in the development and function of macrophages. Therefore, the anti-CEACAM1 antibodies or antigen-binding fragments described herein could inhibit macrophage-mediated tumor aggression.

Accordingly, an anti-CEACAM1 antibody or antigen-binding fragment that exhibits one or more of these functional properties (e.g., biochemical, immunochemical, cellular, physiological or other biological activities, or the like) as determined according to methodologies known to the art and described herein, will be understood to exhibit a statistically significant difference in the particular activity relative to that seen in the absence of the antibody (e.g., or when a control antibody of irrelevant specificity is present). In some embodiments, anti-CEACAM1 antibody or antigen-binding fragment-induced increases in a measured parameter (e.g., T cell proliferation, cytokine production) in a given assay effects a statistically significant increase by at least 10% of the measured parameter, e.g., by at least 20%, 30%, 40%, 50%, 60%, 70%, 80%, 90%, 95%, 100% (i.e., 2 fold), 3 fold, 5 fold or 10 fold, and in some embodiments, an antibody described herein can increase the measured parameter, e.g., by greater than 92%, 94%, 95%, 97%, 98%, 99%, 100% (i.e., 2 fold), 3 fold, 5 fold or 10 fold, relative to the same assay conducted in the absence of the antibody. Conversely, anti-CEACAM1 antibody-induced decreases in a measured parameter (e.g., tumor volume, CEACAM1 ligand binding to human CEACAM1) in a given assay effects a statistically significant decrease by at least 10% of the measured parameter, e.g., by at least 20%, 30%, 40%, 50%, 60%, 70%, 80% or 90%, and in some embodiments, an antibody described herein can decrease the measured parameter, e.g., by greater than 92%, 94%, 95%, 97%, 98% or 99%, relative to the same assay conducted in the absence of the antibody.

### 6.3 Variants and Conjugates

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations can be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant can be determined by systematically making insertions, deletions, or substitutions in the sequence and testing the resulting variant proteins for activity as compared to the parent protein.

In some embodiments, provided herein are variants of anti-CEACAM1 antibodies or antigen-binding fragments described herein. In some embodiments, provided herein are variants of anti-CEACAM1 antibody clone 6H9, 7D4, 9D12, P23B6, 2C10, 4D10, 4H11, 2G12, or 5D5. In some embodiments, provided herein are variants of humanized 6H9, humanized 7D4, humanized 9D12, humanized P23B6, humanized 2C10, humanized 4D10, humanized 4H11, humanized 2G12, or humanized 5D5. In some embodiments, a variant comprises one to 30 amino acid substitutions, additions, and/or deletions in the parent antibody or antigen-binding fragment. In some embodiments, a variant comprises one to 25 amino acid substitutions, additions, and/or deletions in the parent antibody or antigen-binding fragment. In some embodiments, a variant comprises one to 20 substitutions, additions, and/or deletions in the parent antibody or antigen-binding fragment. In some embodiments, a variant comprises one to 15 substitutions, additions, and/or deletions in the parent antibody or antigen-binding fragment. In some embodiments, a variant comprises one to 10 substitutions, additions, and/or deletions in the parent antibody or antigen-binding fragment. In some embodiments, a variant comprises one to five amino acid substitutions, additions, and/or deletions in the parent antibody or antigen-binding fragment. In some embodiments, a variant comprises one to three amino acid substitutions, additions, and/or deletions in the parent antibody or antigen-binding fragment. In some embodiments, the amino acid substitution(s) is in a CDR of the antibody or antigen-binding fragment. In some embodiments, the amino acid substitution(s) is not in a CDR of the antibody or antigen-binding fragment. In some embodiments, the amino acid substitution(s) is in a framework region of the antibody or antigen-binding fragment. In some embodiments, the amino acid substitutions, additions, and/or deletions are conservative amino acid substitutions.

It is known in the art that the constant region(s) of an antibody mediates several effector functions and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, the anti-CEACAM1 antibodies or antigen-binding fragments described herein comprise at least one constant region of a human IgA antibody. In some embodiments, the anti-CEACAM1 antibodies or antigen-binding fragments described herein comprise at least one constant region of a human IgD antibody. In some embodiments, the anti-CEACAM1 antibodies or antigen-binding fragments described herein comprise at least one constant region of a human IgE antibody. In some embodiments, the anti-CEACAM1 antibodies or antigen-binding fragments described herein comprise at least one constant region of a human IgG antibody. In some embodiments, the anti-CEACAM1 antibodies or antigen-binding fragments described herein comprise at least one constant region of a human IgM antibody. In some embodiments, the anti-CEACAM1 antibodies or antigen-binding fragments described herein comprise at least one constant region of a human IgG1 antibody. In some embodiments, the anti-CEACAM1 antibodies or antigen-binding fragments described herein comprise at least one constant region of a human IgG2 antibody. In some embodiments, the anti-CEACAM1 antibodies or antigen-binding fragments described herein comprise at least one constant region of a human IgG3 antibody. In some embodiments, the anti-CEACAM1 antibodies or antigen-binding fragments described herein comprise at least one constant region of a human IgG4 antibody.

In some embodiments, at least one or more of the constant regions has been modified or deleted in the anti-CEACAM1 antibody or antigen-binding fragment described herein. In some embodiments, the antibodies comprise modifications to one or more of the three heavy chain constant regions (CH1, CH2 or CH3) and/or to the light chain constant region (CL).

In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

In some embodiments, an anti-CEACAM1 antibody or antigen-binding fragment comprises a Fc region. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fc regions with amino acid variations have been identified in native antibodies. In some embodiments, the modified antibodies (e.g., modified Fc region) provide for altered effector functions that, in turn, affect the biological profile of the antibody. For example, in some embodiments, the deletion or inactivation (through point mutations or other means) of a constant region reduces Fc receptor binding of the modified antibody as it circulates. In some embodiments, the constant region modifications reduce the immunogenicity of the antibody. In some embodiments, the constant region modifications increase the serum half-life of the antibody. In some embodiments, the constant region modifications reduce the serum half-life of the antibody. In some embodiments, the constant region modifications enhance ADCC and/or complement dependent cytotoxicity (CDC) of the antibody. In some embodiments, the constant region modifications enhance antibody-dependent cellular phagocytosis (ADCP) of the antibody. In some embodiments, the constant region modifications decrease or remove ADCC and/or CDC of the antibody. In some embodiments, specific amino acid substitutions in a human IgG1 Fc region with corresponding IgG2 or IgG4 residues reduce effector functions (e.g., ADCC and CDC) in the modified antibody. In some embodiments, an antibody does not have one or more effector functions (e.g., “effectorless” antibodies). In some embodiments, the antibody does not bind an Fc receptor and/or complement factors. In some embodiments, the antibody has no effector function(s). In some embodiments, the constant region modifications increase or enhance ADCC and/or ADCP of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites. In some embodiments, an anti-CEACAM1 antibody or antigen-binding fragment comprises a variant Fc region that is engineered with substitutions at specific amino acid positions as compared to a native Fc region.

In some embodiments, an anti-CEACAM1 antibody or antigen-binding fragment described herein comprises an IgG1 heavy chain constant region that comprises one or more amino acid substitutions selected from the group of E233, L234, L235, G236, S239, N297, A330, P331, 1332, and P329. In some embodiments, the Fc region includes an amino acid substitution at position L234. The L234 substitution can be, e.g., L234A, or L234Y. In some embodiments, the Fc region includes an amino acid substitution at position L235. The L235 substitution can be, e.g., L235A, L235Q or L235V. The IgG1 heavy chain constant region can comprise a G236 substitution. The G236 substitution can be, e.g., G236A or G236W. The IgG1 heavy chain constant region can comprise an S239 substitution. The S239 substitution can be, e.g., S239D or S239M. The IgG1 heavy chain constant region can comprise an A330 substitution. The A330 substitution can be, e.g., A330M or A330L. The IgG1 heavy chain constant region can comprise an 1332 substitution. The 1332 substitution can be, e.g., I332E. In some embodiments, the Fc region includes an amino acid substitution at position P329. In some embodiments, the amino acid substitution is P329A or P329G. In some embodiments, the Fc region includes the amino acid substitutions L234A and L235A. In some embodiments, the Fc region includes amino acid substitutions at positions P329, L234 and L235. In some embodiments, the Fc region comprises the amino acid mutations L234A, L235A and P329G. In some embodiments, the Fc region comprises the amino acid mutations G236A, S239D, and I332E. In some embodiments, the Fc region comprises the amino acid mutations G236A, A330L, and I332E. In some embodiments, the Fc region comprises the amino acid mutations S239D, A330L, and I332E. In some embodiments, the Fc region includes an amino acid substitution at position P329 and a further amino acid substitution at a position selected from E233, L234, L235, N297 and P331. In some embodiments, the further amino acid substitution is E233P, L234A, L235A, L235E, N297A, N297D or P331S. All are numbered according to the EU Index.

In some embodiments, the anti-CEACAM1 antibodies or antigen-binding fragments described herein comprise an IgG4 heavy chain constant region that comprises one or more amino acid substitutions that increase the antibody or antigen-binding fragment's terminal half-life. As understood in the art, the terminal half-life, also referred to as the biological half-life, means the time required for plasma/blood concentration to decrease by 50% after administration and after pseudo-equilibrium of distribution has been reached. The one or more amino acid substitutions can be selected from the group consisting of S228, F234, L235, M252, S254, T256, K288, T307, M428, N434, H435, and Y436, numbered according to the EU Index. In some embodiments, the IgG4 heavy chain constant region has an amino acid substitution at S228 (e.g., S228P). In some embodiments, the IgG4 heavy chain constant region has an amino acid substitution at F234 (e.g., F234A). In some embodiments, the IgG4 heavy chain constant region has an amino acid substitution at L235 (e.g., L235A). In some embodiments, the IgG4 heavy chain constant region has an amino acid substitution at M252 (e.g., M252Y). In some embodiments, the IgG4 heavy chain constant region has an amino acid substitution at S254 (e.g., S254T). In some embodiments, the IgG4 heavy chain constant region has an amino acid substitution at T256 (e.g., T256E). In some embodiments, the IgG4 heavy chain constant region has an amino acid substitution at K288 (e.g., K288A). In some embodiments, the IgG4 heavy chain constant region has an amino acid substitution at T307 (e.g., T307Q or T307H). In some embodiments, the IgG4 heavy chain constant region has an amino acid substitution at M428 (e.g., M428L). In some embodiments, the IgG4 heavy chain constant region has an amino acid substitution at N434 (e.g., N434A). In some embodiments, the IgG4 heavy chain constant region has an amino acid substitution at H435 (e.g., H435A or H435R). In some embodiments, the IgG4 heavy chain constant region has an amino acid substitution at Y436 (e.g., Y436T or Y436F). All are numbered according to the EU Index.

In some embodiments, variants can include addition of amino acid residues at the amino- and/or carboxyl-terminal end of the antibody or polypeptide. The length of additional amino acids residues can range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and can comprise a detectable label and/or protein (e.g., a fluorescent tag or an enzyme).

The variant antibodies or antigen-binding fragments described herein can be generated using methods known in the art, including but not limited to, site-directed mutagenesis, alanine scanning mutagenesis, and PCR mutagenesis. Methods for mutagenesis and nucleotide sequence alterations are well known in the art. See, for example, Walker and Gaastra, eds. (1983) TECHNIQUES IN MOLECULAR BIOLOGY (MacMillan Publishing Company, New York); Kunkel, Proc. Natl. Acad. Sci. USA 82:488-492 (1985); Kunkel et al., Methods Enzymol. 54:367-382 (1987); Sambrook et al. (1989) MOLECULAR CLONING: A LABORATORY MANUAL (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest can be found in the model of Dayhoff et al. (1978) in Atlas of Protein Sequence and Structure (Natl. Biomed. Res. Found., Washington, D.C.), pp. 345-352, herein incorporated by reference in its entirety. The model of Dayhoff et al. uses the Point Accepted Mutation (PAM) amino acid similarity matrix (PAM 250 matrix) to determine suitable conservative amino acid substitutions. Conservative substitutions, such as exchanging one amino acid with another having similar properties, can be beneficial. Examples of conservative amino acid substitutions as taught by the PAM 250 matrix of the Dayhoff et al. model include, but are not limited to, Gly→Ala, Val→Ile→Leu, Asp→Glu, Lys→Arg, Asn→Gln, and Phe→Trp→Tyr.

In constructing variants of an anti-CEACAM1 binding molecule, e.g., an antibody or antigen-binding fragment, variant, or derivative thereof, modifications are made such that variants continue to possess the desired properties, e.g., being capable of specifically binding to an CEACAM1, and in certain embodiments being able to activate immune cells against tumor cells. Obviously, any mutations made in the DNA encoding the variant polypeptide must not place the sequence out of reading frame. In some embodiments, mutations made in the DNA do not create complementary regions that could produce secondary mRNA structure.

In some embodiments, a variant of an anti-CEACAM1 antibody or antigen-binding fragment disclosed herein can retain the ability to bind CEACAM1 to a similar extent, the same extent, or to a higher extent, as the parent antibody or antigen-binding fragment. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent antibody or antigen-binding fragment. In certain embodiments, a variant of an anti-CEACAM1 antibody or antigen-binding fragment comprises the amino acid sequence of the parent anti-CEACAM1 antibody or antigen-binding fragment with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of an anti-CEACAM1 antibody or antigen-binding fragment comprises the amino acid sequence of the parent antibody or antigen-binding fragment with one or more non-conservative amino acid substitutions. In some embodiments, a variant of an anti-CEACAM1 antibody or antigen-binding fragment comprises the amino acid sequence of the parent binding antibody or antigen-binding fragment with one or more non-conservative amino acid substitution, wherein the one or more non-conservative amino acid substitutions do not interfere with or inhibit one or more biological activities of the variant (e.g., CEACAM1 binding). In certain embodiments, the one or more conservative amino acid substitutions and/or the one or more non-conservative amino acid substitutions can enhance a biological activity of the variant, such that the biological activity of the functional variant is increased as compared to the parent antibody or antigen-binding fragment.

In some embodiments, the variant can have 1, 2, 3, 4, or 5 amino acid substitutions in the CDRs (e.g., VH CDR1, VH CDR2, VH CDR3, VL CDR1, VL CDR2 and VL CDR3) of the binding moiety.

In some embodiments, anti-CEACAM1 antibodies or antigen-binding fragments described herein are chemically modified naturally or by intervention. In some embodiments, the anti-CEACAM1 antibodies or antigen-binding fragments have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The anti-CEACAM1 antibodies or antigen-binding fragments can comprise one or more analogs of an amino acid (including, for example, unnatural amino acids), as well as other modifications known in the art.

In some embodiments, anti-CEACAM1 antibodies or antigen-binding fragments disclosed herein can be linked to at least one agent to form an antibody conjugate. The conjugate can be, for example, an antibody conjugated to another protein, carbohydrate, lipid, steroids, immunosuppressors, or mixed moiety molecule(s). Such antibody conjugates include, but are not limited to, modifications that include linking the antibody to one or more polymers. For example, an antibody or antigen-binding fragment can be linked to one or more water-soluble polymers. Linkage to a water-soluble polymer reduces the likelihood that the antibody or antigen-binding fragment precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety can be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which can be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric or bioluminescent reaction), a substrate, a solid matrix, such as biotin. An antibody can comprise one, two, or more of any of these labels.

Antibody conjugates can be used to deliver cytotoxic agents to target cells. Cytotoxic agents of this type can improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, calechaemicin, etc.), radioconjugates, enzyme conjugates (e.g., RNase conjugates, granzyme antibody-directed enzyme/prodrug therapy), and the like.

Antibody conjugates are also used as diagnostic agents. In some embodiments, an anti-CEACAM1 antibody or antigen-binding fragment described herein is conjugated to a detectable substance or molecule that allows the agent to be used for diagnosis and/or detection. A detectable substance can include, but is not limited to, enzymes; prosthetic groups (e.g., biotin and flavine(s)); fluorescent materials; bioluminescent materials, such as luciferase; radioactive materials; positron emitting metals; and magnetic metal ions positron emitting metals; and magnetic metal ions.

Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, e.g., U.S. Pat. Nos. 5,021,236, 4,938,948, and 4,472,509). The imaging moieties used can be paramagnetic ions, radioactive isotopes, fluorochromes, NMR-detectable substances, MR hyperpolarized molecules, targeted ultrasound bubbles, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III). Alternative useful isotopes are those used for hyperpolarized MRI, such as carbon-13 and silica-29.

The radioactive isotopes contemplated for use in imaging and radiotherapy as conjugates or covalent incorporation include astatine-211, actinium-225, carbon-14, bismuth-212, chromium-51, chlorine-36, cobalt-57, cobalt-58, copper-64, copper-67, Eu-152, florine-18, gallium-68, gallium-67, gold-198, hydrogen-3, iodine-123, iodine-125, iodine-131, indium-111, iron-52, iron-59, lead-212, lutetium-177, phosphorus-32, rhenium-186, rhenium-188, rubidium-82, rhodium-99, selenium-75, sulphur-35, samarium-153, strontium-92, strontium-89, thallium-201, thorium-227, technetium-94m, technitium-99m, yttrium-86, yttrium-90, zirconium-86, and/or zirconium-89. F-18, Zr-89 and Cu-64 often being preferred for PET imaging. Lu-177, At-211, and Yt-90 often being preferred for radiotherapy. Radioactively labeled monoclonal antibodies and antibody fragments of the present disclosure can be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure can be labeled with technetium-99m by ligand exchange process, for example, by reducing pertechnate with stannous solution, chelating the reduced technetium onto a Sephadex column and applying the antibody to this column. Alternatively, direct labeling techniques can be used, e.g., by incubating pertechnate, a reducing agent such as SNCl₂, a buffer solution such as sodium-potassium phthalate solution, and the antibody. Intermediary functional groups that incorporate chelators, which are often used to bind radioisotopes that exist as metallic ions to an antibody are diethylene-triamine-pentaacetic acid (DTPA), ethylene diamine-tetraacetic acid (EDTA), monomeric or dendrimeric 1,4,7,10-tetraazacyclododecane-1,4,7,10-tetraacetic acid (DOTA), 1,4,7-triazacyclononane-1,4,7-triacetic acid (NOTA), deferoxamine (DFO), or 1-hydroxy-2(1H)-pyridinone derivatives (e.g., 3,4,3-LI(1,2-HOPO) or HOPO).

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, cyanine (Cy3), Cy5,6-FAM, dansyl chloride, dichlorotriazinylamine fluorescein, fluorescein isothiocyanate (FITC), HEX, 6-JOE, Oregon Green 488, Oregon Green 500, Oregon Green 514, Pacific Blue, Phycoerythrin, REG, Rhodamine Green, Rhodamine Red, Renographin, ROX, TAMRA, TET, tetramethylrhodamine isothiocyanate (TRITC), Texas Red, and/or Umbelliferone.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include beta-galactosidase, acetylcholinesterase, urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylene-triamine-pentaacetic acid anhydride (DTP A); ethylene-diamine-tetraacetic acid; monomeric or dendrimeric 1,4,7,10-tetraazacyclododecane-1,4,7,10-tetraacetic acid (DOTA); 1,4,7-triazacyclononane-1,4,7-triacetic acid (NOTA); DFO; HOPO; N-chloro-p-toluenesulfonamide; and/or tetrachloro-3a-6a-diphenylglycouril-3 attached to the antibody (U.S. Pat. Nos. 4,472,509 and 4,938,948). Monoclonal antibodies can also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl) propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction.

Molecules containing azido groups can also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and can be used as antibody binding agents.

Derivatization of immunoglobulins by selectively introducing sulfhydryl groups in the Fc region of an immunoglobulin, using reaction conditions that do not alter the antibody combining site are also contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

In some embodiments, the anti-CEACAM1 antibodies or antigen-binding fragments described herein can be conjugated to a cytotoxic agent or moiety. In some embodiments, the antibody or antigen-binding fragment is conjugated to a cytotoxic agent to form an ADC (antibody-drug conjugate). In some embodiments, antibody drug conjugates, or ADCs, are a class of highly potent biopharmaceutical drugs designed as a targeted therapy. ADCs are composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates. By combining the unique targeting capabilities of monoclonal antibodies with the cancer-killing ability of cytotoxic drugs, ADCs allow sensitive discrimination between healthy and diseased tissue. This means that, in contrast to traditional systemic approaches, ADCs target and attack the diseased cell so that healthy cells are less severely affected.

In some embodiments, the anti-CEACAM1 antibodies or antigen-binding fragments described herein can be conjugated to a cytotoxic agent or moiety. In some embodiments, the antibody or antigen-binding fragment is conjugated to a cytotoxic agent to form an ADC (antibody-drug conjugate). In some embodiments, antibody drug conjugates, or ADCs, are a class of highly potent biopharmaceutical drugs designed as a targeted therapy. ADCs are composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates. By combining the unique targeting capabilities of monoclonal antibodies with cytotoxic drugs, ADCs allow sensitive discrimination between healthy and diseased tissue. This means that, in contrast to traditional systemic approaches, ADCs target and attack the diseased cell so that healthy cells are less severely affected.

In the development of ADC-based anti-tumor therapies, a warhead (e.g., a cell toxin or cytotoxin) is coupled to an antibody that specifically targets a certain cell marker (e.g., a protein that, ideally, is only to be found in or on diseased cells). Antibodies target these proteins in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. In other cases, the linker is cleavable on the surface of the target cell or early endosomes, and as such, full internalization is not required. Due to this targeting, ideally the drug has lower side effects and gives a wider therapeutic window than other agents.

In some embodiments, the cytotoxic moiety the ADCs having the anti-CEACAM1 antibodies or antigen-binding fragments described herein is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DM1 and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody or antigen-binding fragment is conjugated to one or more small molecule toxins, such as calicheamicins, maytansinoids, trichothenes, and CC1065.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. The availability of better and more stable linkers has changed the function of the chemical bond. The type of linker, cleavable or noncleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex—amino acid, linker and cytotoxic agent—now becomes the active drug. In contrast, cleavable linkers are catalyzed by enzymes in or on the host cell, thereby releasing the cytotoxic agent. Commonly used mechanisms for linker cleavage are protease sensitivity, pH sensitivity, and glutathione sensitivity. Another type of cleavable linker adds an extra molecule between the cytotoxic drug and the cleavage site. This linker technology allows researchers to create ADCs with more flexibility without changing cleavage kinetics. A new method of peptide cleavage based on Edman degradation has also been developed. Future direction in the development of ADCs also includes the development of site-specific conjugation (TDCs) to further improve stability and therapeutic index and a-emitting immunoconjugates and antibody-conjugated nanoparticles.

An anti-CEACAM1 antibody or antigen-binding fragment described herein can be attached to a solid support. Such solid supports include, but are not limited to, glass, cellulose, polyacrylamide, nylon, polystyrene, polyvinyl chloride, or polypropylene. In some embodiments, an immobilized anti-CEACAM1 antibody or antigen-binding fragment is used in an immunoassay. In some embodiments, an immobilized anti-CEACAM1 antibody or antigen-binding fragment is used in purification of the target antigen (e.g., human CEACAM1).

### 6.4 Polynucleotides and Vectors

Also provided herein are polynucleotides that encode a polypeptide (e.g., an anti-CEACAM1 antibody or antigen-binding fragment) described herein. The term “polynucleotide that encodes a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA can be cDNA, genomic DNA, or synthetic DNA, and can be double-stranded or single-stranded. Single stranded DNA can be the coding strand or non-coding (anti-sense) strand. The polynucleotides of the disclosure can be mRNA.

Expressly contemplated herein are polynucleotides encode any anti-CEACAM1 antibody or antigen-binding fragment disclosed herein. For illustrative purposes, in some embodiments, the polynucleotides provided herein encode an anti-CEACAM1 antibody or antigen-binding fragment having a VL and a VH, wherein the VL comprises VL CDR1, CDR2 and CDR3 and the VH comprises VH CDR1, CDR2 and CDR3, and wherein the VL CDR1, VL CDR2, VL CDR3, VH CDR1, VH CDR2 and VH CDR3 have the amino acid sequences of (1) SEQ ID NOs:1, 8, 13, 20, 28, and 40, respectively; (2) SEQ ID NOs:2, 9, 14, 21, 29, and 41, respectively; (3) SEQ ID NOs:3, 9, 14, 21, 30, and 42, respectively; (4) SEQ ID NOs:4, 8, 15, 22, 31, and 43, respectively; (5) SEQ ID NOs:5, 10, 16, 23, 32, and 44, respectively; (6) SEQ ID NOs:3, 9, 17, 24, 33, and 45, respectively; (7) SEQ ID NOs:3, 9, 17, 24, 33, and 46, respectively; (8) SEQ ID NOs:3, 9, 17, 24, 33, and 47, respectively; (9) SEQ ID NOs:3, 9, 17, 24, 33, and 48, respectively; (10) SEQ ID NOs:3, 9, 17, 24, 34, and 45, respectively; (11) SEQ ID NOs:3, 9, 17, 24, 34, and 46, respectively; (12) SEQ ID NOs:3, 9, 17, 24, 34, and 47, respectively; (13) SEQ ID NOs:3, 9, 17, 24, 34, and 48, respectively; (14) SEQ ID NOs:3, 9, 17, 24, 35, and 45, respectively; (15) SEQ ID NOs:3, 9, 17, 24, 35, and 46, respectively; (16) SEQ ID NOs:3, 9, 17, 24, 35, and 47, respectively; (17) SEQ ID NOs:3, 9, 17, 24, 35, and 48, respectively; (18) SEQ ID NOs:3, 9, 17, 24, 36, and 45, respectively; (19) SEQ ID NOs:3, 9, 17, 24, 36, and 46, respectively; (20) SEQ ID NOs:3, 9, 17, 24, 36, and 47, respectively; (21) SEQ ID NOs:3, 9, 17, 24, 36, and 48, respectively; (22) SEQ ID NOs:3, 9, 17, 24, 37, and 45, respectively; (23) SEQ ID NOs:3, 9, 17, 24, 37, and 46, respectively; (24) SEQ ID NOs:3, 9, 17, 24, 37, and 47, respectively; (25) SEQ ID NOs:3, 9, 17, 24, 37, and 48, respectively; (26) SEQ ID NOs:6, 11, 18, 25, 38, and 49, respectively; (27) SEQ ID NOs:7, 12, 19, 26, 39, and 50, respectively; or (28) SEQ ID NOs:7, 12, 19, 27, 38, and 51, respectively; or a variant thereof having up to about 5 amino acid substitutions, additions, and/or deletions in the CDRs. For illustrative purposes, in some embodiments, the polynucleotides provided herein encode an anti-CEACAM1 antibody or antigen-binding fragment having a VL and a VH, wherein the VL comprises VL CDR1, CDR2 and CDR3 and the VH comprises VH CDR1, CDR2 and CDR3, and wherein the VL CDR1, VL CDR2, VL CDR3, VH CDR1, VH CDR2 and VH CDR3 have the amino acid sequences of (1) SEQ ID NOs:52, 9, 17, 24, 35, and 46, respectively; (2) SEQ ID NOs:53, 9, 17, 24, 35, and 46, respectively; (3) SEQ ID NOs:3, 54, 17, 24, 35, and 46, respectively; (4) SEQ ID NOs:3, 55, 17, 24, 35, and 46, respectively; (5) SEQ ID NOs:52, 54, 17, 24, 35, and 46, respectively; (6) SEQ ID NOs:52, 55, 17, 24, 35, and 46, respectively; (7) SEQ ID NOs:52, 56, 17, 24, 35, and 46, respectively; or (8) SEQ ID NOs:3, 9, 17, 57, 35, and 46, respectively; or a variant thereof having up to about 5 amino acid substitutions, additions, and/or deletions in the CDRs. The polynucleotides can be in the form of DNA. The polynucleotides can be in the form of mRNA.

In some embodiments, the polynucleotides provided herein encode an anti-CEACAM1 antibody or antigen-binding fragment comprising a VL and a VH, wherein the VL and VH have the amino acid sequences of (1) SEQ ID NOs:61 and 70, respectively; (2) SEQ ID NOs:62 and 71, respectively; (3) SEQ ID NOs:63 and 72, respectively; (4) SEQ ID NOs:64 and 73, respectively; (5) SEQ ID NOs:65 and 74, respectively; (6) SEQ ID NOs:66 and 75, respectively; (7) SEQ ID NOs:67 and 76, respectively; (8) SEQ ID NOs:68 and 77, respectively; or (9) SEQ ID NOs:68 and 78, respectively; or a variant thereof having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the recited VL and/or VH sequences. The polynucleotides can be in the form of DNA. The polynucleotides can be in the form of mRNA.

In some embodiments, the polynucleotides provided herein encode humanized anti-CEACAM1 antibodies or antigen-binding fragments comprising a VL and a VH, wherein VL has an amino acid sequence selected from the group consisting of SEQ ID NOs:81-90; and wherein VH has an amino acid sequence selected from the group consisting of SEQ ID NOs:111-116; or a variant thereof having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the recited VL and/or VH sequences. In some embodiments, the polynucleotides provided herein encode humanized anti-CEACAM1 antibodies or antigen-binding fragments comprising a VL and a VH, wherein VL has an amino acid sequence selected from the group consisting of SEQ ID NOs:91-93 and 107-109; and wherein VH has an amino acid sequence selected from the group consisting of SEQ ID NOs:117-122; or a variant thereof having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the recited VL and/or VH sequences. In some embodiments, the polynucleotides provided herein encode humanized anti-CEACAM1 antibodies or antigen-binding fragments comprising a VL and a VH, wherein VL has an amino acid sequence selected from the group consisting of SEQ ID NOs:94-106; and wherein VH has an amino acid sequence selected from the group consisting of SEQ ID NOs:123-128, 130 and 143-149; or a variant thereof having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the recited VL and/or VH sequences. The polynucleotides can be in the form of DNA. The polynucleotides can be in the form of mRNA.

In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO:140). In some embodiments, the linker has the amino acid sequence of (EAAAK)n, n=1, 2, 3, 4, or 5 (SEQ ID NO:141). In some embodiments, the linker has the amino acid sequence of (PA)nP, n=1, 2, 3, 4, or 5 (SEQ ID NO:142).

The present disclosure also provides variants of the polynucleotides described herein, wherein the variants encode, for example, fragments, analogs, and/or derivatives of an anti-CEACAM1 antibody or antigen-binding fragment disclosed herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-CEACAM1 antibody or antigen-binding fragment described herein.

As used herein, the phrase “a polynucleotide having a nucleotide sequence at least about 95% identical to a polynucleotide sequence” means that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker can be used in conjunction with other markers or tags.

In some embodiments, a polynucleotide is isolated. In some embodiments, a polynucleotide is substantially pure.

Vectors and cells comprising the polynucleotides described herein are also provided. In some embodiments, provided herein are vectors comprising a polynucleotide provided herein. The vectors can be expression vectors. In some embodiments, vectors provided herein comprise a polynucleotide encoding an anti-CEACAM1 antibody or antigen-binding fragment described herein. In some embodiments, vectors provided herein comprise a polynucleotide encoding a polypeptide that is part of an anti-CEACAM1 antibody or antigen-binding fragment described herein.

In some embodiments, provided herein are recombinant expression vectors, which can be used to amplify and express a polynucleotide encoding an anti-CEACAM1 antibody or antigen-binding fragment described herein. For example, a recombinant expression vector can be a replicable DNA construct that includes synthetic or cDNA-derived DNA fragments encoding a polypeptide chain of an anti-CEACAM1 antibody, operatively linked to suitable transcriptional and/or translational regulatory elements derived from mammalian, microbial, viral or insect genes. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in certain expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages. In some embodiments, an anti-CEACAM1 antibody or antigen-binding fragment described herein is expressed from one or more vectors.

Provided herein are suitable host cells comprising vectors described herein. In some embodiments, the host cells can be used for recombination expression of the anti-CEACAM1 antibodies described herein. The host cells can include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art.

Examples of suitable mammalian host cells include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

The present disclosure also provides host cells comprising the polypeptides described herein, polynucleotides encoding polypeptides described herein, or vectors comprising such polynucleotides. In some embodiments, provided herein are host cells comprising a vector comprising a polynucleotide disclosed herein. In some embodiments, host cells provided herein comprise a vector comprising a polynucleotide encoding an anti-CEACAM1 antibody or antigen-binding fragment described herein. In some embodiments, host cells provided herein comprise a vector comprising a polynucleotide encoding a polypeptide that is part of an anti-CEACAM1 antibody or antigen-binding fragment described herein. In some embodiments, host cells provided herein comprise a polynucleotide encoding an anti-CEACAM1 antibody or antigen-binding fragment described herein. In some embodiments, the cells produce the anti-CEACAM1 antibodies or antigen-binding fragments described herein.

### 6.5 Methods of Manufacture

Provided herein are anti-CEACAM1 antibodies and antigen-binding fragments thereof that include but are not limited to monoclonal antibodies, polyclonal antibodies, synthetic antibodies, human antibodies, humanized antibodies, and antigen-binding fragments thereof.

Methods of antibody production are well-known in the art. See for example, in Harlow et al., Antibodies: A Laboratory Manual, (Cold Spring Harbor Laboratory Press, 2nd ed. 1988); Hammerling et al., in: MONOCLONAL ANTIBODIES AND T-CELL HYBRIDOMAS 563 681 (Elsevier, N.Y., 1981), each of which is incorporated herein by reference in its entirety. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a human protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

Polynucleotides of the antibodies or antigen-binding fragments provided herein can be prepared, manipulated, and/or expressed using any of the well-established techniques known and available in the art. In some embodiments, polynucleotides of the antibodies or antigen-binding fragments provided herein can be prepared recombinantly. Many vectors can be used. Examples of vectors are plasmid, autonomously replicating sequences, and transposable elements. Exemplary transposon systems such as Sleeping Beauty and PiggyBac can be used, which can be stably integrated into the genome (e.g., Ivics et al., Cell, 91 (4): 501-510 (1997); Cadiñanos et al., (2007) Nucleic Acids Research. 35 (12): e87). Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells.

In some embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In some embodiments, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements can vary in their strength and specificity. Depending on the vector system and host utilized, any number of suitable transcription and translation elements, including ubiquitous promoters and inducible promoters can be used.

Illustrative ubiquitous expression control sequences that can be used in present disclosure include, but are not limited to, a cytomegalovirus (CMV) immediate early promoter, a viral simian virus 40 (SV40) promoter (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, and a β-actin promoter.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. The anti-CEACAM1 antibodies or antigen-binding fragments described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art.

The polypeptides described herein can be prepared using a wide variety of techniques known in the art including the use of hybridoma and recombinant technologies, or a combination thereof. In some embodiments, a recombinant expression vector is used to express a polynucleotide encoding a polypeptide described herein. For example, a recombinant expression vector can be a replicable DNA construct that includes synthetic or cDNA-derived DNA fragments encoding a polypeptide operatively linked to suitable transcriptional and/or translational regulatory elements derived from mammalian, microbial, viral or insect genes. In some embodiments, coding sequences of polypeptides disclosed herein can be ligated into such expression vectors for their expression in mammalian cells. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Useful expression vectors for eukaryotic hosts include, for example, vectors comprising expression control sequences from SV40, bovine papilloma virus, adenovirus, and cytomegalovirus. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

To make anti-CEACAM1 antibodies and antigen-binding fragments that are afucosylated, host cells that (1) overexpress N-acetylglucosaminyltransferase III (GnTIII), (2) lack a-1,6-fucosyltransferase (FUT8), or (3) have a low fucose content, or any combination of (1)-(3) can be used. In some embodiments, used herein are host cells that overexpresses N-acetylglucosaminyltransferase III (GnTIII). In some embodiments, used herein are host cells that lack a-1,6-fucosyltransferase (FUT8). In some embodiments, used herein are host cells having a low fucose content. In some embodiments, CHO host cells are used.

Peptides can be synthesized, in whole or in part, using chemical methods (see, e.g., Caruthers (1980). Nucleic Acids Res. Symp. Ser. 215; Horn (1980); and Banga, A. K., THERAPEUTIC PEPTIDES AND PROTEINS, FORMULATION, PROCESSING AND DELIVERY SYSTEMS (1995) Technomic Publishing Co., Lancaster, PA). Peptide synthesis can be performed using various solid phase techniques (see, e.g., Roberge, Science 269:202 (1995); Merrifield, Methods. Enzymol. 289:3 (1997)) and automated synthesis may be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., ORGANIC SYNTHESES COLLECTIVE VOLUMES, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions and modifications thereof.

For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

In some embodiments, an anti-CEACAM1 antibody or antigen-binding fragment is a human antibody or antigen-binding fragment. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, can be employed to generate higher affinity human antibodies. In some embodiments, human antibodies are produced in transgenic mice that contain human immunoglobulin loci. Upon immunization these mice can produce the full repertoire of human antibodies in the absence of endogenous immunoglobulin production.

Human antibodies can also be produced using transgenic mice which are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. For example, anti-CEACAM1 antibodies directed against the human CEACAM1 antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson and Chiswell, Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies can also be generated by in vitro activated B cells (see, U.S. Pat. Nos. 5,567,610 and 5,229,275, each of which is incorporated herein by reference in its entirety). Human antibodies can also be generated in vitro using hybridoma techniques such as, but not limited to, that described by Roder et al. (Methods Enzymol., 121:140-167 (1986)).

Alternatively, in some embodiments, a non-human antibody is humanized, where specific sequences or regions of the antibody are modified to increase similarity to an antibody naturally produced in a human. In some embodiment, the antigen binding domain portion is humanized. Various methods for generating humanized antibodies are known in the art. Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant CEACAM1 or an epitope thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant CEACAM1 or an epitope thereof).

A humanized antibody can be produced using techniques including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions can be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. For example, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity as the original antibody, for example, the ability to bind to human CEACAM1 antigen. However, using certain methods of humanization, the affinity and/or specificity of binding of the antibody for a particular antigen can be increased using methods of “directed evolution,” as described by Wu et al., J. Mol. Biol., 294:151 (1999), the contents of which are incorporated herein by reference herein in their entirety.

Anti-CEACAM1 antibodies or antigen-binding fragments described herein can be tested for binding to human CEACAM1 by, for example, standard ELISA. Briefly, microtiter plates are coated with purified CEACAM1, and then blocked with bovine serum albumin. Dilutions of antibody (e.g., dilutions of plasma from CEACAM1-immunized mice) are added to each well and incubated. The plates are washed and incubated with secondary reagent (e.g., for human antibodies, a goat-anti-human IgG Fc-specific polyclonal reagent) conjugated to horseradish peroxidase (HRP). After washing, the plates can be developed and analyzed by a spectrophotometer. Sera from immunized mice can then be further screened by flow cytometry for binding to a cell line expressing human CEACAM1, but not to a control cell line that does not express CEACAM1. Briefly, the binding of anti-CEACAM1 antibodies can be assessed by incubating CEACAM1 expressing CHO cells with the anti-CEACAM1 antibody. The cells can be washed, and binding can be detected with an anti-human IgG Ab. Flow cytometric analyses can be performed using a FACS can flow cytometry (Becton Dickinson, San Jose, CA). Mice which develop the highest titers can be used for fusions.

An ELISA assay as described above can be used to screen for antibodies and, thus, hybridomas that produce antibodies that show positive reactivity with the CEACAM1 immunogen. Hybridomas that produce antibodies that bind with high affinity to CEACAM1 can then be subcloned and further characterized. One clone from each hybridoma, which retains the reactivity of the parent cells (by ELISA), can then be chosen for making a cell bank, and for antibody purification.

To purify anti-CEACAM1 antibodies, selected hybridomas can be grown for monoclonal antibody purification. Supernatants can be filtered and concentrated before affinity chromatography. Eluted IgG can be checked by gel electrophoresis and high-performance liquid chromatography to ensure purity. The buffer solution can be exchanged, and the concentration can be determined. The monoclonal antibodies can be aliquoted and stored.

To determine if the selected anti-CEACAM1 monoclonal antibodies bind to unique epitopes, each antibody can be biotinylated using commercially available reagents (Pierce, Rockford, IL). Biotinylated MAb binding can be detected with a streptavidin labeled probe. Competition studies using unlabeled monoclonal antibodies and biotinylated monoclonal antibodies can be performed using CEACAM1 coated-ELISA plates as described above.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/mL of anti-human immunoglobulin overnight at 4° C. After blocking with 1% BSA, the plates are reacted with 1 f ig/mL or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

To test the binding of monoclonal antibodies to live cells expressing CEACAM1, flow cytometry can be used, as described in the Examples. Briefly, cell lines expressing membrane-bound CEACAM1 (grown under standard growth conditions) are mixed with various concentrations of monoclonal antibodies in PBS containing 0.1% BSA at 4° C. for 1 hour. After washing, the cells are reacted with Fluorescein-labeled anti-IgG antibody under the same conditions as the primary antibody staining. The samples can be analyzed by FACScan instrument using light and side scatter properties to gate on single cells and binding of the labeled antibodies is determined. An alternative assay using fluorescence microscopy can be used (in addition to or instead of) the flow cytometry assay. Cells can be stained exactly as described above and examined by fluorescence microscopy. This method allows visualization of individual cells, but can have diminished sensitivity depending on the density of the antigen.

Anti-CEACAM1 antibodies or antigen-binding fragments can be further tested for reactivity with the CEACAM1 antigen by Western blotting. Briefly, cell extracts from cells expressing CEACAM1 can be prepared and subjected to sodium dodecyl sulfate polyacrylamide gel electrophoresis. After electrophoresis, the separated antigens will be transferred to nitrocellulose membranes, blocked with 20% mouse serum, and probed with the monoclonal antibodies to be tested. IgG binding can be detected using anti-IgG alkaline phosphatase and developed with BCIP/NBT substrate tablets (Sigma Chem. Co., St. Louis, MO).

Methods for analyzing binding affinity, cross-reactivity, and binding kinetics of various anti-CEACAM1 antibodies include standard assays known in the art, for example, biolayer interferometry (BLI) using, for example, Gator system (Probe Life) or the Octet-96 system (Sartorius AG), or BIACORE™ surface plasmon resonance (SPR) analysis using a BIACORE™ 2000 SPR instrument (Biacore AB, Uppsala, Sweden).

Assays for analyzing functional activities of various anti-CEACAM1 antibodies are also well known in the art. For example, T cell activity can be measured by, for example, the INFγ release. Various in vitro assays, such as those described in the experimental sections below, can be adopted to confirm the activities of various anti-CEACAM1 antibodies in enhancing the tumor-killing activities of immune cells, such as T cells, NK cells, NKT cells, monocytes, dendritic cells and neutrophils.

### 6.6 Pharmaceutical Compositions

Provided herein are also pharmaceutical compositions comprising the anti-CEACAM1 antibodies or antigen-binding fragments disclosed herein. In some embodiments, the pharmaceutical composition comprises a therapeutically effective amount of the anti-CEACAM1 antibodies or antigen-binding fragments disclosed herein and a pharmaceutically acceptable carrier. In some embodiments, the pharmaceutical compositions are useful in immunotherapy. In some embodiments, the pharmaceutical compositions are useful in immuno-oncology. In some embodiments, the pharmaceutical compositions are useful in inhibiting tumor growth in a subject (e.g., a human patient). In some embodiments, the pharmaceutical compositions are useful in treating cancer in a subject (e.g., a human patient).

In some embodiments, the pharmaceutical compositions provided herein comprise anti-CEACAM1 antibodies or antigen-binding fragments provided herein. The anti-CEACAM1 antibodies or antigen-binding fragments can be present at various concentrations. In some embodiments, the pharmaceutical compositions provided herein comprise soluble anti-CEACAM1 antibodies or antigen-binding fragments provided herein at 1-1000 mg/mL. Dosages can be readily adjusted by those skilled in the art; for example, a decrease in purity may require an increase in dosage.

Provided herein are also kits for preparation of pharmaceutical compositions having the anti-CEACAM1 antibodies or antigen-binding fragments disclosed herein. In some embodiments, the kit comprises the anti-CEACAM1 antibodies or antigen-binding fragments disclosed herein and a pharmaceutically acceptable carrier in one or more containers. In another embodiment, the kits can comprise anti-CEACAM1 antibodies or antigen-binding fragments disclosed herein for administration to a subject. In specific embodiments, the kits comprise instructions regarding the preparation and/or administration of the anti-CEACAM1 antibodies or antigen-binding fragments.

In some embodiments, provided herein is a pharmaceutical composition comprising anti-CEACAM1 antibodies or antigen-binding fragments or cells provided herein wherein the composition is suitable for local administration. In some embodiments, local administration comprises intratumoral injection, peritumoral injection, juxtatumoral injection, intralesional injection and/or injection into a tumor draining lymph node, or essentially any tumor-targeted injection where the antitumor agent is expected to leak into primary lymph nodes adjacent to targeted solid tumor.

Pharmaceutically acceptable carriers that can be used in compositions provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active ingredient (i.e., anti-CEACAM1 antibodies or antigen-binding fragments) can be coated in a material to protect the active ingredient from the action of acids and other natural conditions that can inactivate the active ingredient.

Provided herein are also pharmaceutical compositions or formulations that improve the stability of the anti-CEACAM1 antibodies or antigen-binding fragments to allow for their long-term storage. In some embodiments, the pharmaceutical composition or formulation disclosed herein comprises: (a) anti-CEACAM1 antibodies or antigen-binding fragments disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also depend on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, rafftnose, or maltose, polyols such as glycerol, mannitol, sorbitol, cyclodextrins or destrans of any kind and molecular weight, or PEG. In some embodiments, the stabilizing agent is chosen to maximize the stability of FIX polypeptide in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

The pharmaceutical compositions disclosed herein can further comprise one or more of a buffer system, a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19^{th} edition, 1995.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In some embodiments, the pharmaceutical compositions disclosed herein are freeze-dried, to which the physician or the patient adds solvents and/or diluents prior to use.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition or formulation can comprise a preservative or can be devoid of a preservative. Supplementary active compounds can be incorporated into the compositions.

Pharmaceutical compositions or formulations typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material in the pharmaceutical compositions or formulations disclosed herein can vary. In some embodiments, the amount of active ingredient which can be combined with a carrier material is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., Sustained and Controlled Release Drug Delivery Systems, J. R. Robinson, ed., Marcel Dekker, Inc., New York, 1978.

In some embodiments, the anti-CEACAM1 antibodies or antigen-binding fragments described herein can be formulated to ensure proper distribution in vivo. For example, the blood-brain barrier (BBB) excludes many highly hydrophilic compounds. To ensure that the activate ingredient described herein cross the BBB, they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al) mannosides (Umezawa et al, (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); surfactant protein A receptor (Briscoe et al. (1995) Am. J. Physiol. 1233: 134); p 120 (Schreier et al. (1994) J. Biol. Chem. 269:9090); see also K. Keinanen; M. L. Laukkanen (1994) FEBS Lett. 346: 123; J. J. Killion; I. J. Fidler (1994) Immunomethods 4:273.

### 6.7 Methods and Uses

The antibodies or antigen-binding fragments, compositions and methods described herein have numerous in vitro and in vivo utilities involving, for example, enhancement of immune response, or inhibiting tumor angiogenesis, by inhibiting (or antagonizing) CEACAM1 (e.g., signaling), or detection CEACAM1. In some embodiments, the anti-CEACAM1 antibodies or antigen-binding fragments described herein are humanized antibodies or antigen-binding fragments. For example, anti-CEACAM1 antibodies or antigen-binding fragments described herein can be administered to cells in culture, in vitro or ex vivo, or to human subjects, e.g., in vivo, to enhance immunity in a variety of diseases. Accordingly, provided herein are methods of modifying an immune response in a subject comprising administering to the subject an anti-CEACAM1 antibody, or antigen binding portion thereof described herein, such that the immune response in the subject is modified. In some embodiments, the response is enhanced, stimulated or up-regulated.

In some embodiments, provided herein are methods of inducing or stimulating immune cell activation comprising contacting an immune cell with an effective amount of an anti-CEACAM1 antibody or antigen binding fragment described herein. In some embodiments, provided herein are methods of inducing or stimulating immune cell proliferation comprising contacting an immune cell with an effective amount of an anti-CEACAM1 antibody or antigen binding fragment described herein. In some embodiments, provided herein are methods of reducing CEACAM1 mediated suppression of an immune cell proliferation comprising contacting an immune cell with an effective amount of an anti-CEACAM1 antibody or antigen binding fragment described herein. In some embodiments, provided herein are methods of inhibiting the interaction between CEACAM1 and a ligand thereof on an immune cell comprising contacting the immune cell with an effective amount of an anti-CEACAM1 antibody or antigen binding fragment described herein. In some embodiments, provided herein are methods of increasing cytokine (e.g., IFN-γ) production by an immune cell comprising contacting an immune cell with an effective amount of an anti-CEACAM1 antibody or antigen binding fragment described herein. The immune cells can be, for example, lymphocytes such as T cells, NK cells and NKT cells. In some embodiments, the T cells can be CD4⁺ T cells, CD8⁺ T cells, T helper (Th) cells (e.g., Th1 cells), T cytotoxic (Tc) cells or TILs. The immune cells can also be myeloid cells. In some embodiments, the immune cells can be monocytes. In some embodiments, the immune cells can be dendritic cells. In some embodiments, the immune cells can be neutrophils. In some embodiments, the immune cells can be vascular endothelial cells (VECs).

Subjects suitable for the present methods include human patients in whom enhancement of an immune response would be desirable. The methods are particularly suitable for treating human patients having a disorder that can be treated by augmenting an immune response (e.g., a T-cell mediated immune response, e.g., an antigen specific T cell response). In some embodiments, the methods are particularly suitable for treatment of cancer in vivo. In some embodiments, provided herein are methods of enhancing an immune response in a subject in need thereof comprising administer to the subject an effective amount of an anti-CEACAM1 antibody or antigen binding fragment described herein. To achieve antigen-specific enhancement of immunity, anti-CEACAM1 antibodies or antigen-binding fragments described herein can be administered together with an antigen of interest or the antigen can already be present in the subject to be treated (e.g., a tumor-bearing or virus-bearing subject).

Given the ability of anti-CEACAM1 antibodies or antigen-binding fragments described herein to stimulate or co-stimulate T cell responses, e.g., antigen-specific T cell responses, such as by inhibiting negative effects of CEACAM1, provided herein are in vitro and in vivo methods of using the anti-CEACAM1 antibodies or antigen-binding fragments described herein to stimulate, enhance or upregulate antigen-specific T cell responses, e.g., anti-tumor T cell responses.

In some embodiments, CD3 stimulation is also provided (e.g., by coincubation with a cell expressing membrane CD3), which stimulation can be provided at the same time, before, or after stimulation with an anti-CEACAM1 antibody or antigen-binding fragment. For example, provided herein are methods of stimulating an antigen-specific T cell response comprising contacting said T cell with an anti-CEACAM1 antibody or antigen-binding fragment described herein, and optionally with an anti-CD3 antibody, such that an antigen-specific T cell response is stimulated.

Any suitable indicator of an antigen-specific T cell response can be used to measure the antigen-specific T cell response. Non-limiting examples of such suitable indicators include increased T cell proliferation in the presence of the antibody and/or increase cytokine production in the presence of the antibody. In some embodiments, interleukin-2 and/or interferon-γ production by the antigen-specific T cell is stimulated.

Further encompassed are methods of stimulating an immune response (e.g., an antigen-specific T cell response) in a subject comprising administering an anti-CEACAM1 antibody or antigen-binding fragment described herein to the subject such that an immune response (e.g., an antigen-specific T cell response) in the subject is stimulated. In some embodiments, the subject is a tumor-bearing subject and an immune response against the tumor is stimulated. A tumor can be a solid tumor or a liquid tumor, e.g., a hematological malignancy. In some embodiments, a tumor is an immunogenic tumor. In some embodiments, a tumor is non-immunogenic. In some embodiments, a tumor is PD-L1 positive. In some embodiments, a tumor is PD-L1 negative. In some embodiments, a tumor is CEACAM1 positive. In some embodiments, a tumor is CEACAM5 positive. In some embodiments, a tumor is CEACAM6 positive. A subject can also be a virus-bearing subject and an immune response against the virus is stimulated.

In some embodiments, provided herein are methods for inhibiting angiogenesis of tumor in a subject comprising administering to the subject an anti-CEACAM1 antibody or antigen-binding fragment described herein such that angiogenesis of tumor is inhibited in the subject. In some embodiments, provided herein are methods for inhibiting metastasis of tumor in a subject comprising administering to the subject an anti-CEACAM1 antibody or antigen-binding fragment described herein such that metastasis of tumor is inhibited in the subject. In some embodiments, provided herein are methods for inhibiting polyploidy of tumor in a subject comprising administering to the subject an anti-CEACAM1 antibody or antigen-binding fragment described herein such that polyploidy of tumor is inhibited in the subject. In some embodiments, provided herein are methods for inhibiting macrophage-mediated tumor aggression (e.g., growth, invasion, migration, metastasis and/or polyploidy) in a subject comprising administering to the subject an anti-CEACAM1 antibody or antigen-binding fragment described herein such that macrophage-mediated tumor aggression is inhibited in the subject. In some embodiments, the subject is a tumor-bearing subject and macrophages that promoting tumor invasion are inhibited. A tumor can be a solid tumor or a liquid tumor, e.g., a hematological malignancy.

Further provided are methods for inhibiting growth of tumor cells in a subject comprising administering to the subject an anti-CEACAM1 antibody or antigen-binding fragment described herein such that growth of the tumor is inhibited in the subject. Also provided are methods of treating a viral infection in a subject comprising administering to the subject an anti-CEACAM1 antibody described herein such that the viral infection is treated in the subject.

In some embodiments, anti-CEACAM1 antibodies or antigen-binding fragments described herein are given to a subject as an adjunctive therapy. Treatments of subjects having cancer with an anti-CEACAM1 antibody described herein can lead to prolonged survival, e.g., long-term durable response relative to the current standard of care; long term survival of at least 3 months, 6 months, 9 months, 1, 2, 3, 4, 5, 10 or more years, or recurrence-free survival of at least 3 months, 6 months, 9 months, 1, 2, 3, 4, 5, or 10 or more years. In some embodiments, treatment of a subject having cancer with an anti-CEACAM1 antibody or antigen-binding fragment described herein prevents recurrence of cancer or delays recurrence of cancer by, e.g., 3 months, 6 months, 9 months, 1, 2, 3, 4, 5, or 10 or more years.

Treatment of a subject having cancer with an anti-CEACAM1 antibody or antigen-binding fragment described herein can result in, e.g., stable disease, partial response, increased overall survival, increased disease-free survival, or enhanced progression free survival.

In some embodiments, the anti-CEACAM1 antibodies or antigen-binding fragments described herein are not significantly toxic. For example, the anti-CEACAM1 antibodies or antigen-binding fragments described herein are not significantly toxic to an organ of a human, e.g., one or more of the liver, kidney, brain, lungs, and heart, as determined, e.g., in clinical trials. In some embodiments, anti-CEACAM1 antibodies or antigen-binding fragments do not significantly trigger an undesirable immune response, e.g., autoimmunity or inflammation. In some embodiments, treatment of a subject with a CEACAM1-targeting agent (e.g., an anti-CEACAM1 antibody or antigen-binding fragment described herein) does not result in overstimulation of the immune system to the extent that the subject's immune system then attacks the subject itself (e.g., autoimmune response) or results in, e.g., anaphylaxis. Thus, in some embodiments, anti-CEACAM1 antibodies do not cause anaphylaxis.

In some embodiments, treatment of a subject with an anti-CEACAM1 antibody or antigen-binding fragment described herein does not cause significant inflammatory reactions, e.g., immune-mediated pneumonitis, immune-mediated colitis, immune mediated hepatitis, immune-mediated nephritis or renal dysfunction, immune-mediated hypophysitis, immune-mediated hypothyroidism and hyperthyroidism, or other immune-mediated adverse reactions. In some embodiments, treatment of a subject with an anti-CEACAM1 antibody described herein does not cause significant cardiac disorders, e.g., ventricular arrhythmia; eye disorders, e.g., iridocyclitis; infusion-related reactions; increased amylase, increased lipase; nervous system disorders, e.g., dizziness, peripheral and sensory neuropathy; skin and subcutaneous tissue disorders, e.g., rash, pruritus, exfoliative dermatitis, erythema multiforme, vitiligo or psoriasis; respiratory, thoracic and mediastinal disorders, e.g., cough; fatigue; nausea; decreased appetite; constipation; arthralgia; or diarrhea.

In some embodiments, an anti-CEACAM1 antibody or antigen-binding fragment provides synergistic anti-tumor effects in combination with another cancer therapy, such as a compound that stimulates the immune system (e.g., an immuno-oncology agent). In some embodiments, immune effector cells (e.g., T cells) targeting CEACAM1 described herein provide synergistic anti-tumor effects in combination with another cancer therapy, such as a compound that stimulates the immune system (e.g., an immuno-oncology agent).

The present disclosure also provides methods of uses of the anti-CEACAM1 antibodies or antigen-binding fragments, polynucleotides encoding such antibodies or cells, vectors comprising such polynucleotides, or pharmaceutical compositions having such antibodies or cells disclosed herein in treating cancer.

In some embodiments, the anti-CEACAM1 antibodies or antigen-binding fragments can specifically target CEACAM1-expressing cancer cells in vivo, thereby delivering their therapeutic effect of eliminating, lysing and/or killing cancer cells. In some embodiments, the anti-CEACAM1 antibodies or antigen-binding fragments can reduce the immunosuppressive effects mediated by CEACAM1 signaling pathway, thereby promoting the activities of immune cells in eliminating, lysing and/or killing cancer cells. In some embodiments, the methods include administering a therapeutically effective amount of the anti-CEACAM1 antibodies or antigen-binding fragments disclosed herein to a subject in need thereof.

In some embodiments, the anti-CEACAM1 antibodies or antigen-binding fragments described herein can block the interaction between CEACAM1 and CEACAM5, as well as the interaction between CEACAM1 and CEACAM6. In some embodiments, the anti-CEACAM1 antibodies or antigen-binding fragments can specifically target CEACAM5-expressing cancer cells in vivo, thereby delivering their therapeutic effect of eliminating, lysing and/or killing cancer cells. In some embodiments, the anti-CEACAM1 antibodies or antigen-binding fragments can specifically target CEACAM6-expressing cancer cells in vivo, thereby delivering their therapeutic effect of eliminating, lysing and/or killing cancer cells.

In some embodiments, provided herein are methods of treating tumor or cancer in a subject in need thereof, comprising administering to the subject a therapeutically effective amount of the anti-CEACAM1 antibodies or antigen-binding fragments disclosed herein. In some embodiments, provided herein are uses of the anti-CEACAM1 antibodies or antigen-binding fragments disclosed herein in the treatment of tumor or cancer. In some embodiments, provided herein are uses of the anti-CEACAM1 antibodies or antigen-binding fragments provided herein for the preparation of a medicament for the treatment of tumor or cancer.

In some embodiments, provided herein are methods of treating tumor or cancer in a subject in need thereof, comprising administering to the subject a therapeutically effective amount of the pharmaceutical composition disclosed herein. In some embodiments, provided herein are uses of the pharmaceutical composition disclosed herein in treatment of tumor or cancer. In some embodiments, provided herein are uses of the pharmaceutical composition provided herein for the preparation of a medicament for the treatment of tumor or cancer.

Actual dosage levels of the active ingredients (i.e., the anti-CEACAM1 antibodies) in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

The anti-CEACAM1 antibodies or antigen-binding fragments described herein can be administered as a sustained release formulation, in which case less frequent administration is required. Dosage and frequency vary depending on the half-life of the anti-CEACAM1 antibodies or antigen-binding fragments in the patient. In therapeutic applications, a relatively high dosage at relatively short intervals is sometimes required until progression of the disease is reduced or terminated, and until the patient shows partial or complete amelioration of symptoms of disease.

The anti-CEACAM1 antibodies or antigen-binding fragments or pharmaceutical compositions provided herein can be administered to a subject by any methods known in the art, including, but not limited to, pleural administration, intravenous administration, subcutaneous administration, intranodal administration, intratumoral administration, intramuscular administration, intradermal administration, intrathecal administration, intrapleural administration, intraperitoneal administration, intracranial administration, spinal or other parenteral routes of administration, for example by injection or infusion, or direct administration to the thymus. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In some embodiments, subcutaneous administration is adopted. In some embodiments, intravenous administration is adopted. In some embodiments, oral administration is adopted. In some embodiments, the anti-CEACAM1 antibodies or antigen-binding fragments provided herein can be administered to a subject by intratumoral injection, peritumoral injection, juxtatumoral injection, intralesional injection and/or injection into a tumor draining lymph node, or essentially any tumor-targeted injection where the antitumor agent is expected to leak into primary lymph nodes adjacent to targeted solid tumor. In some embodiments, the antibodies or antigen-binding fragments provided herein can be delivered regionally to a tumor using well known methods, including but not limited to, hepatic or aortic pump; limb, lung or liver perfusion; in the portal vein; through a venous shunt; in a cavity or in a vein that is nearby a tumor, and the like. In another embodiment, the antibodies or antigen-binding fragments provided herein can be administered systemically. In a preferred embodiment, the antibodies or antigen-binding fragments are administered regionally at the site of a tumor. The antibodies or antigen-binding fragments can also be administered intratumorally, for example, by direct injection of the cells at the site of a tumor and/or into the tumor vasculature. For example, in the case of malignant pleural disease, mesothelioma or lung cancer, administration is preferably by intrapleural administration (see Adusumilli et al., Science Translational Medicine 6(261):261ra151 (2014)). One skilled in the art can select a suitable mode of administration based on the type of cancer and/or location of a tumor to be treated. The antibodies or antigen-binding fragments can be introduced by injection or catheter. In one embodiment, the antibodies or antigen-binding fragments are pleurally administered to the subject in need, for example, using an intrapleural catheter.

Cancers or tumors to be treated using the anti-CEACAM1 antibodies or antigen-binding fragments, or pharmaceutical compositions provided herein comprise those typically responsive to immunotherapy and those that are not typically responsive to immunotherapy. Cancers that can be treated also include CEACAM1 positive cancers. Cancers that can be treated also include CEACAM5 positive cancers or CEACAM6 positive cancers. In some embodiments, the anti-CEACAM1 antibodies or antigen-binding fragments provided herein are used to treat CEACAM1 positive cancers. In some embodiments, the cancer has a high degree of microsatellite instability. In some embodiments, the cancer is a metastatic cancer, refractory cancer, or recurrent cancer.

In some embodiments, cancers or tumors that can be treated with the anti-CEACAM1 antibodies or antigen-binding fragments or pharmaceutical compositions disclosed herein are hematological cancers. The hematological cancers can be CEACAM1-expressing hematological cancers. The hematological cancers can be CEACAM5-expressing hematological cancers. The hematological cancers can be CEACAM6-expressing hematological cancers. In some embodiments, the hematological cancer can be leukemia, lymphoma, multiple myeloma (MM), or myelodysplastic syndrome (MDS). In some embodiments, the hematological cancer can be acute leukemia, acute myeloid leukemia (AML), B-acute lymphoid leukemia (B-ALL), T-acute lymphoid leukemia (T-ALL), B cell precursor acute lymphoblastic leukemia (BCP-ALL), blastic plasmacytoid dendritic cell neoplasm (BPDCN), acute lymphocytic leukemia, acute myeloblastic leukemia, acute promyelocytic leukemia, acute myelomonocytic leukemia, acute monocytic leukemia, acute erythroleukemia, chronic leukemia, chronic myeloid leukemia (CML), chronic myelocytic leukemia, chronic lymphocytic leukemia, chronic myelomonocytic leukemia (CMML), natural killer cell leukemia (NK leukemia), Hodgkin's disease, non-Hodgkin's disease, Waldenstrom's macroglobulinemia, lymphocytic lymphoma, primary CNS lymphoma, T-cell lymphoma, natural killer cell lymphoma (NK lymphoma), cutaneous T-Cell lymphoma (CTCL), or peripheral T-cell lymphoma (PTCL).

In some embodiments, cancers or tumors that can be treated with the anti-CEACAM1 antibodies or antigen-binding fragments or pharmaceutical compositions disclosed herein are solid tumors. The solid tumors can be CEACAM1-expressing solid tumors. The solid tumors can be CEACAM5-expressing solid tumors. The solid tumors can be CEACAM6-expressing solid tumors. In some embodiments, the cancer or tumor can be carcinomas, sarcoma, melanoma (e.g., cutaneous or intraocular malignant melanoma), glioma, glioblastoma, brain and spinal cord tumors, germ cell tumors, neuroendocrine tumors, carcinoid tumors, gastric cancer, esophageal cancer, liver cancer, lung cancer (e.g., small cell lung cancer, or non-small cell lung cancer), head and neck cancer, skin cancer, nasopharyngeal cancer, kidney cancer, colorectal cancer, breast cancer, pancreatic cancer, testicular cancer, cervical cancer, ovarian cancer, uterine cancer, prostate cancer (for example, hormone refractory prostate adenocarcinoma), bladder cancer, colon cancer, endocrine cancer, basal cell cancer, squamous cell cancer, dermatofibrosarcoma protuberans, mesothelioma, Merkel cell carcinoma, bone cancer, intestinal cancer, renal cancer (for example, clear cell carcinoma), throat cancer, rectal cancer, cancer of the anal region, brain cancer, stomach cancer, carcinoma of the fallopian tubes, carcinoma of the endometrium, carcinoma of the cervix, carcinoma of the vagina, carcinoma of the vulva, cancer of the small intestine, cancer of the thyroid gland, cancer of the parathyroid gland, cancer of the adrenal gland, sarcoma of soft tissue, cancer of the urethra, cancer of the penis, solid tumors of childhood, cancer of ureter, carcinoma of the renal pelvis, neoplasm of the central nervous system (CNS), spinal axis tumor, brain stem glioma, pituitary adenoma, Kaposi's sarcoma, epidermoid cancer, fibrosarcoma, myxosarcoma, liposarcoma, chondrosarcoma, osteogenic sarcoma, synovial sarcoma, chordoma, angiosarcoma, endotheliosarcoma, lymphangiosarcoma, lymphangioendotheliosarcoma, synovioma, Ewing's tumor, leiomyosarcoma, rhabdomyosarcoma, squamous cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilm's tumor, epithelial carcinoma, glioma, astrocytoma, medulloblastoma, craniopharyngioma, ependymoma, pinealoma, hemangioblastoma, acoustic neuroma, oligodenroglioma, schwannoma, meningioma, neuroblastoma, or retinoblastoma.

In some embodiments, the anti-CEACAM1 antibodies or antigen binding fragments provided herein are used for treating pancreatic cancer. In some embodiments, the anti-CEACAM1 antibodies or antigen binding fragments provided herein are used for treating melanoma.

In cancer treatment, eliminating cancer or tumor cells in a subject can occur, but any clinical improvement constitutes a benefit. An anti-tumor effect can be manifested by a decrease in tumor volume, a decrease in the number of tumor cells, a decrease in the number of metastases, an increase in life expectancy, or amelioration of various physiological symptoms associated with the cancerous condition. An anti-tumor effect can also be manifested by the ability of the antibodies or antigen binding fragments, or pharmaceutical compositions provided herein in prevention of the occurrence of tumor in the first place. In some embodiments, an “anti-tumor effect” can be manifested by the reduction in cancer-induced immunosuppression. Clinical improvement comprises decreased risk or rate of progression or reduction in pathological consequences of the cancer or tumor. It is also understood that a method of treating cancer can include any effect that ameliorates a sign or symptom associated with cancer. Such signs or symptoms include, but are not limited to, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers.

In some embodiments, the methods or uses provided herein can reduce tumor burden. Thus, administration of the anti-CEACAM1 antibodies or antigen-binding fragments or pharmaceutical compositions disclosed herein can reduce the number of tumor cells, reduce tumor size, and/or eradicate the tumor in the subject. Methods for monitoring patient response to administration of a pharmaceutical composition disclosed herein are known in the art and can be employed in accordance with methods disclosed herein.

In some embodiments, an anti-tumor effect is observed in a subject having a tumor or cancer who has been administered with an anti-CEACAM1 antibody or antigen-binding fragment described herein as a single therapy, namely, not in combination with another therapeutic. In some embodiments, tumor burden is reduced in a subject having a tumor or cancer who has been administered with an anti-CEACAM1 antibody or antigen-binding fragment described herein as a single therapy, namely, not in combination with another therapeutic.

In the methods disclosed herein, a therapeutically effective amount of the anti-CEACAM1 antibodies or antigen-binding fragments or pharmaceutical compositions disclosed herein is administered to a subject in need of cancer treatment. The subject can be a mammal. In some embodiments, the subject is a human. In some embodiments, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts and are suitably defined for different types of cancers. Features typical of high-risk subgroups are those in which the tumor has invaded neighboring tissues, or who show involvement of lymph nodes.

Anti-CEACAM1 antibodies or antigen-binding fragments or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, an anti-CEACAM1 antibodies or antigen-binding fragment is administered to patients having a cancer that exhibited an inadequate response to, or progressed on, a prior treatment, e.g., a prior treatment with an immuno-oncology or immunotherapy drug, or patients having a cancer that is refractory or resistant, either intrinsically refractory or resistant, or a wherein the resistance or refractory state is acquired. For example, subjects who are not responsive or not sufficiently responsive to a first therapy or who see disease progression following treatment, can be treated by administration of an anti-CEACAM1 antibody alone or in combination with another therapy.

In some embodiments, an anti-CEACAM1 antibody or antigen-binding fragment, is administered to patients who have not previously received (i.e., been treated with) an immuno-oncology agent, e.g., a PD-1 pathway antagonist or a PD-L1 pathway antagonist.

In some embodiments, a method of treating cancer in a subject comprises first determining whether the subject is CEACAM1 positive, e.g., has tumor cells or TILs that express CEACAM1, and if the subject has CEACAM1 positive cancer or TIL cells, then administering to the subject an anti-CEACAM1 antibody or antigen binding fragment described herein.

In some embodiments, a method of treating cancer in a subject comprises first determining whether the subject is CEACAM5 positive or CEACAM6 positive, e.g., has tumor cells or TILs that express CEACAM5 or CEACAM6, and if the subject has CEACAM5 positive or CEACAM6 positive cancer or TIL cells, then administering to the subject an anti-CEACAM1 antibody or antigen binding fragment described herein.

A method of treating a subject having cancer with an anti-CEACAM1 antibody or antigen binding fragment can comprise administering to a subject who has cancer cells or TIL cells that express CEACAM1, a therapeutically effective amount of an anti-CEACAM1 antibody or antigen binding fragment. Also provided herein are methods for predicting whether a subject will respond to treatment with an anti-CEACAM1 antibody or antigen binding fragment, wherein the methods comprise determining the level of CEACAM1 in cancer or TIL cells of the patient, and if cancer or TIL cells of the subject are CEACAM1 positive, then the subject is likely to respond to a treatment with an anti-CEACAM1 antibody or antigen binding fragment. In some embodiments, the methods comprise determining the level of CEACAM5 or CEACAM6 in cancer or TIL cells of the patient, and if cancer or TIL cells of the subject are CEACAM5 positive or CEACAM6 positive, then the subject is likely to respond to a treatment with an anti-CEACAM1 antibody or antigen binding fragment.

An anti-CEACAM1 antibody or antigen binding fragment can be administered with a standard of care treatment. An anti-CEACAM1 antibody or antigen binding fragment can be administered as a maintenance therapy, e.g., a therapy that is intended to prevent the occurrence or recurrence of tumors. An anti-CEACAM1 antibody or antigen binding fragment can be administered with another treatment, e.g., radiation, surgery, or chemotherapy. For example, an anti-CEACAM1 antibody or antigen binding fragment adjunctive therapy can be administered when there is a risk that micrometastases can be present and/or in order to reduce the risk of a relapse.

An anti-CEACAM1 antibody or antigen-binding fragment can be administered as a monotherapy, or as the only immuno-stimulating therapy. An anti-CEACAM1 antibody or antigen-binding fragment can also be combined with an immunogenic agent, such as cancerous cells, purified tumor antigens (including recombinant proteins, peptides, and carbohydrate molecules), cells, and cells transfected with genes encoding immune stimulating cytokines (He et al., (2004) J. Immunol. 173:4919-28). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

Combination therapy using agents with different mechanisms of action can result in additive or synergetic effects. Combination therapy can allow for a lower dose of each agent than is used in monotherapy, thereby reducing toxic side effects and/or increasing the therapeutic index of the agent disclosed herein. Combination therapy can decrease the likelihood that resistant cancer cells will develop. In some embodiments, the additional therapy results in an increase in the therapeutic index of the antibodies or antigen-binding fragments, or pharmaceutical compositions described herein. In some embodiments, the additional therapy results in a decrease in the toxicity and/or side effects of the antibodies or antigen-binding fragments or pharmaceutical compositions described herein. In some embodiments, the anti-CEACAM1 antibodies or antigen-binding fragments or pharmaceutical compositions described herein can be administered in combination with an additional therapy. In some embodiments, the additional therapy can be surgical resection, radiotherapy, or chemotherapy.

The additional therapy can be administered prior to, concurrently with, or subsequent to administration of the anti-CEACAM1 antibodies or antigen-binding fragments or pharmaceutical compositions described herein. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

### 6.8 Exemplified Embodiments

Embodiment 1: An antibody or antigen-binding fragment thereof that specifically binds to human CEACAM1, comprising: (a) a light chain variable region (VL) comprising (1) a light chain CDR1 (VL CDR1) having an amino acid sequence selected from the group consisting of SEQ ID NOs:1-7 and 52-53; (2) a light chain CDR2 (VL CDR2) having an amino acid sequence selected from the group consisting of SEQ ID NOs:8-12 and 54-56; and (3) a light chain CDR3 (VL CDR3) having an amino acid sequence selected from the group consisting of SEQ ID NOs:13-19; or a variant thereof having up to about 3 amino acid substitutions, additions, and/or deletions in the VL CDRs; and/or (b) a heavy chain variable region (VH) comprising (1) a heavy chain CDR1 (VH CDR1) having an amino acid sequence selected from the group consisting of SEQ ID NOs:20-27 and 57; (2) a heavy chain CDR2 (VH CDR2) having an amino acid sequence selected from the group consisting of SEQ ID NOs:28-39; and (3) a heavy chain CDR3 (VH CDR3) having an amino acid sequence selected from the group consisting of SEQ ID NOs:40-51; or a variant thereof having up to about 3 amino acid substitutions, additions, and/or deletions in the VH CDRs.

Embodiment 2: The antibody or antigen-binding fragment of Embodiment 1, wherein (a) the VL CDR1, CDR2 and CDR3 have (1) the amino acid sequences of SEQ ID NOs:1, 8, and 13, respectively; (2) the amino acid sequences of SEQ ID NOs:2, 9, and 14, respectively; (3) the amino acid sequences of SEQ ID NOs:3, 9, and 14, respectively; (4) the amino acid sequences of SEQ ID NOs:4, 8, and 15, respectively; (5) the amino acid sequences of SEQ ID NOs:5, 10, and 16, respectively; (6) the amino acid sequences of SEQ ID NOs:3, 9, and 17, respectively; (7) the amino acid sequences of SEQ ID NOs:6, 11, and 18, respectively; or (8) the amino acid sequences of SEQ ID NOs:7, 12, and 19, respectively; or a variant thereof having up to about 3 amino acid substitutions, additions, and/or deletions in the VL CDRs; and/or (b) the VH CDR1, CDR2 and CDR3 have (1) the amino acid sequences of SEQ ID NOs:20, 28, and 40, respectively; (2) the amino acid sequences of SEQ ID NOs:21, 29, and 41, respectively; (3) the amino acid sequences of SEQ ID NOs:21, 30, and 42, respectively; (4) the amino acid sequences of SEQ ID NOs:22, 31, and 43, respectively; (5) the amino acid sequences of SEQ ID NOs:23, 32, and 44, respectively; (6) the amino acid sequences of SEQ ID NOs:24, 33, and 45, respectively; (7) the amino acid sequences of SEQ ID NOs:25, 38, and 49, respectively; (8) the amino acid sequences of SEQ ID NOs:26, 39, and 50, respectively; or (9) the amino acid sequences of SEQ ID NOs:27, 38, and 51, respectively; or a variant thereof having up to about 3 amino acid substitutions, additions, and/or deletions in the VH CDRs.

Embodiment 3: The antibody or antigen-binding fragment of Embodiment 1, wherein (1) the VL CDR1, CDR2, and CDR3 have the amino acid sequences of SEQ ID NOs:1, 8, and 13, respectively; and/or the VH CDR1, CDR2, and CDR3 have the amino acid sequences of SEQ ID NOs:20, 28, and 40, respectively; (2) the VL CDR1, CDR2, and CDR3 have the amino acid sequences of SEQ ID NOs:2, 9, and 14, respectively; and/or the VH CDR1, CDR2, and CDR3 have the amino acid sequences of SEQ ID NOs:21, 29, and 41, respectively; (3) the VL CDR1, CDR2, and CDR3 have the amino acid sequences of SEQ ID NOs:3, 9, and 14, respectively; and/or the VH CDR1, CDR2, and CDR3 have the amino acid sequences of SEQ ID NOs:21, 30, and 42, respectively; (4) the VL CDR1, CDR2, and CDR3 have the amino acid sequences of SEQ ID NOs:4, 8, and 15, respectively; and/or the VH CDR1, CDR2, and CDR3 have the amino acid sequences of SEQ ID NOs:22, 31, and 43, respectively; (5) the VL CDR1, CDR2, and CDR3 have the amino acid sequences of SEQ ID NOs:5, 10, and 16, respectively; and/or the VH CDR1, CDR2, and CDR3 have the amino acid sequences of SEQ ID NOs:23, 32, and 44, respectively; (6) the VL CDR1, CDR2, and CDR3 have the amino acid sequences of SEQ ID NOs:3, 9, and 17, respectively; and/or the VH CDR1, CDR2, and CDR3 have the amino acid sequences of SEQ ID NOs:24, 33, and 45, respectively; (7) the VL CDR1, CDR2, and CDR3 have the amino acid sequences of SEQ ID NOs:6, 11, and 18, respectively; and/or the VH CDR1, CDR2, and CDR3 have the amino acid sequences of SEQ ID NOs:25, 38, and 49, respectively; (8) the VL CDR1, CDR2, and CDR3 have the amino acid sequences of SEQ ID NOs:7, 12, and 19, respectively; and/or the VH CDR1, CDR2, and CDR3 have the amino acid sequences of SEQ ID NOs:26, 39, and 50, respectively; or (9) the VL CDR1, CDR2, and CDR3 have the amino acid sequences of SEQ ID NOs:7, 12, and 19, respectively; and/or the VH CDR1, CDR2, and CDR3 have the amino acid sequences of SEQ ID NOs:27, 38, and 51, respectively.

Embodiment 4: The antibody or antigen-binding fragment of Embodiment 1, comprising a VL CDR1, a VL CDR2, a VL CDR3, a VH CDR1, a VH CDR2 and a VH CDR3 having the amino acid sequences of SEQ ID NOs:3, 9, 14, 21, 30, and 42, respectively; or a variant thereof having up to about 3 amino acid substitutions, additions, and/or deletions in the CDRs.

Embodiment 5: The antibody or antigen-binding fragment of Embodiment 1, comprising a VL CDR1, a VL CDR2, a VL CDR3, a VH CDR1, a VH CDR2 and a VH CDR3 having the amino acid sequences of SEQ ID NOs:5, 10, 16, 23, 32, and 44, respectively; or a variant thereof having up to about 3 amino acid substitutions, additions, and/or deletions in the CDRs.

Embodiment 6: The antibody or antigen-binding fragment of Embodiment 1, comprising a VL CDR1, a VL CDR2, a VL CDR3, a VH CDR1, a VH CDR2 and a VH CDR3 having the amino acid sequences of SEQ ID NOs:3, 9, 17, 24, 33, and 45, respectively; or a variant thereof having up to about 3 amino acid substitutions, additions, and/or deletions in the CDRs.

Embodiment 7: The antibody or antigen-binding fragment of Embodiment 6, wherein the VL CDR1 has an amino acid sequence selected from the group consisting of SEQ ID NOs:52-53, wherein the VL CDR2 has an amino acid sequence selected from the group consisting of SEQ ID NOs:54-56, wherein the VH CDR1 has the amino acid sequence of SEQ ID NO:57, wherein the VH CDR2 has an amino acid sequence selected from the group consisting of SEQ ID NOs:34-37 and wherein the VH CDR3 has an amino acid sequence selected from the group consisting of SEQ ID NOs:46-48.

Embodiment 8: An antibody or antigen-binding fragment thereof that specifically binds to human CEACAM1, comprising: (a) a VL having at least 85%, at least 90%, at least 95%, at least 98%, or 100% sequence identity to an amino acid sequence selected from the group consisting of SEQ ID NOs:61-68; and/or (b) a VH having at least 85%, at least 90%, at least 95%, at least 98%, or 100% sequence identity to an amino acid sequence selected from the group consisting of SEQ ID NOs:70-78.

Embodiment 9: The antibody or antigen-binding fragment of Embodiment 8, comprising a VL and a VH, wherein the VL and VH each have at least 85%, at least 90%, at least 95%, at least 98%, or 100% sequence identity to the amino acid sequences of (1) SEQ ID NOs:61 and 70, respectively; (2) SEQ ID NOs:62 and 71, respectively; (3) SEQ ID NOs:63 and 72, respectively; (4) SEQ ID NOs:64 and 73, respectively; (5) SEQ ID NOs:65 and 74, respectively; (6) SEQ ID NOs:66 and 75, respectively; (7) SEQ ID NOs:67 and 76, respectively; (8) SEQ ID NOs:68 and 77, respectively; or (9) SEQ ID NOs:68 and 78, respectively.

Embodiment 10: The antibody or antigen-binding fragment of Embodiment 9, comprising a VL and a VH, wherein the VL and VH each have at least 85%, at least 90%, at least 95%, at least 98%, or 100% sequence identity to the amino acid sequences of SEQ ID NOs:63 and 72, respectively.

Embodiment 11: The antibody or antigen-binding fragment of Embodiment 9, comprising a VL and a VH, wherein the VL and VH each have at least 85%, at least 90%, at least 95%, at least 98%, or 100% sequence identity to the amino acid sequences of SEQ ID NOs:65 and 74, respectively.

Embodiment 12: The antibody or antigen-binding fragment of Embodiment 9, comprising a VL and a VH, wherein the VL and VH each have at least 85%, at least 90%, at least 95%, at least 98%, or 100% sequence identity to the amino acid sequences of SEQ ID NOs:66 and 75, respectively.

Embodiment 13: The antibody or antigen-binding fragment of Embodiment 12, wherein the VL has the amino acid sequence of SEQ ID NO:66 with an amino acid substitution at 528, S51, K52, or any combination thereof.

Embodiment 14: The antibody or antigen-binding fragment of Embodiment 13, wherein the S28 substitution is S28F or S28H, wherein the S51 substitution is S51M, and wherein the K52 substitution is K52R.

Embodiment 15: The antibody or antigen-binding fragment of Embodiment 12, wherein the VH has the amino acid sequence of SEQ ID NO:75 with an amino acid substitution at H35, N56, Y100, or any combination thereof.

Embodiment 16: The antibody or antigen-binding fragment of Embodiment 15, wherein the H35 substitution is H35M, wherein the N56 substitution is N56I, N56Q, N56V, or N56W, and wherein the Y100 substitution is Y100F, Y100W, or Y100V.

Embodiment 17: An antibody or antigen-binding fragment thereof that specifically binds to human CEACAM1, comprising: (a) a VL comprising VL CDR1, CDR2, and CDR3 from a VL having an amino acid sequence selected from the group consisting of SEQ ID NOs:61-68; and/or (b) a VH comprising VH CDR1, CDR2, and CDR3 from a VH having an amino acid sequence selected from the group consisting of SEQ ID NOs:70-78; or a variant thereof having up to about 3 amino acid substitutions, additions, and/or deletions in the CDRs.

Embodiment 18: The antibody or antigen-binding fragment thereof. Embodiment 17 comprising (1) a VL comprising VL CDR1, CDR2, and CDR3 from a VL having the amino acid sequence of SEQ ID NO:61, and/or a VH comprising VH CDR1, CDR2, and CDR3 from a VH having the amino acid sequence of SEQ ID NO:70; (2) a VL comprising VL CDR1, CDR2, and CDR3 from a VL having the amino acid sequence of SEQ ID NO:62, and/or a VH comprising VH CDR1, CDR2, and CDR3 from a VH having the amino acid sequence of SEQ ID NO:71; (3) a VL comprising VL CDR1, CDR2, and CDR3 from a VL having the amino acid sequence of SEQ ID NO:63, and/or a VH comprising VH CDR1, CDR2, and CDR3 from a VH having the amino acid sequence of SEQ ID NO:72; (4) a VL comprising VL CDR1, CDR2, and CDR3 from a VL having the amino acid sequence of SEQ ID NO:64, and/or a VH comprising VH CDR1, CDR2, and CDR3 from a VH having the amino acid sequence of SEQ ID NO:73; (5) a VL comprising VL CDR1, CDR2, and CDR3 from a VL having the amino acid sequence of SEQ ID NO:65, and/or a VH comprising VH CDR1, CDR2, and CDR3 from a VH having the amino acid sequence of SEQ ID NO:74; (6) a VL comprising VL CDR1, CDR2, and CDR3 from a VL having the amino acid sequence of SEQ ID NO:66, and/or a VH comprising VH CDR1, CDR2, and CDR3 from a VH having the amino acid sequence of SEQ ID NO:75; (7) a VL comprising VL CDR1, CDR2, and CDR3 from a VL having the amino acid sequence of SEQ ID NO:67, and/or a VH comprising VH CDR1, CDR2, and CDR3 from a VH having the amino acid sequence of SEQ ID NO:76; (8) a VL comprising VL CDR1, CDR2, and CDR3 from a VL having the amino acid sequence of SEQ ID NO:68, and/or a VH comprising VH CDR1, CDR2, and CDR3 from a VH having the amino acid sequence of SEQ ID NO:77; or (9) a VL comprising VL CDR1, CDR2, and CDR3 from a VL having the amino acid sequence of SEQ ID NO:68, and/or a VH comprising VH CDR1, CDR2, and CDR3 from a VH having the amino acid sequence of SEQ ID NO:78; or a variant thereof having up to about 3 amino acid substitutions, additions, and/or deletions in the CDRs.

Embodiment 19: An antibody or antigen-binding fragment thereof that specifically binds to human CEACAM1 comprising a VL and a VH, wherein the VL comprises VL CDR1, CDR2, and CDR3 from a VL having the amino acid sequence of SEQ ID NO:63, and the VH comprises VH CDR1, CDR2, and CDR3 from a VH having the amino acid sequence of SEQ ID NO:72; or a variant thereof having up to about 3 amino acid substitutions, additions, and/or deletions in the CDRs.

Embodiment 20: An antibody or antigen-binding fragment thereof that specifically binds to human CEACAM1 comprising a VL and a VH, wherein the VL comprises VL CDR1, CDR2, and CDR3 from a VL having the amino acid sequence of SEQ ID NO:65, and the VH comprises VH CDR1, CDR2, and CDR3 from a VH having the amino acid sequence of SEQ ID NO:74; or a variant thereof having up to about 3 amino acid substitutions, additions, and/or deletions in the CDRs.

Embodiment 21: An antibody or antigen-binding fragment thereof that specifically binds to human CEACAM1 comprising a VL and a VH, wherein the VL comprises VL CDR1, CDR2, and CDR3 from a VL having the amino acid sequence of SEQ ID NO:66, and the VH comprises VH CDR1, CDR2, and CDR3 from a VH having the amino acid sequence of SEQ ID NO:75; or a variant thereof having up to about 3 amino acid substitutions, additions, and/or deletions in the CDRs.

Embodiment 22: An antibody or antigen-binding fragment thereof that competes with the antibody or antigen-binding fragment of any one of Embodiments 1 to 21 for binding to human CEACAM1.

Embodiment 23: The antibody or antigen-binding fragment of any one of Embodiments 1 to 22 that is a chimeric antibody or antigen-binding fragment, a humanized antibody or antigen-binding fragment, or a human antibody or antigen-binding fragment.

Embodiment 24: The antibody or antigen-binding fragment of Embodiment 23 that is a humanized antibody or antigen-binding fragment.

Embodiment 25: The antibody or antigen-binding fragment of Embodiment 24 that has (a) a VL having at least 85%, at least 90%, at least 95%, at least 98%, or 100% sequence identity to an amino acid sequence selected from the group consisting of SEQ ID NOs:81-90; and/or (b) a VH having at least 85%, at least 90%, at least 95%, at least 98%, or 100% sequence identity to an amino acid sequence selected from the group consisting of SEQ ID NOs:111-116.

Embodiment 26: The antibody or antigen-binding fragment of Embodiment 24 that has (a) a VL having at least 85%, at least 90%, at least 95%, at least 98%, or 100% sequence identity to an amino acid sequence selected from the group consisting of SEQ ID NOs:91-93 and 107-109; and/or (b) a VH having at least 85%, at least 90%, at least 95%, at least 98%, or 100% sequence identity to an amino acid sequence selected from the group consisting of SEQ ID NOs:117-122.

Embodiment 27: The antibody or antigen-binding fragment of Embodiment 24 that has (a) a VL having at least 85%, at least 90%, at least 95%, at least 98%, or 100% sequence identity to an amino acid sequence selected from the group consisting of SEQ ID NOs:94-106; and/or (b) a VH having at least 85%, at least 90%, at least 95%, at least 98%, or 100% sequence identity to an amino acid sequence selected from the group consisting of SEQ ID NOs:123-128, 130 and 143-149.

Embodiment 28: The antibody or antigen-binding fragment of Embodiment 27, wherein the VL has an amino acid sequence selected from the group consisting of SEQ ID NOs:94-99, with an amino acid substitution at S28, S51, K52, or any combination thereof.

Embodiment 29: The antibody or antigen-binding fragment of Embodiment 28, wherein the S28 substitution is S28F or S28H, wherein the S51 substitution is S51M, and wherein the K52 substitution is K52R.

Embodiment 30: The antibody or antigen-binding fragment of Embodiment 29, wherein the VL has an amino acid sequence selected from the group consisting of SEQ ID NOs:100-106.

Embodiment 31: The antibody or antigen-binding fragment of Embodiment 27 wherein the VH has an amino acid sequence selected from the group consisting of SEQ ID NOs:123-128, with an amino acid substitution at H35, N56, Y100, or any combination thereof.

Embodiment 32: The antibody or antigen-binding fragment of Embodiment 31, wherein the H35 substitution is H35M, wherein the N56 substitution is N56I, N56Q, N56V, or N56W, and wherein the Y100 substitution is Y100F, Y100W, or Y100V.

Embodiment 33: The antibody or antigen-binding fragment of Embodiment 32, wherein the VH has an amino acid sequence selected from the group consisting of SEQ ID NOs:130 and 143-149.

Embodiment 34: The antibody or antigen-binding fragment of any one of Embodiments 1 to 33, that binds to human CEACAM1 with a Ko that is 10⁻⁸ M or less.

Embodiment 35: The antibody or antigen-binding fragment of Embodiments 1 to 33, that binds to human CEACAM1 with a K_{D} that ranges from 10⁻¹¹ M to 10⁻⁸ M.

Embodiment 36: The antibody or antigen-binding fragment of any one of Embodiments 1 to 35 that promotes internalization of CEACAM1 on cell surface.

Embodiment 37: The antibody or antigen-binding fragment of any one of Embodiments 1 to 36 that activates T cells, NK cells, or both.

Embodiment 38: The antibody or antigen-binding fragment of Embodiment 37 that enhances IFN-γ release by T cells.

Embodiment 39: The antibody or antigen-binding fragment of Embodiment 37 that enhances the activity of T cells, NK cells, or both, against tumor cells.

Embodiment 40: The antibody of antigen-binding fragment of any one of Embodiments 1 to 39 that is selected from the group consisting of an IgG1 antibody, an IgG2 antibody, an IgG3 antibody, and an IgG4 antibody.

Embodiment 41: The antibody or antigen-binding fragment of Embodiment 40 that is an IgG1 antibody.

Embodiment 42: The antibody or antigen-binding fragment of any one of Embodiments 1 to 39 that is selected from the group consisting of a Fab, a Fab′, a F(ab′)₂, a Fv, a scFv, a (scFv)₂, a single domain antibody (sdAb), and a heavy chain antibody (HCAb).

Embodiment 43: The antibody or antigen-binding fragment of any one of Embodiments 1 to 42 that is a monoclonal antibody or antigen-binding fragment.

Embodiment 44: A polynucleotide encoding the antibody or antigen-binding fragment of any one of Embodiments 1 to 43.

Embodiment 45: A vector comprising the polynucleotide of Embodiment 44.

Embodiment 46: A cell comprising the polynucleotide of Embodiment 44, or the vector of Embodiment 45.

Embodiment 47: A method of making an antibody or antigen-binding fragment thereof that specifically binds to human CEACAM1, comprising culturing the cell of Embodiment 46 under conditions that allow expression of the antibody or antibody fragment.

Embodiment 48: The method of Embodiment 47 that comprises isolating the antibody or antigen-binding fragment from the culture.

Embodiment 49: A pharmaceutical composition comprising a therapeutically effective amount of the antibody or antigen-binding fragment of any one of Embodiments 1 to 43, and a pharmaceutically acceptable carrier.

Embodiment 50: A method of inducing or stimulating immune cell activation and/or proliferation, comprising contacting an immune cell with an effective amount of the antibody or antigen-binding fragment of any one of Embodiments 1 to 43.

Embodiment 51: A method of reducing the suppression of an immune cell comprising contacting the immune cell with an effective amount of the antibody or antigen-binding fragment of any one of Embodiments 1 to 43.

Embodiment 52: The method of Embodiment 50 or 51, wherein the immune cell is a lymphocyte of T cell or NK cell.

Embodiment 53: A method of increasing IFN-γ production in a T cell comprising contacting the T cell with an effective amount of the antibody or antigen-binding fragment of any one of Embodiments 1 to 43.

Embodiment 54: A method of inhibiting cancer cell growth, angiogenesis and/or metastasis in a subject in need thereof, comprising administering to the subject a therapeutically effective amount of the antibody or antigen-binding fragment of any one of Embodiments 1 to 43.

Embodiment 55: A method of treating cancer in a subject in need thereof, comprising administering to the subject a therapeutically effective amount of the antibody or antigen-binding fragment of any one of Embodiments 1 to 43.

Embodiment 56: The method of Embodiment 54 or 55, further comprising administering an additional therapy to the subject.

Embodiment 57: The method of any one of Embodiments 54 to 56, wherein the subject is a human.

Embodiment 58: Use of the antibody or antigen-binding fragment of any one of Embodiments 1 to 43 in cancer treatment.

Embodiment 59: Use of the antibody or antigen-binding fragment of any one of Embodiments 1 to 43 for the preparation of a medicament for the treatment of cancer.

Embodiment 60: The method or use of any one of Embodiments 54 to 59, wherein the cancer is a CEACAM1-expressing cancer, a CEACAM5-expressing cancer or a CEACAM6-expressing cancer.

Embodiment 61: The method or use of any one of Embodiments 54 to 60, wherein the cancer is a hematological cancer.

Embodiment 62: The method or use of any one of Embodiments 54 to 60, wherein the cancer is a solid tumor.

Embodiment 63: The method or use of Embodiment 62, wherein the solid tumor is pancreatic cancer.

Embodiment 64: The method or use of Embodiment 62, wherein the solid tumor is melanoma.

The practice of the invention employs, unless otherwise indicated, conventional techniques in molecular biology, cell biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) MOLECULAR CLONING: A LABORATORY MANUAL, Cold Spring Harbor Laboratory Press; Sambrook et al. (1989), MOLECULAR CLONING: A LABORATORY MANUAL, Second Edition, Cold Spring Harbor Laboratory Press; Sambrook et al. (2001) MOLECULAR CLONING: A LABORATORY MANUAL, Cold Spring Harbor Laboratory Press, Cold Spring Harbor, NY; Ausubel et al., CURRENT PROTOCOLS IN MOLECULAR BIOLOGY, John Wiley & Sons (1987 and annual updates); CURRENT PROTOCOLS IN IMMUNOLOGY, John Wiley & Sons (1987 and annual updates) Gait (ed.) (1984) OLIGONUCLEOTIDE SYNTHESIS: A PRACTICAL APPROACH, IRL Press; Eckstein (ed.) (1991) OLIGONUCLEOTIDES AND ANALOGUES: A PRACTICAL APPROACH, IRL Press; Birren et al. (eds.) (1999) GENOME ANALYSIS: A LABORATORY MANUAL, Cold Spring Harbor Laboratory Press; Borrebaeck (ed.) (1995) ANTIBODY ENGINEERING, Second Edition, Oxford University Press; Lo (ed.) (2006) ANTIBODY ENGINEERING: METHODS AND PROTOCOLS (METHODS IN MOLECULAR BIOLOGY); Vol. 248, Humana Press, Inc; each of which is incorporated herein by reference in its entirety.

### 6.9 Experimental

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

Briefly, results from the studies described below demonstrate that candidate anti-hCEACAM1 antibodies bound to human CEACAM1 with high affinity and reduced CEACAM1-mediated immune suppression through inducing CEACAM1 internalization and blocking interaction between CEACAM1 and CEACAM5/6. The candidate anti-hCEACAM1 antibodies were also shown to restore T cell activation, enhance NK and T cell killing efficacy, inhibit macrophage-mediated tumor aggression and inhibit tumor growth in mouse cancer models.

### 6.9.1 Example 1: Production of Anti-hCEACAM1 Antibodies

Methods: Balb/C mice were immunized with recombinant extracellular domain of human CCM1 by intraperitoneal administration of 100 μg per injection, followed by PEG-fusion. Mice received 3 consecutive immunizations at days 0, 14, and 28. Blood was taken at days 21 and 35 and sera were used for titer determination by ELISA. Animals with highest titers were selected for boosting at day 36 by intraperitoneal injection of 100 μg of recombinant human CCM1-Fc chimera, and hybridoma cells were obtained by fusion of splenocytes with myeloma cell line 3 days after boost, subjected to FACS and ELISA screening. The positive hybridoma cells were selected for breed conservation and a series of subcloning. Antibodies produced by candidate clones, namely, 6H9, 7D4, 9D12, P23B6, 2C10, 4D10, 4H11, 2G12, and 5D5, were sequenced, and the sequences of respective antibodies are provided in Tables 1 to 3 above.

### 6.9.2 Example 2: Binding Affinities of Candidate Antibodies to Human CEACAM1

Methods: To confirm the binding of the selected clones to human CEACAM1, serially diluted anti-CEACAM1 antibodies and isotype control were incubated with RAJI-CEACAM1 stable cells (10\hat{ }5 cells/well in 96-round well plate) at 4° C. for 1h. Then, 2 μg/ml PE anti-mouse Ig light chain κ Antibody (Biolengend, 409506) was used as the detection antibody. The fluorescence was detected by the flow cytometer Canto II (BD, USA).

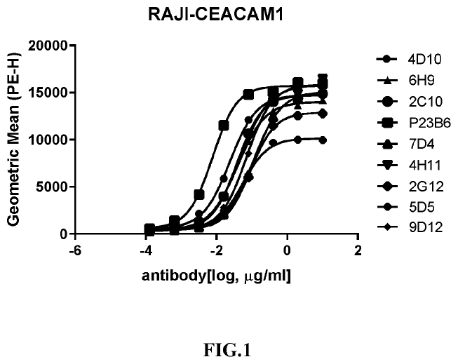

Results: As shown in FIG. 1, the specific binding of the candidate clones to CEACAM1-expressing cells was confirmed.

### 6.9.3 Example 3: Binding Kinetics of Anti-CEACAM1 Antibodies

Methods: Antibodies were coupled to the surface of the sensor through anti-Fc, and human CEACAM1 recombinant protein, as the mobile phase, was 1:3 serially diluted with a maximum concentration of 40 nM. The association and dissociation between CEACAM1 protein and the antibody on the surface of the sensor were measured. Specifically, the HFC sensor was prewetted in SD buffer (PBS containing 0.02% Tween-20 and 0.2% BSA) for 5 minutes, and then balanced in a sample buffer containing antibodies for 5 minutes, allowing antibodies to be coupled to the sensor surface. The antibody-coupled HFC sensor was balanced in the SD buffer solution for 1-5 minutes, and then incubated in the buffer solution containing different concentrations of CEACAM1-his to measure the association of CEACAM1-his protein to captured antibody. Finally, the sensor combining the antigen and antibody was placed in SD buffer and waited for 1-8 mins to measure dissociation of CEACAM1-his protein from the antibodies captured on sensor. Data was fitted using a 1:1 model, and the association rate (kₒₙ) and the dissociation rate (k_{off}) were calculated. The equilibrium dissociation constant (K_{D}) is calculated by the ratio k_{off}/Kₒₙ.

The binding affinities of the humanized antibodies were provided in Table 7. Note that 4D10.G4 and 5D5.G4 were chimeric IgG4 antibodies, and the rest of the tested antibodies were hybridoma clones.

TABLE 7
Binding affinities of candidate chimeric antibodies

| Loading | koff (1/s) | kon (1/Ms) | KD (M) |
|---|---|---|---|
| 2G12 | 7.07E-04 | 3.23E+05 | 2.19E-09 |
| 4D10.G4 | 1.56E-03 | 8.22E+05 | 1.89E-09 |
| 5D5.G4 | 9.41E-04 | 4.46E+05 | 2.11E-09 |
| 9D12 | 1.08E-03 | 3.71E+05 | 2.90E-09 |
| P23B6 | 2.97E-03 | 4.70E+05 | 6.31E-09 |
| 2C10 | 5.28E-04 | 5.53E+05 | 9.55E-10 |
| 4H11 | 1.61E-03 | 4.60E+05 | 3.51E-09 |
| 7D4 | 1.60E-03 | 2.29E+05 | 6.97E-09 |
| 3D6 | 4.87E-03 | 7.38E+05 | 6.59E-09 |
| 2G12 | 7.07E-04 | 3.23E+05 | 2.19E-09 |

### 6.9.4 Example 4: Humanization of Anti-hCEACAM1 Antibodies

### Methods:

(1) Selection of the FR regions for humanization: The germlines closest to Clones 9D12, 2C10, and 4D10 were selected from IMGT database to serve as the templates for humanization. CDRs (Kabat numbering) from Clones 9D12, 2C10, 4D10 were grafted to the corresponding human templates with zero to several amino acids back mutation. The sequences of humanized VH and VL regions of Clones 9D12, 2C10, 4D10 are provided in Tables 4-6 above.

(2) Cloning and expression of humanized antibodies: Primers were designed for genetic engineering or gene cloning into expression vectors; VH and VL regions were obtained by PCR and cloned into expression vectors that contained the nucleic acid sequences encoding the signal peptide and the constant regions (CH1-FC/CL). The heavy chain and light chain of the antibodies were transfected into 293F cells for expression. Affinities were measured using Gator. The binding affinities of the humanized antibodies were provided in Table 8.

As used herein (including the drawings and tables), the candidate antibodies are named in the following format: [clone name].[Ig subtype].[specific humanized variable domains]. For example, Antibody h2C10.G4.H0aL0a as listed in Table 8 below refers to the IgG4 that comprises the following humanized variable domains of clone 2C10, L0a (SEQ ID NO:91) and H0a (SEQ ID NO: 117). For another example, Antibody h4D10.G4.H3L2 refers to IgG4 that comprises the following humanized variable domains of clone 4D10, L2 (VL; SEQ ID NO:97) and H3 (VH; SEQ ID NO:127).

TABLE 8
Binding affinities of humanized antibodies

| Loading | koff (1/s) | kon (1/Ms) | KD (M) |
|---|---|---|---|
| h2C10.G4.HOaLOa | 1.17E-03 | 5.42E+05 | 2.15E-09 |
| h2C10.G4.HOaLOb | 1.15E-03 | 5.17E+05 | 2.23E-09 |
| h2C10.G4.HOaL1 | 1.01E-03 | 5.58E+05 | 1.81E-09 |
| h2C10.G4.H3L1 | 1.29E-03 | 5.90E+05 | 2.18E-09 |
| h2C10.G4.HOaL2 | 5.04E-04 | 5.93E+05 | 8.50E-10 |
| h2C10.G4.HOaL3 | 5.07E-04 | 5.37E+05 | 9.44E-10 |
| h2C10.G4.HOaL4 | 1.05E-03 | 5.72E+05 | 1.83E-09 |
| h2C10.G4.H3L2 | 5.69E-04 | 6.14E+05 | 9.27E-10 |
| h2C10.G4.H3L3 | 6.59E-04 | 5.83E+05 | 1.13E-09 |
| h2C10.G4.H3L4 | 1.22E-03 | 6.08E+05 | 2.00E-09 |
| h4D10.G4.H0bL1 | 1.64E-03 | 7.62E+05 | 2.16E-09 |
| h4D10.G4.H3L1 | 1.24E-03 | 8.45E+05 | 1.46E-09 |
| h4D10.G4.H4L1 | 1.29E-03 | 8.38E+05 | 1.53E-09 |
| h4D10.G4.H0bL2 | 1.88E-03 | 8.24E+05 | 2.28E-09 |
| h4D10.G4.H3L2 | 1.53E-03 | 8.63E+05 | 1.77E-09 |
| h4D10.G4.H4L2 | 1.49E-03 | 8.80E+05 | 1.70E-09 |
| h9D12.G4.H4L7 | 1.10E-03 | 4.48E+05 | 2.46E-09 |
| h9D12.G4.HOaL7 | 1.52E-03 | 4.49E+05 | 3.39E-09 |
| h9D12.G4.H0bL7 | 1.79E-03 | 4.86E+05 | 3.68E-09 |

### 6.9.5 Example 5: Clone 4D10 Optimization

Methods: 4D10 has potential deamidation site (YFNPYNNGTNYNENFKG; SEQ ID NO:33) in HCDR2, and poly Y (YYYASGSLYFDY; SEQ ID NO:45) in HCDR3. Site-saturation mutagenesis was introduced to these amino acids. Certain single mutations were selected and combined. These variants were produced, and affinities were measured using biolayer interferometry Gator. The binding affinities of humanized 4D10.G4.H4L2 after site mutagenesis were provided in Table 9. Note that N56 corresponds to the 56^{th} amino acid in 4D10 VH (SEQ ID NO:75), which corresponds to N7 in VH CDR2 (SEQ ID NO:33); Y100 corresponds to the 100^{th} amino acid in 4D10 VH (SEQ ID NO:75), which corresponds to Y2 in VH CDR3 (SEQ ID NO:45).

TABLE 9
Binding affinities of optimized humanized 4D10.G4.H4L2

| Loading | koff (1/s) | kon (1/Ms) | KD (M) | FullR2 |
|---|---|---|---|---|
| h4D10.G4.H4L2 | 2.65E-03 | 1.00E+06 | 2.65E-09 | 0.995 |
| h4D10.G4.H4 | 2.82E-03 | 9.00E+05 | 3.14E-09 | 0.997 |
| (N561 Y100F)L2 | | | | |
| h4D10.G4.H4 | 7.00E-03 | 1.12E+06 | 6.22E-09 | 0.994 |
| (N56I Y100V)L2 | | | | |
| h4D10.G4.H4 | 4.86E-03 | 8.99E+05 | 5.41E-09 | 0.998 |
| (N561 Y100W)L2 | | | | |
| h4D10.G4.H4 | 3.10E-03 | 9.37E+05 | 3.31E-09 | 0.996 |
| (N56QY100F)L2 | | | | |
| h4D10.G4.H4 | 7.46E-03 | 1.27E+06 | 5.86E-09 | 0.992 |
| (N56Q Y100V)L2 | | | | |
| h4D10.G4.H4 | 2.90E-03 | 7.96E+05 | 3.65E-09 | 0.997 |
| (N56Q Y100W)L2 | | | | |
| h4D10.G4.H4 | 2.86E-03 | 1.04E+06 | 2.75E-09 | 0.993 |
| (N56VY100F)L2 | | | | |
| h4D10.G4.H4 | 7.12E-03 | 1.53E+06 | 4.67E-09 | 0.992 |
| (N56VY100V)L2 | | | | |
| h4D10.G4.H4 | 4.53E-03 | 9.30E+05 | 4.87E-09 | 0.997 |
| (N56VY100W)L2 | | | | |
| h4D10.G4.H4 | 1.60E-03 | 1.41E+06 | 1.13E-09 | 0.865 |
| (N56W Y100F)L2 | | | | |
| h4D10.G4.H4 | 5.92E-03 | 1.19E+06 | 4.97E-09 | 0.992 |
| (N56WY100V)L2 | | | | |
| h4D10.G4.H4 | 2.74E-03 | 8.07E+05 | 3.39E-09 | 0.996 |
| (N56WY100W)L2 | | | | |
| 4D10.G4 | 2.21E-03 | 1.21E+06 | 1.82E-09 | 0.993 |

Further site-saturation mutagenesis was introduced to the CDRs and mutations at the following sites were identified to further improve affinity: LCDR1 (SASSSVSYMH; SEQ ID NO:3), LCDR2 (EISKLAS; SEQ ID NO:9), and HCDR1 (SYVIH; SEQ ID NO:24). Certain single mutations were selected and combined. These variants were produced, and affinities were measured using biolayer interferometry Gator. The binding affinities of these variants were provided in Table 9A. S28 corresponds to the 28^{th} amino acid in 4D10 VL (SEQ ID NO:66), which corresponds to S5 in VL CDR1 (SEQ ID NO:3); S51 corresponds to the 51^{th} amino acid in 4D10 VL (SEQ ID NO:66), which corresponds to S3 in VL CDR2 (SEQ ID NO:9); K52 corresponds to the 52^{th} amino acid in 4D10 VL (SEQ ID NO:66), which corresponds to K4 in VL CDR2 (SEQ ID NO:9); H35 corresponds to the 35^{th} amino acid in 4D10 VH (SEQ ID NO:75), which corresponds to H5 in VH CDR1 (SEQ ID NO:24).

TABLE 9A
Binding affinities of further optimized humanized 4D10.G4.H4L2

| Loading | koff (1/s) | kon (1/Ms) | KD (M) | FullR2 |
|---|---|---|---|---|
| h4D10.G4.H4(N56Q Y100F)L2(S28F) | 2.66E-04 | 8.88E+05 | 3.00E-10 | 0.993 |
| h4D10.G4.H4(N56Q Y100F)L2(S28H) | 9.64E-04 | 1.01E+06 | 9.56E-10 | 0.997 |
| h4D10.G4.H4(N56QY100F)L2(S51M) | 7.82E-04 | 9.98E+05 | 7.84E-10 | 0.994 |
| h4D10.G4.H4(N56QY100F)L2(K52R) | 8.50E-04 | 1.06E+06 | 8.01E-10 | 0.996 |
| h4D10.G4.H4(N56QY100F)L2(S28F S51M) | 2.31E-04 | 6.38E+05 | 3.62E-10 | 0.997 |
| h4D10.G4.H4(N56Q Y100F)L2(S28F K52R) | 1.75E-04 | 6.55E+05 | 2.68E-10 | 0.998 |
| h4D10.G4.H4(N56Q Y100F)L2(S28F S51M K52R) | 9.19E-05 | 6.08E+05 | 1.51E-10 | 0.998 |
| h4D10.G4.H4(H35MN56QY100F)L2 | 3.76E-04 | 9.56E+05 | 3.94E-10 | 0.994 |

### 6.9.6 Example 6: Binding Kinetics of Anti-CEACAM1 Antibodies to Human CEACAM1

Methods: Antibodies were coupled to the surface of the HFC sensor (Probe Life), and human CEACAM1 recombinant protein, as the mobile phase, was 1:2 serially diluted with a maximum concentration of 50 nM. The association and dissociation between CEACAM1 protein and the anti-CEACAM1 antibodies on the surface of the sensor were measured. Specifically, the HFC sensor was prewetted in SD buffer (PBS containing 0.02% Tween-20 and 0.2% BSA) for 5 minutes, and then balanced in a sample buffer containing antibodies for 5 minutes, allowing antibodies to be coupled to the sensor surface. The antibody-coupled HFC sensor was balanced in the SD buffer solution for 1 minute, and then incubated in the buffer solution containing different concentrations of CEACAM1-his for 200s to measure the association of CEACAM1-his protein to captured antibody. Finally, the sensor combining the antigen and antibody was placed in SD buffer for 300s to measure dissociation of CEACAM1-his protein from the antibodies captured on sensor. Data were fitted using a 1:1 model, and the association rate (kₒₙ) and the dissociation rate (k_{off}) were calculated. The equilibrium dissociation constant (K_{D}) was calculated by the ratio k_{off}/Kₒₙ. The binding kinetics of CEACAM1 protein and candidate antibodies were provided in Table 10.

TABLE 10
Binding kinetics of CEACAMI protein and candidate antibodies

| Loading | koff (1/s) | kon (1/Ms) | KD (M) |
|---|---|---|---|
| h2C10.G4.HOaL2 | 5.04E-04 | 5.93E+05 | 8.50E-10 |
| h2C10.G4.HOaL4 | 1.05E-03 | 5.72E+05 | 1.83E-09 |
| h4D10.G4.H4L2 | 1.49E-03 | 8.80E+05 | 1.70E-09 |
| h9D12.G4.H4L7 | 1.10E-03 | 4.48E+05 | 2.46E-09 |
| h9D12.G4.HOaL7 | 1.52E-03 | 4.49E+05 | 3.39E-09 |
| h9D12.G4.H0bL7 | 1.79E-03 | 4.86E+05 | 3.68E-09 |
| h4D10.G4.H4 | 1.67E-03 | 8.25E+05 | 2.02E-09 |
| (N56Q Y100F)L2 | | | |

### 6.9.7 Example 7: Binding of Candidate Antibodies to Cells Expressing CEACAM1

Methods: In this assay, serially diluted anti-CEACAM1 antibodies and isotype control were incubated with RAJI-CEACAM1 stable cells, or cells with natural expressing of CEACAM1, namely, HepG2 or BXPC-3 (10≡cells/well in 96-round well plate) at 4° C. for 1h. Then, AF647 Anti-Human IgG (Jackson, 109-607-008, diluted at a ratio of 1:1000) was used as detection antibody. The fluorescence was detected by the flow cytometer Advanteon (Agilent, USA). The binding EC50 of candidate antibodies to CEACAM1 expressing cells is provided in Table 11.

TABLE 11
Binding EC50 of candidate antibodies to
CEACAM1-expressing cells

| EC50 (μg/ml) | Raji-CEACAM1 | HEPG2 | BXPC-3 |
|---|---|---|---|
| h2C10.G4.HOaL2 | 0.07463 | 0.06843 | 0.04636 |
| h2C10.G4.HOaL4 | 0.08618 | 0.0491 | 0.03102 |
| h4D10.G4.H4L2 | 0.06995 | 0.03899 | 0.02626 |
| h9D12.G4.H0bL7 | | 0.09739 | 0.07232 |
| h4D10.G4.H4 | | | 0.02114 |
| (N56Q Y100F)L2 | | | |

### 6.9.8 Example 8: Cross-Binding to Other CEACAM Family Members

Methods: CEACAM3, CEACAM5 and CEACAM6 shared high similarity of sequences and structures to CEACAM1 in IgV domain. The sequence identity reaches 85%. The biomolecular interaction system Gator was used to detect the binding affinities between antibodies and CEACAM 3/5/6. CEACAM1 antibodies were coupled to the surface of the HFC sensor (Probe Life), and human CEACAM1, CEACAM3, CEACAM5 and CEACAM6 recombinant protein, as the mobile phase, was 1:2 serially diluted with a maximum concentration of 40 nM, 200 nM, 100 nM and 200 nM respectively. The association and dissociation between CEACAMs protein and the antibody on the surface of the sensor were measured as described above in Example 6.

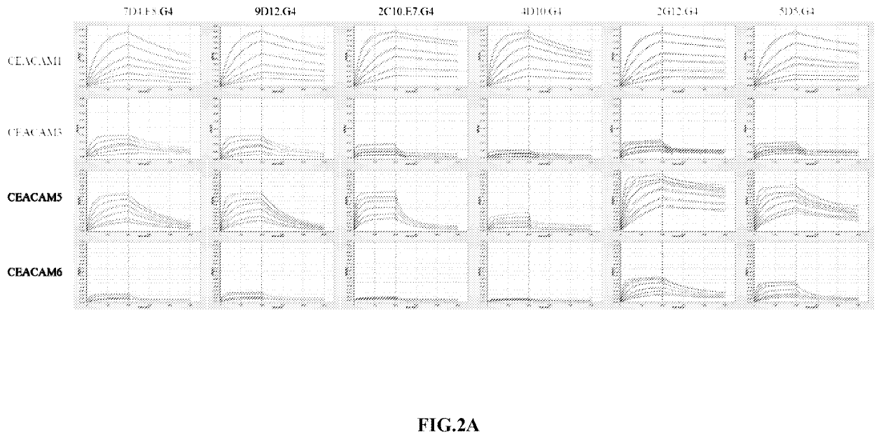

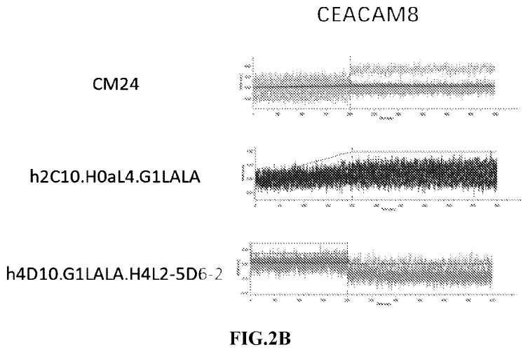

Results and conclusions: As shown in FIG. 2A, antibodies 7D4 and 9D12 cross-reacted with CEACAM3 and CEACAM5, but not with CEACAM6; 2C10 cross-reacted with CEACAM5, but not with CEACAM3 or CEACAM6; 2G12 and 5D5 cross-reacted with CEACAM5 and CEACAM6; and 4D10 did not cross-react with CEACAM3, CEACAM5 or CEACAM6.

Further, cross-binding capacity of candidate antibodies to CEACAM8 was measured. CEACAM8 was selected in this assay due to the high similarity of sequence and structure to CEACAM1 in IgV domain. The sequence identity reaches more than 70%. The biomolecular interaction system Gator was used to detect the binding affinities between candidate antibodies and CEACAM8. Candidate antibodies were coupled to the surface of the HFC sensor (Probe Life), and human CEACAM8 recombinant protein, as the mobile phase, was 1:2 serially diluted with a maximum concentration of 100 nM. CM24 that solely binds to CEACAM1 was used as reference antibody. The association and dissociation between CEACAM8 protein and the antibody on the surface of the sensor were measured as described above in Example 6.

Results and conclusions: As shown in FIG. 2B, humanized antibodies h2C10 and h4D10 did not cross-react with CEACAM8.

### 6.9.9 Example 9: Candidate Anti-CEACAM1 Antibodies Induced CEACAM1 Internalization

Methods: Two CEACAM1 expressing tumor cell lines were used in this assay, including a pancreatic cancer cell line BXPC-3 and a human hepatoma cell line HepG2. Briefly, pHrodo™ iFL red labeled antibodies were incubated with HepG2 and BXPC-3 for 24 h at 37° C. Antibody internalization was detected by measuring fluorescence intensity using flow cytometry.

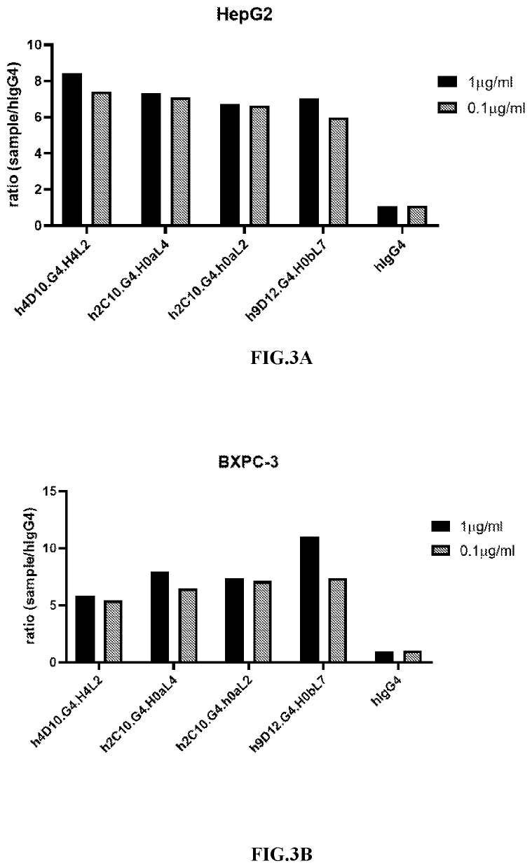

Results and conclusions: As shown in FIGS. 3A-3B, internalization of candidate anti-CEACAM1 antibodies disclosed herein was observed in both HepG2 (FIG. 3A) and BXPC-3 cells (FIG. 3B), indicating that these antibodies could induce internalization of CEACAM1, thus reducing its biological function.

### 6.9.10 Example 10: Candidate Anti-CEACAM1 Antibodies Restored T Cell Activation

Methods: Both Jurkat-NFAT-CEACAM1 bioassay and T cell stimulation assay were used to confirm the anti-CEACAM1 antibodies' effect on T cell activation. In Jurkat-NFAT-CEACAM1 bioassay, T cell leukemia cell line Jurkat was stably transfected with human CEACAM1 and transcription factor NFAT with luciferase gene in tandem. Serially diluted CEACAM1 antibodies were incubated overnight with Jurkat stable cells in the present of anti-CD3 and anti-CD28. Luminescence was measured after substrate was added and incubated for several minutes.

In T cell stimulation assay, PBMC derived T-cells were used. Plates were coated with 1 μg/ml OKT3 (anti-CD3 mAb) overnight at 4° C. On the next day, coating solution containing OKT3 was discarded, and PBMCs were added to OKT3-coated plates. After several days of culture, activated PBMC-derived T cells were mixed with stable transfected cells 293T-CEACAM1/OKT3 (293T cells with stable expression of CEACAM1 and OKT3 scfv on cell membrane to activate T cells) at a ratio of 5:1 (T: 293T-OKT3) and serially diluted antibodies were added. After overnight incubation, IFN-γ secretion was detected by ELISA.

Results and conclusions: As shown in FIGS. 4A-4B, candidate anti-CEACAM1 antibodies enhanced luciferase expression in Jurkat reporter cells (FIG. 4A) and increased IFN-γ release (FIG. 4B), indicating that those antibodies could stimulate T cell activation.

### 6.9.11 Example 11: Candidate Anti-CEACAM1 Antibodies Enhanced the Killing Efficacy of Both NK Cells and T Cells

Methods: Three effector cells were used in this assay, including activated NK cells, a natural killer cell line NK-92 and activated T cells. In the NK cells killing assay, NK cells were isolated from PBMCs of healthy donor with NK Cell Isolation Kit from Miltenyi Biotec according to manufacturer's instruction. To prepare activated NK cells, isolated NK cells were incubated with 1000U/mL IL-2 at 37° C. for several days. The CEACAM1 expression on activated NK cells was confirmed by cell cytometry. Target cells HepG2 with high expression of CEACAM1 were pre-incubated with antibodies at room temperature. Then, NK-92 or activated NK cells were added to the mixture and incubated overnight at 37° C. LDH expression was detected by measuring the absorbance at 490 nm after adding substrate.

In the T cells killing assay, to prepare activated T cells, isolated CD3+ T cells were stimulated by immobilized anti-CD3 and anti-CD28 for several days at 37° C. 293T cells which were stably transfected with human CEACAM1 were used as target cells and labeled with violet dye. Activated T cells were co-cultured with violet-labeled 293T-CEACAM1 cells in the presence of 10 μg/ml candidate antibodies. After overnight incubation, cells were stained with PI propidium iodide, and the percent of dead 293T-CEACAM1 was detected by flow cytometry.

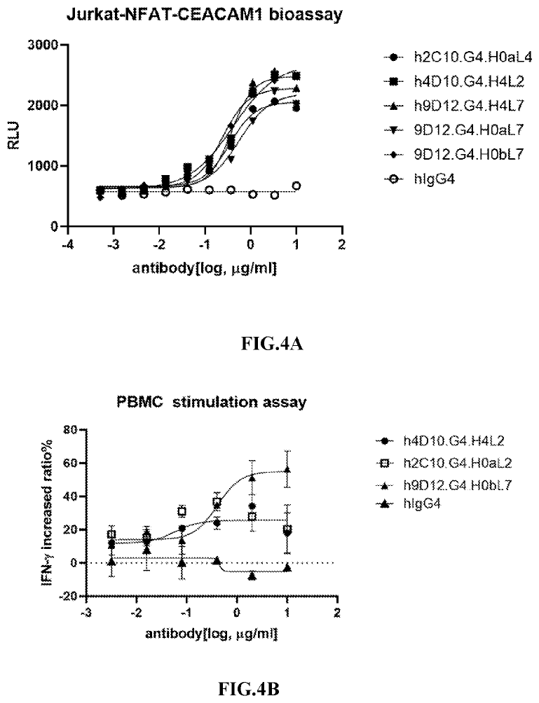

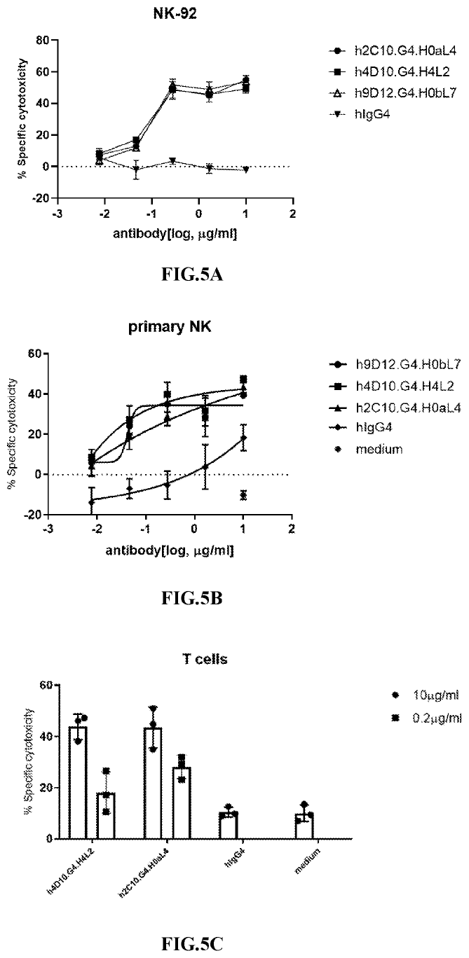

Results and conclusions: As shown in FIGS. 5A-5C, candidate anti-CEACAM1 antibodies enhanced NK-92 cells killing efficacy in a dose-dependent manner (FIG. 5A), which was also observed in primary NK cells (FIG. 5B). The candidate anti-CEACAM1 antibodies also enhanced T cell killing efficacy in a dose-dependent manner (FIG. 5C).

### 6.9.12 Example 12: Candidate Anti-CEACAM1 Antibodies Inhibited Macrophage-Mediated Tumor Aggression

Methods: In this assay, the aggression of tumor cells was monitored through Wound Healing/Cell-Migration Assay and Polyploidy of tumor cells detection. The effect of candidate anti-CEACAM1 antibodies on tumor cells migration was examined by an electrical cell-substrate impedance sensing system (ECIS). The BXPC-3 cells were plated (1×10⁵/well/400 μL) on 10 mM-cysteine-balanced, 8-well, gold-plated electrode arrays (Applied Biophysics, USA). After overnight incubation, the cell confluence reached more than 90%. An elevated electric shock was applied to the wells, wounding the cells on the small electrode and causing the impedance to drop to that of an open electrode. The medium in wells was discarded, and CSFE-labeled monocyte-derived macrophage and the candidate antibodies at 10 μg/ml final work concentration were added to plate. The time required for the healthy cells outside of the small electrode to migrate inward to complete repopulation of the wounded area and replacement of their dead cohorts was measured using ECIS (Applied Biophysics, USA), which was hereinafter referred to as “time to plateau.” After overnight incubation, BXPC-3 cells were collected and fixed in 70% ice cold alcohol. Cells were stained with propidium iodide in the present of RNase A inhibitor. The cell cycle was detected by flow cytometry.

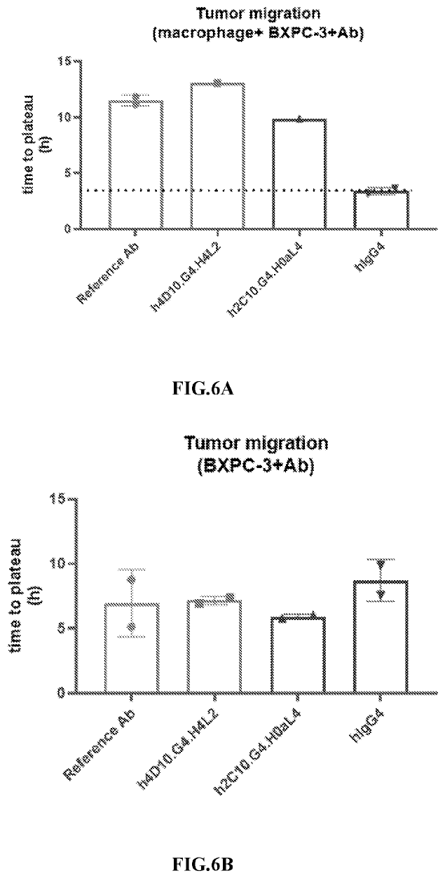

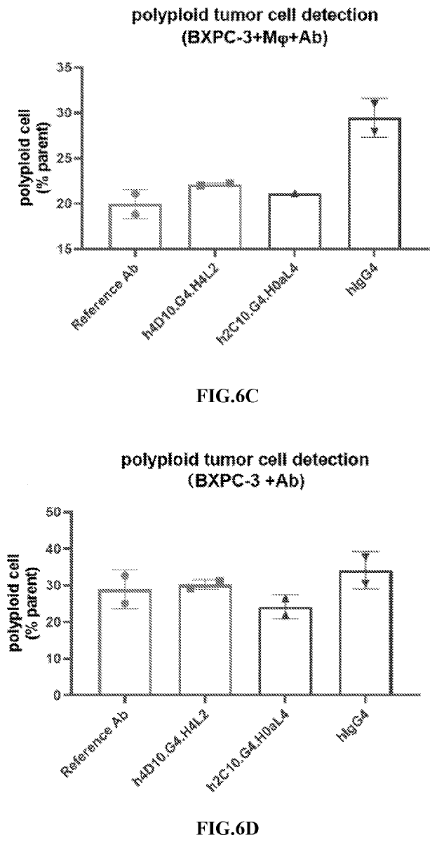

Results and conclusions: As shown in FIGS. 6A-6D, the candidate anti-CEACAM1 antibodies, but not the control IgG, significantly prolonged the time to plateau (FIG. 6A) and reduced the number of polyploid tumor cells (FIG. 6C) when tumor cells were influenced by macrophages. Neither effect was observed when tumor cells were not influenced by macrophages (FIGS. 6B and 6D). These results demonstrate that the candidate anti-CEACAM1 antibodies effectively inhibited macrophage-mediated tumor aggression.

### 6.9.13 Example 13: Candidate Anti-CEACAM1 Antibodies Inhibited Tumor Growth in Mouse Models

Methods: iHu-PBMC-NK immune system humanized mice were implanted with human pancreatic cancer cells or human melanoma cells subcutaneously. Treatment with candidate anti-CEACAM1 antibodies was initiated after tumors had reached a size of approx. 90-100 mm³ in volume. The candidate antibodies were administered twice a week for 2-3 weeks. Tumor diameters were measured twice a week. Tumor volumes were calculated according to the formula lss/2 with “l” being the longest diameter and “s” the short diameter thereto. Inhibition of tumor volume in the test groups relative to the vehicle control group was calculated as the ratio of the median relative tumor volumes (T/C) or TGI %=(1−T/C)×100%.

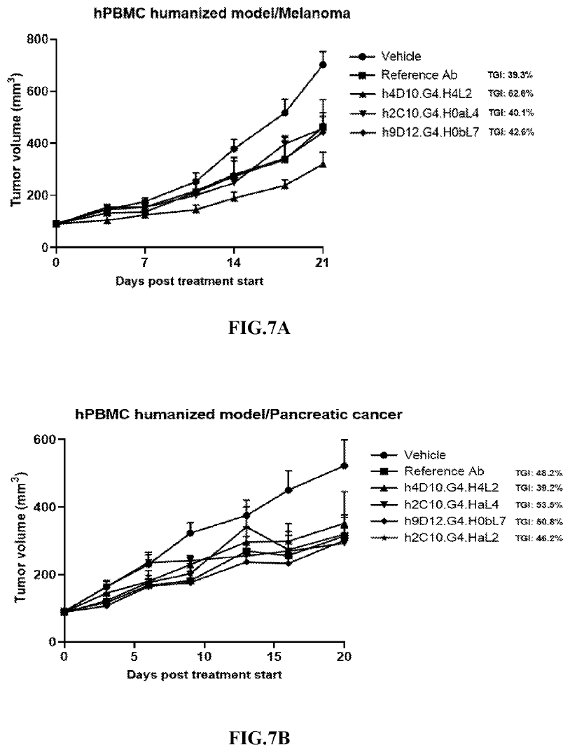

Results and conclusions: As shown in FIGS. 7A-7B, the administration of candidate anti-CEACAM1 antibodies significantly reduced tumor volumes in both melanoma model (FIG. 7A) and the pancreatic cancer model (FIG. 7B).

### 6.9.14 Example 14: Candidate Anti-CEACAM1 Antibodies Blocked CEACAM1 Interaction with CEACAM5 and CEACAM6

Methods: Interaction assay was conducted to detect the blocking of the candidate antibodies on the CEACAM1&CEACAM6 interaction and the CEACAM1&CEACAM5 interaction. Specifically, serially diluted antibodies were added to CEACAM-1 coated 96-well plate and incubated for 1h. The plate was washed twice; and 15 μg/ml biotinylated CEACAM6 was added to plate and incubated for 1h. The plate was washed again, and a secondary antibody (1:2000 strep HRP) was added and incubated for 1 h at room temperature. Detection was performed using substrate TMB and stopping solution 1 mol/L HCL. Plates were measured at 450/540 nm with a plate reader.

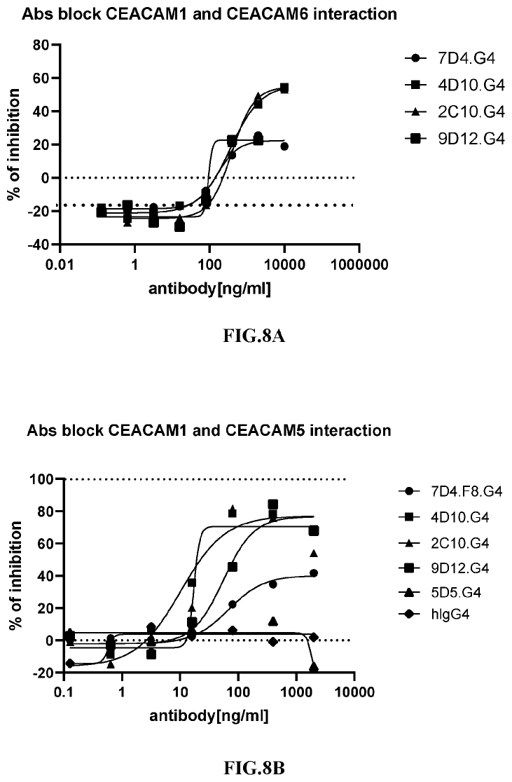

As shown in FIG. 8A, chimeric IgG4 antibodies 7D4, 4D10, 2C10 and 9D12 blocked CEACAM1 and CEACAM6 interaction. As shown in FIG. 8B, chimeric IgG4 antibodies 7D4, 4D10, 2C10 almost completely blocked CEACAM1 and CEACAM5 binding, whereas 7D4 blocked the interaction to a lesser degree.

### 6.9.15 Example 15: Candidate Anti-CEACAM1 Antibodies Increased NK Killing Efficiency in CEACAM1-CEACAM5+ Tumor Cells

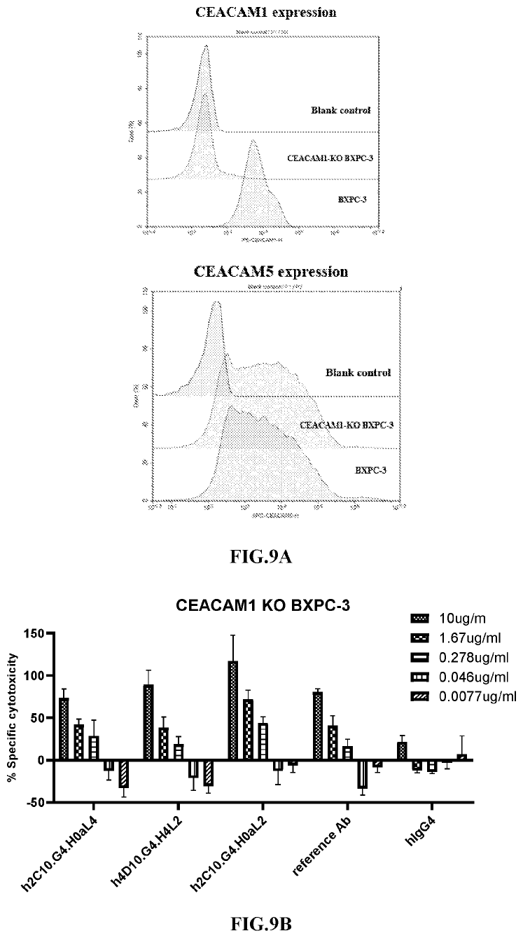

Methods: The killing efficacy of the candidate anti-CEAMCAM1 antibodies toward tumor cells expressing CEACAM5 but not CEACAM1 was measured in this assay. Specifically, CEACAM1-KO BXPC-3 cells were used as target cells, in which endogenous CEACAM-1 was knocked-out using CRISPR-Cas9 technology. The expression of CEACAM5 and lack of expression of CEACAM1 in CEACAM1-KO BXPC-3 cells were confirmed by flow cytometry (FIG. 9A), with parent BXPC-3 cells serving as positive control. In the killing assay, CEACAM1-KO BXPC-3 cells were co-cultured with NK-92 cells in the present of serially diluted antibodies overnight at 37° C. LDH in supernatant was detected by LDH detection kit. The % specific cytotoxicity was calculated according to format: % Specific cytotoxicity=(Experimental value−T+E)×100/(TMax−T+E).

Results: As shown in FIG. 9B, candidate humanized anti-CEACAM1 antibodies 2C10 and 4D10 promoted tumor killing by NK cells in dose-dependent manners.

### 6.9.16 Example 16: 2C10 Showed Better Binding to CEACAM1+/CEACAM5+ Tumor than Reference Anti-CEACAM1

Methods: The PBMC humanized huIL-15 NOD mice were subcutaneously implanted with BxPC-3 cells. After 33 days of growth, the tumors reached a volume of approximately 400-500 mm³, and were explanted from animals and fixed using PFA. In order to assess the binding ability of the candidate anti-CEACAM1 antibody, IHC staining was performed on sections from BxPC-3/PBMC tumor tissues. CM24 was used as reference antibody.

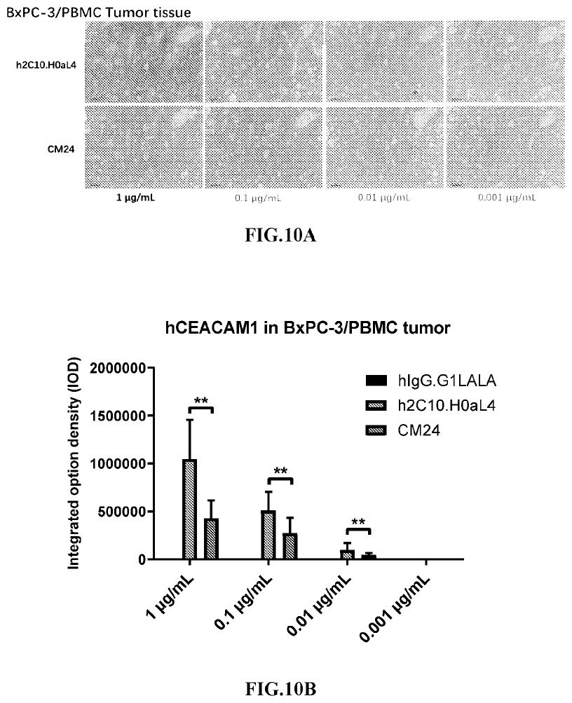

Results: As shown in FIGS. 10A-10B, as antibody concentration decreased (1, 0.1, 0.01, and 0.001 μg/mL), so did the staining intensity of tumor tissue by 2C10 and CM24. In the same sample sections, 2C10 exhibited stronger immunostaining intensity than CM24, likely because it targeted both CEACAM1+ and CEACAM5+ tumors compared to CM24, which solely targeted CEACAM1. In view of the broad expression of CEACAM5 on tumor cells, the target cancer types of 2C10 could be wider compared to an antibody that targets only CEACAM1.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

What is claimed is:

1. An antibody or antigen-binding fragment thereof that specifically binds to human CEACAM1, comprising:
(a) a light chain variable region (VL) comprising
(1) a light chain CDR1 (VL CDR1) having an amino acid sequence selected from the group consisting of SEQ ID NOs:1-7 and 52-53;
(2) a light chain CDR2 (VL CDR2) having an amino acid sequence selected from the group consisting of SEQ ID NOs:8-12 and 54-56; and
(3) a light chain CDR3 (VL CDR3) having an amino acid sequence selected from the group consisting of SEQ ID NOs:13-19; or a variant thereof having up to about 3 amino acid substitutions, additions, and/or deletions in the VL CDRs; and/or
(b) a heavy chain variable region (VH) comprising
(1) a heavy chain CDR1 (VH CDR1) having an amino acid sequence selected from the group consisting of SEQ ID NOs:20-27 and 57;
(2) a heavy chain CDR2 (VH CDR2) having an amino acid sequence selected from the group consisting of SEQ ID NOs:28-39; and
(3) a heavy chain CDR3 (VH CDR3) having an amino acid sequence selected from the group consisting of SEQ ID NOs:40-51; or a variant thereof having up to about 3 amino acid substitutions, additions, and/or deletions in the VH CDRs.

2. The antibody or antigen-binding fragment of claim 1, wherein
(a) the VL CDR1, CDR2 and CDR3 have
(1) the amino acid sequences of SEQ ID NOs:1, 8, and 13, respectively;
(2) the amino acid sequences of SEQ ID NOs:2, 9, and 14, respectively;
(3) the amino acid sequences of SEQ ID NOs:3, 9, and 14, respectively;
(4) the amino acid sequences of SEQ ID NOs:4, 8, and 15, respectively;
(5) the amino acid sequences of SEQ ID NOs:5, 10, and 16, respectively;
(6) the amino acid sequences of SEQ ID NOs:3, 9, and 17, respectively;
(7) the amino acid sequences of SEQ ID NOs:6, 11, and 18, respectively; or
(8) the amino acid sequences of SEQ ID NOs:7, 12, and 19, respectively; or a variant thereof having up to about 3 amino acid substitutions, additions, and/or deletions in the VL CDRs; and/or
(b) the VH CDR1, CDR2 and CDR3 have
(1) the amino acid sequences of SEQ ID NOs:20, 28, and 40, respectively;
(2) the amino acid sequences of SEQ ID NOs:21, 29, and 41, respectively;
(3) the amino acid sequences of SEQ ID NOs:21, 30, and 42, respectively;
(4) the amino acid sequences of SEQ ID NOs:22, 31, and 43, respectively;
(5) the amino acid sequences of SEQ ID NOs:23, 32, and 44, respectively;
(6) the amino acid sequences of SEQ ID NOs:24, 33, and 45, respectively;
(7) the amino acid sequences of SEQ ID NOs:25, 38, and 49, respectively;
(8) the amino acid sequences of SEQ ID NOs:26, 39, and 50, respectively; or
(9) the amino acid sequences of SEQ ID NOs:27, 38, and 51, respectively; or a variant thereof having up to about 3 amino acid substitutions, additions, and/or deletions in the VH CDRs.

3. The antibody or antigen-binding fragment of claim 1, wherein
(1) the VL CDR1, CDR2, and CDR3 have the amino acid sequences of SEQ ID NOs:1, 8, and 13, respectively; and/or the VH CDR1, CDR2, and CDR3 have the amino acid sequences of SEQ ID NOs:20, 28, and 40, respectively;
(2) the VL CDR1, CDR2, and CDR3 have the amino acid sequences of SEQ ID NOs:2, 9, and 14, respectively; and/or the VH CDR1, CDR2, and CDR3 have the amino acid sequences of SEQ ID NOs:21, 29, and 41, respectively;
(3) the VL CDR1, CDR2, and CDR3 have the amino acid sequences of SEQ ID NOs:3, 9, and 14, respectively; and/or the VH CDR1, CDR2, and CDR3 have the amino acid sequences of SEQ ID NOs:21, 30, and 42, respectively;
(4) the VL CDR1, CDR2, and CDR3 have the amino acid sequences of SEQ ID NOs:4, 8, and 15, respectively; and/or the VH CDR1, CDR2, and CDR3 have the amino acid sequences of SEQ ID NOs:22, 31, and 43, respectively;
(5) the VL CDR1, CDR2, and CDR3 have the amino acid sequences of SEQ ID NOs:5, 10, and 16, respectively; and/or the VH CDR1, CDR2, and CDR3 have the amino acid sequences of SEQ ID NOs:23, 32, and 44, respectively;
(6) the VL CDR1, CDR2, and CDR3 have the amino acid sequences of SEQ ID NOs:3, 9, and 17, respectively; and/or the VH CDR1, CDR2, and CDR3 have the amino acid sequences of SEQ ID NOs:24, 33, and 45, respectively;
(7) the VL CDR1, CDR2, and CDR3 have the amino acid sequences of SEQ ID NOs:6, 11, and 18, respectively; and/or the VH CDR1, CDR2, and CDR3 have the amino acid sequences of SEQ ID NOs:25, 38, and 49, respectively;
(8) the VL CDR1, CDR2, and CDR3 have the amino acid sequences of SEQ ID NOs:7, 12, and 19, respectively; and/or the VH CDR1, CDR2, and CDR3 have the amino acid sequences of SEQ ID NOs:26, 39, and 50, respectively; or
(9) the VL CDR1, CDR2, and CDR3 have the amino acid sequences of SEQ ID NOs:7, 12, and 19, respectively; and/or the VH CDR1, CDR2, and CDR3 have the amino acid sequences of SEQ ID NOs:27, 38, and 51, respectively.

4. The antibody or antigen-binding fragment of claim 1, comprising a VL CDR1, a VL CDR2, a VL CDR3, a VH CDR1, a VH CDR2 and a VH CDR3 having the amino acid sequences of SEQ ID NOs:3, 9, 14, 21, 30, and 42, respectively; or a variant thereof having up to about 3 amino acid substitutions, additions, and/or deletions in the CDRs.

5. The antibody or antigen-binding fragment of claim 1, comprising a VL CDR1, a VL CDR2, a VL CDR3, a VH CDR1, a VH CDR2 and a VH CDR3 having the amino acid sequences of SEQ ID NOs:5, 10, 16, 23, 32, and 44, respectively; or a variant thereof having up to about 3 amino acid substitutions, additions, and/or deletions in the CDRs.

6. The antibody or antigen-binding fragment of claim 1, comprising a VL CDR1, a VL CDR2, a VL CDR3, a VH CDR1, a VH CDR2 and a VH CDR3 having the amino acid sequences of SEQ ID NOs:3, 9, 17, 24, 33, and 45, respectively; or a variant thereof having up to about 3 amino acid substitutions, additions, and/or deletions in the CDRs.

7. The antibody or antigen-binding fragment of claim 6, wherein the VL CDR1 has an amino acid sequence selected from the group consisting of SEQ ID NOs:52-53, wherein the VL CDR2 has an amino acid sequence selected from the group consisting of SEQ ID NOs:54-56, wherein the VH CDR1 has the amino acid sequence of SEQ ID NO:57, wherein the VH CDR2 has an amino acid sequence selected from the group consisting of SEQ ID NOs:34-37 and wherein the VH CDR3 has an amino acid sequence selected from the group consisting of SEQ ID NOs:46-48.

8. An antibody or antigen-binding fragment thereof that specifically binds to human CEACAM1, comprising:
(a) a VL having at least 85%, at least 90%, at least 95%, at least 98%, or 100% sequence identity to an amino acid sequence selected from the group consisting of SEQ ID NOs:61-68; and/or
(b) a VH having at least 85%, at least 90%, at least 95%, at least 98%, or 100% sequence identity to an amino acid sequence selected from the group consisting of SEQ ID NOs:70-78.

9. The antibody or antigen-binding fragment of claim 8, comprising a VL and a VH, wherein the VL and VH each have at least 85%, at least 90%, at least 95%, at least 98%, or 100% sequence identity to the amino acid sequences of (1) SEQ ID NOs:61 and 70, respectively; (2) SEQ ID NOs:62 and 71, respectively; (3) SEQ ID NOs:63 and 72, respectively; (4) SEQ ID NOs:64 and 73, respectively; (5) SEQ ID NOs:65 and 74, respectively; (6) SEQ ID NOs:66 and 75, respectively; (7) SEQ ID NOs:67 and 76, respectively; (8) SEQ ID NOs:68 and 77, respectively; or (9) SEQ ID NOs:68 and 78, respectively.

10. The antibody or antigen-binding fragment of claim 9, comprising a VL and a VH, wherein the VL and VH each have at least 85%, at least 90%, at least 95%, at least 98%, or 100% sequence identity to the amino acid sequences of SEQ ID NOs:63 and 72, respectively.

11. The antibody or antigen-binding fragment of claim 9, comprising a VL and a VH, wherein the VL and VH each have at least 85%, at least 90%, at least 95%, at least 98%, or 100% sequence identity to the amino acid sequences of SEQ ID NOs:65 and 74, respectively.

12. The antibody or antigen-binding fragment of claim 9, comprising a VL and a VH, wherein the VL and VH each have at least 85%, at least 90%, at least 95%, at least 98%, or 100% sequence identity to the amino acid sequences of SEQ ID NOs:66 and 75, respectively.

13. The antibody or antigen-binding fragment of claim 12, wherein the VL has the amino acid sequence of SEQ ID NO:66 with an amino acid substitution at S28, S51, K52, or any combination thereof.

14. The antibody or antigen-binding fragment of claim 13, wherein the S28 substitution is S28F or S28H, wherein the S51 substitution is S51M, and wherein the K52 substitution is K52R.

15. The antibody or antigen-binding fragment of claim 12, wherein the VH has the amino acid sequence of SEQ ID NO:75 with an amino acid substitution at H35, N56, Y100, or any combination thereof.

16. The antibody or antigen-binding fragment of claim 15, wherein the H35 substitution is H35M, wherein the N56 substitution is N56I, N56Q, N56V, or N56W, and wherein the Y100 substitution is Y100F, Y100W, or Y100V.

17. An antibody or antigen-binding fragment thereof that specifically binds to human CEACAM1, comprising:
(a) a VL comprising VL CDR1, CDR2, and CDR3 from a VL having an amino acid sequence selected from the group consisting of SEQ ID NOs:61-68; and/or
(b) a VH comprising VH CDR1, CDR2, and CDR3 from a VH having an amino acid sequence selected from the group consisting of SEQ ID NOs:70-78; or a variant thereof having up to about 3 amino acid substitutions, additions, and/or deletions in the CDRs.

18. The antibody or antigen-binding fragment thereof claim 17 comprising
(1) a VL comprising VL CDR1, CDR2, and CDR3 from a VL having the amino acid sequence of SEQ ID NO:61, and/or a VH comprising VH CDR1, CDR2, and CDR3 from a VH having the amino acid sequence of SEQ ID NO:70;
(2) a VL comprising VL CDR1, CDR2, and CDR3 from a VL having the amino acid sequence of SEQ ID NO:62, and/or a VH comprising VH CDR1, CDR2, and CDR3 from a VH having the amino acid sequence of SEQ ID NO:71;
(3) a VL comprising VL CDR1, CDR2, and CDR3 from a VL having the amino acid sequence of SEQ ID NO:63, and/or a VH comprising VH CDR1, CDR2, and CDR3 from a VH having the amino acid sequence of SEQ ID NO:72;
(4) a VL comprising VL CDR1, CDR2, and CDR3 from a VL having the amino acid sequence of SEQ ID NO:64, and/or a VH comprising VH CDR1, CDR2, and CDR3 from a VH having the amino acid sequence of SEQ ID NO:73;
(5) a VL comprising VL CDR1, CDR2, and CDR3 from a VL having the amino acid sequence of SEQ ID NO:65, and/or a VH comprising VH CDR1, CDR2, and CDR3 from a VH having the amino acid sequence of SEQ ID NO:74;
(6) a VL comprising VL CDR1, CDR2, and CDR3 from a VL having the amino acid sequence of SEQ ID NO:66, and/or a VH comprising VH CDR1, CDR2, and CDR3 from a VH having the amino acid sequence of SEQ ID NO:75;
(7) a VL comprising VL CDR1, CDR2, and CDR3 from a VL having the amino acid sequence of SEQ ID NO:67, and/or a VH comprising VH CDR1, CDR2, and CDR3 from a VH having the amino acid sequence of SEQ ID NO:76;
(8) a VL comprising VL CDR1, CDR2, and CDR3 from a VL having the amino acid sequence of SEQ ID NO:68, and/or a VH comprising VH CDR1, CDR2, and CDR3 from a VH having the amino acid sequence of SEQ ID NO:77; or
(9) a VL comprising VL CDR1, CDR2, and CDR3 from a VL having the amino acid sequence of SEQ ID NO:68, and/or a VH comprising VH CDR1, CDR2, and CDR3 from a VH having the amino acid sequence of SEQ ID NO:78; or a variant thereof having up to about 3 amino acid substitutions, additions, and/or deletions in the CDRs.

19. An antibody or antigen-binding fragment thereof that specifically binds to human CEACAM1 comprising a VL and a VH, wherein the VL comprises VL CDR1, CDR2, and CDR3 from a VL having the amino acid sequence of SEQ ID NO:63, and the VH comprises VH CDR1, CDR2, and CDR3 from a VH having the amino acid sequence of SEQ ID NO:72; or a variant thereof having up to about 3 amino acid substitutions, additions, and/or deletions in the CDRs.

20. An antibody or antigen-binding fragment thereof that specifically binds to human CEACAM1 comprising a VL and a VH, wherein the VL comprises VL CDR1, CDR2, and CDR3 from a VL having the amino acid sequence of SEQ ID NO:65, and the VH comprises VH CDR1, CDR2, and CDR3 from a VH having the amino acid sequence of SEQ ID NO:74; or a variant thereof having up to about 3 amino acid substitutions, additions, and/or deletions in the CDRs.

21. An antibody or antigen-binding fragment thereof that specifically binds to human CEACAM1 comprising a VL and a VH, wherein the VL comprises VL CDR1, CDR2, and CDR3 from a VL having the amino acid sequence of SEQ ID NO:66, and the VH comprises VH CDR1, CDR2, and CDR3 from a VH having the amino acid sequence of SEQ ID NO:75; or a variant thereof having up to about 3 amino acid substitutions, additions, and/or deletions in the CDRs.

22. An antibody or antigen-binding fragment thereof that competes with the antibody or antigen-binding fragment of any one of claims 1 to 21 for binding to human CEACAM1.

23. The antibody or antigen-binding fragment of any one of claims 1 to 22 that is a chimeric antibody or antigen-binding fragment, a humanized antibody or antigen-binding fragment, or a human antibody or antigen-binding fragment.

24. The antibody or antigen-binding fragment of claim 23 that is a humanized antibody or antigen-binding fragment.

25. The antibody or antigen-binding fragment of claim 24 that has
(a) a VL having at least 85%, at least 90%, at least 95%, at least 98%, or 100% sequence identity to an amino acid sequence selected from the group consisting of SEQ ID NOs:81-90; and/or
(b) a VH having at least 85%, at least 90%, at least 95%, at least 98%, or 100% sequence identity to an amino acid sequence selected from the group consisting of SEQ ID NOs:111-116.

26. The antibody or antigen-binding fragment of claim 24 that has
(a) a VL having at least 85%, at least 90%, at least 95%, at least 98%, or 100% sequence identity to an amino acid sequence selected from the group consisting of SEQ ID NOs:91-93 and 107-109; and/or
(b) a VH having at least 85%, at least 90%, at least 95%, at least 98%, or 100% sequence identity to an amino acid sequence selected from the group consisting of SEQ ID NOs:117-122.

27. The antibody or antigen-binding fragment of claim 24 that has
(a) a VL having at least 85%, at least 90%, at least 95%, at least 98%, or 100% sequence identity to an amino acid sequence selected from the group consisting of SEQ ID NOs:94-106; and/or
(b) a VH having at least 85%, at least 90%, at least 95%, at least 98%, or 100% sequence identity to an amino acid sequence selected from the group consisting of SEQ ID NOs:123-128, 130 and 143-149.

28. The antibody or antigen-binding fragment of claim 27, wherein the VL has an amino acid sequence selected from the group consisting of SEQ ID NOs:94-99, with an amino acid substitution at S28, S51, K52, or any combination thereof.

29. The antibody or antigen-binding fragment of claim 28, wherein the S28 substitution is S28F or S28H, wherein the S51 substitution is S51M, and wherein the K52 substitution is K52R.

30. The antibody or antigen-binding fragment of claim 29, wherein the VL has an amino acid sequence selected from the group consisting of SEQ ID NOs:100-106.

31. The antibody or antigen-binding fragment of claim 27, wherein the VH has an amino acid sequence selected from the group consisting of SEQ ID NOs:123-128, with an amino acid substitution at H35, N56, Y100, or any combination thereof.

32. The antibody or antigen-binding fragment of claim 31, wherein the H35 substitution is H35M, wherein the N56 substitution is N56I, N56Q, N56V, or N56W, and wherein the Y100 substitution is Y100F, Y100W, or Y100V.

33. The antibody or antigen-binding fragment of claim 32, wherein the VH has an amino acid sequence selected from the group consisting of SEQ ID NOs:130 and 143-149.

34. The antibody or antigen-binding fragment of any one of claims 1 to 33, that binds to human CEACAM1 with a K_{D} that is 10⁻⁸ M or less.

35. The antibody or antigen-binding fragment of claims 1 to 33, that binds to human CEACAM1 with a K_{D} that ranges from 10⁻¹¹ M to 10⁻⁸ M.

36. The antibody or antigen-binding fragment of any one of claims 1 to 35 that promotes internalization of CEACAM1 on cell surface.

37. The antibody or antigen-binding fragment of any one of claims 1 to 36 that activates T cells, NK cells, or both.

38. The antibody or antigen-binding fragment of claim 37 that enhances IFN-γ release by T cells.

39. The antibody or antigen-binding fragment of claim 37 that enhances the activity of T cells, NK cells, or both, against tumor cells.

40. The antibody of antigen-binding fragment of any one of claims 1 to 39 that is selected from the group consisting of an IgG1 antibody, an IgG2 antibody, an IgG3 antibody, and an IgG4 antibody.

41. The antibody or antigen-binding fragment of claim 40 that is an IgG1 antibody.

42. The antibody or antigen-binding fragment of any one of claims 1 to 39 that is selected from the group consisting of a Fab, a Fab′, a F(ab′)₂, a Fv, a scFv, a (scFv)₂, a single domain antibody (sdAb), and a heavy chain antibody (HCAb).

43. The antibody or antigen-binding fragment of any one of claims 1 to 42 that is a monoclonal antibody or antigen-binding fragment.

44. A polynucleotide encoding the antibody or antigen-binding fragment of any one of claims 1 to 43.

45. A vector comprising the polynucleotide of claim 44.

46. A cell comprising the polynucleotide of claim 44, or the vector of claim 45.

47. A method of making an antibody or antigen-binding fragment thereof that specifically binds to human CEACAM1, comprising culturing the cell of claim 46 under conditions that allow expression of the antibody or antibody fragment.

48. The method of claim 47 that comprises isolating the antibody or antigen-binding fragment from the culture.

49. A pharmaceutical composition comprising a therapeutically effective amount of the antibody or antigen-binding fragment of any one of claims 1 to 43, and a pharmaceutically acceptable carrier.

50. A method of inducing or stimulating immune cell activation and/or proliferation, comprising contacting an immune cell with an effective amount of the antibody or antigen-binding fragment of any one of claims 1 to 43.

51. A method of reducing the suppression of an immune cell comprising contacting the immune cell with an effective amount of the antibody or antigen-binding fragment of any one of claims 1 to 43.

52. The method of claim 50 or 51, wherein the immune cell is a lymphocyte of T cell or NK cell.

53. A method of increasing IFN-γ production in a T cell comprising contacting the T cell with an effective amount of the antibody or antigen-binding fragment of any one of claims 1 to 43.

54. A method of inhibiting cancer cell growth, angiogenesis and/or metastasis in a subject in need thereof, comprising administering to the subject a therapeutically effective amount of the antibody or antigen-binding fragment of any one of claims 1 to 43.

55. A method of treating cancer in a subject in need thereof, comprising administering to the subject a therapeutically effective amount of the antibody or antigen-binding fragment of any one of claims 1 to 43.

56. The method of claim 54 or 55, further comprising administering an additional therapy to the subject.

57. The method of any one of claims 54 to 56, wherein the subject is a human.

58. Use of the antibody or antigen-binding fragment of any one of claims 1 to 43 in cancer treatment.

59. Use of the antibody or antigen-binding fragment of any one of claims 1 to 43 for the preparation of a medicament for the treatment of cancer.

60. The method or use of any one of claims 54 to 59, wherein the cancer is a CEACAM1-expressing cancer, a CEACAM5-expressing cancer or a CEACAM6-expressing cancer.

61. The method or use of any one of claims 54 to 60, wherein the cancer is a hematological cancer.

62. The method or use of any one of claims 54 to 60, wherein the cancer is a solid tumor.

63. The method or use of claim 62, wherein the solid tumor is pancreatic cancer.

64. The method or use of claim 62, wherein the solid tumor is melanoma.
